SEQUENCE LISTING (1 sequence)

SEQ ID NO: 1 - protein, 824 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

(19) United States
(12) Patent Application Publication
(10) Pub. No.: US 2023/0391781 A1
(43) Pub. Date: Dec. 7, 2023

(54) MALT1 MODULATORS AND USES THEREOF

(71) Applicant: Rheos Medicines, Inc., Cambridge, MA (US)

(72) Inventors: Brian Addison DeChristopher, Belmont, MA (US); Guillaume Barbe, Milton, MA (US)

(73) Assignee: Rheos Medicines, Inc., Foxborough, MA (US)

(21) Appl. No.: 18/031,981

(22) PCT Filed: Oct. 15, 2021
(86) PCT No.: PCT/US2021/055173

§ 371 Date: Apr. 21, 2022

Related U.S. Application Data
(60) Provisional application No. 63/092,768, filed on Oct. 16, 2020.

(51) Int. Cl. C07D 487/04

(52) U.S. Cl. CPC C07D 487/04

(57) ABSTRACT

Provided herein are compounds, compositions, and methods useful for modulating MALT1 and for treating related diseases, disorders, and conditions.

### CROSS-REFERENCE TO RELATED APPLICATIONS

This application claims priority to and the benefit of U.S. Provisional Patent Application No. 63/092,768, filed on Oct. 16, 2020, the contents of which are incorporated herein by reference in their entirety.

### BACKGROUND

Mucosa associated lymphoid tissue lymphoma translocation protein 1 (MALT1) is an intracellular signaling protein, known from innate (e.g., natural killer cells NK, dendritic cells DC, and mast cells) and adaptive immune cells (e.g., T cells and B cells). MALT1 plays an essential role in influencing immune responses. For example, in T cell receptor signaling, MALT1 mediates nuclear factor κB (NFKB) signaling, leading to T cell activation and proliferation. Accordingly, MALT1 is of interest in the mechanism of autoimmune and inflammatory pathologies. In addition, constitutive (dysregulated) MALT activity is associated with cancers such as MALT lymphoma and activated B cell-like diffuse large B Cell lymphoma (ABC-DLBCL). Modulators of MALT1 activity may be useful as potential therapeutics.

### SUMMARY

Provided herein are compounds designed to act as MALT1 modulators. In some embodiments, such compounds are envisioned to be useful as therapeutic agents for treating autoimmune and inflammatory diseases, disorders, or conditions or cancers.

In one aspect, provided herein is a compound represented by Formula (I):

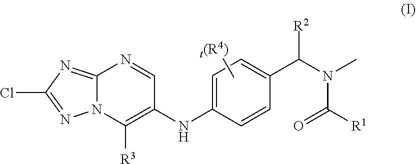

or a pharmaceutically acceptable salt thereof, wherein:
R¹ is selected from the group consisting of C₁₋₆alkyl, C₁₋₆alkoxy, C₃₋₆cycloalkyl, and 5-10 membered heterocyclyl, wherein the C₁₋₆alkyl, C₁₋₆cycloalkyl, and 5-10 membered heterocyclyl may be optionally substituted on one or more available carbons by one, two, three, or more substituents each independently selected from R^{1a}, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH₃ or CF₃;
R³ is hydrogen; or
R³ is selected from the group consisting of C₁₋₆alkyl, C₁₋₆alkoxy, C₃₋₇cycloalkyl, 5-6 membered heterocyclyl, 5-6 membered heterocyclyl-C₁₋₃alkyl-, 5-6 membered heterocyclyl-O—, phenyl, and 5-6 membered heteroaryl, any of which may be optionally substituted with one, two or three substituents each independently selected from R^{3a};
R⁴ is C₁₋₆alkyl;
R^{1a} is independently, for each occurrence, selected from the group consisting of cyano, halogen, hydroxyl, oxo, C₁₋₆alkyl, —C(O)OR^{A}, —C(O)N(R^{A})₂, —N(R^{A})₂, C₁₋₆alkoxy, 5-6 membered heterocyclyl, and 5-6 membered heteroaryl, wherein the C₁₋₆alkyl is optionally substituted with —N(R^{A})₂, and wherein if the 5-6 membered heterocyclyl contains a substitutable ring nitrogen atom, that ring nitrogen atom may optionally be substituted by R^{B};
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl;
R^{3a} is independently, for each occurrence, selected from the group consisting of halogen, C₁₋₄alkyl, C₁₋₄haloalkyl, C₁₋₄alkoxy, C₁₋₄haloalkoxy, hydroxy, C₁₋₄alkenyl, cyano, azido, —NR^{C}R^{D}, C₃₋₆cycloalkyl, C₁₋₄alkoxyC₁₋₄alkoxy, 5-6 membered heterocyclyl-O—, 5-6 membered heterocyclyl, and phenyl, wherein C₃₋₆cycloalkyl, 5-6 membered heterocyclyl-O—, 5-6 membered heterocyclyl, and phenyl are optionally substituted with one, two or three substituents each independently selected from R^{p};
R^{p} is independently, for each occurrence, selected from the group consisting of halogen, C₁₋₄alkyl, C₁₋₄haloalkyl, hydroxy, C₁₋₄alkoxy, C₁₋₄alkoxyC₁₋₄alkyl, NR^{C}R^{D}, and aminoC₁₋₃alkyl;
R^{A} is independently, for each occurrence, selected from the group consisting of hydrogen, C₁₋₆alkyl, —C(O)C₁₋₆alkyl, and —C(O)OC₁₋₆alkyl;

R^{B} is selected from the group consisting of C₁₋₆alkyl, C₃₋₆cycloalkyl, and —C(O)OC₁₋₆alkyl;
R^{C} and R^{D} are independently, for each occurrence, selected from the group consisting of hydrogen, C₁₋₆alkyl, haloC₁₋₆alkyl, and C₃₋₄cycloalkyl, or
R^{C} and R^{D} together with the nitrogen atom to which they are attached form 4-6 membered heterocyclyl or 4-6 membered heteroaryl, wherein the 4-6 membered heterocyclyl or 4-6 membered heteroaryl may contain a further nitrogen atom or an oxygen atom and is optionally substituted with one or two fluoro; and
t is 0 or 1.

In another aspect, provided herein are compounds represented by Formula (Ib):

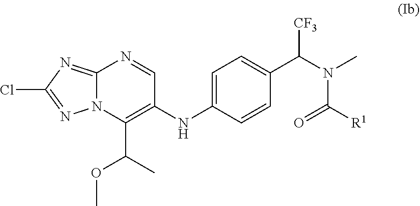

or a pharmaceutically acceptable salt thereof, wherein:
R¹ is C₁₋₆alkyl, C₃₋₆cycloalkyl, or 5-10 membered heterocyclyl, wherein the C₃₋₆cycloalkyl may be optionally substituted on one or more available carbons by one, two, three, or more substituents each independently selected from R^{1a}, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R^{1a} is independently, for each occurrence, selected from the group consisting of cyano, halogen, hydroxyl, C₁₋₆alkyl, —C(O)OR^{A}, —C(O)N(R^{A})₂, —N(R^{A})₂, C₁₋₆alkoxy, and 5-6 membered heteroaryl, wherein the C₁₋₆alkyl is optionally substituted with —N(R^{A})₂;
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl; and

R^{A} is independently, for each occurrence, selected from the group consisting of hydrogen, C₁₋₆alkyl, —C(O)C₁₋₆alkyl, and —C(O)OC₁₋₆alkyl.

In some embodiments, a compound provided herein is selected from a compound set forth in Table 1, or a pharmaceutically acceptable salt thereof.

In another aspect, provided herein is a pharmaceutical composition comprising a compound disclosed herein and a pharmaceutically acceptable carrier.

In another aspect, provided herein is a method of treating a cancer in a subject in need thereof, the method comprising administering to the subject a therapeutically effective amount of a compound or a pharmaceutical composition disclosed herein.

In another aspect, provided herein is a method of treating an autoimmune or inflammatory disorder or disease in a subject in need thereof, the method comprising administering to the subject a therapeutically effective amount of a compound or a pharmaceutical composition disclosed herein.

### DETAILED DESCRIPTION

As generally described herein, the present invention provides compounds designed, for example, to act as MALT modulators. In certain embodiments, such compounds are envisioned to be useful as therapeutic agents for treating autoimmune and inflammatory diseases, disorders, or conditions or cancers.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S compound in such compositions can, for example, comprise, at least about 95% by weight 5-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

The following terms are intended to have the meanings presented therewith below and are useful in understanding the description and intended scope of the present invention. When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example. “C₁₋₆alkyl” is intended to encompass, C₁, C₂, C₃, C₄, C₅, C₆. C₁₋₆, C₁₋₅. C₁₋₄, C₁₋₃, C₁₋₂, C₂₋₆, C₂₋₅. C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein. “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butenyl). Examples of C₂₋₄ alkynyl groups include, without limitation, ethenyl (C₂), 1-propenyl (C₃), 2-propynyl (C₃), 1-butenyl (C₄), 2-butenyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” “alkynylene,” “cycloalkylene,” “heterocyclylene,” “heteroarylene,” and “phenylene” refer to a divalent radical of an alkyl, alkenyl, alkynyl, cycloalkyl, heterocyclyl (e.g., saturated and partially saturated), heteroaryl, and phenyl group respectively.

When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indene, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

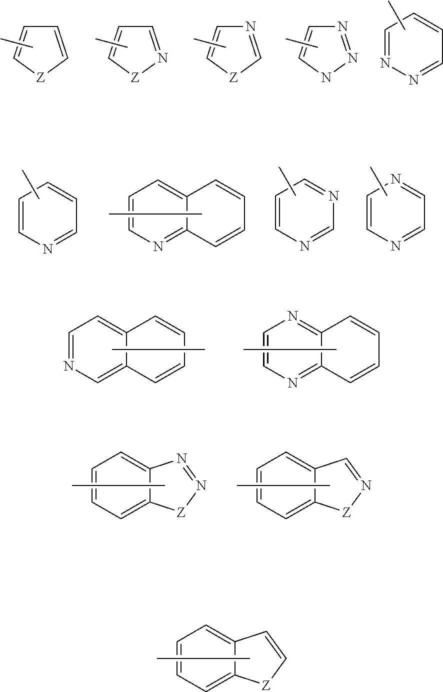

wherein each Z is selected from carbonyl, N, NR⁶⁵, O, and S; and R⁶⁵ is independently hydrogen, C₁₋₈ alkyl, C₃₋₁₀ carbocyclyl, 4-10 membered heterocyclyl, C₆-C₁₀ aryl, and 5-10 membered heteroaryl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes.

As used herein, “C₃₋₆ monocyclic cycloalkyl” or “monocyclic C₃₋₆ cycloalkyl” refers to a 3- to 7-membered monocyclic hydrocarbon ring system that is saturated. 3- to 7-membered monocyclic cycloalkyl groups include, without limitation, cyclopropyl, cyclobutyl, cyclopentyl, and cyclohexyl. Where specified as being optionally substituted or substituted, substituents on a cycloalkyl (e.g., in the case of an optionally substituted cycloalkyl) may be present on any substitutable position and, include, e.g., the position at which the cycloalkyl group is attached.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group.” “heterocyclic moiety,” and “heterocyclic radical,” may be used interchangeably.

In some embodiments, a heterocyclyl group is a 4-7 membered non-aromatic ring system having ring carbon atoms and 1-4 ring heteroatoms, wherein each heteroatom is independently selected from nitrogen, oxygen, and sulfur (“4-7 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Examples of saturated or partially unsaturated heterocyclic radicals include, without limitation, tetrahydrofuranyl, tetrahydrothienyl, tetrahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. Where specified as being optionally substituted or substituted, substituents on a heterocyclyl (e.g., in the case of an optionally substituted heterocyclyl) may be present on any substitutable position and, include, e.g., the position at which the heterocyclyl group is attached.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl; carbocyclyl, e.g., heterocyclyl; aryl, e.g., heteroaryl; and the like having from 1 to 5, and particularly from 1 to 3 heteroatoms.

As used herein, “cyano” refers to —CN.

The terms “halo” and “halogen” as used herein refer to an atom selected from fluorine (fluoro, —F), chlorine (chloro, —Cl), bromine (bromo, —Br), and iodine (iodo, —I). In certain embodiments, the halo group is either fluoro or chloro.

The term “alkoxy,” as used herein, refers to an alkyl group which is attached to another moiety via an oxygen atom (—O(alkyl)). Non-limiting examples include e.g., methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “haloalkyl” includes mono, poly, and perhaloalkyl groups substituted with one or more halogen atoms where the halogens are independently selected from fluorine, chlorine, bromine, and iodine. For the group C₁₋₄haloalkyl-O—C₁₋₄alkyl, the point of attachment occurs on the alkyl moiety which is halogenated.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkenyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, malefic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

### Compounds

In one aspect, provided herein are compounds represented by Formula (I):

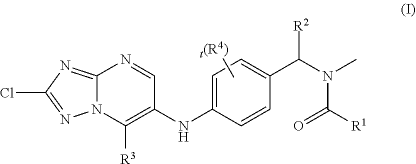

or a pharmaceutically acceptable salt thereof, wherein:
R¹ is selected from the group consisting of C₁₋₆alkyl, C₁₋₆alkoxy, C₃₋₆cycloalkyl, and 5-10 membered heterocyclyl, wherein the C₁₋₆alkyl, C₃₋₆cycloalkyl, and 5-10 membered heterocyclyl may be optionally substituted on one or more available carbons by one, two, three, or more substituents each independently selected from R^{1a}, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH₃ or CF₃;
R³ is hydrogen; or
R³ is selected from the group consisting of C₁₋₆alkyl, C₁₋₆alkoxy, C₃₋₇cycloalkyl, 5-6 membered heterocyclyl, 5-6 membered heterocyclyl-C₁₋₃alkyl-, 5-6 membered heterocyclyl-O—, phenyl, and 5-6 membered heteroaryl, any of which may be optionally substituted with one, two or three substituents each independently selected from R^{3a};
R⁴ is C₁₋₆alkyl; R^{1a} is independently, for each occurrence, selected from the group consisting of cyano, halogen, hydroxyl, oxo, C₁₋₆alkyl, —C(O)OR^{A}, —C(O)N(R^{A})₂, —N(R^{A})₂, C₁₋₆alkoxy, 5-6 membered heterocyclyl, and 5-6 membered heteroaryl, wherein the C₁₋₆alkyl is optionally substituted with —N(R^{A})₂, and wherein if the 5-6 membered heterocyclyl contains a substitutable ring nitrogen atom, that ring nitrogen atom may optionally be substituted by R^{B};
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl; R^{3a} is independently, for each occurrence, selected from the group consisting of halogen, C₁₋₄alkyl, C₁₋₄haloalkyl, C₁₋₄alkoxy, C₁₋₄haloalkoxy, hydroxy, C₁₋₄alkenyl, cyano, azido, —NR^{C}R^{D}, C₃₋₆cycloalkyl, C₁₋₄alkoxyC₁₋₄alkoxy, 5-6 membered heterocyclyl-O—, 5-6 membered heterocyclyl, and phenyl, wherein C₃₋₆cycloalkyl, 5-6 membered heterocyclyl-O—, 5-6 membered heterocyclyl, and phenyl are optionally substituted with one, two or three substituents each independently selected from R^{p};
R^{p} is independently, for each occurrence, selected from the group consisting of halogen, C₁₋₄alkyl, C₁₋₄haloalkyl, hydroxy, C₁₋₄alkoxy, NR^{C}R^{D}, and aminoC₁₋₃alkyl;
R^{A} is independently, for each occurrence, selected from the group consisting of hydrogen, C₁₋₆alkyl, —C(O)C₁₋₆alkyl, and —C(O)OC₁₋₆alkyl;
R^{B} is selected from the group consisting of C₁₋₆alkyl, C₃₋₆cycloalkyl, and —C(O)OC₁₋₆alkyl;
R^{C} and R^{D} are independently, for each occurrence, selected from the group consisting of hydrogen, C₁₋₆alkyl, haloC₁₋₆alkyl, and C₃₋₄cycloalkyl, or

R^{C} and R^{D} together with the nitrogen atom to which they are attached form 4-6 membered heterocyclyl or 4-6 membered heteroaryl, wherein the 4-6 membered heterocyclyl or 4-6 membered heteroaryl may contain a further nitrogen atom or an oxygen atom and is optionally substituted with one or two fluoro; and

t is 0 or 1.

In another aspect, provided herein are compounds represented by Formula (I):

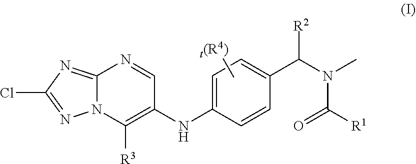

or a pharmaceutically acceptable salt thereof, wherein:
R¹ is C₁₋₆alkyl, C₃₋₆cycloalkyl, or 5-10 membered heterocyclyl, wherein the C₃₋₆cycloalkyl may be optionally substituted on one or more available carbons by one, two, three, or more substituents each independently selected from R^{1a}, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH₃ or CF₃;
R³ is C₁₋₆alkyl, wherein the C₁₋₆alkyl may be optionally substituted with C₁₋₆alkoxy;
R⁴ is C₁₋₆alkyl;
R^{1a} is independently, for each occurrence, selected from the group consisting of cyano, halogen, hydroxyl, C₁₋₆alkyl, —C(O)OR^{A}, —C(O)N(R^{A})₂, —N(R^{A})₂, C₁₋₆alkoxy, and 5-6 membered heteroaryl, wherein the C₁₋₆alkyl is optionally substituted with —N(R^{A})₂;
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl;
R^{A} is independently, for each occurrence, selected from the group consisting of hydrogen, C₁₋₆alkyl, —C(O)C₁₋₆alkyl, and —C(O)OC₁₋₆alkyl; and
t is 0 or 1.

In some embodiments, t=0. In some embodiments, t=1.

In some embodiments, R⁴ is C₁₋₆alkyl. In some embodiments, R⁴ is CH₃.

In another aspect, provided herein are compounds represented by Formula (Ia):

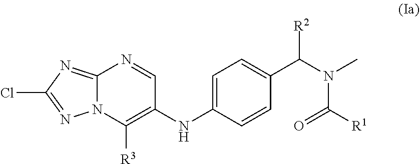

or a pharmaceutically acceptable salt thereof, wherein:
R¹ is selected from the group consisting of C₁₋₆alkyl, C₁₋₆alkoxy, C₃₋₆cycloalkyl, and 5-10 membered heterocyclyl, wherein the C₁₋₆alkyl, C₃₋₆cycloalkyl, and 5-10 membered heterocyclyl may be optionally substituted on one or more available carbons by one, two, three, or more substituents each independently selected from R^{1a}; wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH₃ or CF₃;
R³ is hydrogen; or
R³ is selected from the group consisting of C₁₋₆alkyl, C₁₋₆alkoxy, C₃₋₇cycloalkyl, 5-6 membered heterocyclyl, 5-6 membered heterocyclyl-C₁₋₃alkyl-, 5-6 membered heterocyclyl-O—, phenyl, and 5-6 membered heteroaryl, any of which may be optionally substituted with one, two or three substituents each independently selected from R^{3a};
R^{1a} is independently, for each occurrence, selected from the group consisting of cyano, halogen, hydroxyl, oxo, C₁₋₆alkyl, —C(O)OR^{A}, —C(O)N(R^{A})₂, —N(R^{A})₂, C₁₋₆alkoxy, 5-6 membered heterocyclyl, and 5-6 membered heteroaryl, wherein the C₁₋₆alkyl is optionally substituted with —N(R^{A})₂, and wherein if the 5-6 membered heterocyclyl contains a substitutable ring nitrogen atom, that ring nitrogen atom may optionally be substituted by R^{B}; R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl;
R^{3a} is independently, for each occurrence, selected from the group consisting of halogen, C₁₋₄alkyl, C₁₋₄haloalkyl, C₁₋₄alkoxy, C₁₋₄haloalkoxy, hydroxy, C₁₋₄alkenyl, cyano, azido, NR^{C}R^{D}, C₃₋₆cycloalkyl, C₁₋₄alkoxyC₁₋₄alkoxy, 5-6 membered heterocyclyl-O—, 5-6 membered heterocyclyl, and phenyl, wherein C₃₋₆cycloalkyl, 5-6 membered heterocyclyl-O—, 5-6 membered heterocyclyl, and phenyl are optionally substituted with one, two or three substituents each independently selected from R^{p};

R^{p} is selected from the group consisting of halogen, C₁₋₄alkyl, C₁₋₄haloalkyl, hydroxy, C₁₋₄alkoxy, C₁₋₄alkoxyC₁₋₄alkyl, NR^{C}R^{D}, and aminoC₁₋₃alkyl;
R^{A} is independently, for each occurrence, selected from the group consisting of hydrogen, C₁₋₆alkyl, —C(O)C₁₋₆alkyl, and —C(O)OC₁₋₆alkyl;
R^{B} is selected from the group consisting of C₁₋₆alkyl, C₃₋₆cycloalkyl, and —C(O)OC₁₋₆alkyl; and
R^{C} and R^{D} are independently, for each occurrence, selected from the group consisting of hydrogen, C₁₋₆alkyl, haloC₁₋₆alkyl, and C₃₋₄cycloalkyl, or
R^{C} and R^{D} together with the nitrogen atom to which they are attached form 4-6 membered heterocyclyl or 4-6 membered heteroaryl, wherein the 4-6 membered heterocyclyl or 4-6 membered heteroaryl may contain a further nitrogen atom or an oxygen atom and is optionally substituted with one or two fluoro.

In another aspect, provided herein are compounds represented by Formula (Ia):

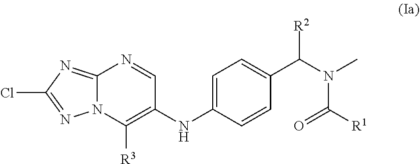

or a pharmaceutically acceptable salt thereof, wherein:
R¹ is C₁₋₆alkyl, C₃₋₆cycloalkyl, and 5-10 membered heterocyclyl, wherein the C₃₋₆cycloalkyl may be optionally substituted on one or more available carbons by one, two, three, or more substituents each independently selected from R^{1a}; wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH₃ or CF₃;
R³ is C₁₋₆alkyl, wherein the C₁₋₆alkyl may be optionally substituted with C₁₋₄alkoxy;
R^{1a} is independently, for each occurrence, selected from the group consisting of cyano, halogen, hydroxyl, C₁₋₆alkyl, —C(O)OR^{A}, —C(O)N(R^{A})₂, —N(R^{A})₂, C₁₋₆alkoxy, and 5-6 membered heteroaryl, wherein the C₁₋₆alkyl is optionally substituted with —N(R^{A})₂;
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl; and
R^{A} is independently, for each occurrence, selected from the group consisting of hydrogen, C₁₋₆alkyl, —C(O)C₁₋₆alkyl, and —C(O)OC₁₋₆alkyl;

In some embodiments, R² is CH₃. In some embodiments, R² is CF₃.

In some embodiments, R³ is C₁₋₆alkyl, wherein R³ is optionally substituted with C₁₋₄alkoxy.

In some embodiments, R³ is C₁₋₆alkyl. In some embodiments, R³ is

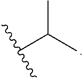 [SMILES: *C(C)C]

In some embodiments, R³ is C₁₋₆alkyl, wherein R³ is substituted with C₁₋₄alkoxy. In some embodiments, R³ is

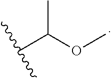 [SMILES: *C(C)OC]

In another aspect, provided herein are compounds represented by Formula (Ib):

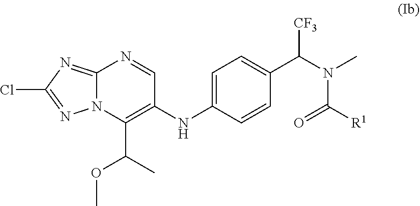

or a pharmaceutically acceptable salt thereof, wherein:
R¹ is C₁₋₆alkyl, C₃₋₆cycloalkyl, or 5-10 membered heterocyclyl, wherein the C₃₋₆cycloalkyl may be optionally substituted on one or more available carbons by one, two, three, or more substituents each independently selected from R^{1a}, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R^{1a} is independently, for each occurrence, selected from the group consisting of cyano, halogen, hydroxyl, C₁₋₆alkyl, —C(O)OR^{A}, —C(O)N(R^{A})₂, —N(R^{A})₂, C₁₋₆alkoxy, and 5-6 membered heteroaryl, wherein the C₁₋₆alkyl is optionally substituted with —N(R^{A})₂;
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl; and
R^{A} is independently, for each occurrence, selected from the group consisting of hydrogen, C₁₋₆alkyl, —C(O)C₁₋₆alkyl, and —C(O)OC₁₋₆alkyl.

In some embodiments, R¹ is C₁₋₆alkyl. In some embodiments, R¹ is CH₃.

In some embodiments, R¹ is C₃₋₆cycloalkyl, wherein R¹ may be optionally substituted on one or more available carbons by one, two, three, or more substituents each independently selected from R^{1a}.

In some embodiments, R¹ is C₃₋₆cycloalkyl. In some embodiments, R¹ is selected from the group consisting of

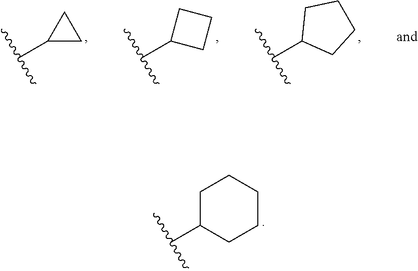

In some embodiments, R¹ is C₃₋₆cycloalkyl, wherein R¹ is substituted on one or more available carbons by one, two, three, or more substituents each independently selected from R^{1a}.

In some embodiments, R¹ is

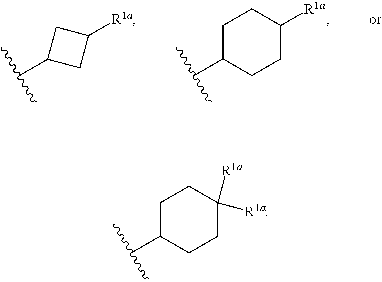

In some embodiments, R^{1a} is selected from the group consisting of cyano, fluoro, hydroxyl, —O—CH₃, —C(O)OH, —C(O)NH₂,

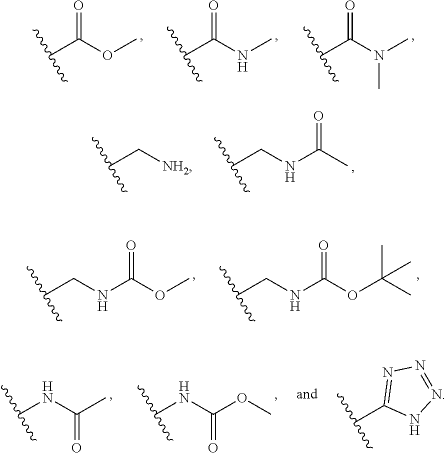

In some embodiments, R¹ is selected from the group consisting of

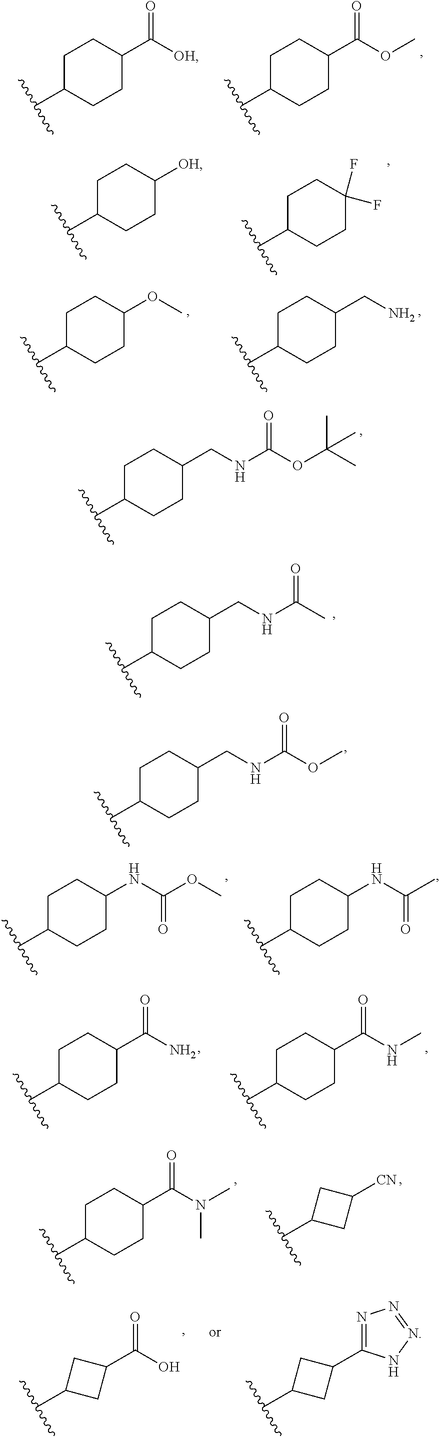

In some embodiments, R¹ is selected from the group consisting of

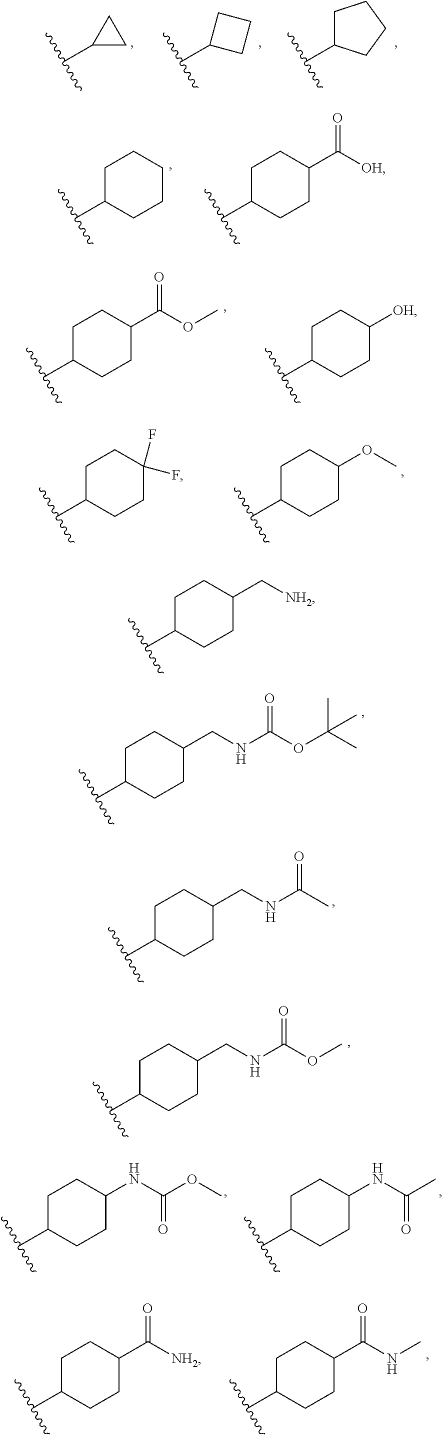

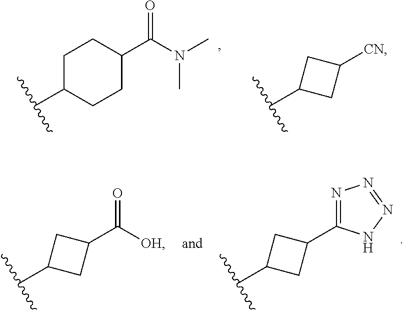

In some embodiments, R¹ is 5-10 membered heterocyclyl, wherein if R¹ contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms.

In some embodiments, R¹ is 5-10 membered heterocyclyl. In some embodiments, R¹ is selected from the group consisting of

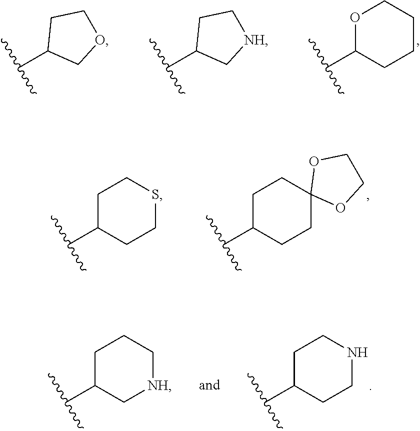

In some embodiments, R¹ is

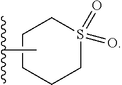

In some embodiments, R¹ is

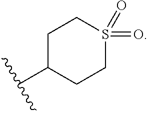 [SMILES: *C1CCS(=O)(=O)CC1]

In some embodiments, R¹ is selected from the group consisting of

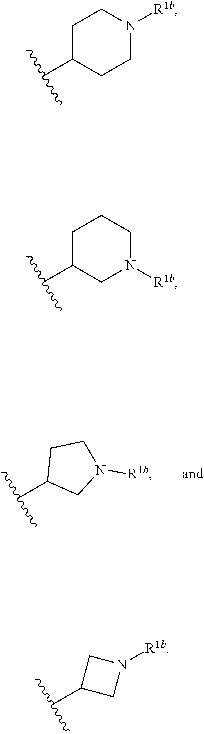

In some embodiments, R^{1b} is selected from the group consisting of CH₃,

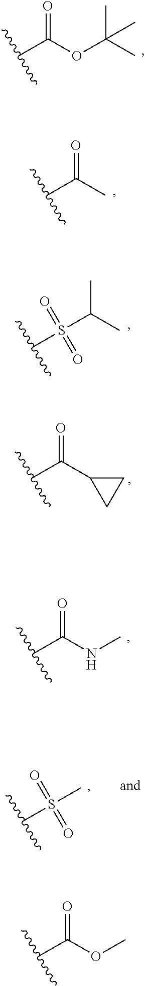

In some embodiments, R¹ is selected from the group consisting of

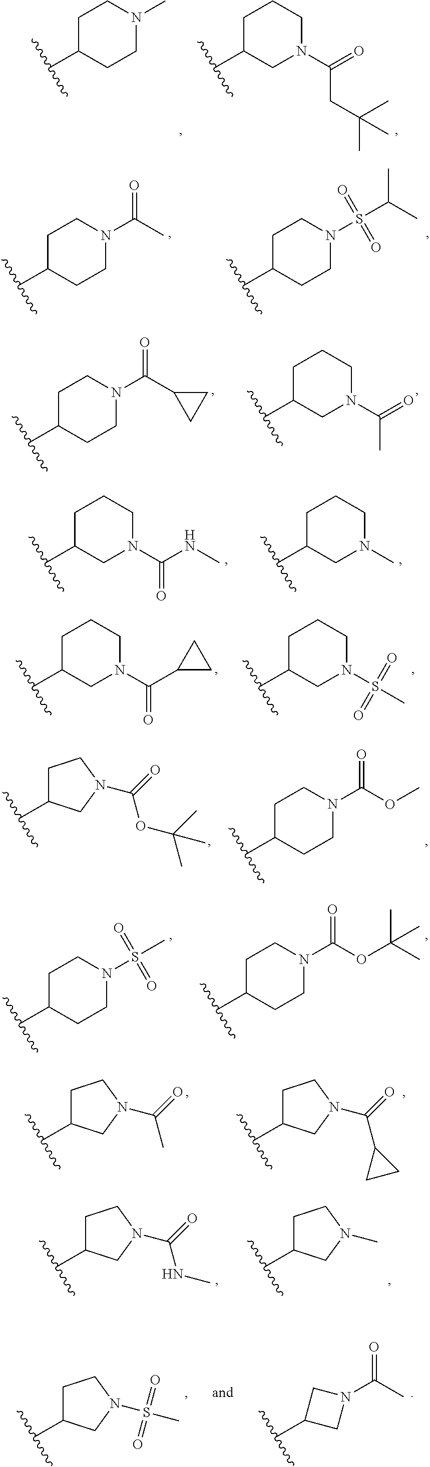

In some embodiments, R¹ is selected from group consisting of

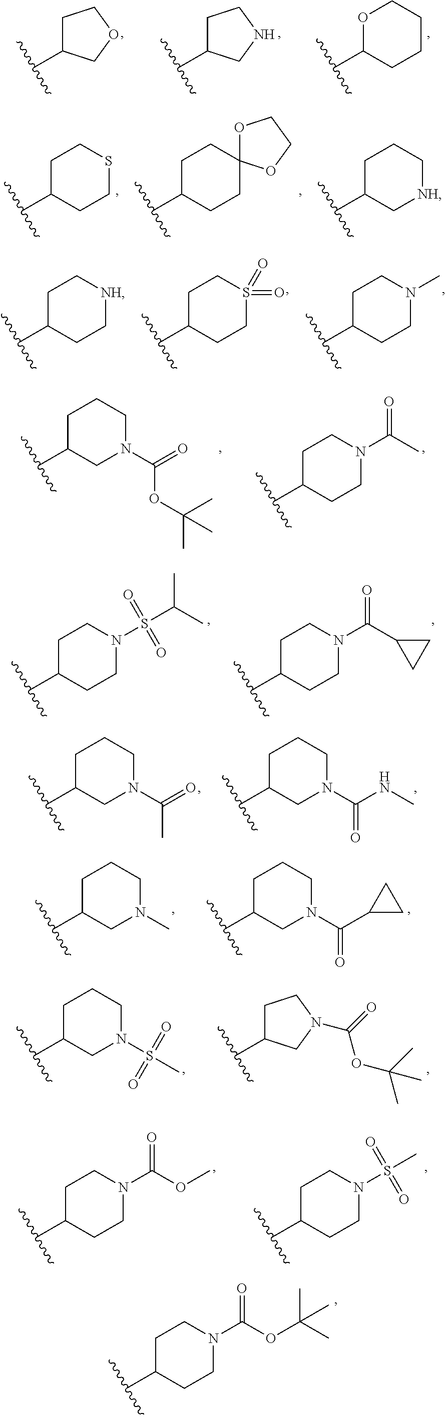

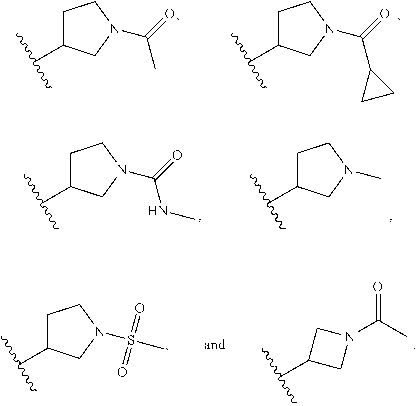

In some embodiments, R¹ is selected from the group consisting of CH₃,

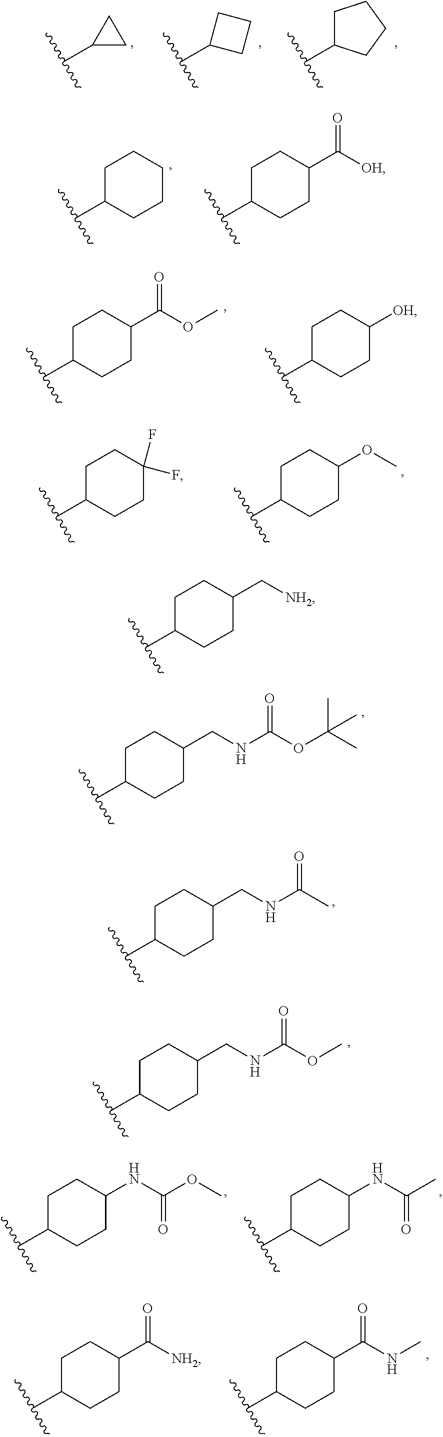

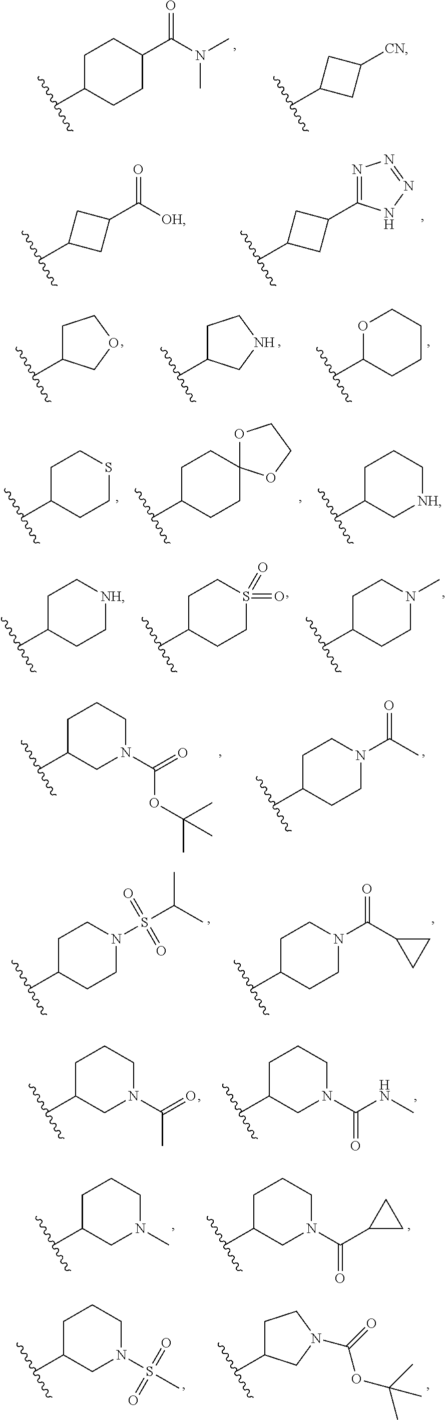

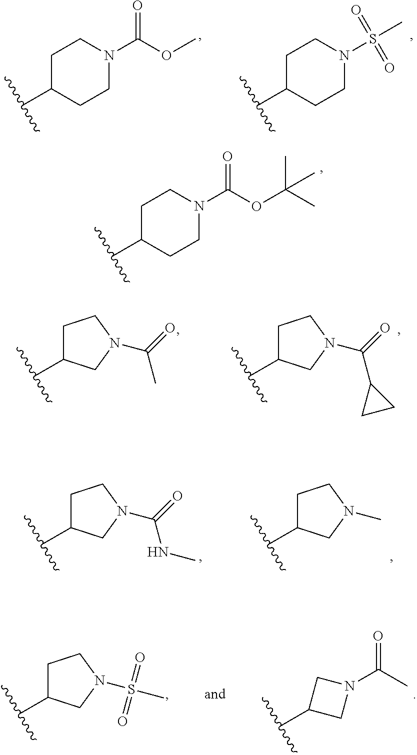

In some embodiments, R¹ is selected from the group consisting of CH₃,

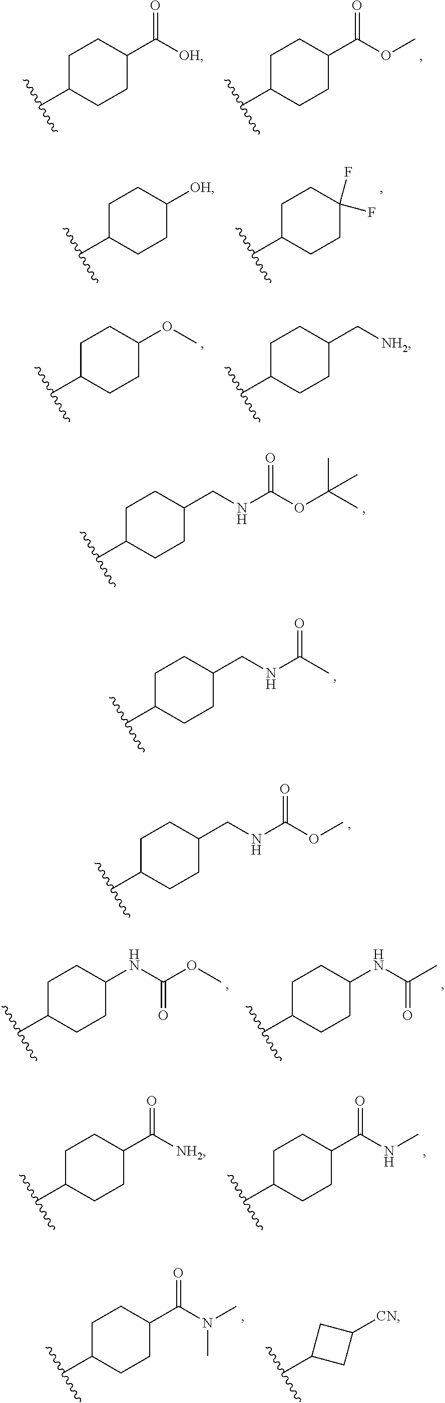

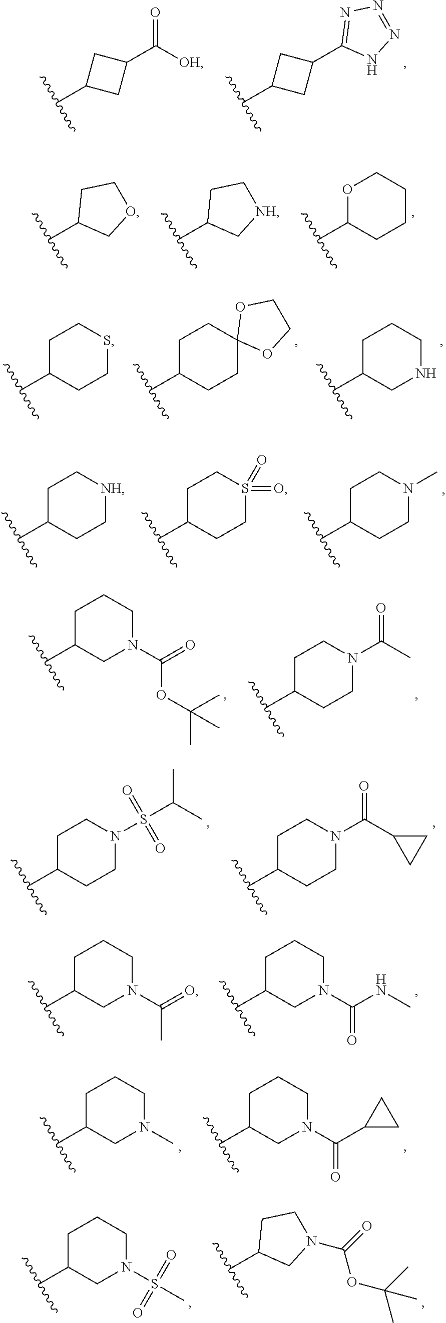

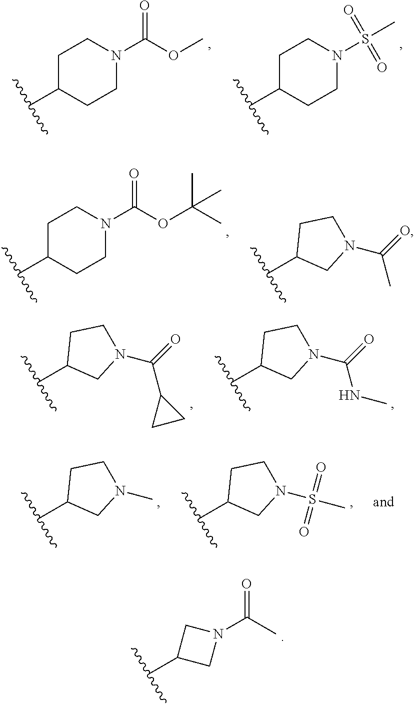

In some embodiments, a compound provided herein is selected from a compound set forth in Table 1, or a pharmaceutically acceptable salt thereof.

In some embodiments, the compound is selected from the group consisting of
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylacetamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyloxane-2-carboxamide;
methyl (1r,4r)-4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl] (methyl)carbamoyl}cyclohexane-1-carboxylate;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylthiane-4-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyloxolane-3-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,4-dioxaspiro[4.5]decane-8-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4,4-difluoro-N-methylcyclohexane-1-carboxamide;
(1r,3S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-3-cyano-N-methylcyclobutane-1-carboxamide;
Tert-butyl 4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate;
Tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate;
Tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}pyrrolidine-1-carboxylate;
tert-butyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate;
(1r,4S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-4-methoxy-N-methylcyclohexane-1-carboxamide;
1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylazetidine-3-carboxamide;
N-(4-{[(1S)-1-[4-({2-chloro-7-[(1S)-1-methoxyethyl]-[1,2,4]triazolo[1,5-a]pyrimidin6-yl}amino)phenyl]-2,2,2-trifluoroethyl](methyl)carbamoyl}cyclohexyl)carbamate;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4-acetamido-N-methylcyclohexane-1-carboxamide;
N-[(1S)-1-(4-{[2-chloro-7-(propan-2-yl)-[1,2,4]triazolo[1,5-a]pyrimidin-6-yl]amino}phenyl)-2,2,2-trifluoroethyl]-N-methyl-1,1-dioxo-1Δ⁶-thiane-4-carboxamide;
1-acetyl-N-[(1S)-1-(4-{[2-chloro-7-(propan-2-yl)-[1,2,4]triazolo[1,5-a]pyrimidin-6-yl]amino}phenyl)-2,2,2-trifluoroethyl]-N-methylpiperidine-4-carboxamide;
N-{1-[4-({2-chloro-7-[(1S)-1-methoxyethyl]-[1,2,4]triazolo[1,5-a]pyrimidin-6-yl}amino)phenyl]ethyl}-N-methylcyclobutanecarboxamide;
N-{1-[4-({2-chloro-7-[(1S)-1-methoxyethyl]-[1,2,4]triazolo[1,5-a]pyrimidin-6-yl}amino)phenyl]ethyl}-N-methylcyclohexanecarboxamide;
N-{1-[4-({2-chloro-7-[(1S)-1-methoxyethyl]-[1,2,4]triazolo[1,5-a]pyrimidin-6-yl}amino)phenyl]ethyl}-N-methylcyclopentanecarboxamide;
N-{1-[4-({2-chloro-7-[(1S)-1-methoxyethyl]-[1,2,4]triazolo[1,5-a]pyrimidin-6-yl}amino)phenyl]ethyl}-N-methylcyclopropanecarboxamide;
N-{1-[4-({2-chloro-7-[(1S)-1-methoxyethyl]-[1,2,4]triazolo[1,5-a]pyrimidin-6-yl}amino)phenyl]ethyl}-N-methylacetamide;
N-{1-[4-({2-chloro-7-[(1S)-1-methoxyethyl]-[1,2,4]triazolo[1,5-a]pyrimidin-6-yl}amino)phenyl]-2,2,2-trifluoroethyl}-N-methylacetamide;
N-[(1R)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide;
N-[(1R)-1-[4-({2-chloro-7-[(1R)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide;
N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methylcyclobutanecarboxamide;
N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methylcyclopropanecarboxamide;
N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methyl-1,1-dioxo-1λ⁶-thiane-4-carboxamide;
N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methylcyclobutanecarboxamide;
N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methylcyclopropanecarboxamide;
N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methyl-1,1-dioxo-1λ⁶-thiane-4-carboxamide;
N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide;
N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpyrrolidine-3-carboxamide;
4-(aminomethyl)-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylcyclohexane-1-carboxamide;
1-acetyl-N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1-(propane-2-sulfonyl)piperidine-4-carboxamide;
methyl 4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpiperidine-4-carboxamide;
1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide;
N³-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N¹,N³-dimethylpiperidine 1,3-dicarboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpiperidine-3-carboxamide;
1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide;
N³-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N¹,N³-dimethylpyrrolidine-1,3-dicarboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpyrrolidine-3-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4-(acetamidomethyl)-N-methylcyclohexane-1-carboxamide;
methyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate;
N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-1-(cyclopropanecarbonyl)-N-methylpiperidine-4-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-cyclopropanecarbonyl-N-methylpiperidine-3-carboxamide;
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-cyclopropanecarbonyl-N-methylpyrrolidine-3-carboxamide;
N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N,1-dimethylpiperidine-4-carboxamide;
N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N,1-dimethylpiperidine-3-carboxamide
N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N,1-dimethylpyrrolidine-3-carboxamide;
(1r,4S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-4-hydroxy-N-methylcyclohexane-1-carboxamide;
(1S,4r)-4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexane-1-carboxylic acid:
(1S,4r)-4-(((S)-1-(4-((2-chloro-7-isopropyl-[1,2,4]triazolo[1,5-a]pyrimidin-6-yl)amino)phenyl)-2,2,2-trifluoroethyl)(methyl)carbamoyl)cyclohexane-1-carboxylic acid;
(1S,3r)-3-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclobutane-1-carboxylic acid;
(1r,4S)—N¹—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N¹-methylcyclohexane-1,4-dicarboxamide;
(1r,4S)—N¹—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N¹,N⁴-dimethylcyclohexane-1,4-dicarboxamide;
(1r,4S)—N¹—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N¹,N⁴,N⁴-trimethylcyclohexane-1,4-dicarboxamide;
(1r,3S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methyl-3-(1H-tetrazol-5-yl)cyclobutane-1-carboxamide;
N—((S)-1-(4-((2-chloro-7-((R)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide;
1-acetyl-N—((S)-1-(4-((2-chloro-7-((R)-1-methoxyethyl)-[1,2,4]triazolo[1,5-a]pyrimidin-6-yl)amino)phenyl)-2,2,2-trifluoroethyl)-N-methylpiperidine-4-carboxamide; and a pharmaceutically acceptable salt thereof.

### Pharmaceutical Compositions and Routes of Administration

Compounds provided in accordance with the present invention are usually administered in the form of pharmaceutical compositions. This invention therefore provides pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprising a disclosed compound, or pharmaceutically acceptable salt thereof, and a pharmaceutically acceptable carrier.

### Methods of Use

Compounds and compositions described herein are generally useful for modulating MALT1 and are useful for in treating diseases or disorders, in particular those susceptible to modulation of proteolytic and/or autoproteolytic activity of MALT1. In some embodiments, the compounds and compositions described herein are useful for inhibiting MALT1. In some embodiments, it is contemplated that the compounds and compositions of the present invention may be useful in the treatment of a disease, a disorder, or a condition characterized by dysregulated NF-kB activation, for example, autoimmune or immunological and inflammatory disorders, allergic disorders, respiratory disorders and oncological disorders.

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein, e.g. a compound of Formula I); such as a compound of Formula named herein.

In some embodiments, the autoimmune and inflammatory disorders are selected from arthritis, ankylosing spondylitis, inflammatory bowel disease, ulcerative colitis, gastritis, pancreatitis, Crohn's disease, celiac disease, multiple sclerosis, systemic lupus erythematosus, lupus nephritis, rheumatoid arthritis, rheumatic fever, gout, organ or transplant rejection, acute or chronic graft-versus-host disease, chronic allograft rejection. Behcet's disease, uveitis, psoriasis, psoriatic arthritis, BENTA disease, polymyositis, dermatitis, atopic dermatitis, dermatomyositis, acne vulgaris, myasthenia gravis, hidradenitis suppurativa, Grave's disease. Hashimoto thyroiditis, Sjogren's syndrome, and blistering disorders (e.g., pemphigus vulgaris), antibody-mediated vasculitis syndromes, including ANCA-associated vasculitides. Henoch-Schonlein Purpura, and immune-complex vasculitides (either primary or secondary to infection or cancers).

In some embodiments, the oncological disorders are selected from carcinoma, sarcoma, lymphoma, leukemia and germ cell tumors, adenocarcinoma, bladder cancer, clear cell carcinoma, skin cancer, brain cancer, cervical cancer, colon cancer, colorectal cancer, endometrial cancer, brain tumors, breast cancer, gastric cancer, germ cell tumors, glioblastoma, hepatic adenomas, Hodgkin's lymphoma, liver cancer, kidney cancer, lung cancer, pancreatic cancer, head/neck/throat cancer, ovarian cancer, dermal tumors, prostate cancer, renal cell carcinoma, stomach cancer, hematologic cancer, medulloblastoma, non-Hodgkin's lymphoma, diffuse large B-cell lymphoma (DLBCL), activated B cell-like diffuse large B Cell lymphoma (ABC-DLBCL), mantle cell lymphoma, marginal zone lymphoma, T cell lymphomas, in particular Sezary syndrome, Mycosis fungoides, cutaneous T-cell lymphoma, T-cell acute lymphoblastic leukemia, melanoma, mucosa-associated lymphoid tissue (MALT) lymphoma, multiple myeloma, plasma cell neoplasm, lentigo maligna melanomas, acral lentignous melanoma, squamous cell carcinoma, chronic myelogenous leukemia, myeloid leukemia, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a Spitz nevus, uveal melanoma, precursor T-cell, leukemia/lymphoma, acute myeloid leukemia, chronic myeloid leukemia, acute lymphocytic leukemia, follicular lymphoma, chronic lymphocytic leukemia/lymphoma, Burkitt's lymphoma, mycosis fungoides, peripheral T-cell lymphoma, nodular sclerosis form of Hodgkin lymphoma, mixed-cellularity subtype of Hodgkin lymphoma, non-small-cell lung cancer, large-cell carcinoma, and small-cell lung carcinoma. In some embodiments, the oncological disorder is a cancer in the form of a tumor or a blood born cancer. In some embodiments, the tumor is a solid tumor. In some embodiments, the tumor is malignant and/or metastatic. In some embodiments, the tumor is selected from an adenoma, an adenocarcinoma, a blastoma (e.g., hepatoblastoma, glioblastoma, neuroblastoma and retinoblastoma), a carcinoma (e.g., colorectal carcinoma or heptatocellular carcinoma, pancreatic, prostate, gastric, esophageal, cervical, and head and neck carcinomas, and adenocarcinoma), a desmoid tumor, a desmoplastic small round cell tumor, an endocrine tumor, a germ cell tumor, a lymphoma, a leukemia, a sarcoma (e.g., Ewing sarcoma, osteosarcoma, rhabdomyosarcoma, or any other soft tissue sarcoma), a Wilms tumor, a lung tumor, a colon tumor, a lymph tumor, a breast tumor or a melanoma.

In some embodiments, the allergic disorder is selected from contact dermatitis, celiac disease, asthma, hypersensitivity to house dust mites, pollen and related allergens, and berylliosis.

In some embodiments, the respiratory disorders is selected from asthma, bronchitis, chronic obstructive pulmonary disease (COPD), cystic fibrosis, pulmonary edema, pulmonary embolism, pneumonia, pulmonary sarcoidosis, silicosis, pulmonary fibrosis, respiratory failure, acute respiratory distress syndrome, primary pulmonary hypertension and emphysema.

In some embodiments, the compounds and compositions of the present invention may be useful in the treatment of rheumatoid arthritis, systemic lupus erythematosus, vasculitic conditions, allergic diseases, asthma, chronic obstructive pulmonary disease (COPD), acute or chronic transplant rejection, graft versus host disease, cancers of hematopoietic origin or solid tumors, chronic myelogenous leukemia, myeloid leukemia, non-Hodgkin lymphoma or other B cell lymphomas.

### Combination Therapy

A compound of composition described herein may be administered in combination with another agent or therapy. A subject to be administered a compound disclosed herein may have a disease, disorder, or condition, or a symptom thereof, that would benefit from treatment with another agent or therapy.

In some embodiments, the compound of composition described herein may be administered either simultaneously with, or before or after, one or more other therapeutic agent. In some embodiments, the compound of composition described herein may be administered separately, by the same or different route of administration, or together in the same pharmaceutical composition as the other agents.

In some embodiments, the compound described herein may be administered as the sole active ingredient or in conjunction with, e.g., as an adjuvant to, other drugs e.g., immunosuppressive or immunomodulating agents or other anti-inflammatory agents, e.g., for the treatment or prevention of alio- or xenograft acute or chronic rejection or inflammatory or autoimmune disorders, or a chemotherapeutic agent, e.g., a malignant cell anti-proliferative agent. For example, the compounds of the invention may be used in combination with a calcineurin inhibitor, e.g., cyclosporin A or FK 506; a rmTOR inhibitor, e.g., rapamycin, 40-0-(2-hydroxyethyl)-rapamycin, biolimus-7 or biolimus-9; an ascomycin having immunosuppressive properties, e.g., ABT-281, ASM981; corticosteroids; cyclophosphamide; azathioprene; methotrexate; leflunomide; mizoribine; mycophenolic acid or salt; mycophenolate mofetil; or IL-1 beta inhibitor.

In some embodiments, the compound described herein is combined with a co-agent which is a PI3K inhibitor.

In some embodiments, the compound described herein is combined with co-agent that influence BTK (Bruton's tyrosine kinase).

For the treatment of oncological diseases, the compound described herein may be used in combination with B-cell modulating agents, e.g., Rituximab, Ofatumumab, BTK or SYK inhibitors, inhibitors of PKC, PI3K, PDK, PIM, JAK and mTOR and BH3 mimetics.

### EXAMPLES

The representative examples that follow are intended to help illustrate the invention, and are not intended to, nor should they be construed to, limit the scope of the invention.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

### LIST OF ABBREVIATIONS

EtOAc ethyl acetate
THF tetrahydrofuran
PE petroleum ether
DMA dimethylacetamide
MeOH methanol
EtOH ethanol
DMF N,N-dimethylformamide
DMSO dimethyl sulfoxide
xphos 2-dicyclohexylphosphino-2′,4′,6′-triisopropylbiphenyl
FA formic acid
DCM dichloromethane
MTBE methyl tert-butyl ether
TFA trifluoroacetic acid
MeCN, ACN acetonitrile
i-PrMgCl iso-propyl magnesium chloride
xantphos 4,5-Bis(diphenylphosphino)-9,9-dimethylxanthene
i-PrOH isopropyl alcohol
TMSCF₃ trifluoromethyltrimethylsilane
MeI iodomethane
LiHMDS Lithium bis(trimethylsilyl)amide
h, hr, hrs hour(s)
Calcd calculated
UV ultraviolet
rt room temperature
DIEA, DIPEA N,N-Diisopropylethylamine
HATU 1-[Bis(dimethylamino)methylene]-1H-1,2,3-triazolo[4,5-b]pyridinium 3-oxid hexafluorophosphate
CDI 1,1′-Carbonyldiimidazole
ee enantiomeric excess
TMS trimethylsilyl
Pd₂(dba)₃ tris(dibenzylideneacetone)dipalladium(0)
Et₃N, TEA triethylamine
Cs₂CO₃ cesium carbonate
INT intermediate
EDCI 1-(3-Dimethylaminopropyl)-3-ethylcarbodiimide hydrochloride
HOBT, HOBt 1-hydroxybenzotriazole
TLC thin layer chromatography
Conc. concentrated
Boc tert-butyloxycarbonyl
AcCl acetyl chloride
NaBH₃CN sodium cyanoborohydride
HO(CH₂O)nH paraformaldehyde
LiOH·H₂O lithium hydroxide monohydrate
NH₄Cl ammonium chloride
MeNH₂·HCl methylamine hydrochloride
Me₂NH·HCl dimethylamine hydrochloride
TMSN₃ trimethylsilyl azide
(C₄H₉)₂SnO dibutyltin oxide
NH₂NH₂·H₂O hydrazine hydrate
t-BuOH tert-butyl alcohol
t-BuOK potassium tert-butoxide
DPPA diphenylphosphoryl azide
NaOH sodium hydroxide
(n-Bu)₄OAc tetrabutylammonium acetate
(COCl)₂ oxalyl chloride
Na₂CO₃ sodium carbonate
POCl₃ phosphorous oxychloride
TBSCl tert-Butyldimethylsilyl chloride
TBS tert-Butyldimethylsilyl
NaH sodium hydride
Ti(OEt)₄ titanium ethoxide
TBAT tetrabutylammonium difluorotriphenylsilicate
o-n overnight
EDTA ethylenediaminetetraacetic acid

### Example 1. Preparation of the Compounds

Methods for preparing compounds described herein are illustrated in the following synthetic schemes. These schemes are given for the purpose of illustrating the invention and should not be regarded in any manner as limiting the scope or the spirit of the invention. Starting materials shown in the schemes may be obtained from commercial sources or can be prepared from commercially available sources based on procedures described in the literature.

### 1. Compounds Prepared Using Scheme 1

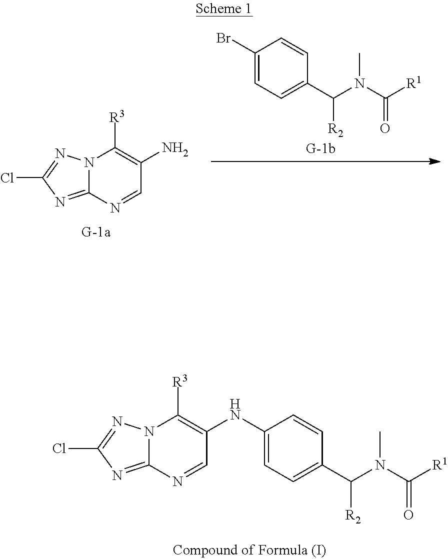

R³-containing triazolopyrimidine G-1a is reacted with R¹- and R²-containing carboxamide G-1b to provide a compound of Formula (I).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide (Also Known as N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide) (Compound 1.1)

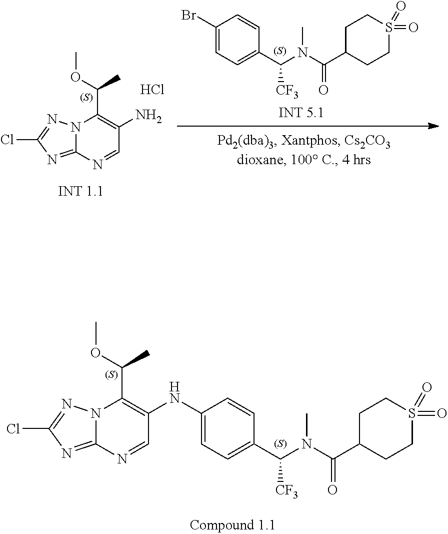

A mixture of 2-chloro-7-[(1S)-1-methoxyethyl]-[1,2,4]triazolo[1,5-a]pyrimidin-6-amine hydrochloride [INT 1.1] (460 mg, 1.74 mmol), N-[(1S)-1-(4-bromophenyl)-2,2,2-trifluoroethyl]-N-methyl-1,1-dioxo-1λ⁶-thiane-4-carboxamide [INT 5.1] (779 mg, 1.82 mmol), Pd₂(dba)₃ (159 mg, 174 μmol), Xantphos (201 mg, 348 μmol) and Cs₂CO₃ (1.70 g, 5.22 mmol) in dioxane (6 mL) was stirred at 100° C. for 4 h. The mixture was concentrated under reduced pressure to afford the crude product which was purified by flash chromatography on silica gel (Methanol/Dichloromethane=0/1 to 1/10) and Prep-HPLC (column: YMC Triart C18 250*50 mm*7 μm, table: 29-58% B (A=water (0.05% ammonia hydroxide v/v)), B=acetonitrile), flow rate: 60 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide [Compound 1.1] (211 mg, 368 μmol) as a dry powder. m/z: [M+H]+ Calcd for C23H27ClF3N6O4S 575.2; Found 575.3. ¹H NMR (400 MHz, DMSO-d₆) δ=8.80 (s, 1H), 8.02-7.94 (m, 1H), 7.26-7.12 (m, 2H), 6.99-6.89 (m, 2H), 6.45-6.02 (m, 1H), 5.12 (q, J=6.8 Hz, 1H), 3.25-3.14 (m, 3H), 3.12 (s, 3H), 3.10-3.05 (m, 2H), 2.86 (s, 3H), 2.08-1.94 (m, 4H), 1.55 (d, J=6.8 Hz, 3H). Compound 1.1 was determined to have a chiral purity of at least 89%.

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylacetamide (Compound 1.2)

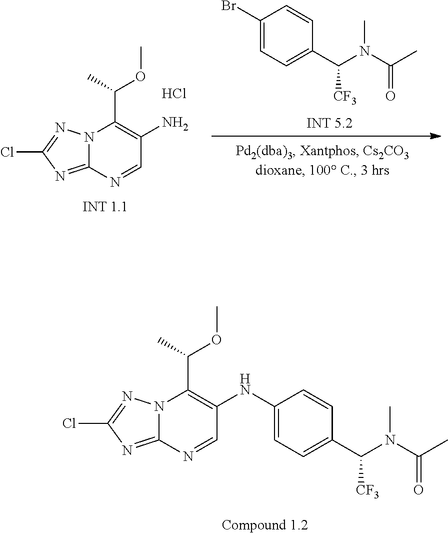 [SMILES: CO[C@@H](C)c1c(N)cnc2nc(Cl)nn12 ; Cl ; CC(=O)N(C)[C@@H](c1ccc(Br)cc1)C(F)(F)F ; CO[C@@H](C)c1c(Nc2ccc([C@H](N(C)C(C)=O)C(F)(F)F)cc2)cnc2nc(Cl)nn12]

A mixture of 2-chloro-7-[(1S)-1-methoxyethyl]-[1,2,4]triazolo[1,5-a]pyrimidin-6-amine hydrochloride [INT 1.1] (100 mg, 378 μmol), N-[(1S)-1-(4-bromophenyl)-2,2,2-trifluoroethyl]-N-methylacetamide [INT 5.2] (117 mg, 378 μmol), Pd₂(dba)₃ (34.6 mg, 37.8 μmol), Xantphos (43.7 mg, 75.6 μmol), and Cs₂CO₃ (368 mg, 1.13 mmol) in dioxane (3 mL) was stirred at 100° C. for 3 hr under N₂ atmosphere. The mixture was concentrated under reduced pressure to afford the crude product, which was purified by prep-HPLC (column: YMC Triart C18 250*50 mm*7 μm, table: 31-71% B (A=water (0.05% ammonia hydroxide v/v)), B=acetonitrile), flow rate: 60 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylacetamide [Compound 1.2] (20.0 mg, 43.7 μmol, 11.6% yield) as a yellow dry powder. m/z: [M+H]+ Calcd for C19H21ClF3N6O2 457.1; Found 457.2. ¹H NMR (400 MHz, CD₃OD) δ=8.90-8.85 (m, 1H), 7.40-7.27 (m, 2H), 7.11-7.01 (m, 2H), 6.50 (q, J=9.2 Hz, 1H), 5.36 (q, J=6.4 Hz, 1H), 3.37-3.35 (m, 3H), 2.95-2.72 (m, 3H), 2.36-2.19 (m, 3H), 1.64 (d, J=6.8 Hz, 3H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyloxane-2-carboxamide (Compound 1.3)

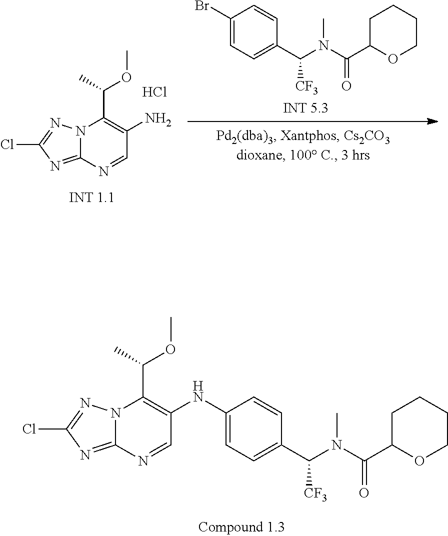 [SMILES: CO[C@@H](C)c1c(N)cnc2nc(Cl)nn12 ; Cl ; CN(C(=O)C1CCCCO1)[C@@H](c1ccc(Br)cc1)C(F)(F)F ; CO[C@@H](C)c1c(Nc2ccc([C@H](N(C)C(=O)C3CCCCO3)C(F)(F)F)cc2)cnc2nc(Cl)nn12]

A mixture of 2-chloro-7-[(1S)-1-methoxyethyl]-[1,2,4]triazolo[1,5-a]pyrimidin-6-amine hydrochloride [INT 1.1] (80 mg, 302 μmol), N-[(1S)-1-(4-bromophenyl)-2,2,2-trifluoroethyl]-N-methyloxane-2-carboxamide [INT 5.3] (114 mg, 302 μmol), Xantphos (34.9 mg, 60.4 μmol), Pd₂(dba)₃ (27.6 mg, 30.2 μmol), and Cs₂CO₃ (1% mg, 604 μmol) in dioxane (2 mL) was stirred at 100° C. for 3 hr under N₂ atmosphere. The reaction was concentrated under reduced pressure to afford the crude product, which was purified by flash chromatography on silica gel (EtOAc/Petroleum ether=1/10 to 1/1) and prep-HPLC (column: YMC Triart C18 250*50 mm*7 μm, table: 26-66% B (A=water (0.05% ammonia hydroxide v/v)), B=acetonitrile), flow rate: 60 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyloxane-2-carboxamide [Compound 1.3] (30.1 mg, 57.1 μmol, 18.9% yield) as a yellow dry powder. m/z: [M+H]+ Calcd for C23H27ClF3N6O3 527.2; Found 527.3. ¹H NMR (400 MHz, CD₃OD) δ=8.91-8.83 (m, 1H), 7.47-7.26 (m, 2H), 7.06 (d, J=8.4 Hz, 2H), 6.54-6.04 (m, 1H), 5.36 (q, J=6.8 Hz, 1H), 4.41-4.25 (m, 1H), 4.08-3.95 (m, 1H), 3.71-3.51 (m, 1H), 3.36 (s, 3H), 2.98-2.69 (m, 3H), 1.98-1.90 (m, 1H), 1.85-1.66 (m, 3H), 1.64 (d, J=6.8 Hz, 3H), 1.63-1.54 (m, 2H).

### Synthesis of methyl(1r,4r)-4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl] (methyl)carbamoyl}cyclohexane-1-carboxylate (Compound 1.4)

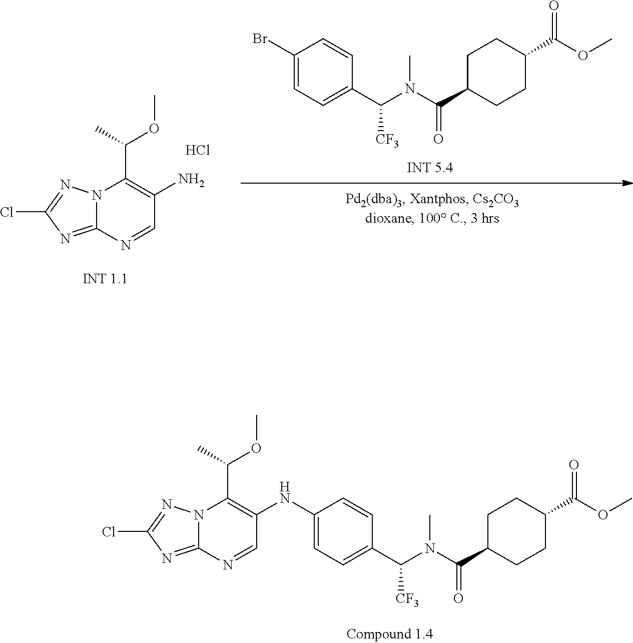 [SMILES: CO[C@@H](C)c1c(N)cnc2nc(Cl)nn12 ; Cl ; COC(=O)[C@H]1CC[C@H](C(=O)N(C)[C@@H](c2ccc(Br)cc2)C(F)(F)F)CC1 ; COC(=O)[C@H]1CC[C@H](C(=O)N(C)[C@@H](c2ccc(Nc3cnc4nc(Cl)nn4c3[C@H](C)OC)cc2)C(F)(F)F)CC1]

A mixture of 2-chloro-7-[(1S)-1-methoxyethyl]-[1,2,4]triazolo[1,5-a]pyrimidin-6-amine hydrochloride [INT 1.1] (50 mg, 189 μmol), methyl (1r,4r)-4-{[(1S)-1-(4-bromophenyl)-2,2,2-trifluoroethyl](methyl)carbamoyl}cyclohexane-1-carboxylate [INT 5.4] (82.4 mg, 189 μmol), Xantphos (21.8 mg, 37.8 μmol), Pd₂(dba)₃ (17.3 mg, 18.9 μmol), and Cs₂CO₃ (123 mg, 378 μmol) in dioxane (2 mL) was stirred at 100° C. for 3 hr under N₂ atmosphere. The reaction was concentrated under reduced pressure to afford the crude product, which was purified by flash chromatography on silica gel (EtOAc/Petroleum ether=1/10 to 1/1) and prep-HPLC (column: YMC Triart C18 250*50 mm*7 μm, table: 27-67% B (A=water (0.05% ammonia hydroxide v/v)), B=acetonitrile), flow rate: 60 mL/min, UV Detector 220 nm) to afford methyl (1r,4r)-4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl] (methyl)carbamoyl}cyclohexane-1-carboxylate [Compound 1.4] (14.2 mg, 24.3 μmol, 12.9% yield) as a yellow dry powder. m/z: [M+H]+ Calcd for C26H31ClF3N6O4 583.2; Found 583.3. ¹H NMR (400 MHz, CDCl₃) δ=8.90 (s, 1H), 7.32 (d, J=8.4 Hz, 2H), 7.13-7.09 (m, 1H), 7.04 (d, J=8.4 Hz, 2H), 6.63 (q, J=8.2 Hz, 1H), 5.48 (q, J=6.8 Hz, 1H), 3.69 (s, 3H), 3.49 (s, 3H), 2.92 (s, 3H), 2.63-2.54 (m, 1H), 2.44-2.35 (m, 1H), 2.17-2.06 (m, 2H), 2.00-1.92 (m, 1H), 1.91-1.83 (m, 1H), 1.72-1.63 (m, 2H), 1.61 (d, J=6.8 Hz, 3H), 1.54-1.42 (m, 2H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylthiane-4-carboxamide (Compound 1.5)

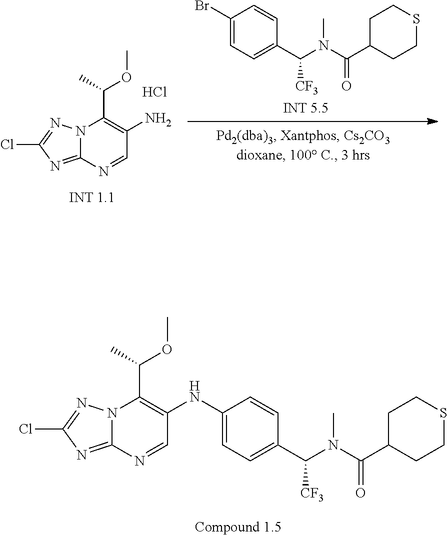 [SMILES: CO[C@@H](C)c1c(N)cnc2nc(Cl)nn12 ; Cl ; CN(C(=O)C1CCSCC1)[C@@H](c1ccc(Br)cc1)C(F)(F)F ; CO[C@@H](C)c1c(Nc2ccc([C@H](N(C)C(=O)C3CCSCC3)C(F)(F)F)cc2)cnc2nc(Cl)nn12]

A mixture of 2-chloro-7-[(1S)-1-methoxyethyl]-[1,2,4]triazolo[1,5-a]pyrimidin-6-amine hydrochloride [INT 1.1] (100 mg, 378 μmol), N-[(1S)-1-(4-bromophenyl)-2,2,2-trifluoroethyl]-N-methylthiane-4-carboxamide [INT 5.5] (149 mg, 378 μmol), Xantphos (43.7 mg, 75.6 μmol), Pd₂(dba)₃ (34.6 mg, 37.8 μmol), and Cs₂CO₃ (246 mg, 756 μmol) in dioxane (5 mL) was stirred at 100° C. for 3 hr under N₂ atmosphere. The reaction was concentrated under reduced pressure to afford the crude product, which was purified by flash chromatography on silica gel (EtOAc/Petroleum ether=1/10 to 1/1) and prep-HPLC (column: YMC Triart C18 250*50 mm*77 μm, table: 35-75% B (A=water (0.05% ammonia hydroxide v/v)), B=acetonitrile), flow rate: 60 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylthiane-4-carboxamide [Compound 1.5] (26.9 mg, 49.5 μmol, 13.1% yield) as a yellow dry powder. m/z: [M+H]+ Calcd for C23H27ClF3N6O2S 543.1; Found 543.4. ¹H NMR (400 MHz, CDCl₃) δ=8.89 (s, 1H), 7.31 (d, J=8.4 Hz, 2H), 7.11 (s, 1H), 7.04 (d, J=8.4 Hz, 2H), 6.62 (q, J=8.4 Hz, 1H), 5.48 (q, J=6.8 Hz, 1H), 3.49 (s, 3H), 2.90 (s, 3H), 2.81-2.70 (m, 4H), 2.70-2.62 (m, 1H), 2.17-2.11 (m, 1H), 2.08-1.99 (m, 3H), 1.61 (d, J=6.8 Hz, 3H).

### Synthesis of N-[(1S)-1-[4-({2-chloro-7-[(1S)-1-methoxyethyl]-[1,2,4]triazolo[1,5-a]pyrimidin-6-yl})amino)phenyl]-2,2,2-trifluoroethyl]-N-methyloxolane-3-carboxamide (Compound 1.6)

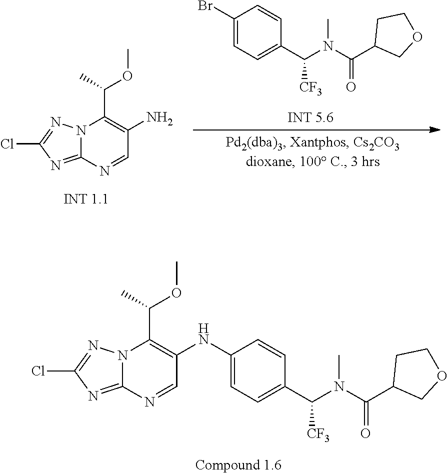 [SMILES: CO[C@@H](C)c1c(N)cnc2nc(Cl)nn12 ; CN(C(=O)C1CCOC1)[C@@H](c1ccc(Br)cc1)C(F)(F)F ; CO[C@@H](C)c1c(Nc2ccc([C@H](N(C)C(=O)C3CCOC3)C(F)(F)F)cc2)cnc2nc(Cl)nn12]

To a mixture of N-[(1S)-1-(4-bromophenyl)-2,2,2-trifluoroethyl]-N-methyloxolane-3-carboxamide [INT 5.6] (80 mg, 218 μmol) and 2-chloro-7-[(1S)-1-methoxyethyl]-[1,2,4]triazolo[1,5-a]pyrimidin-6-amine hydrochloride [INT 1.1] (68.9 mg, 261 μmol) in dioxane (2 mL) were added Pd₂(dba)₃ (19.9 mg, 21.8 μmol), Cs₂CO₃ (213 mg, 654 μmol), and xantphos (12.6 mg, 21.8 μmol). The reaction mixture was stirred at 100° C. under N₂ for 3 hours, after which it was diluted with brine (20 mL) and extracted with EtOAc (10 mL×3). The combined organic layers were dried over anhydrous sodium sulfate and filtered. The filtrate was concentrated to give crude product, which was purified by prep-HPLC (column: Boston Prime C18 150*25 mm*5 μm, table: 32-62% B (A=water (0.05% ammonia hydroxide), B=acetonitrile), flowrate: 25 mL/min, UV Detector 220 nm) to give N-[(1S)-1-[4-({2-chloro-7-[(1S)-1-methoxyethyl]-[1,2,4]triazolo[1,5-a]pyrimidin-6-yl})amino)phenyl]-2,2,2-trifluoroethyl]-N-methyloxolane-3-carboxamide [Compound 1.6] (2.20 mg, 4.28 μmol. 2.0% yield) as an off-white dry powder. m/z: [M+H]+ Calcd for C22H25ClF3N6O3 513.2; Found 513.3. ¹H NMR (400 MHz, CDCl₃) δ=8.90 (s, 1H), 7.37-7.31 (m, 2H), 7.12 (s, 1H), 7.05 (d, J=8.4 Hz, 2H), 6.62 (q, J=8.8 Hz, 1H), 5.49 (q, J=6.8 Hz, 1H), 4.18-4.01 (m, 1H), 4.00-3.86 (m, 3H), 3.49 (s, 3H), 3.40-3.28 (m, 1H), 2.97-2.82 (m, 3H), 2.32-2.09 (m, 2H), 1.62 (d, J=6.8 Hz, 3H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,4-dioxaspiro[4.5]decane-8-carboxamide (Compound 1.7)

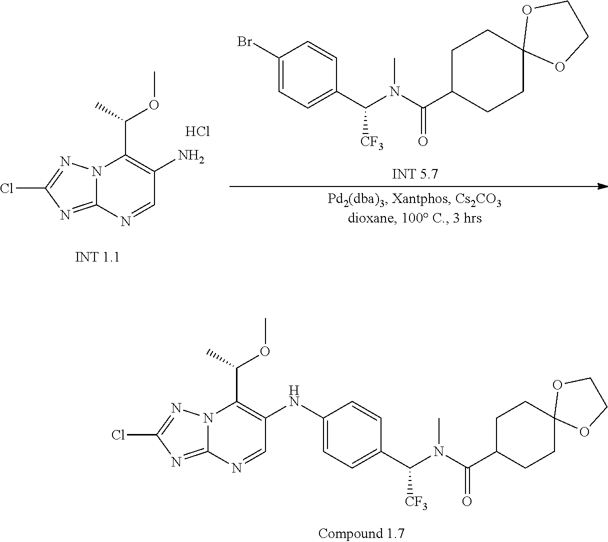 [SMILES: CO[C@@H](C)c1c(N)cnc2nc(Cl)nn12 ; Cl ; CN(C(=O)C1CCC2(CC1)OCCO2)[C@@H](c1ccc(Br)cc1)C(F)(F)F ; CO[C@@H](C)c1c(Nc2ccc([C@H](N(C)C(=O)C3CCC4(CC3)OCCO4)C(F)(F)F)cc2)cnc2nc(Cl)nn12]

A mixture of 2-chloro-7-[(1S)-1-methoxyethyl]-[1,2,4]triazolo[1,5-a]pyrimidin-6-amine hydrochloride [INT 1.1] (60.4 mg, 229 μmol), N-[(1S)-1-(4-bromophenyl)-2,2,2-trifluoroethyl]-N-methyl-1,4-dioxaspiro[4.5]decane-8-carboxamide [INT 5.7] (100 mg, 229 μmol), Xantphos (26.5 mg, 45.8 μmol). Pd₂(dba)₃ (20.9 mg, 22.9 μmol), and Cs₂CO₃ (149 mg, 458 μmol) in dioxane (5 mL) was stirred at 100° C. for 3 hr under N₂ atmosphere. The reaction was concentrated under reduced pressure to afford the crude product, which was purified by flash chromatography on silica gel (EtOAc/Petroleum ether=1/5 to 1/3) and prep-HPLC (column: YMC Triart C18 250*50 mm*7 μm, table: 33-73% B (A=water (0.05% ammonia hydroxide v/v)), B=acetonitrile), flow rate: 60 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,4-dioxaspiro[4.5]decane-8-carboxamide [Compound 1.7] (32.3 mg, 55.4 μmol, 24.2% yield) as a yellow dry powder. m/z: [M+H]+ Calcd for C26H31ClF3N6O4 583.2; Found 583.4. ¹H NMR (400 MHz, CDCl₃) δ=8.89 (s, 1H), 7.32 (d, J=8.4 Hz, 2H), 7.10 (s, 1H), 7.03 (d, J=8.4 Hz, 2H), 6.65 (q, J=8.8 Hz, 1H), 5.48 (q, J=6.8 Hz, 1H), 3.97 (s, 4H), 3.49 (s, 3H), 2.92 (s, 3H), 2.66-2.55 (m, 1H), 1.97-1.93 (m, 1H), 1.93-1.83 (m, 4H), 1.83-1.75 (m, 1H), 1.61 (d, J=6.8 Hz, 3H), 1.57-1.52 (m, 2H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4,4-difluoro-N-methylcyclohexane-1-carboxamide (Compound 1.8)

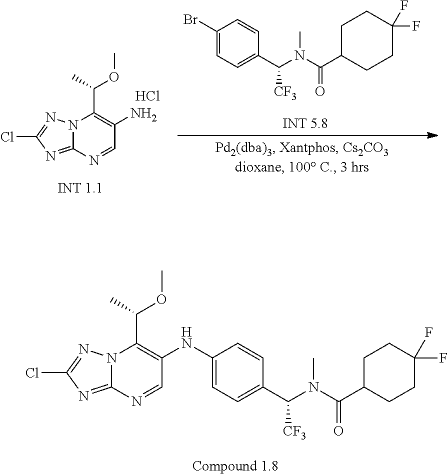 [SMILES: CO[C@@H](C)c1c(N)cnc2nc(Cl)nn12 ; Cl ; CN(C(=O)C1CCC(F)(F)CC1)[C@@H](c1ccc(Br)cc1)C(F)(F)F ; CO[C@@H](C)c1c(Nc2ccc([C@H](N(C)C(=O)C3CCC(F)(F)CC3)C(F)(F)F)cc2)cnc2nc(Cl)nn12]

A mixture of 2-chloro-7-[(1S)-1-methoxyethyl]-[1,2,4]triazolo[1,5-a]pyrimidin-6-amine hydrochloride [INT 1.1] (100 mg, 378 μmol), N-[(1S)-1-(4-bromophenyl)-2,2,2-trifluoroethyl]-4,4-difluoro-N-methylcyclohexane-1-carboxamide [INT 5.8] (156 mg, 378 μmol), Xantphos (43.7 mg, 75.6 μmol), Pd₂(dba)₃ (34.6 mg, 37.8 μmol), and Cs₂CO₃ (368 mg, 1.13 mmol) in dioxane (5 mL) was stirred at 100° C. for 3 hr under N₂ atmosphere. The reaction was filtered through celite. The filter cake was washed with EtOAc (20 mL×2). The filtrate was concentrated under reduced pressure to afford the crude product, which was purified by prep-HPLC (column: Welch Xtimate C18 150*25 mm*5 μm, table: 49-79% B (A=water (10 mM NH₄HCO₃), B=acetonitrile), flow rate: 25 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4,4-difluoro-N-methylcyclohexane-1-carboxamide [Compound 1.8] (61.5 mg, 109 μmol, 29.0% yield) as a yellow solid. m/z: [M+H]+ Calcd for C24H27ClF5N6O2 561.2; Found 561.2. ¹H NMR (400 MHz, DMSO-d6) δ=8.84 (s, 1H), 8.08-7.95 (m, 1H), 7.29-7.13 (m, 2H), 7.04-6.90 (m, 2H), 6.71-6.10 (m, 1H), 5.15 (q, J=6.8 Hz, 1H), 3.16 (s, 3H), 2.90 (s, 4H), 2.13-1.62 (m, 8H), 1.59 (d, J=6.8 Hz, 3H).

### Synthesis of (1r,3S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-3-cyano-N-methylcyclobutane-1-carboxamide (Compound 1.9)

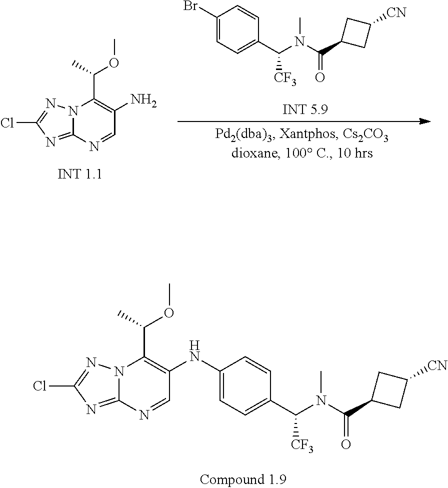 [SMILES: CO[C@@H](C)c1c(N)cnc2nc(Cl)nn12 ; CN(C(=O)[C@H]1C[C@H](C#N)C1)[C@@H](c1ccc(Br)cc1)C(F)(F)F ; CO[C@@H](C)c1c(Nc2ccc([C@H](N(C)C(=O)[C@H]3C[C@H](C#N)C3)C(F)(F)F)cc2)cnc2nc(Cl)nn12]

(1r,3S)—N—((S)-1-(4-bromophenyl)-2,2,2-trifluoroethyl)-3-cyano-N-methylcyclobutane-1-carboxamide [INT 5.9] (0.1 g, 0.2665 mmol), 2-chloro-7-[(1S)-1-methoxyethyl]-[1,2,4]triazolo[1,5-a]pyrimidin-6-amine [free base of INT 1.1] (60.5 mg, 266 μmol), xantphos (15.3 mg, 26.6 μmol), and Cs₂CO₃ (260 mg, 799 μmol) were mixed in dioxane (3 mL) and the reaction mixture was degassed with argon for 5 min. Pd₂(dba)₃ (12.1 mg, 13.3 μmol) was added, after which the reaction mixture was degassed with argon for 5 min and stirred at 100° C. for 10 h. The reaction mixture was cooled to rt and the solid was filtered off. The filtrate was purified by HPLC (see conditions below) to obtain (1r,3S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-3-cyano-N-methylcyclobutane-1-carboxamide [Compound 1.9] (14.9 mg, 0.02857 mmol, 10.7% yield) as a yellow oil. m/z: [M+H]+ Calcd for C23H24ClF3N7O2 522.2; Found 522.2. ¹H NMR (400 MHz, CD₃OD) δ 8.87 (d, J=1.7 Hz, 1H), 7.31 (d, J=8.4 Hz, 2H), 7.07 (d, J=8.4 Hz, 2H), 6.49 (q, J=9.1 Hz, 1H), 5.37 (q, J=6.7 Hz, 1H), 3.76 (h, J=7.3, 6.7 Hz, 1H), 3.37-3.24 (m, 2H), 2.82 (s, 3H), 2.77-2.58 (m, 7H), 1.65 (d, J=6.7 Hz, 3H).

HPLC Conditions; System: Agilent 1260 Infinity II LC coupled to an Agilent 6120B Single Quadrupole LC/MS System⋅Column Description: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water; Mobile phase B: acetonitrile; Flow rate: 30 ml/min; loading pump: 4 ml/min B; Gradient conditions: 20-40-55-100% (B) 0-2-10-11.2 min.

### Synthesis of tert-butyl 4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate (Compound 1.10)

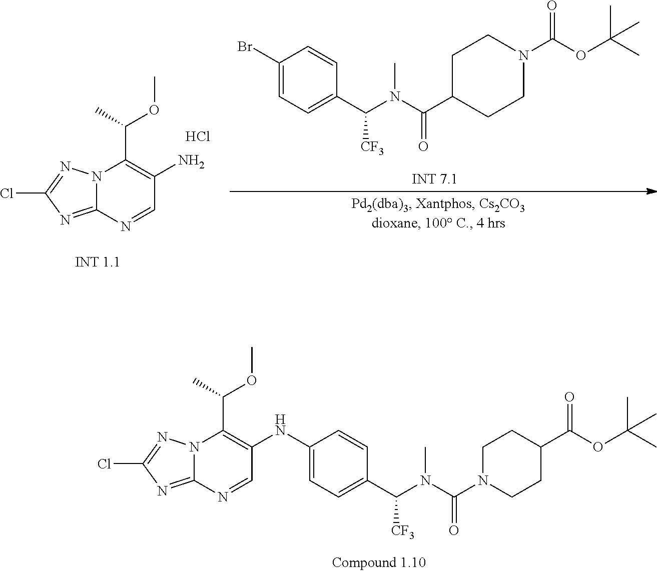 [SMILES: CO[C@@H](C)c1c(N)cnc2nc(Cl)nn12 ; Cl ; CN(C(=O)C1CCN(C(=O)OC(C)(C)C)CC1)[C@@H](c1ccc(Br)cc1)C(F)(F)F ; CO[C@@H](C)c1c(Nc2ccc([C@H](N(C)C(=O)N3CCC(C(=O)OC(C)(C)C)CC3)C(F)(F)F)cc2)cnc2nc(Cl)nn12]

A mixture of tert-butyl 4-{[(1S)-1-(4-bromophenyl)-2,2,2-trifluoroethyl](methyl)carbamoyl}piperidine-1-carboxylate [INT 7.1] (150 mg, 312 μmol), Pd₂(dba)₃ (28.5 mg, 31.2 μmol), Cs₂CO₃ (304 mg, 936 μmol), 2-chloro-7-[(1S)-1-methoxyethyl]-[1,2,4]triazolo[1,5-a]pyrimidin-6-amine hydrochloride [INT 1.1] (82.4 mg, 312 μmol), and xantphos (18.0 mg, 31.2 μmol) in dioxane was stirred at 100° C. for 4 hr under N₂ atmosphere. Brine (20 mL) was added, and the mixture was extracted with EtOAc (30 mL×2). The combined organic layers were washed with brine (20 mL×2), dried over anhydrous Na₂SO₄, filtered, and concentrated under reduced pressure to give the crude product. The crude product was purified by prep-HPLC (column: Boston Prime C18 150*30 mm*5 μm, table: 49-79% B (A=water (0.05% ammonia hydroxide v/v)-ACN), B=acetonitrile), flowrate: 30 mL/min, UV Detector 220 nm) to afford tert-butyl 4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate [Compound 1.10] (9.10 mg, 14.5 μmol, 4.7% yield) as a yellow dry powder. m/z: [M+H−100]+ Calcd for C28H36ClF3N7O4 526.2; Found 526.3. ¹H NMR (400 MHz, CDCl₃) δ=8.90 (s, 1H), 7.32 (d, J=8.8 Hz, 2H), 7.19-7.08 (m, 1H), 7.04 (d, J=8.8 Hz, 2H), 6.63 (q, J=8.8 Hz, 1H), 5.48 (q, J=6.8 Hz, 1H), 4.19 (br d, J=13.2 Hz, 2H), 3.49 (s, 3H), 3.00-2.91 (m, 3H), 2.85-2.69 (m, 3H), 1.82-1.68 (m, 4H), 1.62 (s, 3H), 1.47 (s, 9H).

### Synthesis of Tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate (Compound 1.11)

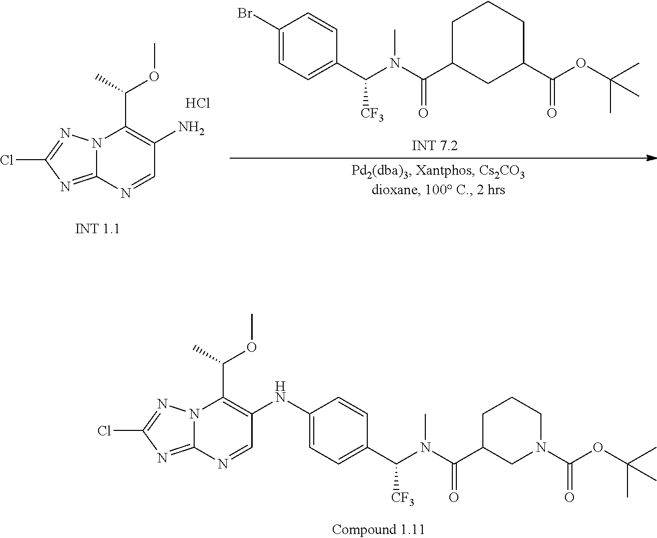 [SMILES: CO[C@@H](C)c1c(N)cnc2nc(Cl)nn12 ; Cl ; CN(C(=O)C1CCCC(C(=O)OC(C)(C)C)C1)[C@@H](c1ccc(Br)cc1)C(F)(F)F ; CO[C@@H](C)c1c(Nc2ccc([C@H](N(C)C(=O)C3CCCN(C(=O)OC(C)(C)C)C3)C(F)(F)F)cc2)cnc2nc(Cl)nn12]

To a solution of tert-butyl 3-{[(1S)-1-(4-bromophenyl)-2,2,2-trifluoroethyl](methyl)carbamoyl}piperidine-1-carboxylate [INT 7.2] (700 mg, 1.46 mmol), 2-chloro-7-[(1S)-1-methoxyethyl]-[1,2,4]triazolo[1,5-a]pyrimidin-6-amine hydrochloride [INT 1.1] (332 mg, 1.46 mmol), Cs₂CO₃ (1.42 g, 4.38 mmol), and xantphos (168 mg, 292 μmol) in dioxane (5 mL) was added Pd₂(dba)₃ (133 mg, 146 μmol) and the reaction mixture was stirred at 100° C. for 2 h. The reaction mixture was concentrated under reduced pressure to give the crude product, which was purified by flash chromatography on silica gel (EtOAc/PE=0/1 to 1/1) to give tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate [Compound 1.11] (480 mg, 766 μmol, 52.5% yield) as a yellow oil. 60.6 mg of this product were purified by prep-HPLC (column: YMC Triart C18 250*50 mm*7 μm, table: 47-87% B (A=water (water (0.225% FA), B=acetonitrile), flow rate: 60 mL/min, UV Detector 220 nm) to afford the tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate [Compound 1.11] (10.6 mg, 16.9 μmol) as an off-white solid. m/z: [M+H]+ Calcd for C28H36ClF3N7O4 626.2; Found 626.3. ¹H NMR (400 MHz, DMSO-d6) δ=8.84 (s, 1H), 8.07-7.92 (m, 1H), 7.31-7.14 (m, 2H), 6.97 (br d, J=8.4 Hz, 2H), 6.43 (q, J=9.6 Hz, 1H), 5.16 (q, J=6.8 Hz, 1H), 4.08-3.84 (m, 2H), 3.17 (s, 3H), 2.91 (s, 3H), 2.77 (br s, 2H), 2.68 (br s, 1H), 1.82 (br d, J=12.4 Hz, 1H), 1.69-1.54 (m, 5H), 1.40 (s, 10H).

### Synthesis of Tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}pyrrolidine-1-carboxylate (Compound 1.12)

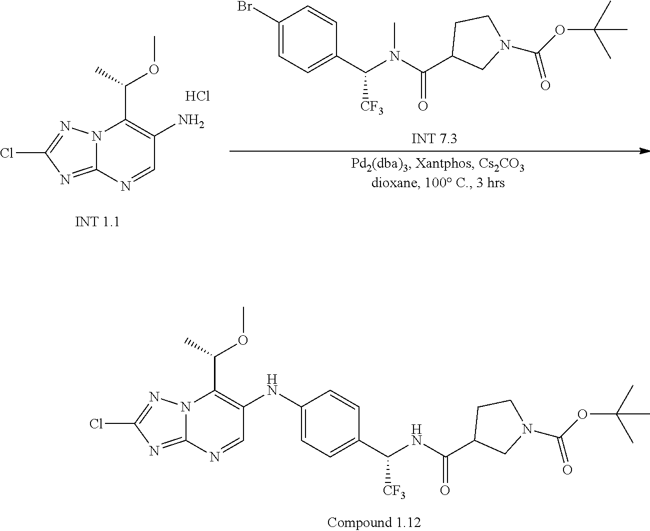 [SMILES: CO[C@@H](C)c1c(N)cnc2nc(Cl)nn12 ; Cl ; CN(C(=O)C1CCN(C(=O)OC(C)(C)C)C1)[C@@H](c1ccc(Br)cc1)C(F)(F)F ; CO[C@@H](C)c1c(Nc2ccc([C@H](NC(=O)C3CCN(C(=O)OC(C)(C)C)C3)C(F)(F)F)cc2)cnc2nc(Cl)nn12]

To a mixture of tert-butyl 3-{[(1S)-1-(4-bromophenyl)-2,2,2-trifluoroethyl](methyl)carbamoyl}pyrrolidine-1-carboxylate [INT 7.3] (140 mg, 300 μmol), 2-chloro-7-[(1S)-1-methoxyethyl]-[1,2,4]triazolo[1,5-a]pyrimidin-6-amine hydrochloride [INT 1.1] (95.0 mg, 360 μmol), xantphos (17.3 mg, 30.0 μmol), and Cs₂CO₃ (390 mg, 1.20 mmol) in dioxane (2 mL) was added Pd₂(dba)₃ (27.4 mg, 30.0 μmol) and the mixture was stirred at 100° C. for 3 hr under N₂. The reaction mixture was diluted with brine (20 mL) and extracted with EtOAc (20 mL×3). The combined organic layers were dried over anhydrous sodium sulfate and filtered. The filtrate was concentrated to give crude product, which was purified by flash chromatography on silica gel (methanol/dichloromethane=1/20). The product was then purified by prep-HPLC (column: Boston Prime C18 150*30 mm*5 μm, table: 44-74% B (A=water (0.05% ammonia hydroxide v/v)-ACN), B=acetonitrile), flowrate: 25 mL/min, UV Detector 220 nm) to afford tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}pyrrolidine-1-carboxylate [Compound 1.12] (2.70 mg, 4.41 μmol, 1.5% yield) as a yellow dry powder. m/z: [M+H]+ Calcd for C27H34ClF3N7O4 612.2; Found 612.4. ¹H NMR (400 MHz, CDCl₃) δ=8.90 (d, J=3.2 Hz, 1H), 7.32 (br d, J=8.0 Hz, 2H), 7.13 (s, 1H), 7.04 (br d, J=8.4 Hz, 2H), 6.66-6.55 (m, 1H), 5.48 (q, J=6.8 Hz, 1H), 3.84-3.51 (m, 3H), 3.49 (s, 3H), 3.45-3.25 (m, 2H), 2.97-2.78 (m, 3H), 2.36-2.05 (m, 2H), 1.62 (d, J=6.8 Hz, 3H), 1.49-1.45 (m, 9H).

### Synthesis of tert-butyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate (Compound 1.13)

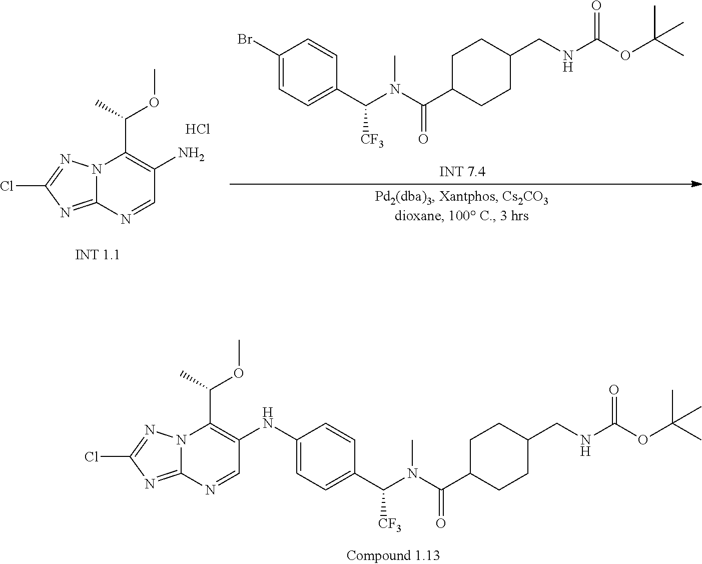 [SMILES: CO[C@@H](C)c1c(N)cnc2nc(Cl)nn12 ; Cl ; CN(C(=O)C1CCC(CNC(=O)OC(C)(C)C)CC1)[C@@H](c1ccc(Br)cc1)C(F)(F)F ; CO[C@@H](C)c1c(Nc2ccc([C@H](N(C)C(=O)C3CCC(CNC(=O)OC(C)(C)C)CC3)C(F)(F)F)cc2)cnc2nc(Cl)nn12]

To a suspension of tert-butyl N-[(4-{[(1S)-1-(4-bromophenyl)-2,2,2-trifluoroethyl](methyl)carbamoyl}cyclohexyl)methyl]carbamate [INT 7.4] (420 mg, 827 μmol), 2-chloro-7-[(1S)-1-methoxyethyl]-[1,2,4]triazolo[1,5-a]pyrimidin-6-amine hydrochloride [INT 1.1] (282 mg, 1.07 mmol), Cs₂CO₃ (537 mg, 1.65 mmol), and xantphos (95.4 mg, 165 μmol) in dioxane (3 mL) was added Pd₂(dba)₃ (75.7 mg, 82.7 μmol). The resulting mixture was stirred at 100° C. for 3 h under N₂. The reaction mixture was concentrated under reduced pressure to give a residue, which was purified by flash chromatography on silica gel (MeOH/dichloromethane=0/1 to 1/99) to give tert-butyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate [Compound 1.13] (500 mg, 765 μmol, 92.5% yield) as a yellow solid. 100 mg of the product was further purified by prep-HPLC (column: Phenomenex Gemini-NX 80*40 mm*3 μm, table: 39-79% B (A=water (0.05% ammonia hydroxide v/v), B=MeOH), flow rate: 25 mL/min, UV Detector 220 nm) to afford tert-butyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate [Compound 1.13] (500 mg, 39.1 μmol) as a yellow dry powder. m/z: [M+H]+ Calcd for C30H40ClF3N7O4 654.3; Found 654.6. ¹H NMR (400 MHz, DMSO-d6) δ=8.85 (s, 1H), 8.05-7.97 (m, 1H), 7.26-7.10 (m, 2H), 7.04-6.92 (m, 2H), 6.82 (br t, J=5.6 Hz, 1H), 6.51-6.07 (m, 1H), 5.16 (q, J=6.8 Hz, 1H), 3.16 (s, 3H), 2.87 (s, 3H), 2.78 (br t, J=6.4 Hz, 2H), 2.70-2.56 (m, 1H), 1.83-1.64 (m, 4H), 1.59 (d, J=6.8 Hz, 3H), 1.37 (s, 12H), 1.04-0.83 (m, 2H).

### Synthesis of (1r,4S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-4-methoxy-N-methylcyclohexane-1-carboxamide (Compound 1.14)

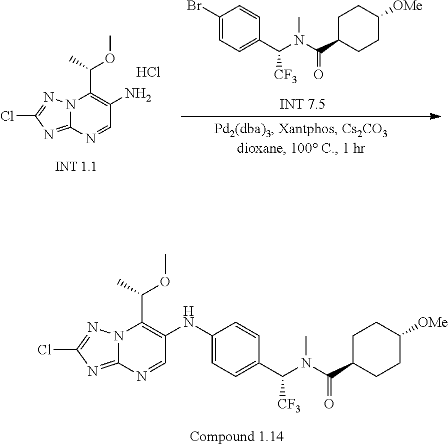 [SMILES: CO[C@@H](C)c1c(N)cnc2nc(Cl)nn12 ; Cl ; CO[C@H]1CC[C@H](C(=O)N(C)[C@@H](c2ccc(Br)cc2)C(F)(F)F)CC1 ; CO[C@@H](C)c1c(Nc2ccc([C@H](N(C)C(=O)[C@H]3CC[C@H](OC)CC3)C(F)(F)F)cc2)cnc2nc(Cl)nn12]

To a mixture of (1r,4S)—N—((S)-1-(4-bromophenyl)-2,2,2-trifluoroethyl)-4-methoxy-N-methylcyclohexane-1-carboxamide [INT 7.5] (90 mg, 220 μmol), 2-chloro-7-[(1S)-1-methoxyethyl]-[1,2,4]triazolo[1,5-a]pyrimidin-6-amine hydrochloride [INT 1.1] (50.0 mg, 220 μmol), Cs₂CO₃ (143 mg, 440 μmol), and Xantphos (25.4 mg, 44.0 μmol) in dioxane (1 mL) was added Pd₂(dba)₃ (20.1 mg, 22.0 μmol) under N₂ and the reaction mixture was stirred at 100° C. for 1 h. The reaction was quenched by adding water (10 mL) and was extracted with EtOAc (2×10 mL). The combined organic layers were washed with brine (2×10 mL), dried over anhydrous Na₂SO₄, and concentrated under reduced pressure to give the crude product, which was purified by prep-HPLC (column: YMC-Actus Triart C18 150*30 mm*5 μm, table: 48-68% water (0.05% ammonia hydroxide v/v)-ACN, flow rate: 35 mL/min, UV Detector 220 nm) to afford (1r,4S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-4-methoxy-N-methylcyclohexane-1-carboxamide [Compound 1.14] (8.60 mg, 15.4 μmol, 7.0% yield) as a yellow dry powder. m/z: [M+H]+ Calcd for C25H31ClF3N6O3 555.2; Found 555.3. ¹H NMR (400 MHz, DMSO-d6) δ=8.84 (s, 1H), 7.98 (s, 1H), 7.16 (br d, J=8.4 Hz, 2H), 6.96 (d, J=8.7 Hz, 2H), 6.50-6.09 (m, 1H), 5.15 (q, J=6.7 Hz, 1H), 3.23 (s, 3H), 3.16 (s, 3H), 3.10 (br s, 1H), 2.91-2.59 (m, 4H), 2.11-1.93 (m, 2H), 1.84-1.66 (m, 2H), 1.59 (d, J=6.7 Hz, 3H), 1.49-1.32 (m, 2H), 1.25-1.08 (m, 2H).

### Synthesis of 1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylazetidine-3-carboxamide (Compound 1.15)

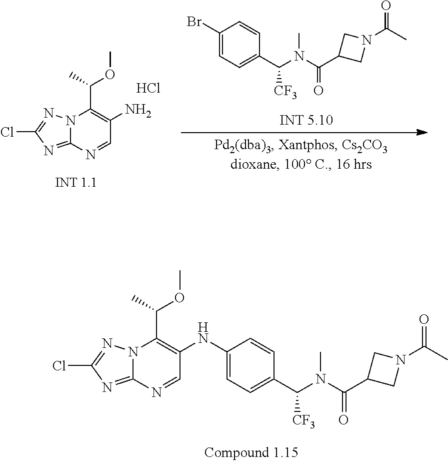 [SMILES: CO[C@@H](C)c1c(N)cnc2nc(Cl)nn12 ; Cl ; CC(=O)N1CC(C(=O)N(C)[C@@H](c2ccc(Br)cc2)C(F)(F)F)C1 ; CO[C@@H](C)c1c(Nc2ccc([C@H](N(C)C(=O)C3CN(C(C)=O)C3)C(F)(F)F)cc2)cnc2nc(Cl)nn12]

To a solution of 1-acetyl-N-[(1S)-1-(4-bromophenyl)-2,2,2-trifluoroethyl]-N-methylazetidine-3-carboxamide [INT 5.10] (100 mg, 254 μmol), 2-chloro-7-[(1S)-1-methoxyethyl]-[1,2,4]triazolo[1,5-a]pyrimidin-6-amine hydrochloride [INT 1.1] (73.6 mg, 279 μmol), Cs₂CO₃ (248 mg, 762 μmol), and xantphos (29.3 mg, 50.8 μmol) in dioxane (3 mL) was added Pd₂(dba)₃ (23.2 mg, 25.4 μmol) and the reaction mixture was stirred at 100° C. for 16 h under N₂. The reaction mixture was concentrated under reduced pressure to give the crude product, which was purified by prep-HPLC (column: YMC Triart C18 250*50 mm*7 μm, table: 24-64% B (A=water (0.05% ammonia hydroxide)), B=acetonitrile), flow rate: 60 mL/min, UV Detector 220 nm) to afford 1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylazetidine-3-carboxamide [Compound 1.15] (21.2 mg, 39.2 μmol, 15.4% yield) as a yellow dry powder. m/z: [M+H]+ Calcd for C23H26ClF3N7O3 540.2; Found 540.2. ¹H NMR (400 MHz, DMSO-d6) δ=8.85 (s, 1H), 8.02 (s, 1H), 7.33-7.15 (m, 2H), 6.97 (br d, J=8.4 Hz, 2H), 6.42 (q, J=9.2 Hz, 1H), 5.16 (q, J=6.8 Hz, 1H), 4.38-4.17 (m, 2H), 4.10-3.99 (m, 1H), 3.92-3.81 (m, 2H), 3.17 (s, 3H), 2.76-2.69 (m, 3H), 1.76 (d, J=2.8 Hz, 3H), 1.62-1.58 (m, 3H).

### Synthesis of N-(4-{[(1S)-1-[4-({2-chloro-7-[(1S)-1-methoxyethyl]-[1,2,4]triazolo[1,5-a]pyrimidin6-yl}amino)phenyl]-2,2,2-trifluoroethyl](methyl)carbamoyl}cyclohexyl)carbamate (Also Known as methyl (4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexyl)carbamate) (Compound 1.16)

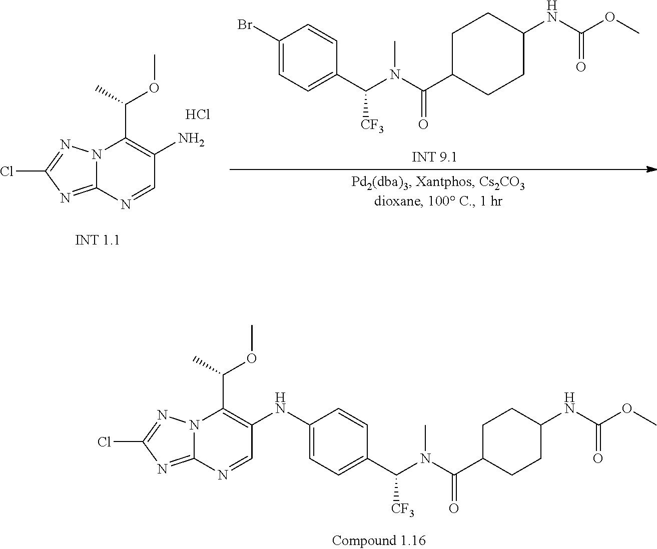 [SMILES: CO[C@@H](C)c1c(N)cnc2nc(Cl)nn12 ; Cl ; COC(=O)NC1CCC(C(=O)N(C)[C@@H](c2ccc(Br)cc2)C(F)(F)F)CC1 ; COC(=O)NC1CCC(C(=O)N(C)[C@@H](c2ccc(Nc3cnc4nc(Cl)nn4c3[C@H](C)OC)cc2)C(F)(F)F)CC1]

To a mixture of methyl N-(4-{[(1S)-1-(4-bromophenyl)-2,2,2-trifluoroethyl](methyl)carbamoyl}cyclohexyl)carbamate [INT 9.1] (40 mg, 88.6 μmol), 2-chloro-7-[(1S)-1-methoxyethyl]-[1,2,4]triazolo[1,5-a]pyrimidin-6-amine hydrochloride [INT 1.1] (20.1 mg, 88.6 μmol), Cs₂CO₃ (57.6 mg, 177 μmol), and Xantphos (10.2 mg, 17.7 μmol) in dioxane (1 mL) was added Pd₂(dba)₃ (8.11 mg, 8.86 μmol) at 20° C. and the mixture was stirred at 100° C. under N₂ for 1 h. The reaction mixture was cooled to 25° C. and combined with another batch (44.3 μmol scale of INT 9.1) of the same reaction. Water (10 mL) was added and the mixture was extracted with EtOAc (2×10 mL). The combined organic layers were washed with brine (2×10 mL), dried over anhydrous Na₂SO₄ and concentrated under reduced pressure to give the crude product, which was purified by prep-HPLC (column: YMC-Actus Triart C18 150*30 mm*5 μm, table: 46-66% water (0.05% ammonia hydroxide v/v)-ACN, flow rate: 35 mL/min, UV Detector 220 nm) to afford methyl N-(4-{[(1S)-1-[4-({2-chloro-7-[(1S)-1-methoxyethyl]-[1,2,4]triazolo[1,5-a]pyrimidin6-yl}amino)phenyl]-2,2,2-trifluoroethyl](methyl)carbamoyl}cyclohexyl)carbamate [Compound 1.16] (12.2 mg, 20.4 μmol, 15.3% yield) as a yellow dry powder. m/z: [M+H]+ Calcd for C26H32ClF3N7O4 598.2; Found 598.3. ¹H NMR (400 MHz, DMSO-d6) δ=8.83 (s, 1H), 7.98 (s, 1H), 7.16 (br d, J=8.4 Hz, 2H), 6.96 (d, J=8.7 Hz, 3H), 6.55-6.07 (m, 1H), 5.15 (q, J=6.7 Hz, 1H), 3.50 (s, 3H), 3.28-3.20 (m, 1H), 3.16 (s, 3H), 2.87 (s, 3H), 2.61 (br s, 1H), 1.90-1.66 (m, 4H), 1.59 (d, J=6.7 Hz, 3H), 1.51-1.32 (m, 2H), 1.31-1.13 (m, 2H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4-acetamido-N-methylcyclohexane1-carboxamide (Compound 1.17)

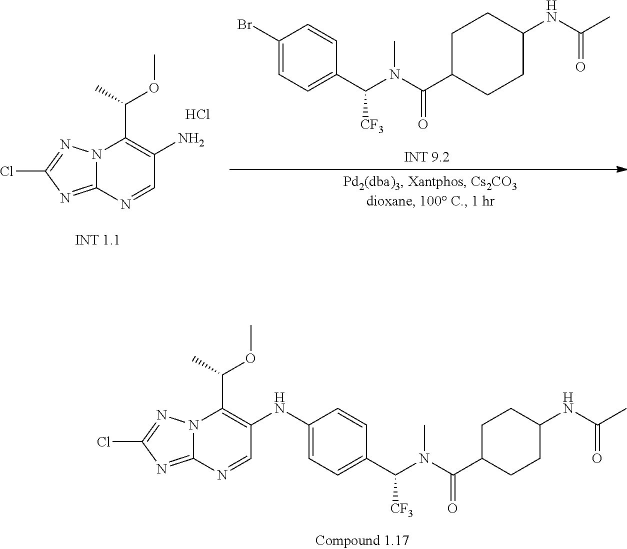 [SMILES: CO[C@@H](C)c1c(N)cnc2nc(Cl)nn12 ; Cl ; CC(=O)NC1CCC(C(=O)N(C)[C@@H](c2ccc(Br)cc2)C(F)(F)F)CC1 ; CO[C@@H](C)c1c(Nc2ccc([C@H](N(C)C(=O)C3CCC(NC(C)=O)CC3)C(F)(F)F)cc2)cnc2nc(Cl)nn12]

To a mixture of N-[(1S)-1-(4-bromophenyl)-2,2,2-trifluoroethyl]-4-acetamido-N-methylcyclohexane-1-carboxamide [INT 9.2] (20 mg, 45.9 μmol), 2-chloro-7-[(1S)-1-methoxyethyl]-[1,2,4]triazolo[1,5-a]pyrimidin-6-amine hydrochloride [INT 1.1] (10.4 mg, 45.9 μmol), Cs₂CO₃ (29.9 mg, 91.8 μmol), and Xantphos (5.31 mg, 9.18 μmol) in dioxane (2 mL) was added Pd₂(dba)₃ (4.20 mg, 4.59 μmol) at 20° C. and the mixture was stirred at 100° C. under N₂ for 1 h. The reaction was combined with the another batch of the same reaction (22.9 μmol scale) and concentrated under reduced pressure to give a crude product, which was purified by prep-HPLC (column: YMCActus Triart C18 150*30 mm*5 μm, table: 42-62% water (0.05% ammonia hydroxide v/v)-ACN, flow rate: 35 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4-acetamido-N-methylcyclohexane1-carboxamide [Compound 1.17] (8.50 mg, 14.6 μmol, 21.2% yield) as a yellow dry powder. m/z: [M+H]+ Calcd for C26H32ClF3N7O3 582.2; Found 582.2. ¹H NMR (400 MHz, DMSO-d6) δ=8.83 (s, 1H), 7.98 (s, 1H), 7.72 (d, J=7.6 Hz, 1H), 7.17 (br d, J=8.4 Hz, 2H), 6.96 (d, J=8.7 Hz, 2H), 6.45 (br d, J=9.5 Hz, 1H), 5.15 (q, J=6.7 Hz, 1H), 3.46 (br s, 1H), 3.16 (s, 3H), 2.88 (s, 3H), 2.63 (br d, J=7.2 Hz, 1H), 1.91-1.66 (m, 7H), 1.59 (d, J=6.6 Hz, 3H), 1.51-1.31 (m, 2H), 1.29-1.11 (m, 2H).

### Synthesis of N-[(1S)-1-(4-{[2-chloro-7-(propan-2-yl)-[1,2,4]triazolo[1,5-a]pyrimidin-6-yl]amino}phenyl)-2,2,2-trifluoroethyl]-N-methyl-1,1-dioxo-1λ⁶-thiane-4-carboxamide (Also Known as (S)—N-(1-(4-((2-chloro-7-isopropyl-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide) (Compound 1.18)

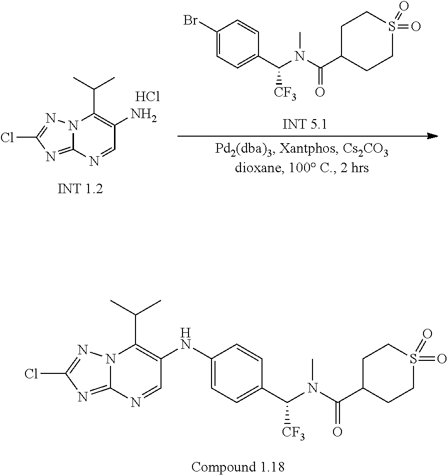 [SMILES: CC(C)c1c(N)cnc2nc(Cl)nn12 ; Cl ; CN(C(=O)C1CCS(=O)(=O)CC1)[C@@H](c1ccc(Br)cc1)C(F)(F)F ; CC(C)c1c(Nc2ccc([C@H](N(C)C(=O)C3CCS(=O)(=O)CC3)C(F)(F)F)cc2)cnc2nc(Cl)nn12]

To a solution of 2-chloro-7-(propan-2-yl)-[1,2,4]triazolo[1,5-a]pyrimidin-6-amine hydrochloride [INT 1.2] (30 mg, 120 μmol), N-[(1S)-1-(4-bromophenyl)-2,2,2-trifluoroethyl]-N-methyl-1,1-dioxo-1λ⁶-thiane-4-carboxamide [INT 5.1] (51.3 mg, 120 μmol), xantphos (13.8 mg, 24.0 μmol), and Cs₂CO₃ (117 mg, 360 μmol) in dioxane (5 mL) was added Pd₂(dba)₃ (10.9 mg, 12.0 μmol). The reaction was stirred at 100° C. for 2 h under N₂. The reaction was combined with another of the same reaction (120 μmol scale), was quenched by adding water (30 mL), and was extracted with EtOAc (30 mL). The combined organic layers were dried over anhydrous Na₂SO₄ and concentrated under reduced pressure to give the crude product, which was purified by prep-HPLC (column: YMC-Actus Triart C18 150*30 mm*5 μm, table:40-60% B (A=water (0.05% ammonia hydroxide), B=ACN), flow rate: 35 mL/min, UV Detector 220 nm) to afford N-[(1S)-1-(4-{[2-chloro-7-(propan-2-yl)-[1,2,4]triazolo[1,5-a]pyrimidin-6-yl]amino}phenyl)-2,2,2-trifluoroethyl]-N-methyl-1,1-dioxo-1λ⁶-thiane-4-carboxamide [Compound 1.18] (22.1 mg, 39.5 μmol, 16.5% yield) as a white dry powder. m/z: [M+H]+ Calcd for C23H27ClF3N6O3S 559.1; Found 559.1. ¹H NMR (400 MHz, DMSO-d6) δ=8.77-8.66 (m, 1H), 8.20-8.10 (m, 1H), 7.26-7.10 (m, 2H), 6.86-6.74 (m, 2H), 6.46-6.04 (m, 1H), 3.71 (q, J=7.2 Hz, 1H), 3.26-3.05 (m, 5H), 2.94-2.61 (m, 3H), 2.08-1.90 (m, 4H), 1.42 (d, J=7.2 Hz, 6H).

### Synthesis of 1-acetyl-N-[(1S)-1-(4-{[2-chloro-7-(propan-2-yl)-[1,2,4]triazolo[1,5-a]pyrimidin-6-yl]amino}phenyl)-2,2,2-trifluoroethyl]-N-methylpiperidine-4-carboxamide (Compound 1.19)

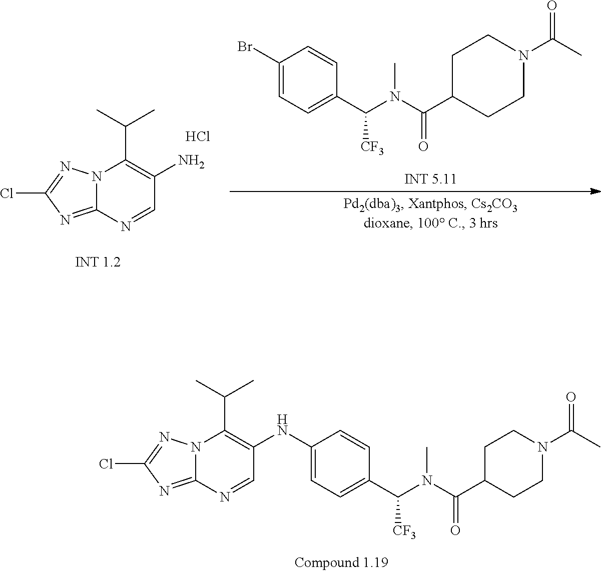 [SMILES: CC(C)c1c(N)cnc2nc(Cl)nn12 ; Cl ; CC(=O)N1CCC(C(=O)N(C)[C@@H](c2ccc(Br)cc2)C(F)(F)F)CC1 ; CC(=O)N1CCC(C(=O)N(C)[C@@H](c2ccc(Nc3cnc4nc(Cl)nn4c3C(C)C)cc2)C(F)(F)F)CC1]

A mixture of 2-chloro-7-(propan-2-yl)-[1,2,4]triazolo[1,5-a]pyrimidin-6-amine hydrochloride [INT 1.2] (50 mg, 201 μmol), 1-acetyl-N-[(1S)-1-(4-bromophenyl)-2,2,2-trifluoroethyl]-N-methylpiperidine-4-carboxamide [INT 5.11] (93.0 mg, 221 μmol), xantphos (11.6 mg, 20.1 μmol), Pd₂(dba)₃ (18.4 mg, 20.1 μmol), and Cs₂CO₃ (196 mg, 602 μmol) in dioxane (1 mL) was stirred at 100° C. for 3 hr under N₂ atmosphere. The mixture was concentrated under reduced pressure to afford the crude product, which was purified by prep-HPLC (column: Boston Prime C18 150*30 mm*5 μm, table: 25-65% B (A=water (0.05% ammonia hydroxide v/v)-ACN), B=acetonitrile), flowrate: 25 mL/min, UV Detector 220 nm) to afford 1-acetyl-N-[(1S)-1-(4-{[2-chloro-7-(propan-2-yl)-[1,2,4]triazolo[1,5-a]pyrimidin-6-yl]amino}phenyl)-2,2,2-trifluoroethyl]-N-methylpiperidine-4-carboxamide [Compound 1.19] (19.1 mg, 34.6 μmol, 17.3% yield) as a white dry powder. m/z: [M+H]+ Calcd for C25H30ClF3N7O2 552.2; Found 552.2. ¹H NMR (400 MHz, CDCl₃) δ=8.68 (s, 1H), 7.24 (br t, J=7.6 Hz, 2H), 6.66 (d, J=8.4 Hz, 2H), 6.57 (q, J=9.2 Hz, 1H), 5.47 (br s, 1H), 4.61 (br d, J=13.6 Hz, 1H), 3.97-3.86 (m, 2H), 3.22-3.08 (m, 1H), 2.92 (s, 3H), 2.85-2.62 (m, 2H), 2.11 (d, J=2.4 Hz, 3H), 1.91-1.81 (m, 2H), 1.80-1.69 (m, 2H), 1.54 (s, 3H), 1.53 (s, 3H).

### Synthesis of N-{1-[4-({2-chloro-7-[(1S)-1-methoxyethyl]-[1,2,4]triazolo[1,5-a]pyrimidin-6-yl}amino)phenyl]ethyl}-N-methylcyclobutanecarboxamide (Compound 1.20)

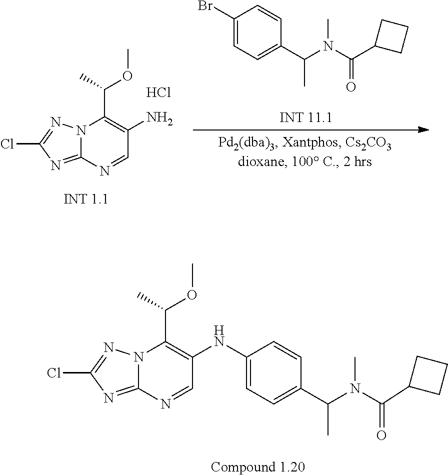 [SMILES: CO[C@@H](C)c1c(N)cnc2nc(Cl)nn12 ; Cl ; CC(c1ccc(Br)cc1)N(C)C(=O)C1CCC1 ; CO[C@@H](C)c1c(Nc2ccc(C(C)N(C)C(=O)C3CCC3)cc2)cnc2nc(Cl)nn12]

To a solution of N-[1-(4-bromophenyl)ethyl]-N-methylcyclobutanecarboxamide [INT 11.1] (60 mg, 202 μmol), 2-chloro-7-[(1S)-1-methoxyethyl]-[1,2,4]triazolo[1,5-a]pyrimidin-6-amine hydrochloride [INT 1.1] (45.9 mg, 202 μmol), Cs₂CO₃ (197 mg, 606 μmol), and xantphos (23.3 mg, 40.4 μmol) in dioxane (2 mL) was added Pd₂(dba)₃ (18.4 mg, 20.2 μmol) and the reaction mixture was stirred at 100° C. for 2 h under N₂. The mixture was concentrated in vacuo to give the crude product, which was purified by prep-HPLC (column: Phenomenex Gemini-NX 80*40 mm*3 μm, table: 20-60% B (A=water (0.05% ammonia hydroxide v/v), B=acetonitrile), flow rate: 25 mL/min, UV Detector 220 nm) to afford N-{1-[4-({2-chloro-7-[(1S)-1-methoxyethyl]-[1,2,4]triazolo[1,5-a]pyrimidin-6-yl}amino)phenyl]ethyl}-N-methylcyclobutanecarboxamide [Compound 1.20] (5.60 mg, 12.6 μmol, 6.3% yield) as a yellow dry power. m/z: [M+H]+ Calcd for C22H28ClN6O2 443.2; Found 443.3. ¹H NMR (400 MHz, DMSO-d6) δ=8.80 (s, 1H), 7.81-7.69 (m, 1H), 7.11-7.05 (m, 2H), 6.94-6.89 (m, 2H), 5.73 (q, J=6.8 Hz, 0.7H), 5.17 (q, J=6.8 Hz, 1H), 4.92 (q, J=6.4 Hz, 0.3H), 3.53-3.35 (m, 1H), 3.17 (s, 3H), 2.52 (s, 2H), 2.48 (s, 1H), 2.34-2.07 (m, 4H), 1.97-1.85 (m, 1H), 1.80-1.71 (m, 1H), 1.58 (d, J=6.8 Hz, 3H), 1.46-1.32 (m, 3H).

### Synthesis of N-{1-[4-({2-chloro-7-[(1S)-1-methoxyethyl]-[1,2,4]triazolo[1,5-a]pyrimidin-6-yl}amino)phenyl]ethyl}-N-methylcyclohexanecarboxamide (Compound 1.21)

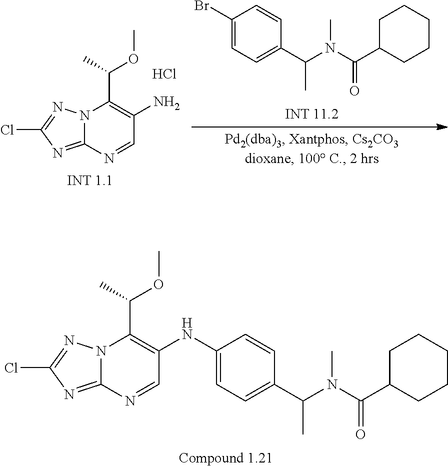 [SMILES: CO[C@@H](C)c1c(N)cnc2nc(Cl)nn12 ; Cl ; CC(c1ccc(Br)cc1)N(C)C(=O)C1CCCCC1 ; CO[C@@H](C)c1c(Nc2ccc(C(C)N(C)C(=O)C3CCCCC3)cc2)cnc2nc(Cl)nn12]

To a solution of N-[1-(4-bromophenyl)ethyl]-N-methylcyclohexanecarboxamide [INT 11.2] (50 mg, 154 μmol), 2-chloro-7-[(1S)-1-methoxyethyl]-[1,2,4]triazolo[1,5-a]pyrimidin-6-amine hydrochloride [INT 1.1] (35.0 mg, 154 μmol), Cs₂CO₃ (150 mg, 462 μmol), and xantphos (17.8 mg, 30.8 μmol) in dioxane (3 mL) was added Pd₂(dba)₃ (14.1 mg, 15.4 μmol) and the reaction mixture was stirred at 100° C. for 2 h under N₂. The mixture was concentrated in vacuo to give the crude product, which was purified by prep-HPLC (column: Phenomenex Gemini-NX C18 75*30 mm*3 μm, table: 35-75% B (A=water (0.05% ammonia hydroxide v/v), B=acetonitrile), flow rate: 25 mL/min, UV Detector 220 nm) to afford N-{1-[4-({2-chloro-7-[(1S)-1-methoxyethyl]-[1,2,4]triazolo[1,5-a]pyrimidin-6-yl}amino)phenyl]ethyl}-N-methylcyclohexanecarboxamide [Compound 1.21] (9.30 mg, 19.7 μmol, 12.8% yield) as yellow dry powder. m/z: [M+H]+ Calcd for C24H32ClN6O2 471.2; Found 471.3. ¹H NMR (400 MHz, DMSO-d6) δ=8.79 (s, 1H), 7.79-7.70 (m, 1H), 7.11-7.03 (m, 2H), 6.96-6.88 (m, 2H), 5.83-5.12 (m, 2H), 3.17 (s, 3H), 2.75-2.53 (m, 3H), 1.74-1.60 (m, 5H), 1.58 (d, J=6.8 Hz, 3H), 1.49 (br d, J=6.8 Hz, 1H), 1.42 (br s, 1H), 1.34 (br d, J=7.2 Hz, 3H), 1.27 (br d, J=12.8 Hz, 2H), 1.24-1.09 (m, 2H).

### Synthesis of N-{1-[4-({2-chloro-7-[(1S)-1-methoxyethyl]-[1,2,4]triazolo[1,5-a]pyrimidin-6-yl}amino)phenyl]ethyl}-N-methylcyclopentanecarboxamide (Compound 1.22)

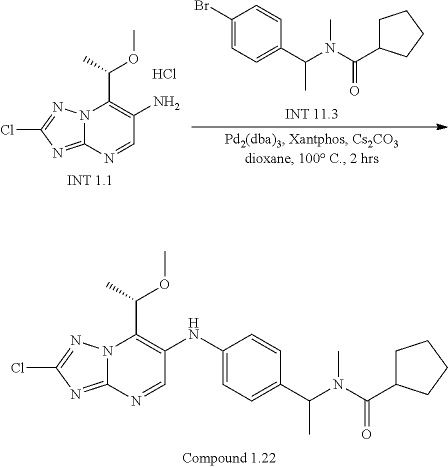 [SMILES: CO[C@@H](C)c1c(N)cnc2nc(Cl)nn12 ; Cl ; CC(c1ccc(Br)cc1)N(C)C(=O)C1CCCC1 ; CO[C@@H](C)c1c(Nc2ccc(C(C)N(C)C(=O)C3CCCC3)cc2)cnc2nc(Cl)nn12]

To a solution of N-[1-(4-bromophenyl)ethyl]-N-methylcyclopentanecarboxamide [INT 11.3] (50 mg, 161 μmol) and 2-chloro-7-[(1S)-1-methoxyethyl]-[1,2,4]triazolo[1,5-a]pyrimidin-6-amine hydrochloride [INT 1.1] (36.6 mg, 161 μmol) in dioxane (3 mL) was added Cs₂CO₃ (157 mg, 482 μmol), xantphos (18.5 mg, 32.1 μmol), and Pd₂(dba)₃ (14.6 mg, 16.0 μmol). The reaction mixture was stirred at 100° C. for 2 h under N₂. The mixture was concentrated in vacuo to give the crude product, which was purified by prep-HPLC (column: Phenomenex Gemini-NX C18 75*30 mm*3 μm, table: 35-75% B (A=water (0.05% ammonia hydroxide v/v), B=acetonitrile), flow rate: 25 mL/min, UV Detector 220 nm) to afford N-{1-[4-({2-chloro-7-[(1S)-1-methoxyethyl]-[1,2,4]triazolo[1,5-a]pyrimidin-6-yl}amino)phenyl]ethyl}-N-methylcyclopentanecarboxamide [Compound 1.22] (10.0 mg, 21.8 μmol, 13.6% yield) as a yellow dry power. m/z: [M+H]+ Calcd for C23H30ClN6O2 457.2; Found 457.3. ¹H NMR (400 MHz, DMSO-d6) δ=8.80 (s, 1H), 7.80-7.70 (m, 1H), 7.14-7.04 (m, 2H), 6.99-6.87 (m, 2H), 5.81-5.13 (m, 2H), 3.17 (s, 3H), 3.13-2.91 (m, 1H), 2.70-2.51 (m, 3H), 1.86-1.62 (m, 6H), 1.58 (d, J=6.8 Hz, 3H), 1.55-1.45 (m, 3H), 1.35 (d, J=7.2 Hz, 2H).

### Synthesis of N-{1-[4-({2-chloro-7-[(1S)-1-methoxyethyl]-[1,2,4]triazolo[1,5-a]pyrimidin-6-yl}amino)phenyl]ethyl}-N-methylcyclopropanecarboxamide (Compound 1.23)

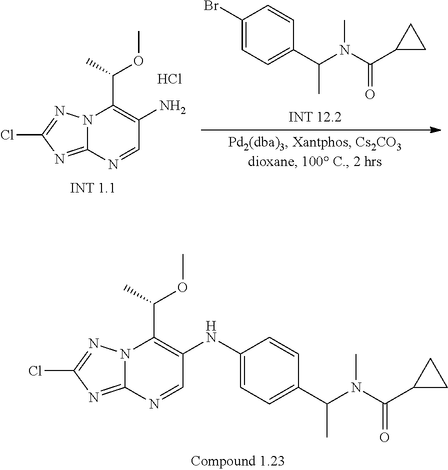 [SMILES: CO[C@@H](C)c1c(N)cnc2nc(Cl)nn12 ; Cl ; CC(c1ccc(Br)cc1)N(C)C(=O)C1CC1 ; CO[C@@H](C)c1c(Nc2ccc(C(C)N(C)C(=O)C3CC3)cc2)cnc2nc(Cl)nn12]

To a mixture of N-[1-(4-bromophenyl)ethyl]-N-methylcyclopropanecarboxamide [INT 12.2](50 mg, 177 μmol) and 2-chloro-7-[(1S)-1-methoxyethyl]-[1,2,4]triazolo[1,5-a]pyrimidin-6-amine hydrochloride [INT 1.1] (40.2 mg, 177 μmol) in dioxane (2 ml) was added Pd₂(dba)₃ (16.2 mg, 17.7 μmol), Cs₂CO₃ (173 mg, 531 μmol), and xantphos (20.4 mg, 35.4 μmol) at 25° C. The mixture was stirred at 100° C. for 12 hr under N₂. The mixture was concentrated in vacuo to give the crude product, which was purified by prep-HPLC (column: YMC Triart C18 250*50 mm*7 μm, table: 25-65% B (A=water (0.05% ammonia hydroxide v/v), B=acetonitrile), flow rate: 60 mL/min, UV Detector 220 nm) to afford N-{1-[4-({2-chloro-7-[(1S)-1-methoxyethyl]-[1,2,4]triazolo[1,5-a]pyrimidin-6-yl}amino)phenyl]ethyl}-N-methylcyclopropanecarboxamide [Compound 1.23] (16.9 mg, 39.4 μmol, 22.2% yield) as a yellow dry power. m/z: [M+H]+ Calcd for C21H26ClN6O2 429.2; Found 429.3. ¹H NMR (400 MHz, DMSO-d6) δ=8.86 (s, 1H), 7.92-7.74 (m, 1H), 7.46-6.96 (m, 4H), 5.86-5.52 (m, 1H), 5.23 (q, J=6.8 Hz, 1H), 3.24 (s, 3H), 2.87-2.58 (m, 3H), 2.19-1.89 (m, 1H), 1.64 (d, J=6.8 Hz, 3H), 1.59 (br d, J=6.4 Hz, 1H), 1.43 (br d, J=7.2 Hz, 2H), 0.85-0.76 (m, 4H).

### Synthesis of N-{1-[4-({2-chloro-7-[(1S)-1-methoxyethyl]-[1,2,4]triazolo[1,5-a]pyrimidin-6-yl}amino)phenyl]ethyl}-N-methylacetamide (Compound 1.24)

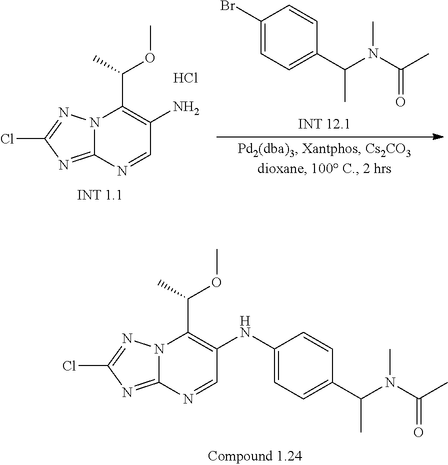 [SMILES: CO[C@@H](C)c1c(N)cnc2nc(Cl)nn12 ; Cl ; CC(=O)N(C)C(C)c1ccc(Br)cc1 ; CO[C@@H](C)c1c(Nc2ccc(C(C)N(C)C(C)=O)cc2)cnc2nc(Cl)nn12]

To a solution of N-[1-(4-bromophenyl)ethyl]-N-methylacetamide [INT 12.11] (30 mg, 117 μmol) and 2-chloro-7-[(1S)-1-methoxyethyl]-[1,2,4]triazolo[1,5-a]pyrimidin-6-amine hydrochloride [INT 1.1] (26.6 mg, 117 μmol) in dioxane (2 mL) was added Pd₂(dba)₃ (10.7 mg, 11.7 μmol), Cs₂CO₃ (114 mg, 351 μmol), and xantphos (13.5 mg, 23.4 μmol). The reaction mixture was stirred at 100° C. for 2 h under N₂. The reaction mixture was concentrated under reduced pressure to give the crude product, which was purified by prep-HPLC (column: YMC-Actus Triart C18 150*30 mm*5 μm, table: 37-57% B (A=water (0.05% ammonia hydroxide)), B=acetonitrile), flow rate: 35 mL/min, UV Detector 220 nm) to afford N-{1-[4-({2-chloro-7-[(1S)-1-methoxyethyl]-[1,2,4]triazolo[1,5-a]pyrimidin-6-yl}amino)phenyl]ethyl}-N-methylacetamide [Compound 1.24] (2.50 mg, 6.20 μmol, 5.3% yield) as a yellow dry powder. m/z: [M+H]+ Calcd for C19H24ClN6O2 403.2; Found 403.2. ¹H NMR (400 MHz, DMSO-d6) δ=8.85-8.53 (m, 1H), 7.89-7.72 (m, 1H), 7.49-6.91 (m, 4H), 5.80-5.01 (m, 2H), 3.18 (s, 3H), 2.65-2.54 (m, 3H), 2.16-2.01 (m, 3H), 1.59 (d, J=6.8 Hz, 3H), 1.50-1.34 (m, 3H).

### Synthesis of N-{1-[4-({2-chloro-7-[(1S)-1-methoxyethyl]-[1,2,4]triazolo[1,5-a]pyrimidin-6-yl}amino)phenyl]-2,2,2-trifluoroethyl}-N-methylacetamide (Compound 1.25)

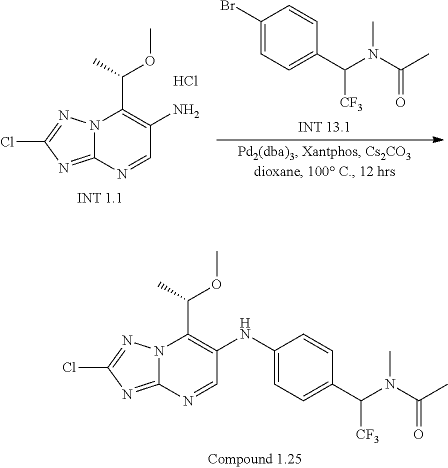 [SMILES: CO[C@@H](C)c1c(N)cnc2nc(Cl)nn12 ; Cl ; CC(=O)N(C)C(c1ccc(Br)cc1)C(F)(F)F ; CO[C@@H](C)c1c(Nc2ccc(C(N(C)C(C)=O)C(F)(F)F)cc2)cnc2nc(Cl)nn12]

To a mixture of 2-chloro-7-[(1S)-1-methoxyethyl]-[1,2,4]triazolo[1,5-a]pyrimidin-6-amine hydrochloride [INT 1.1] (120 mg, 0.5271 mmol). N-[1-(4-bromophenyl)-2,2,2-trifluoroethyl]-N-methylacetamide [INT 13.1] (150 mg, 0.4836 mmol), xantphos (80 mg, 0.1382 mmol), and Cs₂CO₃ (500 mg, 1.53 mmol) in dioxane (2 mL) was added Pd₂(dba)₃ (80 mg, 0.08736 mmol) and the mixture was stirred at 100° C. for 12 hr under N₂. The reaction was diluted with EtOAc (30 mL), filtered and concentrated under reduced pressure. The resulting residue was purified by prep-HPLC (column: Boston Prime C18 150*30 mm*5 μm, condition: 40%-60% CH₃CN in water (0.05% ammonia hydroxide v/v), flow rate: 25 mL/min) to give N-{1-[4-({2-chloro-7-[(1S)-1-methoxyethyl]-[1,2,4]triazolo[1,5-a]pyrimidin-6-yl}amino)phenyl]-2,2,2-trifluoroethyl}-N-methylacetamide [Compound 1.25] (10.2 mg, 0.02232 mmol, 4.6% yield) as a yellow dry powder. m/z: [M+H]+ Calcd for C19H21ClF3N6O2 457.1; Found 457.1. ¹H NMR (400 MHz, DMSO-d6) δ=8.84 (s, 1H), 8.07-7.90 (m, 1H), 7.34-7.13 (m, 2H), 6.97 (br d, J=8.6 Hz, 2H), 6.50-5.82 (m, 1H), 5.16 (q, J=6.7 Hz, 1H), 3.17 (s, 3H), 2.90-2.59 (m, 3H), 2.31-2.10 (m, 3H), 1.60 (d, J=6.7 Hz, 3H).

### Synthesis of N-[(1R)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide (Also Known as N—((R)-1-(4-((2-chloro-7-((S)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide) (Compound 1.26)

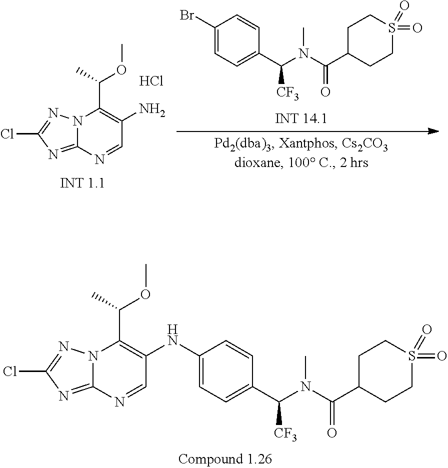 [SMILES: CO[C@@H](C)c1c(N)cnc2nc(Cl)nn12 ; Cl ; CN(C(=O)C1CCS(=O)(=O)CC1)[C@H](c1ccc(Br)cc1)C(F)(F)F ; CO[C@@H](C)c1c(Nc2ccc([C@@H](N(C)C(=O)C3CCS(=O)(=O)CC3)C(F)(F)F)cc2)cnc2nc(Cl)nn12]

To a solution of N-[(1R)-1-(4-bromophenyl)-2,2,2-trifluoroethyl]-N-methyl-1,1-dioxo-1λ⁶-thiane-4-carboxamide [INT 14.1] (50 mg, 116 μmol) and 2-chloro-7-[(1S)-1-methoxyethyl]-[1,2,4]triazolo[1,5-a]pyrimidin-6-amine hydrochloride [INT 1.1] (31.6 mg, 139 μmol) in dioxane (2 mL) was added Pd₂(dba)₃ (10.6 mg, 11.6 μmol), Cs₂CO₃ (113 mg, 348 μmol), and xantphos (13.4 mg, 23.2 μmol). The reaction mixture was stirred at 100° C. for 2 h under N₂. The reaction mixture was concentrated under reduced pressure to give the crude product, which was purified by prep-HPLC (column: Boston Green ODS 150*30 mm*5 μm, table: 24-64% B (A=water (0.05% ammonia hydroxide)), B=acetonitrile), flow rate: 30 mL/min, UV Detector 220 nm) to afford N-[(1R)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide [Compound 1.26] (16.4 mg, 28.5 μmol, 24.6% yield) as a yellow dry powder. m/z: [M+H]+ Calcd for C23H27ClF3N6O4S 575.1; Found 575.3. ¹H NMR (400 MHz, DMSO-d6) δ=8.83 (s, 1H), 8.07-7.97 (m, 1H), 7.29-7.17 (m, 2H), 7.03-6.92 (m, 2H), 6.49-6.05 (m, 1H), 5.16 (q, J=6.8 Hz, 1H), 3.26-3.19 (m, 2H), 3.16 (s, 3H), 3.13-3.08 (m, 2H), 2.90 (s, 3H), 2.65 (s, 1H), 2.10-1.95 (m, 4H), 1.59 (d, J=6.8 Hz, 3H).

### Synthesis of N-[(1R)-1-[4-({2-chloro-7-[(1R)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide (Also Known as N—((R)-1-(4-((2-chloro-7-((R)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide) (Compound 1.27)

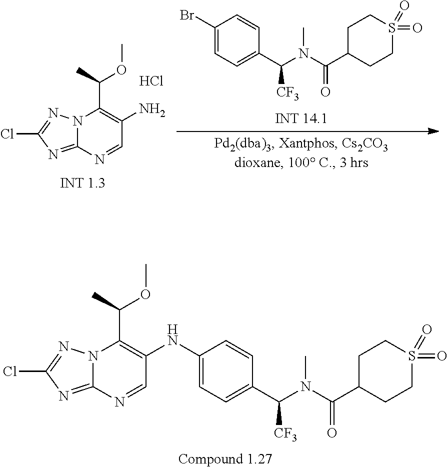 [SMILES: CO[C@H](C)c1c(N)cnc2nc(Cl)nn12 ; Cl ; CN(C(=O)C1CCS(=O)(=O)CC1)[C@H](c1ccc(Br)cc1)C(F)(F)F ; CO[C@H](C)c1c(Nc2ccc([C@@H](N(C)C(=O)C3CCS(=O)(=O)CC3)C(F)(F)F)cc2)cnc2nc(Cl)nn12]

A mixture of 2-chloro-7-[(1R)-1-methoxyethyl]-[1,2,4]triazolo[1,5-a]pyrimidin-6-amine hydrochloride [INT 1.3] (60 mg, 227 μmol), N-[(1R)-1-(4-bromophenyl)-2,2,2-trifluoroethyl]-N-methyl-1,1-dioxo-1λ⁶-thiane-4-carboxamide [INT 14.1] (97.2 mg, 227 μmol), Pd₂(dba)₃ (20.7 mg, 22.7 μmol), Xantphos (26.2 mg, 45.4 μmol), and Cs₂CO₃ (221 mg, 681 μmol) in dioxane (2 mL) was stirred at 100° C. for 3 hr under N₂ atmosphere. The mixture was concentrated under reduced pressure to afford the crude product, which was purified by flash chromatography on silica gel (methanol/dichloromethane=0/1 to 1/20). The resulting product was purified by prep-HPLC (column: YMC Triart C18 250*50 mm*7 μm, table: 26-66% B (A=water (0.05% ammonia hydroxide v/v)), B=acetonitrile), flow rate: 60 mL/min, UV Detector 220 nm) and prep-TLC (SiO₂, dichloromethane:methanol=20:1) to afford N-[(1R)-1-[4-({2-chloro-7-[(1R)-1-methoxyethyl]-[1,2,4]triazolo[1,5-a]pyrimidin-6-yl}amino)phenyl]-2,2,2-trifluoroethyl]-N-methyl-1,1-dioxo-1λ⁶-thiane-4-carboxamide [Compound 1.27] (5.20 mg, 9.04 μmol, 4.0% yield) as a white dry powder. m/z: [M+H]+ Calcd for C23H27ClF3N6O4S 575.1; Found 575.3. ¹H NMR (400 MHz, CD₃OD) δ=8.86 (s, 1H), 7.30 (d, J=8.4 Hz, 2H), 7.06 (d, J=8.4 Hz, 2H), 6.52 (q, J=9.2 Hz, 1H), 5.36 (q, J=6.8 Hz, 1H), 3.36 (s, 3H), 3.25-3.10 (m, 5H), 3.02-2.75 (m, 3H), 2.35-2.12 (m, 4H), 1.64 (d, J=6.8 Hz, 3H).

### Synthesis of N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methylcyclobutanecarboxamide (Compound 1.28)

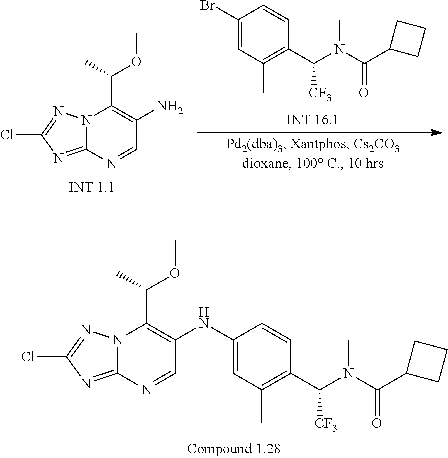 [SMILES: CO[C@@H](C)c1c(N)cnc2nc(Cl)nn12 ; Cc1cc(Br)ccc1[C@H](N(C)C(=O)C1CCC1)C(F)(F)F ; CO[C@@H](C)c1c(Nc2ccc([C@H](N(C)C(=O)C3CCC3)C(F)(F)F)c(C)c2)cnc2nc(Cl)nn12]

N-[(1S)-1-(4-bromo-2-methylphenyl)-2,2,2-trifluoroethyl]-N-methylcyclobutanecarboxamide [INT 16.1] (0.1 g, 0.2745 mmol), 2-chloro-7-[(1S)-1-methoxyethyl]-[1,2,4]triazolo[1,5-a]pyrimidin-6-amine [free base of INT 1.1] (62.3 mg, 274 μmol), Cs₂CO₃ (268 mg, 823 μmol), and xantphos (15.8 mg, 27.4 μmol) were mixed in dioxane (2 mL) and the reaction mixture was degassed with argon for 5 min. Pd₂(dba)₃ (12.5 mg, 13.7 μmol) was added, after which the reaction mixture was degassed with argon for 5 min and stirred at 100° C. for 10 h. The reaction mixture was cooled to rt. The solid was filtered off. The filtrate was purified by HPLC (see conditions below) to obtain N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methylcyclobutanecarboxamide [Compound 1.28] (44.7 mg, 0.08757 mmol, 31.9% yield) as an yellow solid. m/z: [M+H]+ Calcd for C23H27ClF3N6O2 511.2; Found 511.2. ¹H NMR (400 MHz, DMSO-d6) δ=8.83 (d, J=1.4 Hz, 1H), 7.91 (s, 1H), 7.29 (d, J=8.4 Hz, 1H), 6.85-6.78 (m, 2H), 6.43 (q, J=9.3 Hz, 1H), 5.14 (q, J=6.6 Hz, 1H), 3.46 (p, J=8.5 Hz, 1H), 3.16 (s, 3H), 2.63 (s, 3H), 2.14 (dp, J=24.5, 8.1, 7.5 Hz, 4H), 2.05 (s, 3H), 1.93 (dt, J=18.4, 9.2 Hz, 1H), 1.76 (s, 1H), 1.58 (d, J=6.7 Hz, 3H).

HPLC conditions; System: Agilent 1260 Infinity II LC coupled to an Agilent 6120B Single Quadrupole LC/MS System; Column Description: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water.

### Synthesis of N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methylcyclopropanecarboxamide (Compound 1.29)

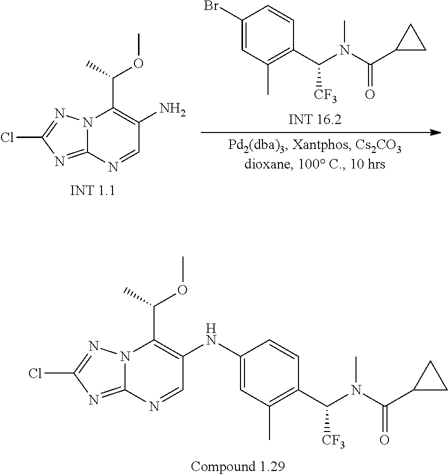 [SMILES: CO[C@@H](C)c1c(N)cnc2nc(Cl)nn12 ; Cc1cc(Br)ccc1[C@H](N(C)C(=O)C1CC1)C(F)(F)F ; CO[C@@H](C)c1c(Nc2ccc([C@H](N(C)C(=O)C3CC3)C(F)(F)F)c(C)c2)cnc2nc(Cl)nn12]

N-[(1S)-1-(4-bromo-2-methylphenyl)-2,2,2-trifluoroethyl]-N-methylcyclopropanecarboxamide [INT 16.2] (0.1 g, 0.2855 mmol), 2-chloro-7-[(1S)-1-methoxyethyl]-[1,2,4]triazolo[1,5-a]pyrimidin-6-amine [free base of INT 1.1] (64.8 mg, 0.2846 mmol), Cs₂CO₃ (277 mg, 853 μmol), and xantphos (16.4 mg, 28.4 μmol) were mixed in dioxane (2 mL). Pd₂(dba)₃ (13.0 mg, 14.2 μmol) was added in an inert atmosphere. The mixture was stirred for 10 hr at 100° C., after which the reaction mixture was cooled to rt. The solid was filtered off and the filtrate was purified by HPLC (see conditions below) to obtain N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methylcyclopropanecarboxamide [Compound 1.29] (15.7 mg, 0.03173 mmol, 11.1% yield) as a yellow solid. m/z: [M+H]+ Calcd for C22H25ClF3N6O2 497.2; Found 497.2. ¹H NMR (400 MHz, CD₃OD) δ=8.89 (d, J=2.4 Hz, 1H), 7.48 (d, J=8.4 Hz, 1H), 6.94 (dt, J=8.7, 4.3 Hz, 2H), 6.51 (q, J=8.9 Hz, 1H), 5.38 (q, J=6.7 Hz, 1H), 3.38 (s, 3H), 3.01 (s, 3H), 2.71-2.58 (m, 1H), 2.33-2.11 (m, 3H), 2.07-1.95 (m, 1H), 1.65 (d, J=6.7 Hz, 3H), 1.03-0.86 (m, 4H).

HPLC conditions; System: Agilent 1260 Infinity II LC coupled to an Agilent 6120B Single Quadrupole LC/MS System; Column Description: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water; Mobile phase B: acetonitrile; Flow rate: 30 ml/min; loading pump: 4 ml/min B; Gradient conditions: 20-40-50-100% (B) 0-2-10-11.2 min.

### Synthesis of N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methyl-1,1-dioxo-1λ⁶-thiane-4-carboxamide (Also Known as N—((S)-1-(4-((2-chloro-7-((1S)-1-methoxyethyl)-[1,2,4]triazolo[1,5-a]pyrimidin-6-yl)amino)-2-methylphenyl)-2,2,2-trifluoroethyl)-N-methyltetrahydro-2H-thiopyran-4-carboxamide 1,1-dioxide) (Compound 1.30)

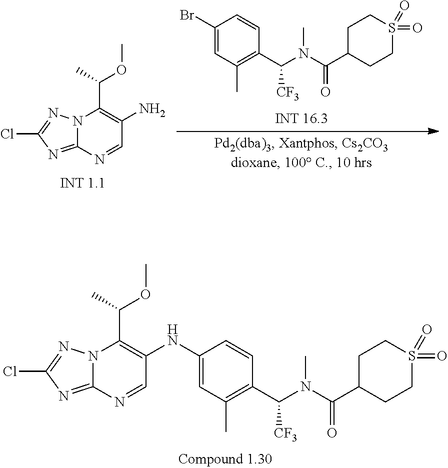 [SMILES: CO[C@@H](C)c1c(N)cnc2nc(Cl)nn12 ; Cc1cc(Br)ccc1[C@H](N(C)C(=O)C1CCS(=O)(=O)CC1)C(F)(F)F ; CO[C@@H](C)c1c(Nc2ccc([C@H](N(C)C(=O)C3CCS(=O)(=O)CC3)C(F)(F)F)c(C)c2)cnc2nc(Cl)nn12]

A mixture of N-[(1S)-1-(4-bromo-2-methylphenyl)-2,2,2-trifluoroethyl]-N-methyl-1,1-dioxo-1λ⁶-thiane-4-carboxamide [INT 16.3] (100 mg, 0.2260 mmol), 2-chloro-7-[(1S)-1-methoxyethyl]-[1,2,4]triazolo[1,5-a]pyrimidin-6-amine [free base of INT 1.1] (51.4 mg, 226 μmol), Cs₂CO₃ (220 mg, 678 μmol), and dioxane (3 mL) was purged with argon. Then xantphos (26.1 mg, 45.2 μmol) and Pd₂(dba)₃ (20.6 mg, 22.6 μmol) were added and the reaction mixture was stirred at 100° C. for 10 h. After cooling, the reaction mixture was diluted with EtOAc (30 mL) and concentrated under reduced pressure. The resulting residue was purified by HPLC (see conditions below) to obtain N-[(1S)-1-[4-({2-chloro-7-[(1S)-1-methoxyethyl]-[1,2,4]triazolo[1,5-a]pyrimidin-6-yl}amino)-2-methylphenyl]-2,2,2-trifluoroethyl]-N-methyl-1,1-dioxo-1λ⁶-thiane-4-carboxamide [Compound 1.30] (12.5 mg, 0.02128 mmol, 9.4% yield) as a yellow solid. m/z: [M+H]+ Calcd for C24H29ClF3N6O4S 589.2; Found 589.0. ¹H NMR (500 MHz, DMSO-d6) δ=8.80 (s, 1H), 7.90 (s, 1H), 7.29 (d, J=8.3 Hz, 1H), 6.87-6.75 (m, 2H), 6.43 (q, J=9.1 Hz, 1H), 5.13 (q, J=6.8 Hz, 1H), 3.27-3.14 (m, 2H), 3.15 (s, 3H), 3.12-3.04 (m, 3H), 2.79 (s, 3H), 2.15-2.03 (m, 2H), 2.01 (s, 3H), 1.99-1.94 (m, 2H), 1.57 (d, J=6.7 Hz, 3H).

HPLC conditions; System: Agilent 1260 Infinity II LC coupled to an Agilent 6120B Single Quadrupole LC/MS System; Column Description: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water; Mobile phase B: acetonitrile; Flow rate: 30 ml/min; loading pump: 4 ml/min B; Gradient conditions: 20-35-50-100% (B) 0-2-10-11.2 min.

### Synthesis of N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methylcyclobutanecarboxamide (Compound 1.31)

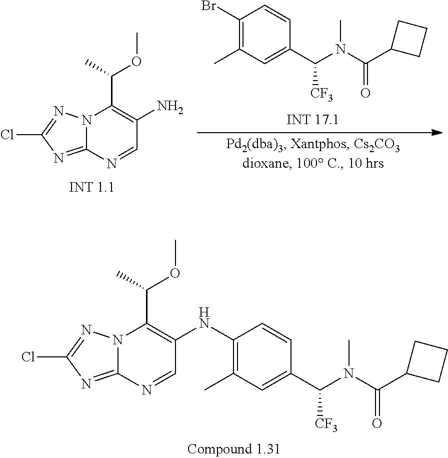 [SMILES: CO[C@@H](C)c1c(N)cnc2nc(Cl)nn12 ; Cc1cc([C@H](N(C)C(=O)C2CCC2)C(F)(F)F)ccc1Br ; CO[C@@H](C)c1c(Nc2ccc([C@H](N(C)C(=O)C3CCC3)C(F)(F)F)cc2C)cnc2nc(Cl)nn12]

N-[(1S)-1-(4-bromo-3-methylphenyl)-2,2,2-trifluoroethyl]-N-methylcyclobutanecarboxamide [INT 17.1] (0.12 g, 0.3294 mmol), 2-chloro-7-[(1S)-1-methoxyethyl]-[1,2,4]triazolo[1,5-a]pyrimidin-6-amine [free base of INT 1.1] (74.8 mg, 329 μmol), Cs₂CO₃ (321 mg, 988 μmol), and xantphos (19.0 mg, 32.9 μmol) were mixed in dioxane (2 mL) and the reaction mixture was degassed with argon for 5 min. Pd₂(dba)₃ (15.0 mg, 16.4 μmol) was added, then the reaction mixture was degassed with argon for 5 min and stirred at 100° C. for 10 h. The reaction mixture was cooled to rt and the solid was filtered off. The filtrate was purified by HPLC (see conditions below) to obtain N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methylcyclobutanecarboxamide [Compound 1.31] (7.31 mg, 0.01431 mmol, 4.4% yield) as a yellow solid. m/z: [M+H]+ Calcd for C23H27ClF3N6O2 511.2; Found 511.2. ¹H NMR (500 MHz, DMSO-d6) δ=8.74 (s, 1H), 7.23 (s, 1H), 7.14 (s, 1H), 7.02 (d, J=8.5 Hz, 1H), 6.83 (d, J=8.4 Hz, 1H), 6.40 (q, J=9.5 Hz, 1H), 5.15 (q, J=6.7 Hz, 1H), 3.50-3.43 (m, 1H), 3.23 (s, 3H), 2.71 (s, 3H), 2.30 (s, 3H), 2.21-2.06 (m, 4H), 1.91 (q, J=9.5 Hz, 1H), 1.79-1.73 (m, 1H), 1.54 (d, J=6.7 Hz, 3H).

HPLC conditions; System: Agilent 1260 Infinity II LC coupled to an Agilent 6120B Single Quadrupole LC/MS System; Column Description: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water Mobile phase B: acetonitrile; Flow rate: 30 ml/min; loading pump: 4 ml/min B; Gradient conditions: 30-45-60-100% (B) 0-2-10-11.2 min.

### Synthesis of N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methylcyclopropanecarboxamide (Compound 1.32)

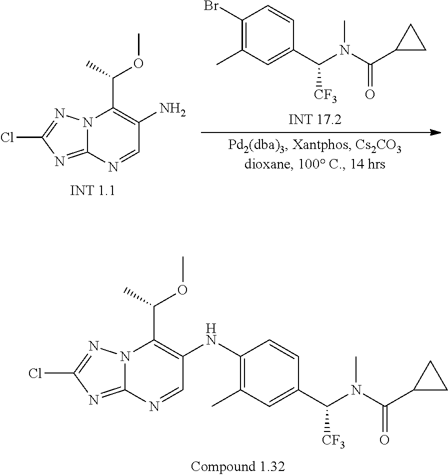 [SMILES: CO[C@@H](C)c1c(N)cnc2nc(Cl)nn12 ; Cc1cc([C@H](N(C)C(=O)C2CC2)C(F)(F)F)ccc1Br ; CO[C@@H](C)c1c(Nc2ccc([C@H](N(C)C(=O)C3CC3)C(F)(F)F)cc2C)cnc2nc(Cl)nn12]

N-[(1S)-1-(4-bromo-3-methylphenyl)-2,2,2-trifluoroethyl]-N-methylcyclopropanecarboxamide [INT 17.2] (50 mg, 0.1427 mmol), 2-chloro-7-[(1S)-1-methoxyethyl]-[1,2,4]triazolo[1,5-a]pyrimidin-6-amine [free base of INT 1.1] (32.3 mg, 142 μmol), and Cs₂CO₃ (139 mg, 428 μmol) were mixed in dioxane (5 mL). Argon was bubbled through the reaction mixture for 15 min. Then xantphos (16.4 mg, 28.5 μmol) and Pd₂(dba)₃ (13.0 mg, 14.2 μmol) were added, and the reaction mixture was stirred at 100° C. for 14 h. The reaction mixture was cooled, filtered, and concentrated in vacuo. The resulting residue was purified by HPLC (see conditions below) to give N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methylcyclopropanecarboxamide [Compound 1.32] (21.6 mg, 0.04358 mmol, 30.6% yield) as a yellow solid. m/z: [M+H]+ Calcd for C22H25ClF3N6O2 497.2; Found 497.0. ¹H NMR (400 MHz, DMSO-d6) δ=8.81-8.75 (m, 1H), 7.26 (s, 1H), 7.17 (s, 1H), 7.05 (d, J=8.4 Hz, 1H), 6.90-6.83 (m, 1H), 6.43 (q, J=9.5 Hz, 1H), 5.17 (q, J=6.6 Hz, 1H), 3.25 (s, 3H), 3.01 (s, 3H), 2.32 (s, 3H), 2.06-1.97 (m, 1H), 1.56 (d, J=6.7 Hz, 3H), 0.90-0.81 (m, 4H).

HPLC Conditions; System: Agilent 1260 Infinity II LC coupled to an Agilent 6120B Single Quadrupole LC/MS System; Column Description: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water Mobile phase B: acetonitrile; Flow rate: 30 ml/min; loading pump: 4 ml/min B; Gradient conditions: 20-40-65-100% (B) 0-2-10-11.2 min.

### Synthesis of N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methyl-1,1-dioxo-1λ⁶-thiane-4-carboxamide (Also Known as N—((S)-1-(4-((2-chloro-7-((S)-1-methoxyethyl)-[1,2,4]triazolo[1,5-a]pyrimidin-6-yl)amino)-3-methylphenyl)-2,2,2-trifluoroethyl)-N-methyltetrahydro-2H-thiopyran-4-carboxamide 1,1-dioxide) (Compound 1.33)

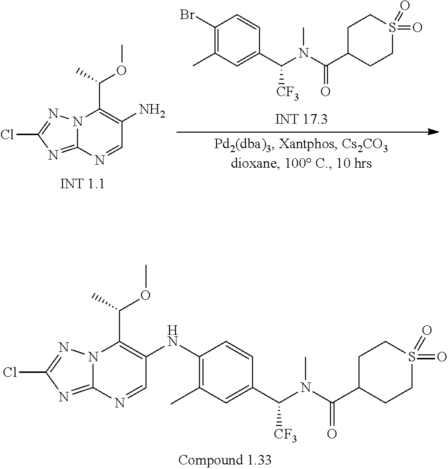 [SMILES: CO[C@@H](C)c1c(N)cnc2nc(Cl)nn12 ; Cc1cc([C@H](N(C)C(=O)C2CCS(=O)(=O)CC2)C(F)(F)F)ccc1Br ; CO[C@@H](C)c1c(Nc2ccc([C@H](N(C)C(=O)C3CCS(=O)(=O)CC3)C(F)(F)F)cc2C)cnc2nc(Cl)nn12]

N-[(1S)-1-(4-bromo-3-methylphenyl)-2,2,2-trifluoroethyl]-N-methyl-1,1-dioxo-1λ⁶-thiane-4-carboxamide [INT 17.3] (0.1 g, 0.2260 mmol), 2-chloro-7-[(1S)-1-methoxyethyl]-[1,2,4]triazolo[1,5-a]pyrimidin-6-amine [free base of INT 1.1] (51.4 mg, 226 μmol), Cs₂CO₃ (220 mg, 678 μmol), and xantphos (13.0 mg, 22.6 μmol) were mixed in dioxane (2 mL) and the reaction mixture was degassed with argon for 5 min. Pd₂(dba)₃ (10.3 mg, 11.3 μmol) was added. Then the reaction mixture was degassed with argon for 5 min and stirred at 100° C. for 10 h. The reaction mixture was cooled to rt and the solid was filtered off. The filtrate was purified by HPLC (see conditions below) to obtain N-[(1S)-1-[4-({2-chloro-7-[(1S)-1-methoxyethyl]-[1,2,4]triazolo[1,5-a]pyrimidin-6-yl}amino)-3-methylphenyl]-2,2,2-trifluoroethyl]-N-methyl-1,1-dioxo-1λ⁶-thiane-4-carboxamide [Compound 1.33] (20.7 mg, 0.03515 mmol, 15.5% yield) as a yellow solid. m/z: [M+H]+ Calcd for C24H29ClF3N6O4S 589.2; Found 589.0. ¹H NMR (600 MHz, DMSO-d6) δ=8.74 (s, 1H), 7.24 (s, 1H), 7.15 (s, 1H), 7.03 (d, J=8.6 Hz, 1H), 6.84 (d, J=8.4 Hz, 1H), 6.41 (q, J=9.4 Hz, 1H), 5.16 (q, J=6.7 Hz, 1H), 3.21 (d, J=13.9 Hz, 3H), 3.18-3.05 (m, 5H), 2.89 (s, 3H), 2.34-2.28 (m, 3H), 2.11-1.95 (m, 4H), 1.54 (d, J=6.7 Hz, 3H).

HPLC conditions; System: Agilent 1260 Infinity II LC coupled to an Agilent 6120B Single Quadrupole LC/MS System; Column Description: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water; Mobile phase B: acetonitrile; Flow rate: 30 ml/min; loading pump: 4 ml/min B; Gradient conditions: 10-30-55-100% (B) 0-2-10-11.2 min.

### 2. Compounds Prepared Using Scheme 2

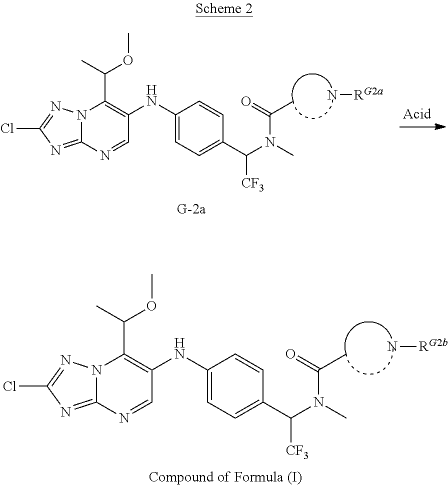

Starting material G-2a is treated with an acid to provide a compound of formula (I). R^{G2a} is either Boc (for cyclic carbamates) or HBoc (for acyclic carbamates). R^{G2b} is H (for secondary amines) or H₂ (for primary amines).

### Synthesis of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide trifluoroacetate (Compound 2.1)

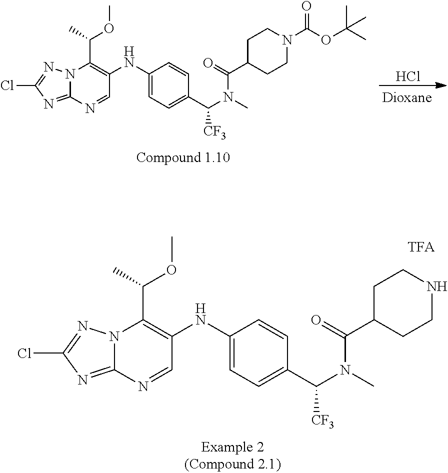 [SMILES: CO[C@@H](C)c1c(Nc2ccc([C@H](N(C)C(=O)C3CCN(C(=O)OC(C)(C)C)CC3)C(F)(F)F)cc2)cnc2nc(Cl)nn12 ; Cl ; CO[C@@H](C)c1c(Nc2ccc([C@H](N(C)C(=O)C3CCNCC3)C(F)(F)F)cc2)cnc2nc(Cl)nn12]

A mixture of tert-butyl 4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)piperidine-1-carboxylate [Compound 1.10] (300 mg, 479 μmol) in 4 M HCl/dioxane (10 mL, 40.0 mmol) was stirred at 25° C. for 4 hr. The mixture was concentrated under reduced pressure to afford N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide hydrochloride [Compound 2.1, HCl salt] (250 mg, 444 μmol, 92.9% yield) as a yellow solid. 50 mg (95.0 μmol) of the crude product was purified by prep-HPLC (column: Boston Green ODS 150*30 mm*5 um, table: 10-50% B (A=water(0.1% TFA)-ACN), B=acetonitrile), flowrate: 30 mL/min, UV Detector 220 nm) to afford N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide trifluoroacetate [Compound 2.1] (17.6 mg, 27.5 μmol) as a yellow dry powder. m/z: [M+H]+ Calcd for C23H28ClF3N7O2 526.2; Found 526.3. ¹H NMR (400 MHz, CDCl₃) δ=9.66 (br s, 1H), 9.16 (br s, 1H), 8.89 (s, 1H), 7.31 (d, J=8.4 Hz, 2H), 7.14 (s, 1H), 7.05 (d, J=8.8 Hz, 2H), 6.57 (q, J=8.8 Hz, 1H), 5.48 (q, J=6.8 Hz, 1H), 3.57 (br s, 5H), 3.14 (br d, J=5.2 Hz, 2H), 2.99 (br s, 1H), 2.93 (s, 3H), 2.23-1.94 (m, 4H), 1.61 (d, J=6.8 Hz, 3H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide (Compound 2.2)

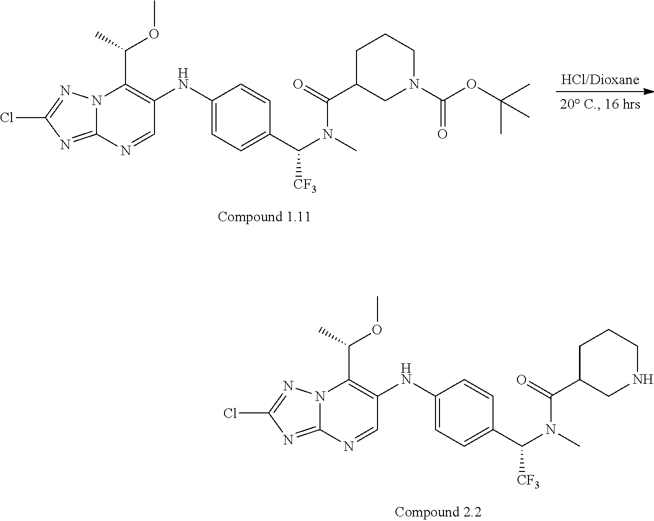 [SMILES: CO[C@@H](C)c1c(Nc2ccc([C@H](N(C)C(=O)C3CCCN(C(=O)OC(C)(C)C)C3)C(F)(F)F)cc2)cnc2nc(Cl)nn12 ; CO[C@@H](C)c1c(Nc2ccc([C@H](N(C)C(=O)C3CCCNC3)C(F)(F)F)cc2)cnc2nc(Cl)nn12]

A solution of tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl] (methyl)carbamoyl}piperidine-1-carboxylate [Compound 1.11] (200 mg, 319 μmol) in 4 M HCl/dioxane (6 mL) was stirred at 20° C. for 16 h. The reaction mixture was concentrated under reduced pressure to give N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide hydrochloride [Compound 2.2, HCl salt] (140 mg, 266 μmol, 83.4% yield) as a yellow solid. 45.3 mg of this crude product were purified by prep-HPLC (column: Phenomenex Gemini-NX 80*40 mm*3 μm, table: 22-62% B (A=water (0.05% ammonia hydroxide)), B=acetonitrile), flow rate: 25 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide [Compound 2.2] (5.30 mg, 10.0 μmol, 11.7% yield) as a yellow solid. m/z: [M+H]+ Calcd for C23H28ClF3N7O2 526.2; Found 526.3. ¹H NMR (400 MHz, CDCl₃) δ=8.89 (s, 1H), 7.31 (d, J=8.4 Hz, 2H), 7.11 (s, 1H), 7.04 (d, J=8.8 Hz, 2H), 6.61 (q, J=9.2 Hz, 1H), 5.48 (q, J=6.8 Hz, 1H), 3.51-3.45 (m, 3H), 3.15-2.98 (m, 2H), 2.93 (s, 3H). 2.85-2.67 (m, 2H), 2.62 (s, 2H), 2.05-1.96 (m, 1H), 1.82-1.70 (m, 2H), 1.61 (d, J=6.8 Hz, 3H), 1.59-1.50 (m, 1H).

### Synthesis of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpyrrolidine-3-carboxamide hydrochloride (Compound 2.3)

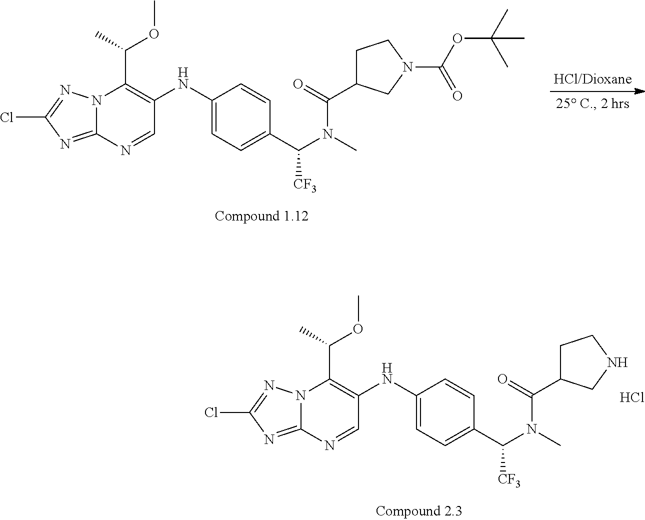 [SMILES: CO[C@@H](C)c1c(Nc2ccc([C@H](N(C)C(=O)C3CCN(C(=O)OC(C)(C)C)C3)C(F)(F)F)cc2)cnc2nc(Cl)nn12 ; CO[C@@H](C)c1c(Nc2ccc([C@H](N(C)C(=O)C3CCNC3)C(F)(F)F)cc2)cnc2nc(Cl)nn12 ; Cl]

A mixture of tert-butyl 3-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl] (methyl)carbamoyl}pyrrolidine-1-carboxylate [Compound 1.12] (200 mg, 326 μmol) in 4 M HCl/dioxane (10 mL, 40.0 mmol) was stirred at 25° C. for 2 hr. The mixture was concentrated under reduced pressure to afford the crude product, which was purified by prep-HPLC (column: Boston Green ODS 150*30 mm*5 μm, table: 16-56% B (A=water (0.05% HCl)-ACN), B=acetonitrile), flowrate: 30 mL/min, UV Detector 220 nm) to afford N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpyrrolidine-3-carboxamide hydrochloride [Compound 2.3] (31.3 mg, 57.1 μmol, 17.5% yield) as a yellow powder. m/z: [M+H]+ Calcd for C22H26ClF3N7O2 512.1, 514.1; Found 512.3, 514.2. ¹H NMR (400 MHz, CDCl₃) δ=10.16 (br s, 1H), 9.42 (br s, 1H), 8.90 (br s, 1H), 7.47-7.27 (m, 2H), 7.14-7.00 (m, 2H), 6.62-6.43 (m, 1H), 5.48 (br d, J=6.0 Hz, 1H), 3.68 (br d, J=14.8 Hz, 4H), 3.49 (s, 3H), 3.04-2.79 (m, 3H), 2.47 (br s, 1H), 2.22-2.09 (m, 2H), 1.61 (br d, J=6.0 Hz, 3H).

### Synthesis of 4-(aminomethyl)-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylcyclohexane-1-carboxamide; Formic Acid (Compound 2.4)

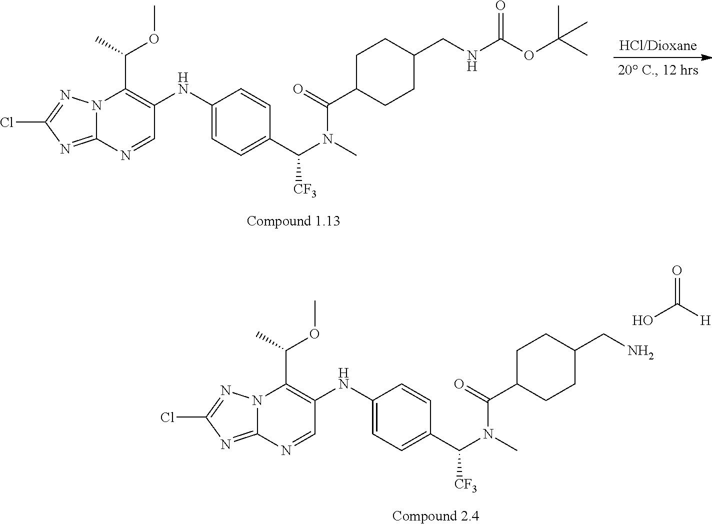

A solution of tert-butyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate [Compound 1.13] (450 mg, 625 μmol) in 4 N HCl/Dioxane (5 mL) was stirred at 20° C. for 12 h. The mixture was concentrated under reduced pressure to give 4-(aminomethyl)-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylcyclohexane-1-carboxamide hydrochloride [Compound 2.4, HCl salt] (370 mg, 616 μmol, 98.6% yield) as a yellow solid. 100 mg of the product was further purified by prep-HPLC (column: YMC Triart C18 250*50 mm*7 μm, table: 12-52% B (A=water (0.225% FA), B=acetonitrile), flow rate: 60 mL/min, UV Detector 220 nm) to afford 4-(aminomethyl)-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylcyclohexane-1-carboxamide; formic acid [Compound 2.4] (21.7 mg, 36.1 μmol, 21.7% yield) as a yellow dry powder. m/z: [M+H]+ Calcd for C25H32ClF3N7O2 554.2; Found 554.4. ¹H NMR (400 MHz, DMSO-d6) δ=8.84 (s, 1H), 8.43 (s, 1H), 8.13-8.00 (m, 1H), 7.29-7.12 (m, 2H), 7.02-6.93 (m, 2H), 6.52-6.08 (m, 1H), 5.16 (q, J=6.8 Hz, 1H), 3.16 (s, 3H), 2.87 (s, 3H), 2.67-2.56 (m, 2H), 1.88-1.67 (m, 4H), 1.59 (d, J=6.4 Hz, 3H), 1.55-1.21 (m, 4H), 1.10-0.91 (m, 2H).

### 3. Compounds Prepared Using Scheme 3

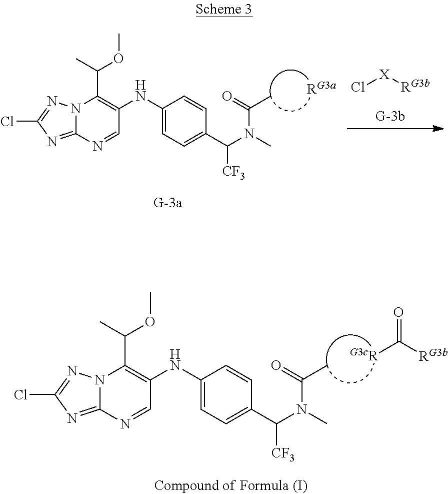

Starting material G-3a is treated with R^{G3b}-containing G-3b to provide a compound of formula (I). R^{G3a} is either NH (for cyclic amines) or NH2 (for primary amines); X is CO or SO₂; R^{G3b} is CH3, iPr, OMe, or NHMe; and R^{G3c} is either N (for cyclic amides, ureas, or carbamates) or NH (for acyclic amides, ureas, or carbamates).

### Synthesis of 1-acetyl-N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide (Compound 3.1)

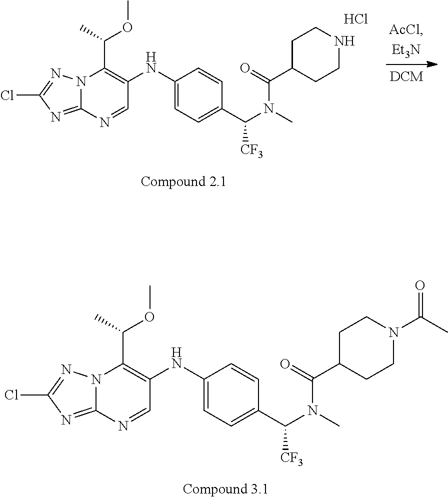 [SMILES: CO[C@@H](C)c1c(Nc2ccc([C@H](N(C)C(=O)C3CCNCC3)C(F)(F)F)cc2)cnc2nc(Cl)nn12 ; Cl ; CO[C@@H](C)c1c(Nc2ccc([C@H](N(C)C(=O)C3CCN(C(C)=O)CC3)C(F)(F)F)cc2)cnc2nc(Cl)nn12]

To a mixture of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide hydrochloride [Compound 2.1, HCl salt] (60 mg, 106 μmol) and triethylamine (53.6 mg, 530 μmol) in DCM (2 mL) was added acetyl chloride (29.1 mg, 371 μmol) at 25° C. and the mixture was stirred at 25° C. for 12 hr. The mixture was concentrated under reduced pressure to afford the crude product. The crude product was purified by prep-HPLC (column: Boston Prime C18 150*30 mm*5 μm, table: 35-65% B (A=water (0.05% ammonia hydroxide v/v), B=acetonitrile), flow rate: 30 mL/min, UV Detector 220 nm) to afford 1-acetyl-N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide [Compound 3.1] (17.7 mg, 31.1 μmol, 29.4% yield) as a yellow dry powder. m/z: [M+H]+ Calcd for C25H30ClF3N7O3 568.2; Found 568.4. ¹H NMR (400 MHz, CDCl₃) δ=8.90 (s, 1H), 7.35-7.29 (m, 2H), 7.17-7.01 (m, 3H), 6.68-6.58 (m, 1H), 5.49 (q, J=6.8 Hz, 1H), 4.71-4.54 (m, 1H), 3.93 (br d, J=13.6 Hz, 1H), 3.49 (s, 3H), 3.20-3.09 (m, 1H), 2.95 (s, 3H), 2.88-2.66 (m, 2H), 2.12 (s, 3H), 1.95-1.67 (m, 4H), 1.62 (d, J=6.7 Hz, 3H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1-(propane-2-sulfonyl)piperidine-4-carboxamide (Compound 3.2)

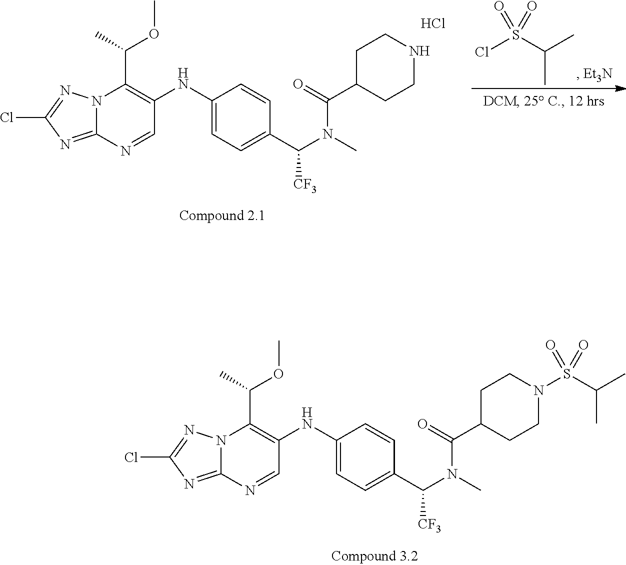

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-4-carboxamide hydrochloride [Compound 2.1, HCl salt] (80 mg, 142 μmol) and triethylamine (57.4 mg, 568 μmol) in DCM (5 mL) was added propane-2-sulfonyl chloride (40.4 mg, 284 μmol) at 25° C. and the mixture was stirred at 25° C. for 12 hr. The mixture was concentrated under reduced pressure to afford the crude product. The crude product was purified by prep-HPLC (column: Boston Prime C18 150*30 mm*5 μm, table: 40-70% B (A=water (0.05% ammonia hydroxide v/v)-ACN), B=acetonitrile), flowrate: 30 mL/min, UV Detector 220 run) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methyl-1-(propane-2-sulfonyl)piperidine-4-carboxamide [Compound 3.2] (9.10 mg, 14.3 μmol, 10.1% yield) as a yellow dry powder. m/z: [M+H]+ Calcd for C26H34ClF3N7O4S 632.2; Found 632.3. ¹H NMR (400 MHz, CDCl₃) δ=8.89 (s, 1H), 7.32 (br d, J=8.4 Hz, 2H), 7.18-7.01 (m, 3H), 6.62 (q, J=9.2 Hz, 1H), 5.48 (q, J=6.8 Hz, 1H), 3.85 (dt, J=4.4, 8.8 Hz, 2H), 3.49 (s, 3H), 3.19 (td, J=6.8, 13.6 Hz, 1H), 3.08-2.99 (m, 2H), 2.93 (s, 3H), 2.85-2.67 (m, 1H), 1.98-1.87 (m, 3H), 1.87-1.79 (m, 1H), 1.62 (s, 3H), 1.37 (d, J=6.8 Hz, 6H).

### Synthesis of methyl 4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate (Compound 3.3)

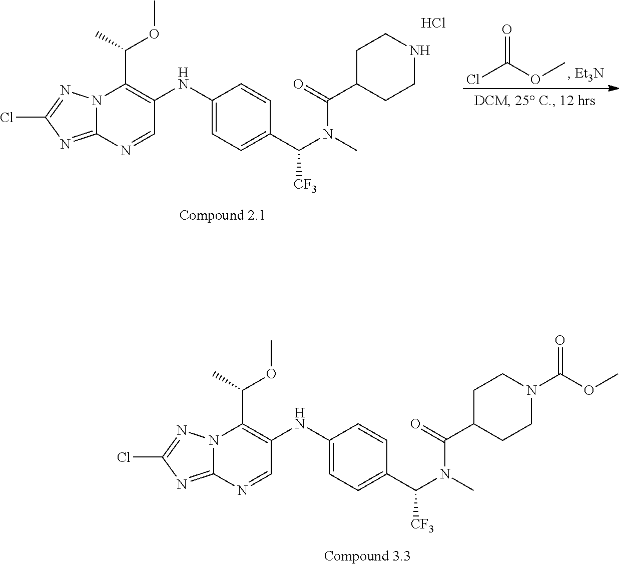

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-4-carboxamide hydrochloride [Compound 2.1, HCl salt] (100 mg, 177 μmol) and triethylamine (71.6 mg, 708 μmol) in DMF (3 mL) was added methyl carbonochloridate (329 mg, 3.48 mmol) and the mixture was stirred at 25° C. for 12 hr. The crude product was purified by prep-HPLC (column: YMC Triart C18 250*50 mm*7 μm, table: 33-73% B (A=water (0.05% ammonia hydroxide v/v)-ACN), B=acetonitrile), flowrate: 25 mL/min, UV Detector 220 nm) to afford methyl 4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}piperidine-1-carboxylate [Compound 3.3] (46.0 mg, 78.7 μmol, 44.6% yield) as a yellow dry powder. m/z: [M+H]+ Calcd for C25H30ClF3N7O4 584.2; Found 584.5. ¹H NMR (400 MHz, CDCl₃) δ=8.89 (s, 1H), 7.32 (d, J=8.4 Hz, 2H), 7.12 (s, 1H), 7.04 (d, J=8.8 Hz, 2H), 6.62 (q, J=9.2 Hz, 1H), 5.48 (q, J=6.8 Hz, 1H), 4.22 (br s, 2H), 3.72 (s, 3H), 3.49 (s, 3H), 2.94 (s, 3H), 2.91-2.69 (m, 3H), 1.89-1.69 (m, 4H), 1.62 (d, J=6.7 Hz, 3H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpiperidine-4-carboxamide (Compound 3.4)

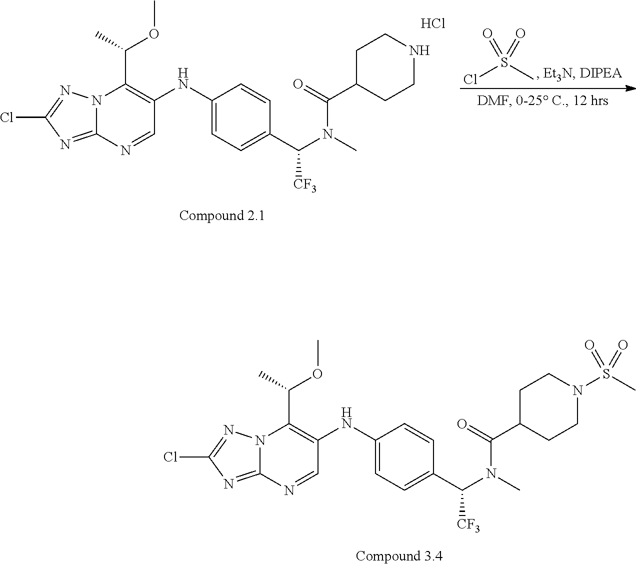

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-4-carboxamide hydrochloride [Compound 2.1, HCl salt] (100 mg, 177 μmol), DIPEA (91.5 mg, 708 μmol), and triethylamine (71.6 mg, 708 μmol) in DMF (3 mL) was added methanesulfonyl chloride (120 mg, 1.04 mmol) at 0° C. and the mixture was stirred at 25° C. for 12 hr. The crude product was purified by prep-HPLC (column: YMC Triart C18 250*50 mm*7 μm, table: 30-70% B (A=water (0.05% ammonia hydroxide v/v)-ACN, B=acetonitrile), flowrate: 60 mL/min, UV Detector 220 run) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpiperidine-4-carboxamide [Compound 3.4] (21.0 mg, 34.7 μmol, 19.8% yield) as a yellow dry powder. m/z: [M+H]+ Calcd for C24H30ClF3N7O4S 604.2; Found 604.3. ¹H NMR (400 MHz, CDCl₃) δ=8.89 (s, 1H), 7.35-7.29 (m, 2H), 7.12 (s, 1H), 7.05 (d, J=8.8 Hz, 2H), 6.62 (q, J=8.8 Hz, 1H), 5.49 (q, J=6.8 Hz, 1H), 3.91-3.75 (m, 2H), 3.54-3.47 (m, 3H), 3.16-2.86 (m, 5H), 2.83 (s, 3H), 2.78-2.71 (m, 1H), 2.05-1.91 (m, 3H), 1.91-1.84 (m, 1H), 1.62 (d, J=6.8 Hz, 3H).

### Synthesis of 1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide (Compound 3.5)

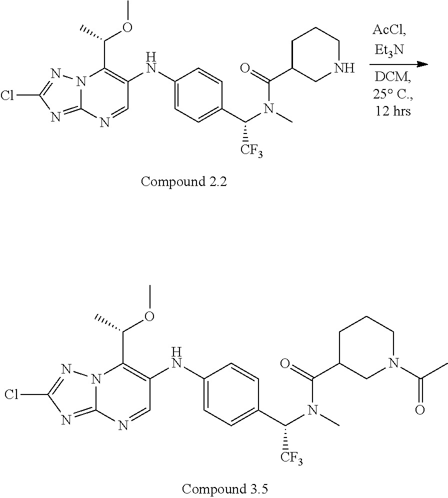 [SMILES: CO[C@@H](C)c1c(Nc2ccc([C@H](N(C)C(=O)C3CCCNC3)C(F)(F)F)cc2)cnc2nc(Cl)nn12 ; CO[C@@H](C)c1c(Nc2ccc([C@H](N(C)C(=O)C3CCCN(C(C)=O)C3)C(F)(F)F)cc2)cnc2nc(Cl)nn12]

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide [Compound 2.2] (40 mg, 76.0 μmol) and acetyl chloride (20.8 mg, 266 μmol) in DCM (2 mL) was added triethylamine (38.4 mg, 380 μmol) at 25° C. and the mixture was stirred at 25° C. for 12 hr. The reaction was concentrated under reduced pressure to give the crude product, which was purified by prep-HPLC (column: YMC Triart C18 250*50 mm*7 μm, table: 31-71% B (A=water (0.05% ammonia hydroxide v/v)), B=acetonitrile), flow rate: 60 mL/min, UV Detector 220 nm) to afford 1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide [Compound 3.5] (8.90 mg, 15.6 μmol, 20.6% yield) as a yellow dry power. m/z: [M+H]+ Calcd for C25H30ClF3N7O3 568.2; Found 568.3. ¹H NMR (400 MHz, CDCl₃) δ=8.97-8.88 (m, 1H), 7.38-7.29 (m, 2H), 7.15-7.10 (m, 1H), 7.08-7.02 (m, 2H), 6.66-6.51 (m, 1H), 5.48 (q, J=6.8 Hz, 1H), 4.73-4.55 (m, 1H), 3.90-3.78 (m, 1H), 3.49 (s, 3H), 3.22-3.03 (m, 1H), 2.94 (s, 3H), 2.82-2.64 (m, 2H), 2.11 (s, 3H), 2.07-1.99 (m, 1H), 1.96-1.82 (m, 2H), 1.81-1.66 (m, 1H), 1.63-1.60 (m, 2H), 1.58-1.45 (m, 1H).

### Synthesis of N3-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N1,N3-dimethylpiperidine 1,3-dicarboxamide (Compound 3.6)

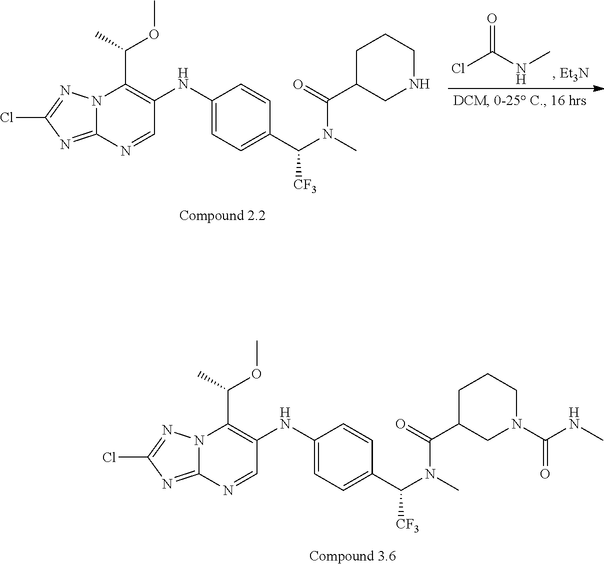

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide [Compound 2.2] (50 mg, 95.0 μmol) in CH₂Cl₂ (3 mL) was added triethylamine (38.4 mg, 380 μmol) followed by N-methylcarbamoyl chloride (10.5 mg, 113 μmol) at 0° C. The mixture was stirred at 25° C. for 16 hr. The reaction mixture was concentrated under reduced pressure to give the crude product, which was purified by prep-HPLC (column: Phenomenex Gemini-NX 80*40 mm*3 μm, table: 22-62% B (A=water (0.05% ammonia hydroxide)), B=acetonitrile), flow rate: 25 mL/min, UV Detector 220 nm) to give N3-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N1,N3-dimethylpiperidine 1,3-dicarboxamide [Compound 3.6] (8.00 mg, 13.7 μmol, 14.4% yield) as a yellow solid. m/z: [M+H]+ Calcd for C25H31ClF3N8O3 583.2; Found 583.4. ¹H NMR (400 MHz, DMSO-d6) δ=8.87-8.83 (m, 1H), 8.05-7.94 (m, 1H), 7.37-7.15 (m, 2H), 7.02-6.94 (m, 2H), 6.53-6.37 (m, 2H), 5.15 (q, J=6.8 Hz, 1H), 4.04 (br d, J=9.6 Hz, 1H), 3.91 (br d, J=13.2 Hz, 1H), 3.16 (s, 3H), 2.90 (s, 3H), 2.69 (br dd, J=3.6, 11.6 Hz, 2H), 2.66-2.61 (m, 1H), 2.57-2.54 (m, 3H), 1.87-1.72 (m, 1H), 1.59 (d, J=6.8 Hz, 3H), 1.57-1.45 (m, 2H), 1.43-1.30 (m, 1H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpiperidine-3-carboxamide (Compound 3.7)

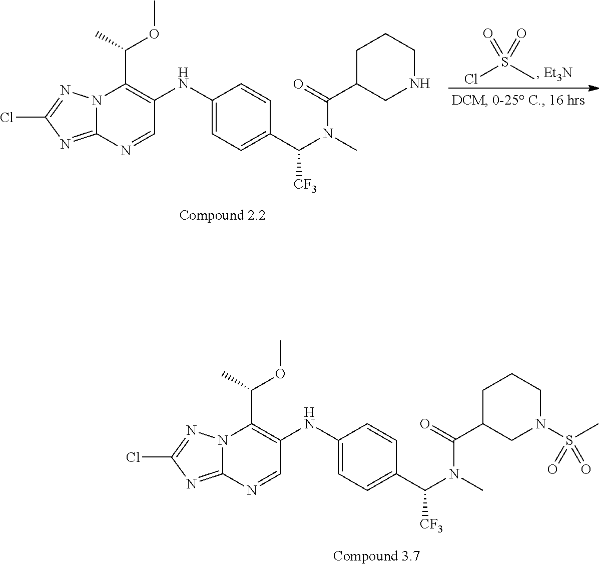

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide [Compound 2.2] (50 mg, 95.0 μmol) in CH₂Cl₂ (2 mL) was added triethylamine (38.4 mg, 380 μmol), followed by methanesulfonyl chloride (90 mg, 785 μmol) at 0° C. The mixture was stirred at 25° C. for 16 hr. The reaction mixture was concentrated under reduced pressure to give the crude product, which was purified by prep-HPLC (column: Boston Prime C18 150*30 mm*5 μm, table: 34-64% B (water (0.05% ammonia hydroxide v/v), B=acetonitrile), flow rate: 25 mL/min, UV Detector 220 nm) to give N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpiperidine-3-carboxamide [Compound 3.7] (4.30 mg, 7.11 μmol, 7.5% yield) as a white dry power. m/z: [M+H]+ Calcd for C24H30ClF3N7O4S 604.2; Found 604.3. ¹H NMR (400 MHz, DMSO-d6) δ=8.86-8.80 (m, 1H), 7.99 (s, 1H), 7.26-7.14 (m, 2H), 7.00-6.90 (m, 2H), 6.50-6.12 (m, 1H), 5.18-4.94 (m, 1H), 3.64-3.52 (m, 2H), 3.33-3.30 (m, 3H), 3.16-3.07 (m, 3H), 2.90 (s, 3H), 2.88-2.84 (m, 1H), 2.82-2.65 (m, 2H), 1.88-1.71 (m, 2H), 1.58 (d, J=6.8 Hz, 3H), 1.55-1.49 (m, 1H), 1.48-1.36 (m, 1H).

### Synthesis of 1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide (Compound 3.8)

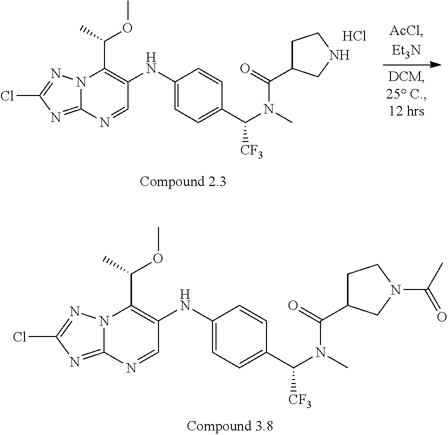 [SMILES: CO[C@@H](C)c1c(Nc2ccc([C@H](N(C)C(=O)C3CCNC3)C(F)(F)F)cc2)cnc2nc(Cl)nn12 ; Cl ; CO[C@@H](C)c1c(Nc2ccc([C@H](N(C)C(=O)C3CCN(C(C)=O)C3)C(F)(F)F)cc2)cnc2nc(Cl)nn12]

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide hydrochloride [Compound 2.3] (150 mg, 273 μmol) and triethylamine (137 mg, 1.36 mmol) in DCM (2 mL) was added acetyl chloride (74.9 mg, 955 μmol) at 25° C. and the mixture was stirred at 25° C. for 12 hr. The mixture was concentrated under reduced pressure to afford the crude product, which was purified by prep-HPLC (column: Boston Prime C18 150*30 mm*5 μm, table: 22-52% B (A=water (0.05% ammonia hydroxide v/v)-ACN), B=acetonitrile), flow rate: 30 mL/min, UV Detector 220 nm) to afford 1-acetyl-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide [Compound 3.8] (8.40 mg, 15.1 μmol, 5.6% yield) as a yellow dry powder. m/z: [M+H]+ Calcd for C24H28ClF3N7O3 554.2; Found 554.4. ¹H NMR (400 MHz, CDCl₃) δ=8.94-8.88 (m, 1H), 7.35-7.29 (m, 2H), 7.14 (s, 1H), 7.05 (dd, J=4.0, 8.4 Hz, 2H), 6.65-6.53 (m, 1H), 5.48 (q, J=6.8 Hz, 1H), 4.01-3.51 (m, 4H), 3.49 (s, 3H), 3.44-3.25 (m, 1H), 3.00-2.91 (m, 3H), 2.53-2.11 (m, 2H), 2.11-2.06 (m, 3H), 1.62 (br s, 3H).

### Synthesis of N3-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N1,N3-dimethylpyrrolidine-1,3-dicarboxamide (Compound 3.9)

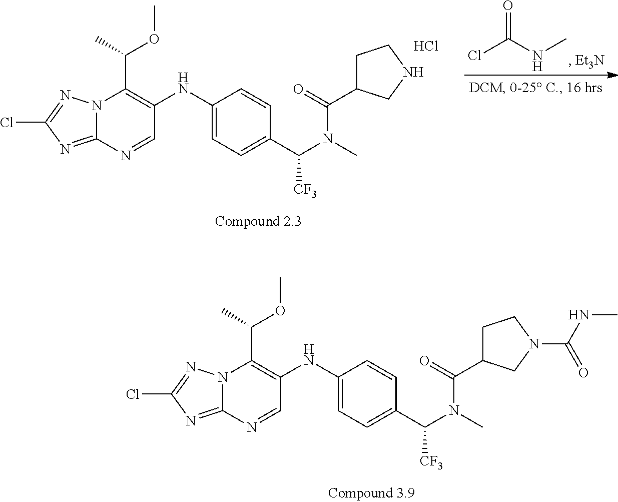

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide hydrochloride [Compound 2.3] (150 mg, 273 μmol) in CH₂Cl₂ (3 mL) was added triethylamine (110 mg, 1.09 mmol) followed by N-methylcarbamoyl chloride (30.5 mg, 327 μmol) at 0° C. The mixture was stirred at 25° C. for 16 hr. The reaction mixture was concentrated under reduced pressure to give the crude product, which was purified by prep-HPLC (column: Boston Prime C18 150*30 mm*5 μm, table: 22-52% B (A=water (0.05% ammonia hydroxide v/v)-ACN)). B=acetonitrile), flow rate: 25 mL/min, UV Detector 220 nm) to give N3-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N1,N3-dimethylpyrrolidine-1,3-dicarboxamide [Compound 3.9] (11.4 mg, 20.0 μmol, 7.4% yield) as a yellow dry powder. m/z: [M+H]+ Calcd for C24H29ClF3N8O3 569.1; Found 569.4. ¹H NMR (400 MHz, CDCl₃) δ=8.91 (d, J=5.6 Hz, 1H), 7.35-7.29 (m, 2H), 7.13 (s, 1H), 7.05 (d, J=8.4 Hz, 2H), 6.64-6.54 (m, 1H), 5.48 (q, J=6.8 Hz, 1H), 4.21 (br d, J=4.0 Hz, 1H), 3.81-3.68 (m, 1H), 3.63-3.54 (m, 2H), 3.49 (d, J=1.2 Hz, 3H), 3.44-3.28 (m, 2H), 2.94 (d, J=4.4 Hz, 3H), 2.84 (dd, J=2.4, 4.8 Hz, 3H), 2.42-2.11 (m, 2H), 1.63 (br s, 3H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpyrrolidine-3-carboxamide (Compound 3.10)

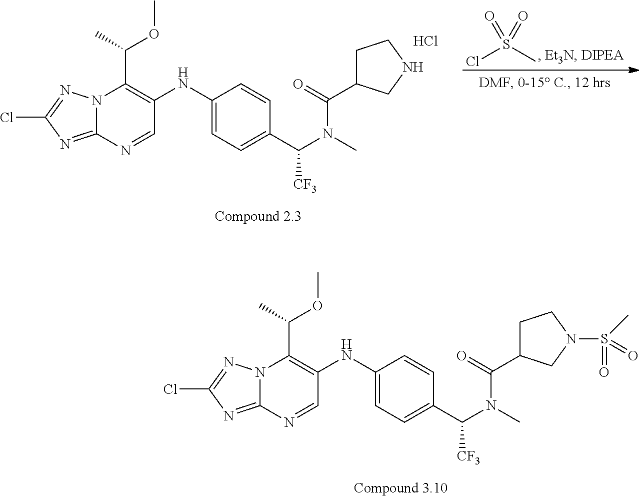

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide hydrochloride [Compound 2.3] (150 mg, 293 μmol), DIPEA (151 mg, 1.17 mmol), and triethylamine (118 mg, 1.17 mmol) in DMF (2 mL) was added methanesulfonyl chloride (310 mg, 2.70 mmol) at 0° C. The mixture was stirred at 15° C. for 12 hr. The crude product was purified by prep-HPLC (column: Boston Green ODS 150*30 mm*5 μm, table: 28-68% B (A=water (0.05% HCl)-ACN, B=acetonitrile), flowrate: 30 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-methanesulfonyl-N-methylpyrrolidine-3-carboxamide [Compound 3.10] (22.5 mg, 35.9 μmol, 12.2% yield) as a yellow dry powder. m/z: [M+H]+ Calcd for C23H28ClF3N7O4S 590.1; Found 590.4. ¹H NMR (400 MHz, CDCl₃) δ=8.90 (s, 1H), 7.35-7.29 (m, 2H), 7.14 (s, 1H), 7.05 (d, J=8.4 Hz, 2H), 6.57 (q, J=9.2 Hz, 1H), 5.49 (q, J=6.8 Hz, 1H), 3.78-3.53 (m, 3H), 3.49 (s, 3H), 3.46-3.35 (m, 2H), 2.97-2.91 (m, 6H), 2.37-2.15 (m, 2H), 1.62 (s, 3H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4-(acetamidomethyl)-N-methylcyclohexane-1-carboxamide (Compound 3.11)

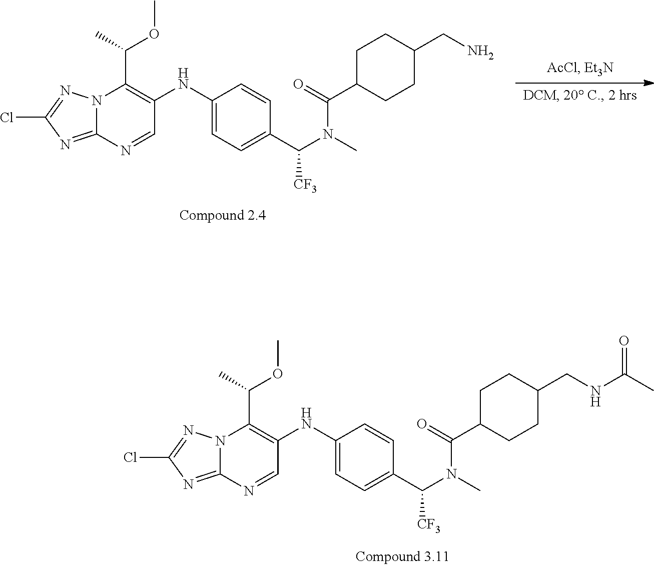 [SMILES: CO[C@@H](C)c1c(Nc2ccc([C@H](N(C)C(=O)C3CCC(CN)CC3)C(F)(F)F)cc2)cnc2nc(Cl)nn12 ; CO[C@@H](C)c1c(Nc2ccc([C@H](N(C)C(=O)C3CCC(CNC(C)=O)CC3)C(F)(F)F)cc2)cnc2nc(Cl)nn12]

To a solution of 4-(aminomethyl)-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylcyclohexane-1-carboxamide [free base of Compound 2.4] (90 mg, 162 μmol) in CH₂Cl₂ (1.5 mL) was added Et₃N (49.0 mg, 485 μmol) and acetyl chloride (63.5 mg, 810 μmol). The resulting mixture was stirred at 20° C. for 2 h. The reaction was concentrated under reduced pressure to give a residue. The residue was purified by prep-HPLC (column: YMC Triart C18 250*50 mm*7 μm, table: 26-66% B (A=water (0.05% ammonia hydroxide v/v), B=acetonitrile), flow rate: 60 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-4-(acetamidomethyl)-N-methylcyclohexane-1-carboxamide [Compound 3.11] (23.8 mg, 39.9 μmol, 24.6% yield) as a yellow dry powder. m/z: [M+H]+ Calcd for C27H34ClF3N7O3 596.2; Found 596.2. ¹H NMR (400 MHz, CDCl₃) δ=8.81 (s, 1H), 7.23 (br d, J=8.4 Hz, 2H), 7.04 (s, 1H), 6.96 (d, J=8.4 Hz, 2H), 6.55 (q, J=9.2 Hz, 1H), 5.64-5.52 (m, 1H), 5.40 (q, J=6.8 Hz, 1H), 3.41 (s, 3H), 3.13-3.00 (m, 2H), 2.83 (s, 3H), 2.52-2.41 (m, 1H), 1.96-1.90 (m, 3H), 1.80-1.66 (m, 3H), 1.60-1.41 (m, 6H), 1.06-0.85 (m, 2H).

### Synthesis of methyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate (Compound 3.12)

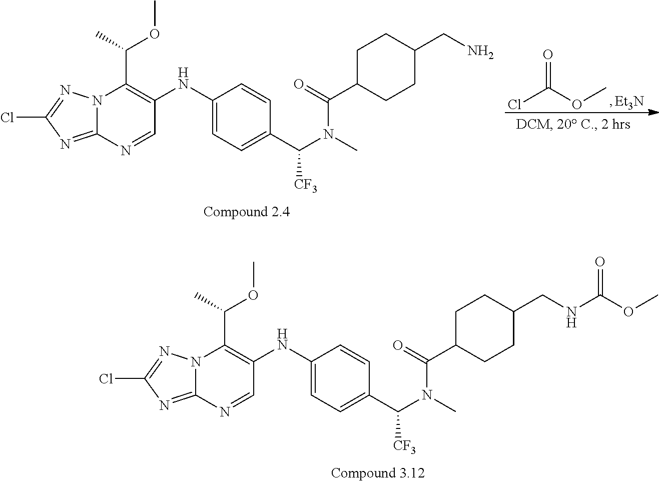

To a solution of 4-(aminomethyl)-N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylcyclohexane-1-carboxamide [free base of Compound 2.4] (90 mg, 162 μmol) in CH₂Cl₂ (1.5 mL) was added Et₃N (49.0 mg, 485 μmol) and methyl carbonochloridate (75.7 mg, 810 μmol). The resulting mixture was stirred at 20° C. for 2 h. The reaction was poured into water (10 mL) and extracted with EtOAc (10 mL×2). The combined organic layers were washed with brine (15 mL×2) and concentrated under reduced pressure to give a residue. The residue was purified by prep-HPLC (column: YMC Triart C18 250*50 mm*7 μm, table: 31-71% B (A=water (0.05% ammonia hydroxide v/v), B=acetonitrile), flow rate: 60 mL/min, UV Detector 220 nm) to afford methyl N-[(4-{[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl](methyl)carbamoyl}cyclohexyl)methyl]carbamate [Compound 3.12] (19.3 mg, 31.5 μmol, 19.4% yield) as a yellow dry powder. m/z: [M+H]+ Calcd for C27H34ClF3N7O4 612.2; Found 612.2. ¹H NMR (400 MHz, CDCl₃) δ=8.81 (s, 1H), 7.23 (br d, J=8.0 Hz, 2H), 7.04 (s, 1H), 6.96 (br d, J=8.4 Hz, 2H), 6.55 (q, J=8.8 Hz, 1H), 5.40 (q, J=6.8 Hz, 1H), 4.72 (br s, 1H), 3.59 (s, 3H), 3.41 (s, 3H), 3.08-2.92 (m, 2H), 2.83 (s, 3H), 2.46 (brt, J=11.6 Hz, 1H), 1.83-1.67 (m, 4H), 1.65-1.38 (m, 6H), 0.95 (brt, J=11.2 Hz, 2H).

### 4. Compounds Prepared Using Scheme 4

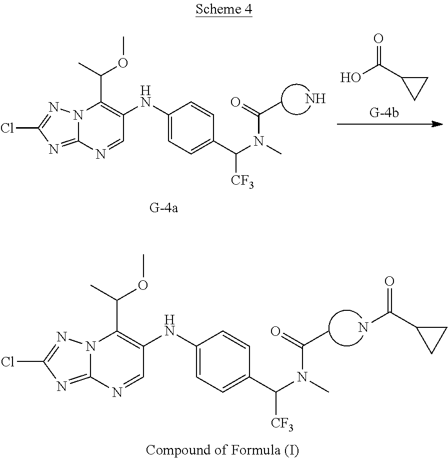

Starting material G-4a is treated with G-4b to provide a compound of formula (I).

### Synthesis of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-1-(cyclopropanecarbonyl)-N-methylpiperidine-4-carboxamide (Compound 4.1)

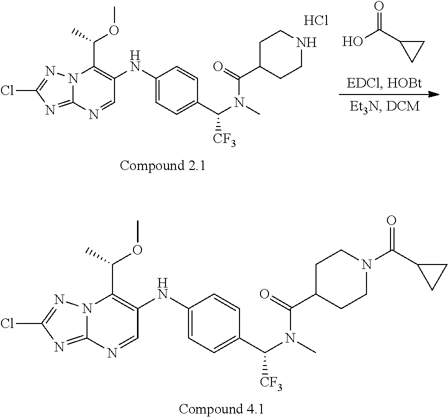 [SMILES: CO[C@@H](C)c1c(Nc2ccc([C@H](N(C)C(=O)C3CCNCC3)C(F)(F)F)cc2)cnc2nc(Cl)nn12 ; Cl ; O=C(O)C1CC1 ; CO[C@@H](C)c1c(Nc2ccc([C@H](N(C)C(=O)C3CCN(C(=O)C4CC4)CC3)C(F)(F)F)cc2)cnc2nc(Cl)nn12]

To a mixture of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide hydrochloride [Compound 2.1, HCl salt] (80 mg, 142 μmol), EDCI (54.4 mg, 284 μmol), hydroxybenzotriazole (19.1 mg, 142 μmol), and triethylamine (43.0 mg, 425 μmol) in DCM (3 mL) was added cyclopropanecarboxylic acid (36.5 mg, 425 μmol). The mixture was stirred at 25° C. for 12 hr. The crude product was purified by prep-HPLC (column: Phenomenex Gemini-NX 80*40 mm*3 μm, table: 21-61% B (A=water (0.05% ammonia hydroxide v/v), B=acetonitrile), flowrate: 25 mL/min, UV Detector 220 nm) to afford N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-1-(cyclopropanecarbonyl)-N-methylpiperidine-4-carboxamide [Compound 4.1] (20.5 mg, 34.5 μmol, 24.3% yield) as yellow dry powder. m/z: [M+H]+ Calcd for C27H32ClF3N7O3 594.2; Found 594.5. ¹H NMR (400 MHz, CDCl₃) δ=8.89 (s, 1H), 7.32 (br d, J=8.0 Hz, 2H), 7.17-7.02 (m, 3H), 6.63 (q, J=9.2 Hz, 1H), 5.48 (q, J=6.8 Hz, 1H), 4.62 (br s, 1H), 4.32 (br s, 1H), 3.49 (s, 3H), 3.29-3.10 (m, 1H), 2.95 (s, 3H), 2.90-2.70 (m, 2H), 1.98-1.67 (m, 5H), 1.62 (d, J=6.8 Hz, 3H), 0.99 (br s, 2H), 0.78 (br d, J=7.8 Hz, 2H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-cyclopropanecarbonyl-N-methylpiperidine-3-carboxamide [Compound 4.2]

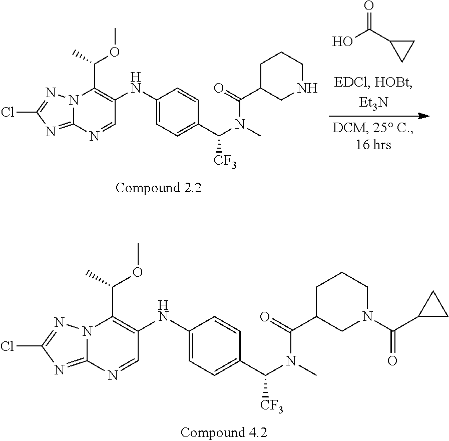 [SMILES: CO[C@@H](C)c1c(Nc2ccc([C@H](N(C)C(=O)C3CCCNC3)C(F)(F)F)cc2)cnc2nc(Cl)nn12 ; O=C(O)C1CC1 ; CO[C@@H](C)c1c(Nc2ccc([C@H](N(C)C(=O)C3CCCN(C(=O)C4CC4)C3)C(F)(F)F)cc2)cnc2nc(Cl)nn12]

To a mixture of cyclopropanecarboxylic acid (12.2 mg, 142 μmol), EDCI (27.2 mg, 142 μmol), and HOBt (19.1 mg, 142 μmol) in CH₂Cl₂ (3 mL) was added N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide [Compound 2.2] (50 mg, 95.0 μmol) and the mixture was stirred at 25° C. for 16 hr under N₂. The reaction was concentrated under reduced pressure to give the crude product, which was purified by prep-HPLC (column: Phenomenex Gemini-NX C18 75*30 mm*3 μm, table: 34-74% B (A=water (0.225% FA), B=acetonitrile), flow rate: 25 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-cyclopropanecarbonyl-N-methylpiperidine-3-carboxamide [Compound 4.2] (2.80 mg, 4.71 μmol, 5.0% yield) as a yellow solid. m/z: [M+H]+ Calcd for C27H32ClF3N7O3 594.2; Found 594.4. ¹H NMR (400 MHz, DMSO-d6) δ=8.85 (s, 1H), 8.02 (br s, 1H), 7.30-7.16 (m, 2H), 6.97 (br d, J=8.0 Hz, 2H), 6.44 (br d, J=8.8 Hz, 1H), 5.16 (br d, J=6.4 Hz, 1H), 4.47-4.19 (m, 2H), 3.16 (s, 3H), 2.89 (br s, 3H), 2.70 (br d, J=18.8 Hz, 1H), 2.62 (br s, 2H), 2.00 (br s, 1H), 1.91-1.72 (m, 2H), 1.71-1.63 (m, 1H), 1.59 (br d, J=6.4 Hz, 3H), 1.24 (br s, 1H), 0.72 (br s, 4H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-cyclopropanecarbonyl-N-methylpyrrolidine-3-carboxamide (Compound 4.3)

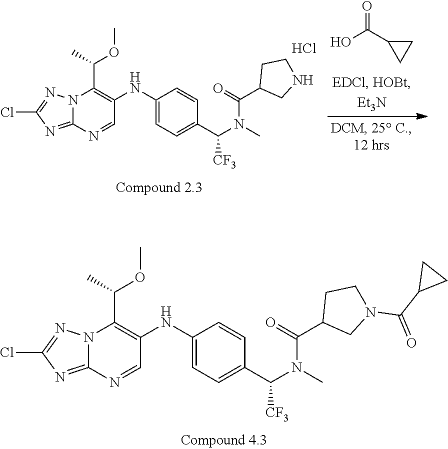 [SMILES: CO[C@@H](C)c1c(Nc2ccc([C@H](N(C)C(=O)C3CCNC3)C(F)(F)F)cc2)cnc2nc(Cl)nn12 ; Cl ; O=C(O)C1CC1 ; CO[C@@H](C)c1c(Nc2ccc([C@H](N(C)C(=O)C3CCN(C(=O)C4CC4)C3)C(F)(F)F)cc2)cnc2nc(Cl)nn12]

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide hydrochloride [Compound 2.3] (150 mg, 273 μmol), EDCI (104 mg, 546 μmol), hydroxybenzotriazole (36.8 mg, 273 μmol), and triethylamine (82.7 mg, 818 μmol) in DCM (3 mL) was added cyclopropanecarboxylic acid (70.4 mg, 818 μmol). The mixture was stirred at 25° C. for 12 hr. The crude product was purified by prep-HPLC (column: Boston Prime C18 150*30 mm*5 μm, table: 39-69% B (A=water (0.05% ammonia hydroxide v/v)-ACN), B=acetonitrile), flowrate: 30 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-1-cyclopropanecarbonyl-N-methylpyrrolidine-3-carboxamide [Compound 4.3] (14.7 mg, 25.3 μmol, 9.3% yield) as a yellow dry powder. m/z: [M+H]+ Calcd for C26H30ClF3N7O3 580.2; Found 580.5. ¹H NMR (400 MHz, CDCl₃) δ=8.92-8.90 (m, 1H), 7.37-7.30 (m, 2H), 7.13 (s, 1H), 7.08-7.02 (m, 2H), 6.67-6.55 (m, 1H), 5.48 (q, J=6.8 Hz, 1H), 4.09-3.53 (m, 4H), 3.49 (s, 3H), 3.45-3.26 (m, 1H), 3.00-2.92 (m, 3H), 2.55-2.02 (m, 2H), 1.64 (br d, J=5.4 Hz, 1H). 1.63 (s, 3H), 1.09-0.96 (m, 2H), 0.79 (br d, J=7.6 Hz, 2H).

### 5. Compounds Prepared Using Scheme 5

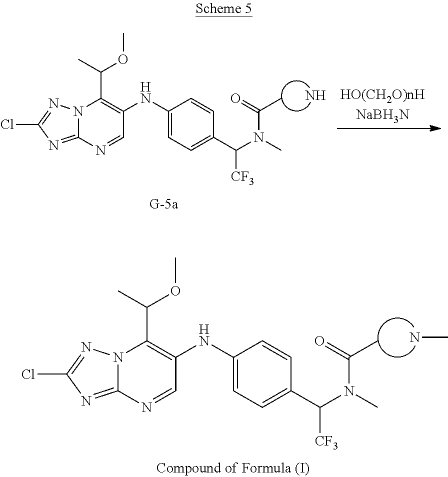

Starting material G-5a is treated with HO(CH₂O)ₙH (paraformaldehyde) and NaBH₃CN to provide a compound of formula (I).

### Synthesis of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N,1-dimethylpiperidine-4-carboxamide (Compound 5.1)

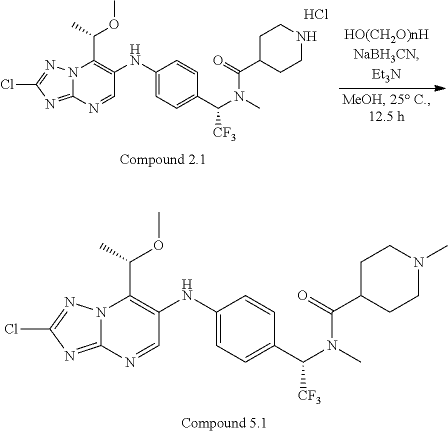 [SMILES: CO[C@@H](C)c1c(Nc2ccc([C@H](N(C)C(=O)C3CCNCC3)C(F)(F)F)cc2)cnc2nc(Cl)nn12 ; Cl ; CO[C@@H](C)c1c(Nc2ccc([C@H](N(C)C(=O)C3CCN(C)CC3)C(F)(F)F)cc2)cnc2nc(Cl)nn12]

A mixture of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylpiperidine-4-carboxamide hydrochloride [Compound 2.1, HCl salt] (80 mg, 152 μmol), sodium cyanoborohydride (14.2 mg, 227 μmol) and triethylamine (30.7 mg, 304 μmol) in MeOH (3 mL) was stirred at 25° C. for 0.5 hr. Paraformaldehyde (22.8 mg, 760 μmol) was added and the mixture was stirred at 25° C. for 12 hr. The crude product was purified by prep-HPLC (column: Phenomenex Gemini-NX 80*40 mm*3 μm, table: 39-79% B (A=water (0.05% ammonia hydroxide v/v), B=acetonitrile), flowrate: 25 mL/min, UV Detector 220 nm) to afford N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N,1-dimethylpiperidine-4-carboxamide [Compound 5.1] (22.0 mg, 40.7 μmol, 26.8% yield) as a yellow dry powder. m/z: [M+H]+ Calcd for C24H30ClF3N7O2 540.2; Found 540.5. ¹H NMR (400 MHz, CDCl₃) δ=8.90 (s, 1H), 7.33 (br d, J=8.4 Hz, 2H), 7.11 (s, 1H), 7.04 (d, J=8.4 Hz, 2H), 6.64 (q, J=9.2 Hz, 1H), 5.48 (q, J=6.8 Hz, 1H), 3.49 (s, 3H), 3.03-2.85 (m, 5H), 2.54 (br s, 1H), 2.30 (s, 3H), 2.07-1.89 (m, 4H), 1.87-1.79 (m, 1H), 1.78-1.70 (m, 1H), 1.62 (s, 3H).

### Synthesis of N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N,1-dimethylpiperidine-3-carboxamide (Compound 5.2)

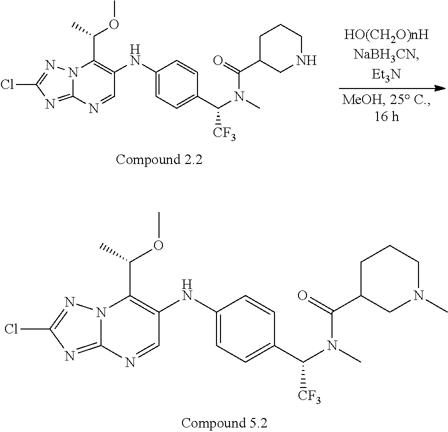 [SMILES: CO[C@@H](C)c1c(Nc2ccc([C@H](N(C)C(=O)C3CCCNC3)C(F)(F)F)cc2)cnc2nc(Cl)nn12 ; CO[C@@H](C)c1c(Nc2ccc([C@H](N(C)C(=O)C3CCCN(C)C3)C(F)(F)F)cc2)cnc2nc(Cl)nn12]

To a mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpiperidine-3-carboxamide [Compound 2.2] (50 mg, 95.0 μmol) and paraformaldehyde (14.2 mg, 474 μmol) in MeOH (1 mL) was added triethylamine (19.2 mg, 190 μmol) and sodium cyanoborohydride (8.92 mg, 142 μmol). The mixture was stirred at 25° C. for 16 hr under N₂. The reaction mixture was concentrated under reduced pressure to give the crude product, which was purified by prep-HPLC (column: YMC Triart C18 250*50 mm*7 μm, table: 11-51% B (A=water(0.225% FA), B=acetonitrile), flow rate: 60 mL/min, UV Detector 220 nm) to give N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N,1-dimethylpiperidine-3-carboxamide [Compound 5.2] (4.70 mg, 8.70 μmol, 9.2% yield) as a yellow dry powder. m/z: [M+H]+ Calcd for C24H30ClF3N7O2 540.2; Found 540.3. ¹H NMR (400 MHz, DMSO-d6) δ=8.83 (s, 1H), 8.20 (s, 1H), 8.04-7.94 (m, 1H), 7.28-7.12 (m, 2H), 6.97 (br d, J=8.4 Hz, 2H), 6.43 (q, J=9.2 Hz, 1H), 5.15 (q, J=6.8 Hz, 1H), 3.16 (s, 3H), 3.00-2.78 (m, 6H), 2.25 (s, 3H), 2.19-1.91 (m, 2H), 1.87-1.74 (m, 1H), 1.73-1.60 (m, 2H), 1.59 (d, J=6.8 Hz, 3H), 1.40-1.24 (m, 1H).

### Synthesis of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N,1-dimethylpyrrolidine-3-carboxamide hydrochloride (Compound 5.3)

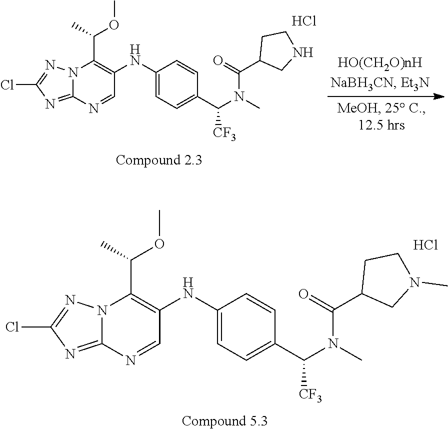 [SMILES: CO[C@@H](C)c1c(Nc2ccc([C@H](N(C)C(=O)C3CCNC3)C(F)(F)F)cc2)cnc2nc(Cl)nn12 ; Cl ; CO[C@@H](C)c1c(Nc2ccc([C@H](N(C)C(=O)C3CCN(C)C3)C(F)(F)F)cc2)cnc2nc(Cl)nn12 ; Cl]

A mixture of N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N-methylpyrrolidine-3-carboxamide hydrochloride [Compound 2.3] (150 mg, 273 μmol), sodium cyanoborohydride (25.7 mg, 409 μmol), and triethylamine (55.2 mg, 546 μmol) in MeOH (3 mL) was stirred at 25° C. for 0.5 hr. Paraformaldehyde (40.8 mg, 1.36 mmol) was then added and the reaction was stirred at 25° C. for 12 hr. The crude product was purified by prep-HPLC (column: Boston Green ODS 150*30 mm*5 μm, table: 16-56% B (A=water (0.05% HCl)-ACN), B=acetonitrile), flowrate: 30 mL/min, UV Detector 220 nm) to afford N-[(1S)-1-[4-({2-chloro-7-[(1S)-1-methoxyethyl]-[1,2,4]triazolo[1,5-a]pyrimidin-6-yl}amino)phenyl]-2,2,2-trifluoroethyl]-N,1-dimethylpyrrolidine-3-carboxamide hydrochloride [Compound 5.3] (14.3 mg, 25.4 μmol, 9.3% yield) as a yellow dry powder. m/z: [M+H]+ Calcd for C23H28ClF3N7O2 526.2; Found 526.3. ¹H NMR (400 MHz, CDCl₃) δ=12.84 (br s, 1H), 8.91 (s, 1H), 7.33-7.28 (m, 2H), 7.15 (br d, J=3.6 Hz, 1H), 7.05 (br d, J=8.4 Hz, 2H), 6.58-6.43 (m, 1H), 5.48 (q, J=6.8 Hz, 1H), 4.04-3.87 (m, 1H), 3.80 (br s, 2H), 3.49 (s, 3H), 3.48-3.36 (m, 1H), 3.04 (br s, 1H), 3.00-2.96 (m, 3H), 2.94 (s, 3H), 2.71 (br d, J=17.2 Hz, 1H), 2.11 (br s, 1H), 1.60 (s, 3H).

### 6. Compounds Prepared Using Scheme 6

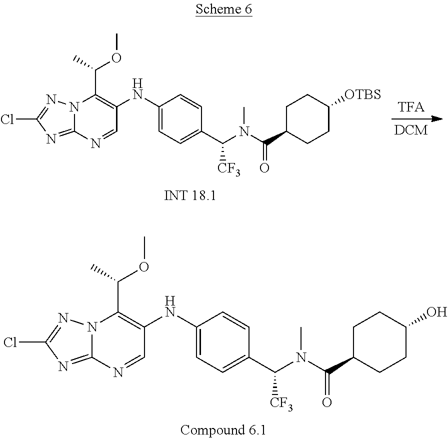 [SMILES: CO[C@@H](C)c1c(Nc2ccc([C@H](N(C)C(=O)[C@H]3CC[C@H](O[Si](C)(C)C(C)(C)C)CC3)C(F)(F)F)cc2)cnc2nc(Cl)nn12 ; CO[C@@H](C)c1c(Nc2ccc([C@H](N(C)C(=O)[C@H]3CC[C@H](O)CC3)C(F)(F)F)cc2)cnc2nc(Cl)nn12]

### Synthesis of (1r,4S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-4-hydroxy-N-methylcyclohexane-1-carboxamide (Compound 6.1)

A solution of (1r,4S)-4-((tert-butyldimethylsilyl)oxy)-N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylcyclohexane-1-carboxamide [INT 18.1] (100 mg, 152 μmol) in TFA (0.5 mL) and CH₂Cl₂ (0.5 mL) was stirred at 20° C. for 0.5 h. The reaction mixture was concentrated under reduced pressure to give a residue. The residue was purified by prep-HPLC (column: Boston Green ODS 150*30 mm*5 μm, table: 23-63% B (A=water (0.05% HCl), B=acetonitrile), flow rate: 30 mL/min, UV Detector 220 nm) to afford (1r,4S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-4-hydroxy-N-methylcyclohexane-1-carboxamide [Compound 6.1] (6.50 mg, 12.0 μmol. 7.9% yield) as a yellow dry powder. m/z: [M+H]+ Calcd for C24H29ClF3N6O3 541.2; Found 541.3. ¹H NMR (400 MHz, CDCl₃) δ=9.05 (s, 1H), 7.47 (br d, J=8.0 Hz, 2H), 7.43 (s, 1H), 7.20 (br d, J=8.4 Hz, 2H), 6.78 (q, J=8.8 Hz, 1H), 5.64 (q, J=6.4 Hz, 1H), 3.93-3.81 (m, 1H), 3.65 (s, 3H), 3.08 (s, 3H), 2.78-2.64 (m, 1H), 2.27 (br dd, J=3.6, 8.4 Hz, 2H), 2.13-2.05 (m, 1H), 2.03-1.96 (m, 1H), 1.91-1.85 (m, 2H), 1.77 (d, J=6.8 Hz, 3H), 1.57-1.42 (m, 2H).

### 7. Compounds Prepared Using Scheme 7

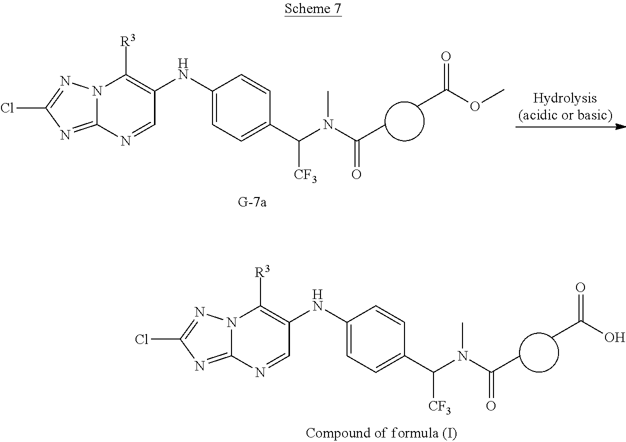

Starting material G-7a is treated with a hydrolyzing agent to provide a compound of formula (I).

### Synthesis of (1S,4r)-4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexane-1-carboxylic acid (Compound 7.1)

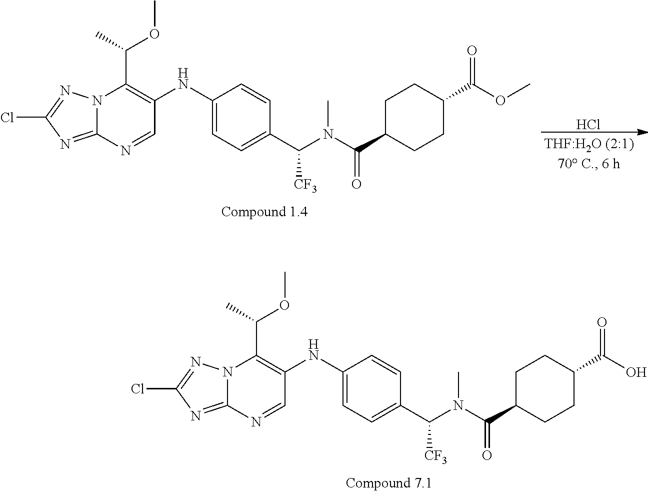 [SMILES: COC(=O)[C@H]1CC[C@H](C(=O)N(C)[C@@H](c2ccc(Nc3cnc4nc(Cl)nn4c3[C@H](C)OC)cc2)C(F)(F)F)CC1 ; Cl ; CO[C@@H](C)c1c(Nc2ccc([C@H](N(C)C(=O)[C@H]3CC[C@H](C(=O)O)CC3)C(F)(F)F)cc2)cnc2nc(Cl)nn12]

To a mixture of methyl (1S,4r)-4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexane-1-carboxylate [Compound 1.4] (100 mg, 171 μmol) in THF (2 mL) and H₂O (1 mL) was added 1 N HCl (1 mL, 1 mmol) and the mixture was stirred at 70° C. for 6 hr. Water (10 mL) was added and the mixture was extracted with EtOAc (10 mL×2). The combined organic layers were dried over anhydrous Na₂SO₄, filtered and concentrated under reduced pressure to give the crude product, which was purified by prep-HPLC (column: Boston Green ODS 150*30 mm*5 μm, table: 28-68% B (A=water (0.05% HCl v/v)), B=acetonitrile), flow rate: 30 mL/min, UV Detector 220 nm) and lyophilization to afford (1S,4r)-4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexane-1-carboxylic acid [Compound 7.1] (42.5 mg, 74.6 μmol, 43.7% yield) as a yellow dry powder. m/z: [M+H]+ Calcd for C25H29ClF3N6O4 569.2; Found 569.2. ¹H NMR (400 MHz, DMSO-d6) δ=12.06 (br s, 1H), 8.84 (s, 1H), 8.03-7.97 (m, 1H), 7.25-7.13 (m, 2H), 7.01-6.93 (m, 2H), 6.51-6.10 (m, 1H), 5.15 (q, J=6.4 Hz, 1H), 3.16 (s, 3H), 2.90-2.68 (m, 3H), 2.49-2.38 (m, 1H), 2.27-2.13 (m, 1H), 2.01-1.87 (m, 2H), 1.84-1.68 (m, 2H), 1.59 (d, J=6.8 Hz, 3H), 1.49-1.29 (m, 4H).

### Synthesis of (1S,4r)-4-(((S)-1-(4-((2-chloro-7-isopropyl-[1,2,4]triazolo[1,5-a]pyrimidin-6-yl)amino)phenyl)-2,2,2-trifluoroethyl)(methyl)carbamoyl)cyclohexane-1-carboxylic acid ammonia salt (Compound 7.2)

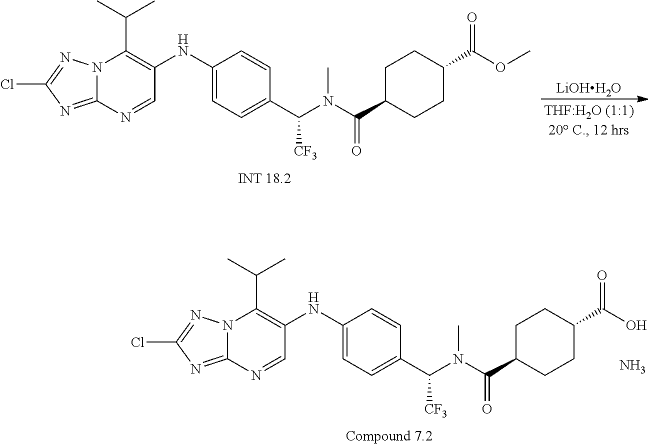

A mixture of methyl (1S,4r)-4-(((S)-1-(4-((2-chloro-7-isopropyl-[1,2,4]triazolo[1,5-a]pyrimidin-6-yl)amino)phenyl)-2,2,2-trifluoroethyl)(methyl)carbamoyl)cyclohexane-1-carboxylate [INT 18.2] (50 mg, 88.1 μmol) and lithium hydroxide monohydrate (7.38 mg, 176 μmol) in H₂O (2 mL) and THF (2 mL) was stirred at 20° C. for 12 h. The mixture was concentrated under reduced pressure to give a residue which was diluted with water (5 mL). The resulting mixture was adjusted to pH=4 with 1 N HCl and extracted with EtOAc (5 mL×2). The combined organic layers were washed with brine (10 mL) and concentrated to give a yellow solid. The solid was purified by prep-HPLC (column: Boston Prime C18 150*30 mm*5 μm, table: 15-45% B (A=water (0.05% ammonia hydroxide v/v), B=acetonitrile), flow rate: 30 mL/min, UV Detector 220 nm) to afford (1S,4r)-4-(((S)-1-(4-((2-chloro-7-isopropyl-[1,2,4]triazolo[1,5-a]pyrimidin-6-yl)amino)phenyl)-2,2,2-trifluoroethyl)(methyl)carbamoyl)cyclohexane-1-carboxylic acid ammonia salt [Compound 7.2] (2.80 mg, 4.91 μmol, 5.6% yield) as a white dry powder. m/z: [M+H]+ Calcd for C25H29ClF3N6O3 553.2; Found 553.1. ¹H NMR (400 MHz, DMSO-d6) δ=8.73 (s, 1H), 8.13 (s, 1H), 7.13 (br d, J=8.4 Hz, 2H), 6.79 (br d, J=8.8 Hz, 2H), 6.51-6.36 (m, 1H), 3.72 (td, J=7.2, 14.0 Hz, 1H), 2.87 (s, 2H), 2.71-2.66 (m, 2H), 2.20 (br s, 1H), 1.98-1.86 (m, 2H), 1.83-1.68 (m, 2H), 1.52-1.32 (m, 10H).

### Synthesis of (1S,3r)-3-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclobutane-1-carboxylic acid (Compound 7.3)

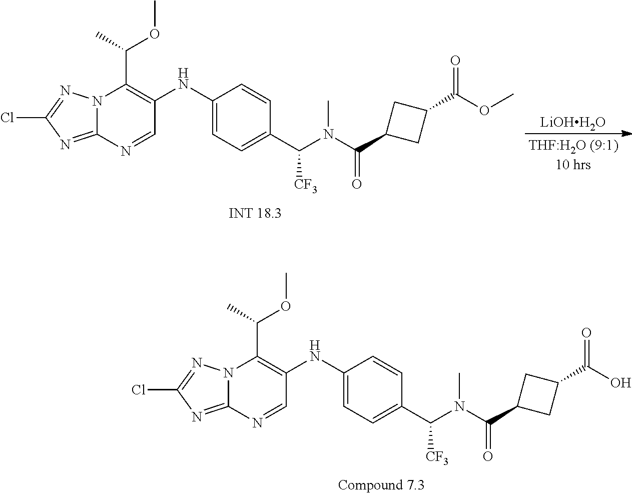

Methyl (1S,3r)-3-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclobutane-1-carboxylate [INT 18.3] (0.136 g, 0.2450 mmol) was dissolved in a mixture of tetrahydrofuran (0.9 mL) and water (0.1 mL). Lithium hydroxide monohydrate (10.2 mg, 245 μmol) was added to the reaction mixture and it was stirred for 10 h. The pH of the reaction mixture was adjusted to 7 with diluted aqueous HCl. Solvents were distilled off and the residue was purified by HPLC (see conditions below) to obtain (1S,3r)-3-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclobutane-1-carboxylic acid [Compound 7.3] (5.70 mg, 0.01053 mmol, 4.3% yield) as a yellow solid. m/z: [M+H]+ Calcd for C23H25ClF3N6O4 541.2; Found 541.0. ¹H NMR (400 MHz, CD₃CN) δ=8.81 (s, 1H), 7.32-7.20 (m, 2H), 7.09-7.00 (m, 3H), 6.59-6.47 (m, 1H), 5.39-5.30 (m, 1H), 3.46-3.32 (m, 4H), 2.97-2.84 (m, 1H), 2.71 (s, 3H), 2.54 (s, 1H), 2.37-2.32 (m, 2H), 2.13 (s, 2H), 1.58-1.52 (m, 3H).

HPLC conditions; System: Agilent 1260 Infinity II LC coupled to an Agilent 6120B Single Quadrupole LC/MS System Column Description: Chromatorex SBM 100-5T C18 100 Å, LC Column 100×19 mm, Waters, Sun Fire; Stationary Phase: C18; Solid Support: Fully Porous Silica; Separation Mode: Reversed Phase: Mobile phase A: water; Mobile phase B: acetonitrile; Flow rate: 30 ml/min; loading pump: 4 ml/min B; Gradient conditions: 0-0-25-100% (B) 0-2-10-11.2 min.

### 8. Compounds Prepared Using Scheme 8

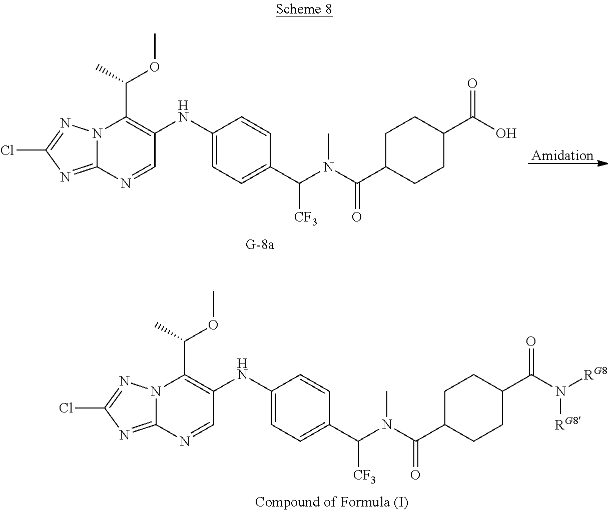

Starting material G-8a is treated with amine-containing compound to produce a compound of formula (I). R^{G8} and R^{G8′} are either H or Me.

### Synthesis of (1r,4S)—N1-((S)-1-(4-((2-chloro-7-((S)-1-methoxyethyl)-[1,2,4]triazolo[1,5-a]pyrimidin-6-yl)amino)phenyl)-2,2,2-trifluoroethyl)-N1-methylcyclohexane-1,4-dicarboxamide (Compound 8.1)

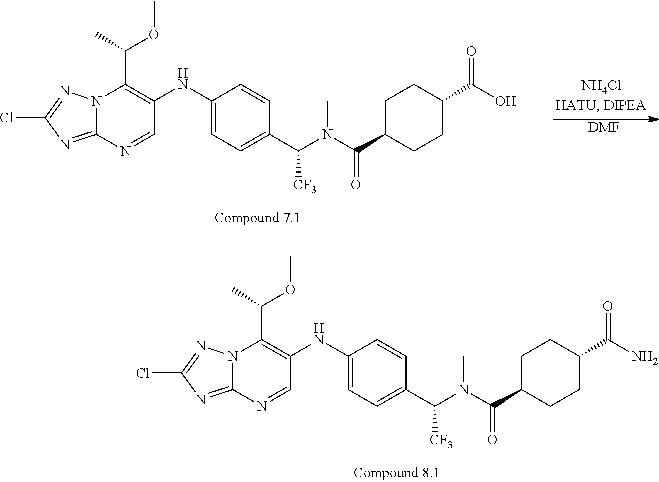

To a mixture of (1S,4r)-4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexane-1-carboxylic acid [Compound 7.1] (50 mg, 87.8 μmol) in DMF (1 mL) was added HATU (49.8 mg, 131 μmol) and DIEA (33.9 mg, 263 μmol) and the mixture was stirred at 25° C. for 0.5 hr. Then NH₄Cl (9.36 mg, 175 μmol) was added, and the mixture was stirred at 25° C. for 12 hr. The mixture was purified by prep-HPLC (column: YMC Triart C18 250*50 mm*7 μm, table: 25-65% B (A=water (0.05% ammonia hydroxide v/v)), B=acetonitrile), flow rate: 60 mL/min, UV Detector 220 nm) to afford (1r,4S)—N1-((S)-1-(4-((2-chloro-7-((S)-1-methoxyethyl)-[1,2,4]triazolo[1,5-a]pyrimidin-6-yl)amino)phenyl)-2,2,2-trifluoroethyl)-N1-methylcyclohexane-1,4-dicarboxamide [Compound 8.1] (4.00 mg, 7.04 μmol, 8.0% yield) as a yellow dry powder. m/z: [M+H]+ Calcd for C25H30ClF3N7O3 568.2; Found 568.1. ¹H NMR (400 MHz, MeOD) δ=8.87 (s, 1H), 7.29 (d, J=8.4 Hz, 2H), 7.06 (d, J=8.8 Hz, 2H), 6.59-6.48 (m, 1H), 5.36 (q, J=6.8 Hz, 1H), 3.36 (s, 3H), 2.96 (s, 3H), 2.79-2.74 (m, 1H), 2.35-2.23 (m, 1H), 1.98-1.83 (m, 4H), 1.64 (d, J=6.8 Hz, 3H), 1.61-1.51 (m, 4H).

### Synthesis of (1r,4S)—N1-((S)-1-(4-((2-chloro-7-((S)-1-methoxyethyl)-[1,2,4]triazolo[1,5-a]pyrimidin-6-yl)amino)phenyl)-2,2,2-trifluoroethyl)-N1,N4-dimethylcyclohexane-1,4-dicarboxamide (Compound 8.2)

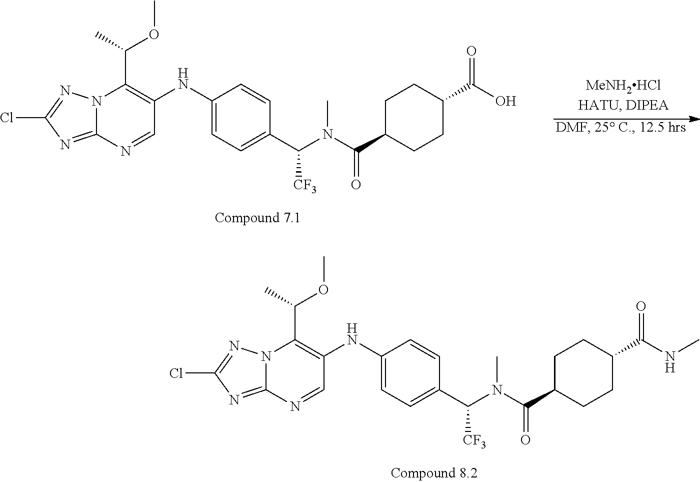 [SMILES: CO[C@@H](C)c1c(Nc2ccc([C@H](N(C)C(=O)[C@H]3CC[C@H](C(=O)O)CC3)C(F)(F)F)cc2)cnc2nc(Cl)nn12 ; "DMF, 25° C., 12.5 hrs" ; CNC(=O)[C@H]1CC[C@H](C(=O)N(C)[C@@H](c2ccc(Nc3cnc4nc(Cl)nn4c3[C@H](C)OC)cc2)C(F)(F)F)CC1]

To a mixture of (1S,4r)-4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexane-1-carboxylic acid [Compound 7.1] (50 mg, 87.8 μmol) in DMF (1 mL) was added HATU (49.8 mg, 131 μmol) and DIPEA (51.1 mg, 396 μmol) and the mixture was stirred at 25° C. for 0.5 hr. Methanamine hydrochloride (8.84 mg, 131 μmol) was then added and the mixture was stirred at 25° C. for 12 hr. The mixture was purified by prep-HPLC (column: YMC Triart C18 250*50 mm*7 μm, table: 26-66% B (A=water (0.05% ammonia hydroxide v/v)), B=acetonitrile), flow rate: 60 mL/min, UV Detector 220 nm) to afford (1r,4S)—N1-((S)-1-(4-((2-chloro-7-((S)-1-methoxyethyl)-[1,2,4]triazolo[1,5-a]pyrimidin-6-yl)amino)phenyl)-2,2,2-trifluoroethyl)-N1,N4-dimethylcyclohexane-1,4-dicarboxamide [Compound 8.2] (19.8 mg, 34.0 μmol, 38.7% yield) as a yellow dry powder. m/z: [M+H]+ Calcd for C26H32ClF3N7O3 582.2; Found 582.2. ¹H NMR (400 MHz, CD₃OD) δ=8.87 (s, 1H), 7.38-7.25 (m, 2H), 7.06 (d, J=8.8 Hz, 2H), 6.53 (q, J=8.8 Hz, 1H), 5.36 (q, J=6.8 Hz, 1H), 3.36 (s, 3H), 2.96 (s, 3H), 2.82-2.72 (m, 1H), 2.70 (s, 3H), 2.29-2.17 (m, 1H), 1.96-1.78 (m, 4H), 1.64 (d, J=6.8 Hz, 3H), 1.62-1.51 (m, 4H).

### Synthesis of (1r,4S)—N1-((S)-1-(4-((2-chloro-7-((S)-1-methoxyethyl)-[1,2,4]triazolo[1,5-a]pyrimidin-6-yl)amino)phenyl)-2,2,2-trifluoroethyl)-N1,N4-dimethylcyclohexane-1,4-dicarboxamide (Compound 8.3)

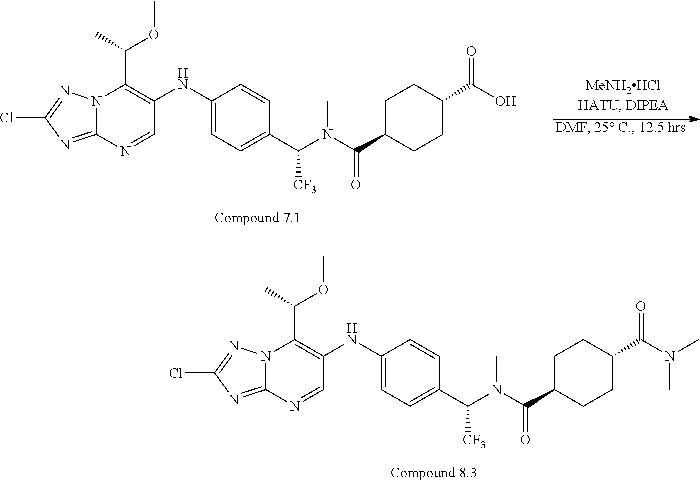 [SMILES: CO[C@@H](C)c1c(Nc2ccc([C@H](N(C)C(=O)[C@H]3CC[C@H](C(=O)O)CC3)C(F)(F)F)cc2)cnc2nc(Cl)nn12 ; "DMF, 25° C., 12.5 hrs" ; CO[C@@H](C)c1c(Nc2ccc([C@H](N(C)C(=O)[C@H]3CC[C@H](C(=O)N(C)C)CC3)C(F)(F)F)cc2)cnc2nc(Cl)nn12]

To a mixture of (1S,4r)-4-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclohexane-1-carboxylic acid [Compound 7.1] (50 mg, 87.8 μmol) in DMF (1 mL) was added HATU (49.8 mg, 131 μmol) and DIPEA (51.1 mg, 396 μmol) and the mixture was stirred at 25° C. for 0.5 hr. Then dimethylamine hydrochloride (10.6 mg, 131 μmol) was added and the mixture was stirred at 25° C. for 12 hr. The mixture was purified by prep-HPLC (column: YMC Triart C18 250*50 mm*7 μm, table: 30-70% B (A=water (0.05% ammonia hydroxide v/v)), B=acetonitrile), flow rate: 60 mL/min, UV Detector 220 nm) to afford (1r,4S)—N1-((S)-1-(4-((2-chloro-7-((S)-1-methoxyethyl)-[1,2,4]triazolo[1,5-a]pyrimidin-6-yl)amino)phenyl)-2,2,2-trifluoroethyl)-N1,N4,N4-trimethylcyclohexane-1,4-dicarboxamide [Compound 8.3] (12.4 mg, 20.8 μmol, 23.7% yield) as a yellow dry powder. m/z: [M+H]+ Calcd for C27H34ClF3N7O3 596.2; Found 596.2. ¹H NMR (400 MHz, CD₃OD) δ=8.87 (s, 1H), 7.40-7.25 (m, 2H), 7.13-7.01 (m, 2H), 6.53 (q, J=8.8 Hz, 1H), 5.36 (q, J=6.8 Hz, 1H), 3.36 (s, 3H), 3.12 (s, 3H), 2.98-2.95 (m, 3H), 2.93 (s, 3H), 2.82-2.73 (m, 2H), 1.97-1.80 (m, 4H), 1.64 (d, J=6.8 Hz, 3H), 1.63-1.51 (m, 4H).

### 9. Compounds Prepared Using Scheme 9

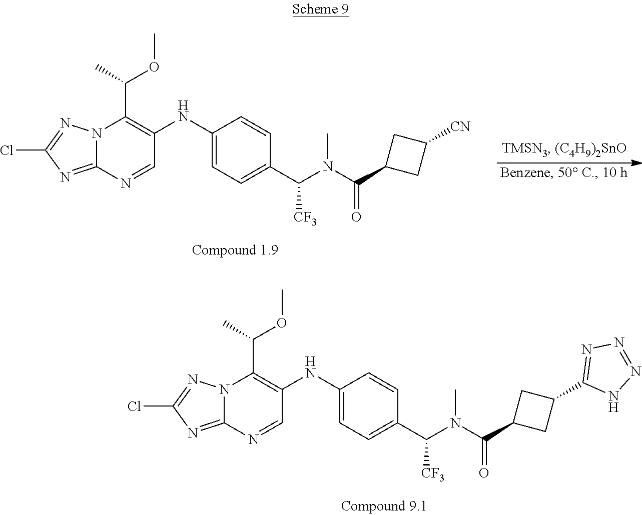 [SMILES: CO[C@@H](C)c1c(Nc2ccc([C@H](N(C)C(=O)[C@H]3C[C@H](C#N)C3)C(F)(F)F)cc2)cnc2nc(Cl)nn12 ; "Benzene, 50° C., 10 h" ; CO[C@@H](C)c1c(Nc2ccc([C@H](N(C)C(=O)[C@H]3C[C@H](c4nnn[nH]4)C3)C(F)(F)F)cc2)cnc2nc(Cl)nn12]

### Synthesis of (1r,3S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methyl-3-(1H-tetrazol-5-yl)cyclobutane-1-carboxamide (Compound 9.1)

(1r,3S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-3-cyano-N-methylcyclobutane-1-carboxamide [Compound 1.9] (0.03 g, 0.0575 mmol) was dissolved in benzene (2 mL). Trimethylsilyl azide (26.3 mg, 229 μmol) and dibutyltin oxide (28.3 mg, 114 μmol) were added. The mixture was stirred for 10 hr at 50° C. EtOAc (10 mL) and H₂O (5 mL) were added, and the organic phase was evaporated in vacuo at 45° C. The residue was purified by HPLC (see conditions below) to obtain (1r,3S)—N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methyl-3-(1H-tetrazol-5-yl)cyclobutane-1-carboxamide [Compound 9.1] (16.3 mg, 0.029 mmol, 50.3% yield) as a yellow solid. m/z: [M+H]+ Calcd for C23H25ClF3N1O02 565.2; Found 565.0. ¹H NMR (400 MHz, CD₃OD) δ=8.90-8.88 (m, 1H), 7.36-7.32 (m, 2H), 7.10-7.05 (m, 2H), 6.60-6.51 (m, 1H), 5.37 (q, J=6.6 Hz, 1H), 3.92-3.70 (m, 2H), 3.37 (s, 3H), 2.91-2.79 (m, 4H), 2.73-2.59 (m, 3H), 1.67-1.63 (m, 3H).

HPLC conditions; System: Agilent 1260 Infinity II LC coupled to an Agilent 6120B Single Quadrupole LC/MS System; Column: Chromatorex SBM 100-5T 5 μm C18(2) 100 Å, LC Column 100×19 mm, Waters, Sun Fire; Stationary Phase: C18; Solid Support: Fully Porous Silica; Separation Mode: Reversed Phase; Mobile phase A: water; Mobile phase B: acetonitrile; Flow rate: 30 ml/min; loading pump: 4 ml/min B; Gradient conditions: 20-30-70-100% (B) 0-2-10-11.2 min.

10. Separation and Isolation of Compounds 1.1 and N—((S)-1-(4-((2-chloro-7-((R)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide (Compound 10.1)

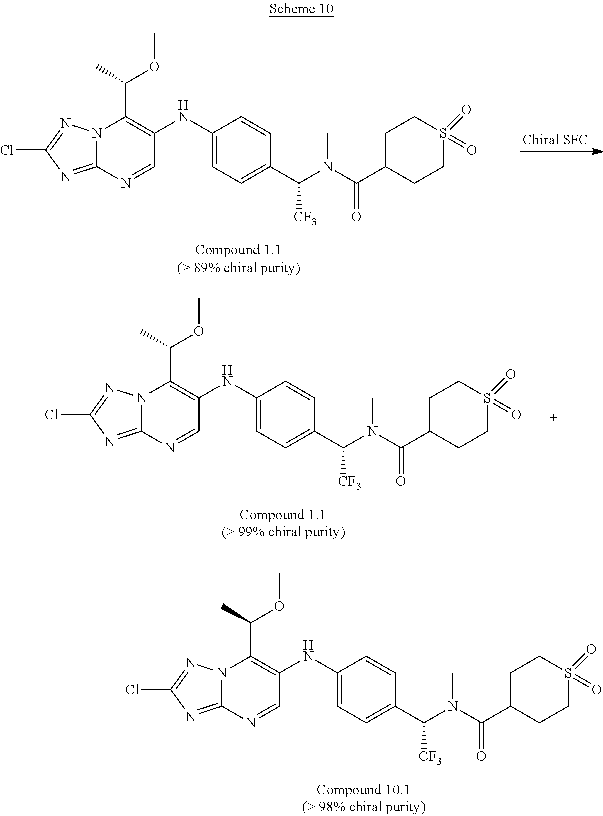 [SMILES: CO[C@@H](C)c1c(Nc2ccc([C@H](N(C)C(=O)C3CCS(=O)(=O)CC3)C(F)(F)F)cc2)cnc2nc(Cl)nn12 ; CO[C@@H](C)c1c(Nc2ccc([C@H](N(C)C(=O)C3CCS(=O)(=O)CC3)C(F)(F)F)cc2)cnc2nc(Cl)nn12 ; CO[C@H](C)c1c(Nc2ccc([C@H](N(C)C(=O)C3CCS(=O)(=O)CC3)C(F)(F)F)cc2)cnc2nc(Cl)nn12]

N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide [Compound 1.1] can be further purified by chiral SFC (column: DAICEL CHIRALPAK AD 250 mm*50 mm, 10 μm, table: 30-30% 0.1% NH₃H₂O ETOH), flow rate: 200 mL/min, UV Detector 220 nm), resulting in the separation and isolation of [Compound 1.1] (>98% chiral purity) and N—((S)-1-(4-((2-chloro-7-((R)-1-methoxyethyl)-[1,2,4]triazolo[1,5-a]pyrimidin-6-yl)amino)phenyl)-2,2,2-trifluoroethyl)-N-methyltetrahydro-2H-thiopyran-4-carboxamide 1,1-dioxide [Compound 10.1] (>98% chiral purity). For Compound 10.1; m/z: [M+H]+ Calcd for C23H27ClF3N6O4S 575.1; Found 575.0. ¹H NMR (400 MHz, DMSO) δ=8.83 (s, 1H), 7.99 (s, 1H), 7.19 (d, J=8.0 Hz, 2H), 6.97 (d, J=8.0 Hz, 2H), 6.43 (q, J=8.0 Hz, 1H), 5.15 (q, J=8.0 Hz, 1H), 3.26-3.05 (m, 8H), 2.90 (s, 3H), 2.14-1.93 (m, 4H), 1.59 (d, J=8.0 Hz, 3H).

11. Separation and Isolation of Compounds 3.1 and 1-acetyl-N—((S)-1-(4-((2-chloro-7-((R)-1-methoxyethyl)-[1,2,4]triazolo[1,5-a]pyrimidin-6-yl)amino)phenyl)-2,2,2-trifluoroethyl)-N-methylpiperidine-4-carboxamide (Compound 11.1)

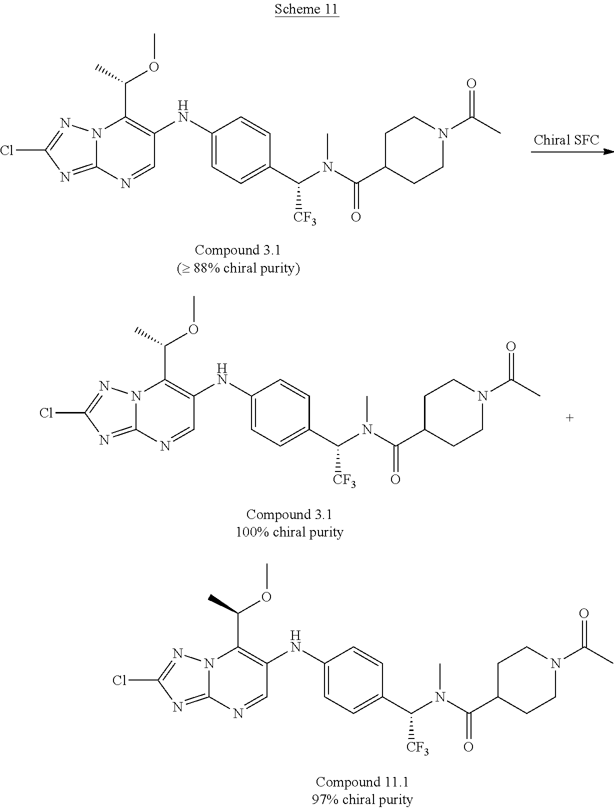 [SMILES: CO[C@@H](C)c1c(Nc2ccc([C@H](N(C)C(=O)C3CCN(C(C)=O)CC3)C(F)(F)F)cc2)cnc2nc(Cl)nn12 ; CO[C@@H](C)c1c(Nc2ccc([C@H](N(C)C(=O)C3CCN(C(C)=O)CC3)C(F)(F)F)cc2)cnc2nc(Cl)nn12 ; CO[C@H](C)c1c(Nc2ccc([C@H](N(C)C(=O)C3CCN(C(C)=O)CC3)C(F)(F)F)cc2)cnc2nc(Cl)nn12]

1-acetyl-N-((1S)-1-(4-((2-chloro-7-(1-methoxyethyl)-[1,2,4]triazolo[1,5-a]pyrimidin-6-yl)amino)phenyl)-2,2,2-trifluoroethyl)-N-methylpiperidine-4-carboxamide [Compound 3.1] can be further purified by chiral SFC (column: DAICEL CHIRALPAK IG (250 mm*50 mm, 10 um), table: 50-50% B (A=water (0.1% ammonia hydroxide), B=MeOH), flowrate: 200 mL/min) resulting in the separation and isolation of Compound 3.1 (100% chiral purity) and 1-acetyl-N—((S)-1-(4-((2-chloro-7-((R)-1-methoxyethyl)-[1,2,4]triazolo[1,5-a]pyrimidin-6-yl)amino)phenyl)-2,2,2-trifluoroethyl)-N-methylpiperidine-4-carboxamide [Compound 11.1] (97% chiral purity). For Compound 11.1; m/z: [M+H]+ Calcd for C25H30ClF3N7O3 568.2; Found 568.1. ¹H NMR (400 MHz, CDCl₃) δ=8.90 (s, 1H), 7.35-7.29 (m, 2H), 7.11 (s, 1H), 7.04 (d, J=8.8 Hz, 2H), 6.62 (q, J=8.8 Hz, 1H), 5.48 (q, J=6.8 Hz, 1H), 4.69-4.55 (m, 1H), 3.99-3.87 (m, 1H), 3.49 (s, 3H), 3.22-3.08 (m, 1H), 2.95 (s, 3H), 2.88-2.66 (m, 2H), 2.12 (s, 3H), 1.96-1.70 (m, 4H), 1.63-1.61 (m, 3H).

### 12. Synthesis of Intermediates

The following intermediates were synthesized and used for the synthesis of the exemplified compounds.

### Synthesis of 2-chloro-7-[(1S)-1-methoxyethyl]-[1,2,4]triazolo[1,5-a]pyrimidin-6-amine hydrochloride (Intermediate 1.1)

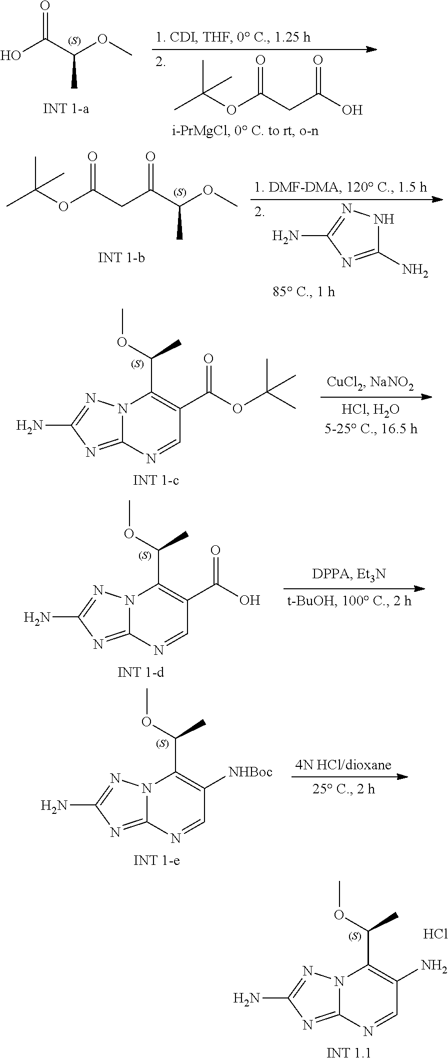

Synthesis of tert-butyl (S)-4-methoxy-3-oxopentanoate (INT 1-b):

A solution of (S)-2-methoxypropanoic acid [INT 1-a] (20 g, 192 mmol) in anhydrous tetrahydrofuran (342 mL) was cooled to 0° C. Carbonyldiimidazole (30.6 g, 189 mmol) was added at 0° C. by several portions and the mixture was stirred at this temperature for 1.25 h. In a separate flask, to a solution of 3-(tert-butoxy)-3-oxopropanoic acid (46.1 g, 288 mmol) in anhydrous tetrahydrofuran (342 mL) was added magnesium(1+) 1-methylethyl chloride (249 mL, 499 mmol, 2M in THF) at 0° C. and the mixture was stirred at room temperature for 1.25 h. Then, this solution was added to the acyl imidazole solution via a cannula at 0° C. and the resulting mixture was stirred at room temperature overnight. The reaction mixture was cooled to 0° C. and quenched by adding 10% aqueous citric acid, extracted with EtOAc, washed with saturated aqueous NaHCO₃ dried over anhydrous Na₂SO₄ and concentrated under reduced pressure to give the crude product which was purified by flash chromatography on silica gel (Acetone/hexane=0/1 to 1/9) to give tert-butyl (S)-4-methoxy-3-oxopentanoate [INT 1-b] (30.0 g, 148 mmol, 55.6%) as an oil.

### Synthesis of tert-butyl (S)-2-amino-7-(1-methoxyethyl)-[1,2,4]triazolo[1,5-a]pyrimidine-6-carboxylate (INT 1-c)

A solution of tert-butyl (S)-4-methoxy-3-oxopentanoate [INT 1-b] (25 g, 123 mmol) and (dimethoxymethyl)dimethylamine (11.1 mL, 83.6 mmol) was heated at 120° C. for 1.5 h. The mixture was cooled to room temperature and 4H-1,2,4-triazole-3,5-diamine (12.1 g, 123 mmol) followed by ethanol (123 mL) were added, and the mixture was heated at 85° C. for 1 h. After completion, the mixture was concentrated under reduced pressure and recrystallized from EtOH/water (1:1, 600 mL), filtered, and the filter cake was washed with 30% EtOH/water, followed by MTBE to give tert-butyl 2-amino-7-(1-methoxyethyl)-[1,2,4]triazolo[1,5-a]pyrimidine-6-carboxylate (12.7 g, 43.2 mmol, 52%) as a solid. The filtrate was concentrated under reduced pressure to remove MTBE and the solid was filtered and washed with hexane to get more give tert-butyl (S)-2-amino-7-(1-methoxyethyl)-[1,2,4]triazolo[1,5-a]pyrimidine-6-carboxylate [INT 1-c] (5.3 g, 18.0 mmol, 23%) as an solid. The total 18.0 g, 75% yield. The chiral HPLC showed 96.9% ee. ¹H NMR (400 MHz, CDCl₃) δ=8.75 (s, 1H), 5.40 (q, J=6.8 Hz, 1H), 4.95 (br s, 2H), 3.30 (s, 3H), 1.75 (d, J=6.8 Hz, 3H), 1.62 (s, 9H).

### Synthesis of 2-chloro-7-[(1S)-1-methoxyethyl]-[1,2,4]triazolo[1,5-a]pyrimidine-6-carboxylic acid (INT 1-d)

To a mixture of tert-butyl 2-amino-7-[(1S)-1-methoxyethyl]-[1,2,4]triazolo[1,5-a]pyrimidine-6-carboxylate [INT 1-c] (1.2 g, 4.09 mmol) and Copper(II) chloride dihydrate (173 mg, 1.02 mmol) in conc. HCl (20 mL) was added a solution of sodium nitrite (338 mg, 4.90 mmol) in H₂O (5 mL) at 5° C. with ice bath and the mixture was stirred at 5° C. for 30 min. Then the mixture was warmed to 25° C. and stirred for 16 hr. Water (100 mL) was added and 1 N NaOH aqueous solution was added to adjust the pH to 3-4. The mixture was extracted with CHCl₃:i-PrOH=3:1 (100 mL×3) and the combined organic layers were dried over anhydrous Na₂SO₄ and concentrated under reduced pressure to give 2-chloro-7-[(1S)-1-methoxyethyl]-[1,2,4]triazolo[1,5-a]pyrimidine-6-carboxylic acid [INT 1-d] (962 mg, 92.4% yield) as a solid. m/z: [M+H]+ Calcd for C9H10ClN4O3 257.0; Found 256.9. ¹H NMR (400 MHz, DMSO-d₆) δ=14.00 (br s, 1H), 9.07 (s, 1H), 5.39 (q, J=6.4 Hz, 1H), 3.21 (s, 3H), 1.63 (d, J=6.4 Hz, 3H).

### Synthesis of tert-butyl N-{2-chloro-7-[(1S)-1-methoxyethyl]-[1,2,4]triazolo[1,5-a]pyrimidin-6-yl}carbamate (INT 1-e)

To a solution of 2-chloro-7-[(1S)-1-methoxyethyl]-[1,2,4]triazolo[1,5-a]pyrimidine-6-carboxylic acid [INT 1-d] (1.3 g, 5.06 mmol) in t-BuOH (10 mL) was added {[azido(phenoxy)phosphoryl]oxy}benzene (2.08 g, 7.58 mmol) and triethylamine (1.02 g, 10.1 mmol) and the mixture was stirred at 100° C. for 2 h under N₂ atmosphere. The mixture was concentrated under reduced pressure to afford the crude product which was purified by flash chromatography on silica gel (EtOAc/Petroleum ether=1/10 to 1/5) to afford tert-butyl N-{2-chloro-7-[(1S)-1-methoxyethyl]-[1,2,4]triazolo[1,5-a]pyrimidin-6-yl}carbamate [INT 1-e] (420 mg, 25.4% yield) as a solid. m/z: [M+H]+ Calcd for C13H19ClN5O3 328.1; Found 328.0. ¹H NMR (400 MHz, CDCl₃) δ=9.62 (br s, 1H), 8.05 (s, 1H), 5.45 (q, J=6.8 Hz, 1H), 3.48 (s, 3H), 1.63 (d, J=6.8 Hz, 3H), 1.56 (s, 9H).

### Synthesis of 2-chloro-7-[(1S)-1-methoxyethyl]-[1,2,4]triazolo[1,5-a]pyrimidin-6-amine hydrochloride (INT 1.1)

A mixture of tert-butyl N-{2-chloro-7-[(1S)-1-methoxyethyl]-[1,2,4]triazolo[1,5-a]pyrimidin-6-yl}carbamate [INT 1-e] (420 mg, 1.28 mmol) in 4N HCl/dioxane (5 mL) was stirred at 25° C. for 2 h. LCMS showed the reaction was completed and one new peak with desired MS was detected (Rt=0.611 min. m/z: 227.8 [M+H]+). The mixture was concentrated under reduced pressure to afford 2-chloro-7-[(1S)-1-methoxyethyl]-[1,2,4]triazolo[1,5-a]pyrimidin-6-amine hydrochloride [INT 1.1] (400 mg, crude) as a solid.

### Synthesis of 2-chloro-7-isopropyl-[1,2,4]triazolo[1,5-a]pyrimidin-6-amine hydrochloride (Intermediate 1.2)

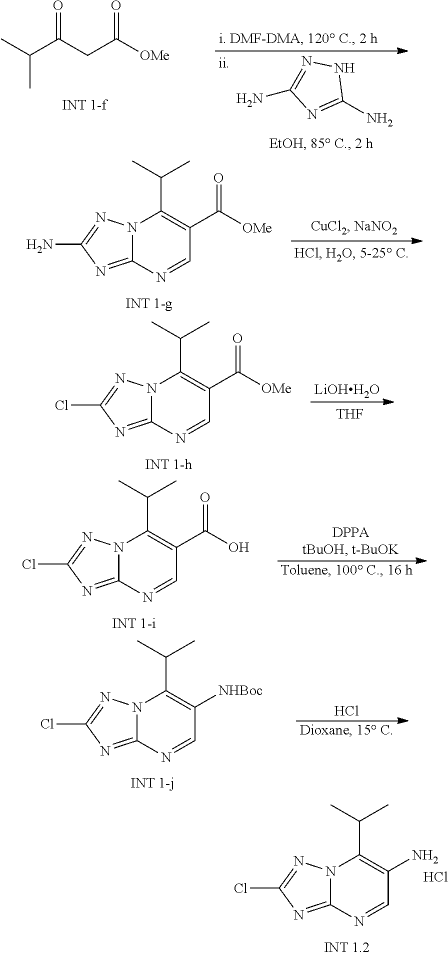

### Synthesis of methyl 2-amino-7-isopropyl-[1,2,4]triazolo[1,5-a]pyrimidine-6-carboxylate (INT 1-g)

A mixture of methyl 4-methyl-3-oxopentanoate [INT 1-f] (23.2 g, 160 mmol) and DMF-DMA (19.0 g, 160 mmol) was stirred at 120° C. for 2 hr under N₂ atmosphere. Then a mixture of 1H-1,2,4-triazole-3,5-diamine (15.8 g, 160 mmol) in EtOH (30 mL) was added and the mixture was stirred at 85° C. for 2 hr. The mixture was concentrated under reduced pressure to afford the crude product which was purified by flash chromatography on silica gel (EtOAc/Petroleum ether=1/10 to 3/2) to give methyl 2-amino-7-isopropyl-[1,2,4]triazolo[1,5-a]pyrimidine-6-carboxylate [INT 1-g] (20.6 g, 87.7 mmol, 54.7% yield) as an off-white solid. m/z: [M+H]+ Calcd for C10H14N5O2 236.1; Found 236.2. ¹H NMR (400 MHz, CDCl₃) δ=8.88 (s, 1H), 4.93 (br s, 2H), 4.48-4.39 (m, 1H), 3.89 (s, 3H), 1.49 (s, 3H), 1.47 (s, 3H).

### Synthesis of methyl 2-chloro-7-isopropyl-[1,2,4]triazolo[1,5-a]pyrimidine-6-carboxylate (INT 1-h)

To a mixture of methyl 2-amino-7-isopropyl-[1,2,4]triazolo[1,5-a]pyrimidine-6-carboxylate [INT 1-g] (10.17 g, 34.5 mmol) and Copper(II) chloride dihydrate (1.46 g, 8.62 mmol) in cone. HCl (30 mL) was added a solution of sodium nitrite (2.85 g, 41.4 mmol) in H₂O (5 mL) at 5° C. The mixture was stirred at 5° C. (ice bath cooling) for 30 min. Then the mixture was warmed to 25° C. and stirred for 12 hr. 2 N NaOH aqueous solution was added to adjust the pH to 7 and the mixture was extracted with EtOAc (200 mL×2). The combined organic layers were washed with brine (200 mL×3), dried over anhydrous Na₂SO₄ and concentrated under reduced pressure to give the crude product, which was purified by flash chromatography on silica gel (EtOAc/Petroleum ether=0/1 to 1/1) to give methyl 2-chloro-7-isopropyl-[1,2,4]triazolo[1,5-a]pyrimidine-6-carboxylate [INT 1-h] (3.00 g, 11.7 mmol, 34.1% yield) as a yellow solid. m/z: [M+H]+ Calcd for C10H12ClN4O2 255.1; Found 255.0. ¹H NMR (400 MHz, CDCl₃) δ=9.16 (d, J=1.2 Hz, 1H), 4.60-4.44 (m, 1H), 4.03 (d, J=0.4 Hz, 3H), 1.61 (dd, J=1.2, 7.2 Hz, 6H).

### Synthesis of 2-chloro-7-isopropyl-[1,2,4]triazolo[1,5-a]pyrimidine-6-carboxylic acid (INT 1-i)

To a mixture of methyl 2-chloro-7-isopropyl-[1,2,4]triazolo[1,5-a]pyrimidine-6-carboxylate [INT 1-h] (1 g, 3.92 mmol) in THF (10 mL) was added lithium(1+) hydrate hydroxide (2 M in H₂O, 2.94 mL, 5.88 mmol) and the mixture was stirred at 25° C. for 3 hr. THF was removed under reduced pressure and water (10 mL) was added. 1 N HCl was added to adjust the pH to 3-4 and the mixture was filtered. The filter cake was washed with water (20 mL×2), collected and concentrated under reduced pressure to give a solid. The solid was purified by prep-HPLC (column: Boston Green ODS 150*30 mm*5 μm, table: 8-48% B (A=water (0.05% HCl), B=acetonitrile), flow rate: 30 mL/min, UV Detector 220 nm) to afford 2-chloro-7-isopropyl-[1,2,4]triazolo[1,5-a]pyrimidine-6-carboxylic acid [INT 1-i] (300 mg, 1.24 mmol, 31.8% yield) as a white dry powder. m/z: [M+H]+ Calcd for C9H10ClN4O2 241.1; Found 241.0. ¹H NMR (400 MHz, DMSO-d6) δ=14.14 (br s, 1H), 9.14 (s, 1H), 4.48 (spt, J=6.8 Hz, 1H), 1.51 (s, 3H), 1.49 (s, 3H).

### Synthesis of tert-butyl (2-chloro-7-isopropyl-[1,2,4]triazolo[1,5-a]pyrimidin-6-yl)carbamate (INT 1-j)

To a solution of 2-chloro-7-isopropyl-[1,2,4]triazolo[1,5-a]pyrimidine-6-carboxylic acid [INT 1-i] (150 mg, 623 μmol) in toluene (5 mL) was added diphenylphosphoryl azide (257 mg, 934 μmol), t-BuOH (2 mL) and potassium tert-butoxide (208 mg, 1.86 mmol). The reaction mixture was stirred at 100° C. for 16 h under N₂. The mixture was concentrated under reduced pressure to give the crude product which was purified by flash chromatography on silica gel (0-50% EtOAc in PE) to give tert-butyl (2-chloro-7-isopropyl-[1,2,4]triazolo[1,5-a]pyrimidin-6-yl)carbamate [INT 1-j] (80.0 mg, 256 μmol, 41.2% yield) as a yellow solid. m/z: [M+H]+ Calcd for C13H19ClN5O2 312.1; Found 312.1.

### Synthesis of 2-chloro-7-isopropyl-[1,2,4]triazolo[1,5-a]pyrimidin-6-amine hydrochloride (INT 1.2)

A solution of tert-butyl (2-chloro-7-isopropyl-[1,2,4]triazolo[1,5-a]pyrimidin-6-yl)carbamate [INT 1-j] (80 mg, 256 μmol) in 4 M HCl/dioxane (10 mL) was stirred at 15° C. for 16 h. The reaction was concentrated under reduced pressure to give 2-chloro-7-isopropyl-[1,2,4]triazolo[1,5-a]pyrimidin-6-amine hydrochloride [INT 1.2] (63.0 mg, 253 μmol, 99.2% yield) as a white solid. m/z: [M+H]+ Calcd for C8H11ClN5 212.1; Found 211.7.

### Synthesis of (R)-2-chloro-7-(1-methoxyethyl)-[1,2,4]triazolo[1,5-a]pyrimidin-6-amine hydrochloride (Intermediate 1.3)

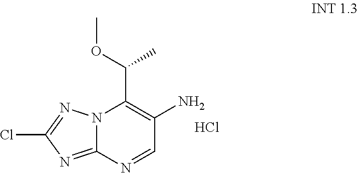

(R)-2-chloro-7-(1-methoxyethyl)-[1,2,4]triazolo[1,5-a]pyrimidin-6-amine [INT 1.3] can be prepared by the same synthetic route as outlined for INT 1.1 using (2R)-2-methoxypropanoic acid as a starting material. m/z: [M+H]+ Calcd for C8H11ClN5O 228.1; Found 228.1.

### Synthesis of (R)—N—((S)-1-(4-bromophenyl)-2,2,2-trifluoroethyl)-2-methylpropane-2-sulfinamide (Intermediate 2.1)

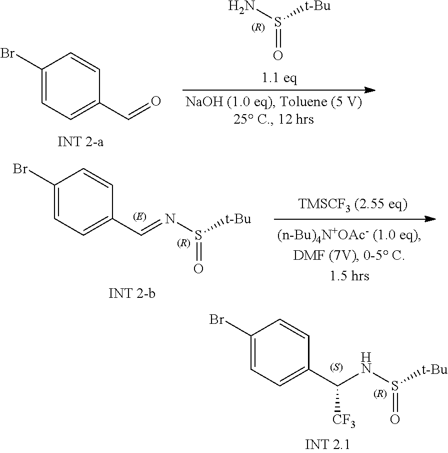

### Synthesis of (R,E)-N-(4-bromobenzylidene)-2-methylpropane-2-sulfinamide (INT 2-b)

To a solution of 4-bromobenzaldehyde [INT 2-a] (100 g, 541 mmol, 1.0 eq) in toluene (500 mL) was added (R)-2-methylpropane-2-sulfinamide (72.1 g, 595 mmol, 1.1 eq) at 25° C. The mixture was stirred at 25° C. for 15 mins. Then to above reaction was added NaOH (21.6 g, 541 mmol, 1.0 eq) and the mixture was stirred at 25° C. for 12 h. Na₂SO₄ (50 g) was added to the mixture and stirred for 20 mins. Four reaction mixtures were combined and filtered through celite to give the filtrate which was concentrated in vacuum to give the crude product as an oil. The crude product was dissolved in Petroleum ether (1.0 L) and stirred at −50° C. for 1.0 h, filtered to give (R,E)-N-(4-bromobenzylidene)-2-methylpropane-2-sulfinamide [INT 2-b] (620 g, 2.15 mol, 99.5% yield) as a solid.

### Synthesis of (R)—N—((S)-1-(4-bromophenyl)-2,2,2-trifluoroethyl)-2-methylpropane-2-sulfinamide (INT 2.1)

To a solution of (R,E)-N-(4-bromobenzylidene)-2-methylpropane-2-sulfinamide [INT 2-b] (206 g, 715 mmol, 1.0 eq) and tetrabutylammonium acetate (216 g, 715 mmol, 218 mL, 1.0 eq) in DMF (1.4 L) was added TMSCF₃ (259 g, 1.82 mol, 2.5 eq) at 0° C. The mixture was stirred at 5° C. for 1.5 h. This process was repeated 2 times and the three reaction mixtures were combined for work-up. The mixture was poured into saturated NH₄Cl solution (13.0 L) and stirred for 10 mins to give the suspension. The suspension was filtered to give the filter cake and eluted with water (5.0 L). The filter cake was triturated with MTBE/Petroleum ether (v/v=1:4, 2.0 L) and to give the product as a solid and the mother liquid was concentrated in vacuum to give the crude product as an oil which was purified by column chromatography on silica gel with petroleum ether/ethyl acetate (10/1˜1/1) to give (R)—N—((S)-1-(4-bromophenyl)-2,2,2-trifluoroethyl)-2-methylpropane-2-sulfinamide [INT 2.1] (389 g, 1.09 mol, 50.6% yield) as a solid. ¹H NMR (400 MHz, CDCl₃) δ=1.25 (s, 9H), 3.64 (d, J=6.40 Hz, 1H), 4.79-4.83 (m, 1H), 7.32 (d, J=8.40 Hz, 2H), 7.56 (d, J=6.40 Hz, 2H).

### Synthesis of (S)-1-(4-bromophenyl)-2,2,2-trifluoro-N-methylethan-1-amine hydrochloride (Intermediate 3.1)

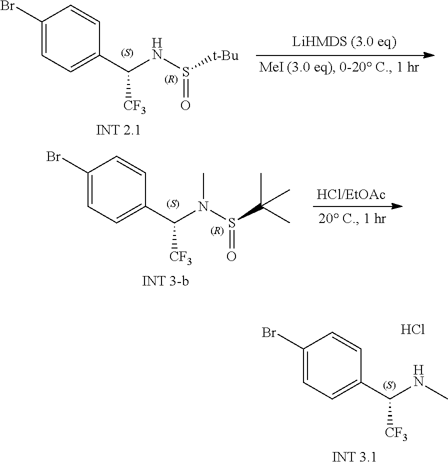

### Synthesis of (R)—N—((S)-1-(4-bromophenyl)-2,2,2-trifluoroethyl)-N,2-dimethylpropane-2-sulfinamide [INT 3-b]

To a solution of LiHMDS (1.0 M, 838 mL, 3.0 eq) was added (R)—N—((S)-1-(4-bromophenyl)-2,2,2-trifluoroethyl)-2-methylpropane-2-sulfinamide [INT 2.1] (100 g, 279 mmol, 1.0 eq) at 0-10° C. and stirred at 0-10° C. for 0.5 h. To the above mixture was added MeI (119 g, 838 mmol, 52.1 mL, 3.0 eq) at 0-10° C. and stirred at 25° C. for 1 h. The process was repeated 2 times and the three combined reaction mixtures was poured to saturated NH₄Cl (3.0 L) and diluted with EtOAc (1.0 L). The mixture was separated to give the organic layer and the aqueous layer was extracted with EtOAc (500 mL). The combined organic layer was washed with saturated NaCl (1.0 L) and dried with Na₂SO₄, filtered and concentrated in vacuum to give the crude product as an oil. The crude product was purified by column chromatography on silica gel with petroleum ether/ethyl acetate (15/1˜1/1) to give (R)—N—((S)-1-(4-bromophenyl)-2,2,2-trifluoroethyl)-N,2-dimethylpropane-2-sulfinamide [INT 3-b] (161 g, 432.5 mmol, 51.6% yield) as an oil.

### Synthesis of (S)-1-(4-bromophenyl)-2,2,2-trifluoro-N-methylethan-1-amine hydrochloride [INT 3.1]

To the mixture of (R)—N—((S)-1-(4-bromophenyl)-2,2,2-trifluoroethyl)-N,2-dimethylpropane-2-sulfinamide [INT 3-b] (202 g, 543 mmol, 1.0 eq) in EtOAc (600 mL) was added HCl/EtOAc (4.0 M. 2.02 L, 14.9 eq) slowly. The above mixture was stirred at 20° C. for 1 h. The reaction mixture was filtered to give a solid and eluted with EtOAc (200 mL) and the mother liquid was concentrated in vacuum to give a solid. The solid was purified by column chromatography on silica gel with petroleum ether/ethyl acetate (10/1˜1/0) and combined with the filter cake and concentrated by oil pump at 45° C. for 1 h to remove the solvent residue to give (S)-1-(4-bromophenyl)-2,2,2-trifluoro-N-methylethan-1-amine hydrochloride [INT 3.1] (115 g, 378 mmol, 69.6% yield, 100% purity, HCl) as a solid. ¹H NMR (400 MHz, DMSO-d₆) δ=2.45 (s, 3H), 5.51 (s, 1H), 7.62 (d, J=8.4 Hz, 2H), 7.78 (d, J=8.40 Hz, 2H), 10.59 (s, 2H).

SFC: Rt=0.776 min, 99.98% ee; Column: Chiralpak AD-3, 100×4.6 mm, I.D., 3 um; Mobile phase: A: CO₂, B: MeOH (0.05% IPA); Gradient: A:B=97:3; Flow rate: 3 mL/min; Column temp.: 35° C.

LCMS: Rt=1.755 min, 100.0% purity, m/z=268.0, 270.0 (M+1)+. The gradient was 5% B in 0.40 min and 5-95% B at 0.4-3.0 min, hold on 95% B for 1.00 min, and then 95-5% B in 0.01 min, the flow rate was 1.0 mL/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) as well as positive electrospray ionization. MS range was 100-1000.

### Synthesis of tetrahydro-2H-thiopyran-4-carbonyl chloride 1,1-dioxide (Intermediate 4.1)

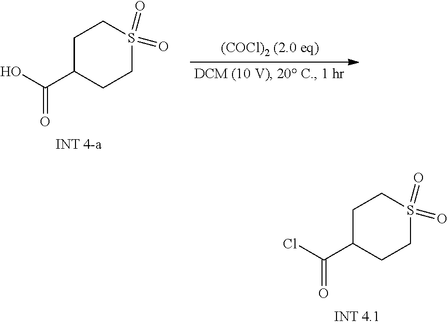 [SMILES: O=C(O)C1CCS(=O)(=O)CC1 ; O=C(Cl)C1CCS(=O)(=O)CC1]

To a solution of tetrahydro-2H-thiopyran-4-carboxylic acid 1,1-dioxide [INT 4-a] (41.0 g, 230 mmol, 1.0 eq) in DCM (410 mL) was added (COCl)₂ (58.4 g, 460 mmol, 40.3 mL, 2.0 eq) and DMF (168 mg, 2.30 mmol, 177 μL, 0.01 eq) at 0° C. under N₂. The mixture was warmed to 20° C. and stirred at 20° C. for 2 h. The suspension turned to clear, which showed the most of starting material was consumed. The reaction mixture was concentrated in vacuum to give the crude product as a solid, which was concentrated by oil pump to remove the solvent residue to give tetrahydro-2H-thiopyran-4-carbonyl chloride 1,1-dioxide [INT 4.1] (46.5 g, crude) as a solid.

### Synthesis of tetrahydro-2H-pyran-2-carbonyl chloride (Intermediate 4.2)

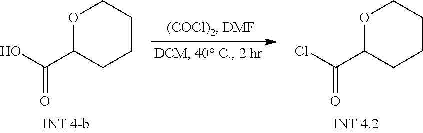 [SMILES: O=C(O)C1CCCCO1 ; O=C(Cl)C1CCCCO1]

To a mixture of tetrahydro-2H-pyran-2-carboxylic acid [INT 4-b] (330 mg, 2.53 mmol) in dichloromethane (4 mL) was added oxalyl dichloride (478 mg, 3.79 mmol) and DMF (18.4 mg, 252 μmol) slowly and the mixture was stirred at 40° C. for 2 hr. The mixture was concentrated under reduced pressure to afford the tetrahydro-2H-pyran-2-carbonyl chloride [INT 4.2] (370 mg, 2.49 mmol, 98.6% yield) as a yellow gum.

### Synthesis of Methyl (1r,4r)-4-(chlorocarbonyl)cyclohexane-1-carboxylate (Intermediate 4.3)

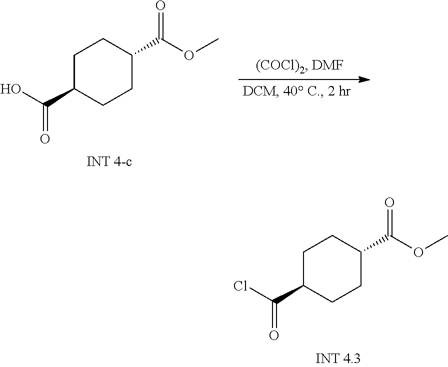

To a mixture of (1r,4r)-4-(methoxycarbonyl)cyclohexane-1-carboxylic acid [INT 4-c] (1.45 g, 7.78 mmol) in dichloromethane (10 mL) was added oxalyl dichloride (2.93 g, 23.3 mmol) and DMF (56.8 mg, 778 μmol) slowly and the mixture was stirred at 40° C. for 2 hr. The mixture was concentrated under reduced pressure to afford the crude methyl (1r,4r)-4-(chlorocarbonyl)cyclohexane-1-carboxylate [INT 4.3] (1.59 g, 7.76 mmol) as a yellow gum.

### Synthesis of thiane-4-carbonyl chloride (Intermediate 4.4)

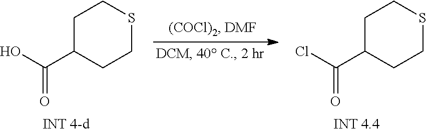 [SMILES: O=C(O)C1CCSCC1 ; O=C(Cl)C1CCSCC1]

To a mixture of thiane-4-carboxylic acid [INT 4-d] (370 mg, 2.53 mmol) in dichloromethane (2 mL) was added oxalyl dichloride (478 mg, 3.79 mmol) and DMF (18.4 mg, 252 μmol) slowly. The mixture was stirred at 40° C. for 2 hr. The mixture was concentrated under reduced pressure to afford the crude thiane-4-carbonyl chloride [INT 4.4] (415 mg, 2.52 mmol) as a yellow gum.

### Synthesis of oxolane-3-carbonyl chloride (Intermediate 4.5)

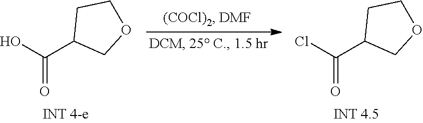 [SMILES: O=C(O)C1CCOC1 ; O=C(Cl)C1CCOC1]

To a mixture of oxolane-3-carboxylic acid [INT 4-e] (1 g, 8.61 mmol) in CH₂Cl₂ (10 mL) were added oxalic dichloride (2.18 g, 17.2 mmol) and N,N-dimethylformamide (62.9 mg, 861 μmol). The reaction mixture was stirred at 25° C. for 1.5 hours. The reaction mixture was concentrated to give oxolane-3-carbonyl chloride [INT 4.5] (1.10 g, 8.17 mmol) as yellow oil.

### Synthesis of 1,4-dioxaspiro[4.5]decane-8-carbonyl chloride (Intermediate 4.6)

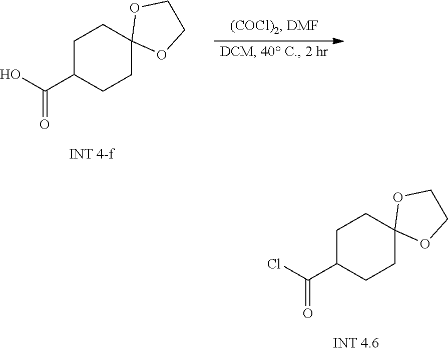

To a mixture of 1,4-dioxaspiro[4.5]decane-8-carboxylic acid [INT 4-f] (560 mg, 3.00 mmol) in dichloromethane (2 mL) was added oxalyl dichloride (567 mg, 4.50 mmol) and DMF (21.9 mg, 300 μmol) slowly and the mixture was stirred at 40° C. for 2 hr. The mixture was concentrated under reduced pressure to afford the crude 1,4-dioxaspiro[4.5]decane-8-carbonyl chloride [INT 4.6] (610 mg, 2.98 mmol) as a yellow gum.

### Synthesis of 4,4-difluorocyclohexane-1-carbonyl chloride (Intermediate 4.7)

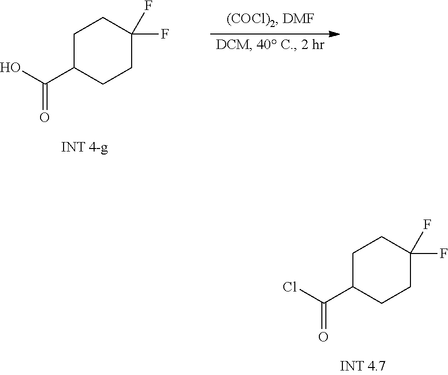

To a mixture of 4,4-difluorocyclohexane-1-carboxylic acid [INT 4-g] (1 g, 6.09 mmol) in dichloromethane (15 mL) was added oxalyl dichloride (2.29 g, 18.2 mmol) and DMF (44.5 mg, 609 μmol) slowly and the mixture was stirred at 40° C. for 2 hr. The mixture was concentrated under reduced pressure to afford the crude 4,4-difluorocyclohexane-1-carbonyl chloride [INT 4.7] (1.11 g, 6.07 mmol) as a yellow gum.

### Synthesis of 1-acetylazetidine-3-carbonyl chloride (Intermediate 4.8)

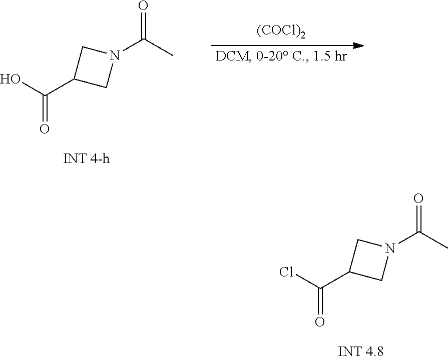

To a solution of 1-acetylazetidine-3-carboxylic acid [INT 4-h] (300 mg, 2.09 mmol) in DCM (4 mL) was added oxalyl chloride (397 mg, 3.13 mmol) at 0° C. The mixture was stirred at 20° C. for 1.5 hr. The reaction was concentrated under reduced pressure to give 1-acetylazetidine-3-carbonyl chloride [INT 4.8] (337 mg, 2.08 mmol).

### Synthesis of 1-acetylpiperidine-4-carbonyl chloride (Intermediate 4.9)

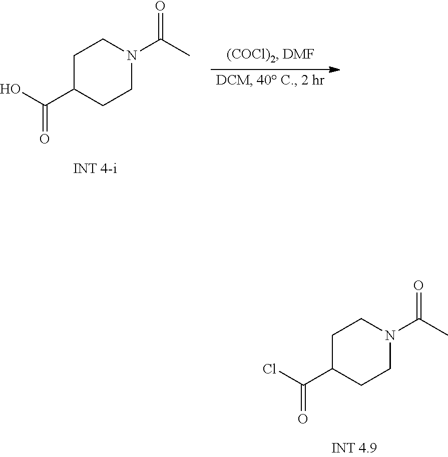

To a mixture of 1-acetylpiperidine-4-carboxylic acid [INT 4-i] (1 g, 5.84 mmol) in dichloromethane (10 mL) was added oxalyl dichloride (2.20 g, 17.5 mmol) and DMF (42.6 mg, 584 μmol) slowly and the mixture was stirred at 40° C. for 2 hr. The mixture was concentrated under reduced pressure to afford the crude 1-acetylpiperidine-4-carbonyl chloride [INT 4.9] (1.10 g, 5.80 mmol) as a green oil.

### Synthesis of methyl (1r,3r)-3-(carbonochloridoyl)cyclobutane-1-carboxylate (Intermediate 4.10)

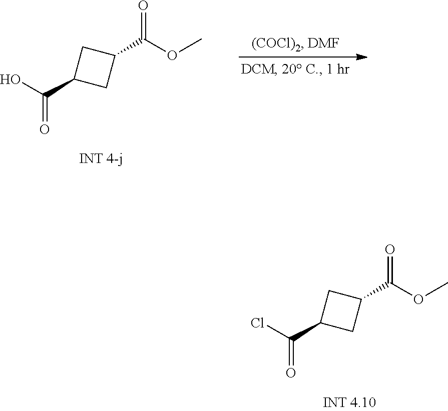

To a mixture of (1r,3r)-3-(methoxycarbonyl)cyclobutane-1-carboxylic acid [INT 4-j] (100 mg, 632 μmol) in CH₂Cl₂ (2 mL) was added DMF (one drop) and oxalic dichloride (239 mg, 1.89 mmol) at 20° C. The mixture was stirred at 20° C. for 1 h. The reaction was concentrated under reduced pressure to give the crude methyl (1r,3r)-3-(carbonochloridoyl)cyclobutane-1-carboxylate [INT 4.10] (111 mg, 628 μmol) as a pale-yellow oil.

### Synthesis of (S)—N-(1-(4-bromophenyl)-2,2,2-trifluoroethyl)-N-methyltetrahydro-2H-thiopyran-4-carboxamide 1,1-dioxide (Intermediate 5.1)

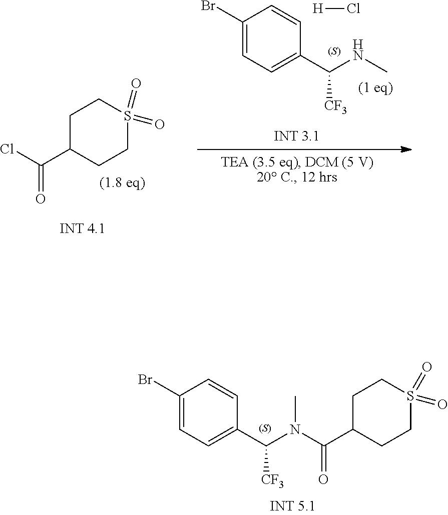

To a solution of (S)-1-(4-bromophenyl)-2,2,2-trifluoro-N-methylethan-1-amine hydrochloride [INT 3.1] (39.0 g, 128 mmol, 1.0 eq. HCl) and TEA (45.7 g, 451 mmol, 62.8 mL, 3.5 eq) in DCM (200 mL) was added tetrahydro-2H-thiopyran-4-carbonyl chloride 1,1-dioxide [INT 4.1] (45.3 g, 231 mmol, 1.8 eq) at 0-10° C. The mixture was stirred at 20° C. for 12 h. The mixture was separated to give the organic layer and the aqueous layer was extracted with DCM (100 mL). The combined organic layer was concentrated in vacuum to give the crude product as an oil. The crude product was purified by column chromatography on silica gel with petroleum ether/ethyl acetate (15/1˜3/1) to give (S)—N-(1-(4-bromophenyl)-2,2,2-trifluoroethyl)-N-methyltetrahydro-2H-thiopyran-4-carboxamide 1,1-dioxide [INT 5.1] (26.0 g, 60.7 mmol, 47.4% yield, 100% purity) as a solid. ¹H NMR (400 MHz, CDCl₃) δ 2.25-2.37 (m, 1H), 2.38-2.40 (m, 3H), 2.88-3.00 (m, 6H), 3.30-3.31 (m, 1H), 3.22-3.45 (m, 1H), 6.56-6.63 (m, 1H), 7.23 (d, J=8.00 Hz, 2H), 7.55 (d, J=8.40 Hz, 2H).

SFC: Rt=1.21 min, 100.0% ee; Column: Chiralpak AD-3, 50×4.6 mm I.D., 3 um; Mobile phase: A: CO₂, B: MeOH (0.05% IPAm, v/v); Flow rate: 3.4 mL/min; Column temp.: 35° C.

LCMS: Rt=2.431 min, 100% purity, m/z=428.0, 430.0 (M+1)+. The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.037% Trifluoroacetic Acid in water, mobile phase B was 0.018% Trifluoroacetic Acid in acetonitrile. The column used for chromatography was a Kinetex C18 50*2.1 mm column (5 um particles). Detection methods are diode array (DAD) as well as positive electrospray ionization. MS range was 100-1000.

### Synthesis of N-[(1S)-1-(4-bromophenyl)-2,2,2-trifluoroethyl]-N-methylacetamide (Intermediate 5.2)

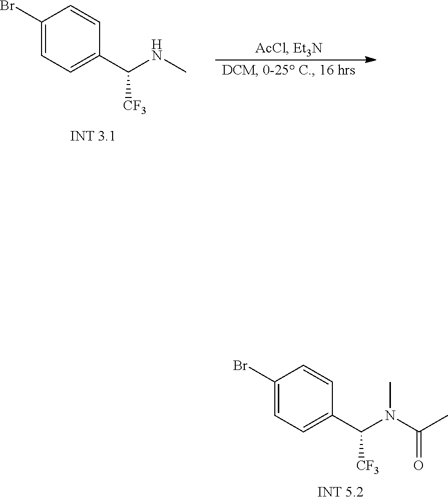

To a mixture of [(1S)-1-(4-bromophenyl)-2,2,2-trifluoroethyl](methyl)amine [free base of INT 3.1] (1 g, 3.73 mmol) and Et₃N (754 mg, 7.46 mmol) in dichloromethane (10 mL) was added acetyl chloride (396 μL, 5.59 mmol) at 0° C. and the mixture was stirred at 25° C. for 16 hr. The mixture was concentrated under reduced pressure to afford the crude product, which was purified by flash chromatography on silica gel (EtOAc/Petroleum ether=0/1 to 1/5) to give N-[(1S)-1-(4-bromophenyl)-2,2,2-trifluoroethyl]-N-methylacetamide [INT 5.2] (750 mg, 2.41 mmol, 64.6% yield) as a colorless oil. m/z: [M+H]+ Calcd for C11H12BrF3NO 310.0, 312.0; Found 309.8, 311.8. ¹H NMR (400 MHz, CDCl₃) δ=7.54 (d, J=8.4 Hz, 2H), 7.28-7.25 (m, 2H), 6.61 (q, J=8.8 Hz, 1H), 2.85 (s, 3H), 2.21 (s, 3H).

### Synthesis of N—((S)-1-(4-bromophenyl)-2,2,2-trifluoroethyl)-N-methyltetrahydro-2H-pyran-2-carboxamide (Intermediate 5.3)

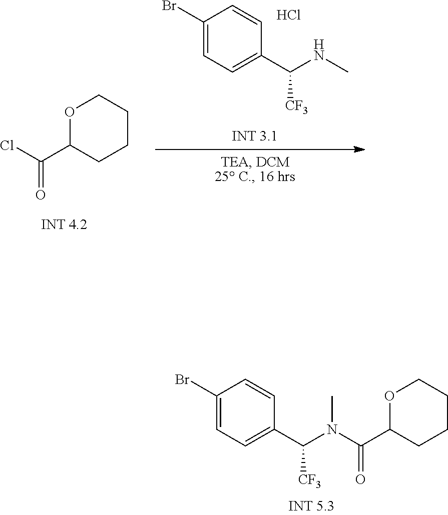 [SMILES: O=C(Cl)C1CCCCO1 ; CN[C@@H](c1ccc(Br)cc1)C(F)(F)F ; "TEA, DCM25° C., 16 hrs" ; Cl ; CN(C(=O)C1CCCCO1)[C@@H](c1ccc(Br)cc1)C(F)(F)F]

To a mixture of (S)-1-(4-bromophenyl)-2,2,2-trifluoro-N-methylethan-1-amine hydrochloride [INT 3.1] (400 mg, 1.31 mmol) and Et₃N (662 mg, 6.55 mmol) in dichloromethane (2 mL) was added a solution of tetrahydro-2H-pyran-2-carbonyl chloride [INT 4.2] (370 mg, 2.49 mmol) in dichloromethane (2 mL). The mixture was stirred at 25° C. for 16 hr. Water (10 mL) was added and the mixture was extracted with dichloromethane (10 mL×2). The combined organic layers were dried over anhydrous Na₂SO₄, filtered and concentrated under reduced pressure to give the crude product which was purified by flash chromatography on silica gel (EtOAc/petroleum ether=1/10 to 1/5) to give N—((S)-1-(4-bromophenyl)-2,2,2-trifluoroethyl)-N-methyltetrahydro-2H-pyran-2-carboxamide [INT 5.3] (400 mg, 1.05 mmol, 80.3% yield) as a yellow oil. m/z: [M+H]+ Calcd for C15H18BrF3NO2 380.0, 382.0; Found 380.0.

### Synthesis of Methyl (1S,4r)-4-(((S)-1-(4-bromophenyl)-2,2,2-trifluoroethyl)(methyl)carbamoyl)cyclohexane-1-carboxylate (Intermediate 5.4)

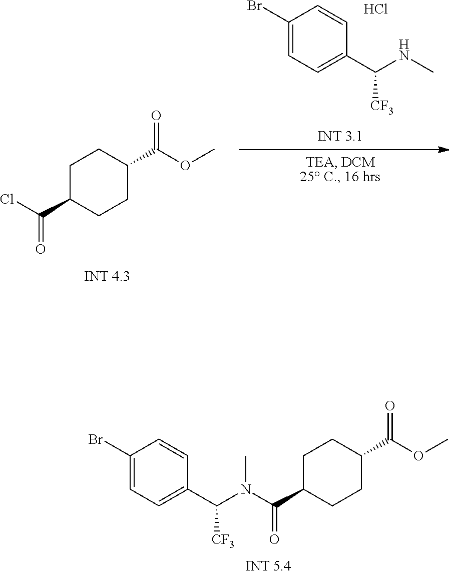 [SMILES: CN[C@@H](c1ccc(Br)cc1)C(F)(F)F ; Cl ; COC(=O)[C@H]1CC[C@H](C(=O)Cl)CC1 ; "TEA, DCM25° C., 16 hrs" ; COC(=O)[C@H]1CC[C@H](C(=O)N(C)[C@@H](c2ccc(Br)cc2)C(F)(F)F)CC1]

To a mixture of methyl (1r,4r)-4-(carbonochloridoyl)cyclohexane-1-carboxylate [INT 4.3] (1.59 g, 7.76 mmol) and Et₃N (2.65 g, 26.2 mmol) in dichloromethane (6 mL) was added a solution of [(1S)-1-(4-bromophenyl)-2,2,2-trifluoroethyl](methyl)amine hydrochloride [INT 3.1] (1.6 g, 5.25 mmol) in dichloromethane (6 mL) and the mixture was stirred at 25° C. for 16 hr. Water (30 mL) was added and the mixture was extracted with dichloromethane (30 mL×2). The combined organic layers were washed with brine (30 mL), dried over anhydrous Na₂SO₄, filtered and concentrated under reduced pressure to give the crude product which was purified by flash chromatography on silica gel (EtOAc/Petroleum ether=1/10 to 1/5) to give methyl (1S,4r)-4-(((S)-1-(4-bromophenyl)-2,2,2-trifluoroethyl)(methyl)carbamoyl)cyclohexane-1-carboxylate [INT 5.4] (1.10 g, 2.52 mmol, 32.5% yield) as yellow oil. m/z: [M+H]+ Calcd for C18H22BrF3NO3 436.1, 438.1; Found 438.0.

### Synthesis of N-[(1S)-1-(4-bromophenyl)-2,2,2-trifluoroethyl]-Nmethylthiane-4-carboxamide (Intermediate 5.5)

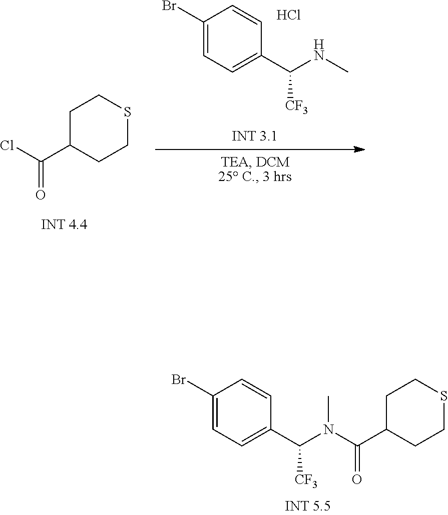 [SMILES: O=C(Cl)C1CCSCC1 ; CN[C@@H](c1ccc(Br)cc1)C(F)(F)F ; "TEA, DCM25° C., 3 hrs" ; Cl ; CN(C(=O)C1CCSCC1)[C@@H](c1ccc(Br)cc1)C(F)(F)F]

To a mixture of [(1S)-1-(4-bromophenyl)-2,2,2-trifluoroethyl](methyl)amine hydrochloride [INT 3.1] (400 mg, 1.31 mmol) and Et₃N (662 mg, 6.55 mmol) in dichloromethane (2 mL) was added a solution of thiane-4-carbonyl chloride [INT 4.4] (415 mg, 2.52 mmol) in dichloromethane (2 mL) and the mixture was stirred at 25° C. for 3 hr. The mixture was purified by flash chromatography on silica gel (EtOAc/petroleum ether=1/10 to 1/5) to give N-[(1S)-1-(4-bromophenyl)-2,2,2-trifluoroethyl]-Nmethylthiane-4-carboxamide [INT 5.5] (132 mg, 333 μmol, 25.4% yield) as a colorless oil. m/z: [M+H]+ Calcd for C15H18BrF3NOS 396.0, 398.0; Found 397.9.

### Synthesis of N-[(1S)-1-(4-bromophenyl)-2,2,2-trifluoroethyl]-N-methyloxolane-3-carboxamide (Intermediate 5.6)

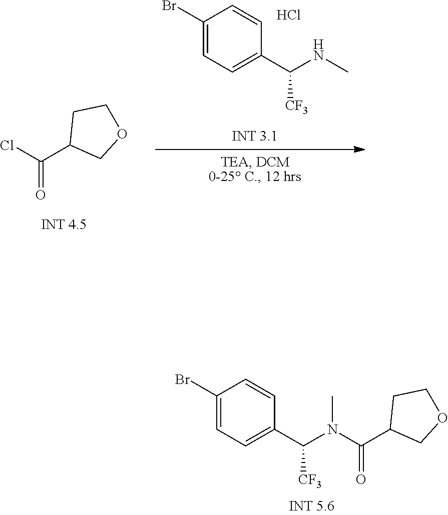 [SMILES: O=C(Cl)C1CCOC1 ; CN[C@@H](c1ccc(Br)cc1)C(F)(F)F ; "TEA, DCM0-25° C., 12 hrs" ; Cl ; CN(C(=O)C1CCOC1)[C@@H](c1ccc(Br)cc1)C(F)(F)F]

To a mixture of [(1S)-1-(4-bromophenyl)-2,2,2-trifluoroethyl](methyl)amine hydrochloride [INT 3.1] (1 g, 3.28 mmol) and triethylamine (1.65 g, 16.4 mmol) in CH₂Cl₂ (10 mL) was added oxolane-3-carbonyl chloride [INT 4.5] (662 mg, 4.92 mmol) at 0° C. The reaction mixture was stirred at 25° C. for 12 hours. The reaction mixture was concentrated, diluted with water (30 mL) and extracted with EtOAc (20 mL×3). The combined organic layers were washed with brine (30 mL×2), dried over anhydrous Na₂SO₄ and filtered. The filtrate was concentrated under reduced pressure to give the crude product, which was purified by flash chromatography on silica gel (0-15% EtOAc in petroleum ether) to give N-[(1S)-1-(4-bromophenyl)-2,2,2-trifluoroethyl]-N-methyloxolane-3-carboxamide [INT 5.6] (360 mg, 983 μmol, 30.0% yield) as a yellow oil. m/z: [M+H]+ Calcd for C14H16BrF3NO2 366.0; Found 365.7. ¹H NMR (400 MHz, CDCl₃) δ=7.55 (d, J=8.4 Hz, 2H), 7.27-7.23 (m, 2H), 6.62 (q, J=8.8 Hz, 1H), 4.14-4.01 (m, 1H), 3.99-3.86 (m, 3H), 3.38-3.28 (m, 1H), 2.88 (s, 3H), 2.30-2.09 (m, 2H).

### Synthesis of N-[(1S)-1-(4-bromophenyl)-2,2,2-trifluoroethyl]-N-methyl-1,4-dioxaspiro[4.5]decane-8-carboxamide (Intermediate 5.7)

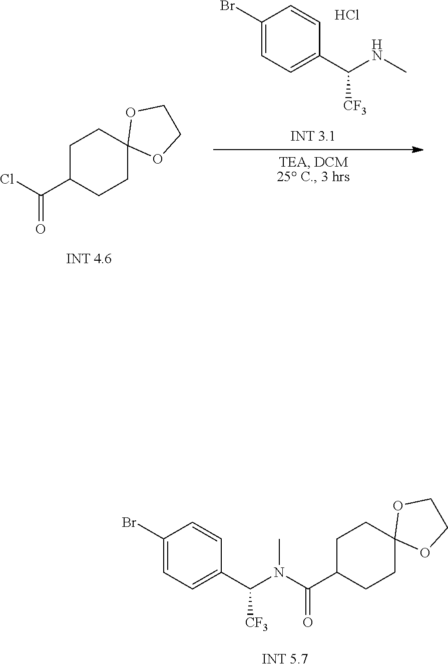 [SMILES: O=C(Cl)C1CCC2(CC1)OCCO2 ; CN[C@@H](c1ccc(Br)cc1)C(F)(F)F ; "TEA, DCM25° C., 3 hrs" ; Cl ; CN(C(=O)C1CCC2(CC1)OCCO2)[C@@H](c1ccc(Br)cc1)C(F)(F)F]

To a mixture of [(1S)-1-(4-bromophenyl)-2,2,2-trifluoroethyl](methyl)amine hydrochloride [INT 3.1] (450 mg, 1.47 mmol) and Et % N (743 mg, 7.35 mmol) in dichloromethane (2 mL) was added a solution of 1,4-dioxaspiro[4.5]decane-8-carbonyl chloride [INT 4.6] (430 mg, 2.10 mmol) in dichloromethane (2 mL) and the mixture was stirred at 25° C. for 3 hr. Water (10 mL) was added and the mixture was extracted with dichloromethane (10 mL×2). The combined organic layers were dried over anhydrous Na₂SO₄, filtered and concentrated under reduced pressure to give the crude product, which was purified by flash chromatography on silica gel (EtOAc/petroleum ether=1/10 to 1/5) to give N-[(1S)-1-(4-bromophenyl)-2,2,2-trifluoroethyl]-N-methyl-1,4-dioxaspiro[4.5]decane-8-carboxamide [INT 5.7] (210 mg, 481 μmol, 32.7% yield) as a yellow oil. m/z: [M+H]+ Calcd for C18H22BrF3NO3 436.1, 438.1; Found 437.9.

### Synthesis of N-[(1S)-1-(4-bromophenyl)-2,2,2-trifluoroethyl]-4,4-difluoro-N-methylcyclohexane-1-carboxamide (Intermediate 5.8)

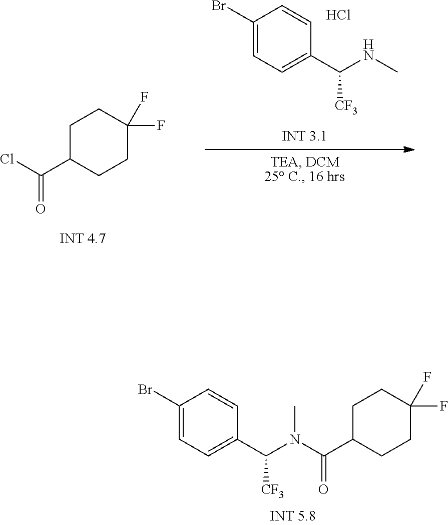 [SMILES: O=C(Cl)C1CCC(F)(F)CC1 ; CN[C@@H](c1ccc(Br)cc1)C(F)(F)F ; "TEA, DCM25° C., 16 hrs" ; Cl ; CN(C(=O)C1CCC(F)(F)CC1)[C@@H](c1ccc(Br)cc1)C(F)(F)F]

To a mixture of [(1S)-1-(4-bromophenyl)-2,2,2-trifluoroethyl](methyl)amine hydrochloride [INT 3.1] (500 mg, 1.64 mmol) and Et₃N (829 mg, 8.20 mmol) in dichloromethane (10 mL) was added a solution of 4,4-difluorocyclohexane-1-carbonyl chloride [INT 4.7] (598 mg, 3.28 mmol) in dichloromethane (10 mL) and the mixture was stirred at 25° C. for 16 hr. Water (10 mL) was added and the mixture was extracted with Dichloromethane (30 mL×2). The combined organic layers were dried over anhydrous Na₂SO₄, filtered and concentrated under reduced pressure to give the crude product, which was purified by flash chromatography on silica gel (EtOAc/petroleum ether=0/1 to 1/3) to give N-[(1S)-1-(4-bromophenyl)-2,2,2-trifluoroethyl]-4,4-difluoro-N-methylcyclohexane-1-carboxamide [INT 5.8] (539 mg, 1.30 mmol, 79.3% yield) as a colorless gum. m/z: [M+H]+ Calcd for C16H18BrF5NO 414.0; Found 414.0.

### Synthesis of (1r,3S)—N—((S)-1-(4-bromophenyl)-2,2,2-trifluoroethyl)-3-cyano-N-methylcyclobutane-1-carboxamide (Intermediate 5.9)

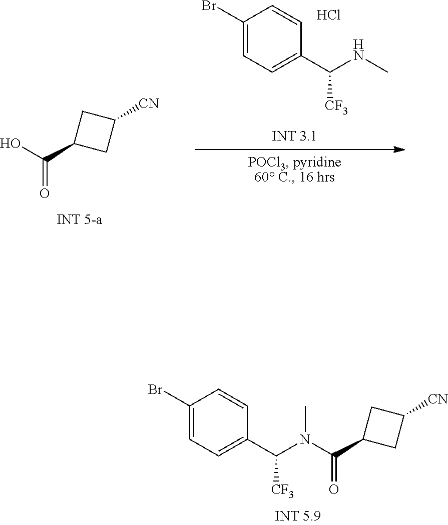 [SMILES: N#C[C@H]1C[C@H](C(=O)O)C1 ; CN[C@@H](c1ccc(Br)cc1)C(F)(F)F ; "POCl3, pyridine60° C., 16 hrs" ; Cl ; CN(C(=O)[C@H]1C[C@H](C#N)C1)[C@@H](c1ccc(Br)cc1)C(F)(F)F]

(1r,3r)-3-cyanocyclobutane-1-carboxylic acid [INT 5-a] (1 g, 7.99 mmol) and (S)-1-(4-bromophenyl)-2,2,2-trifluoro-N-methylethan-1-amine hydrochloride [INT 3.1] (2.43 g, 7.99 mmol) were mixed in pyridine (10 mL). Phosphorus oxychloride (1.22 g, 7.99 mmol) was added in one portion and the reaction mixture was stirred at 60° C. for 16 h. The reaction was quenched by adding saturated sodium bicarbonate solution (20 mL) and extracted with EtOAc (3×50 mL). The combined organic layers were dried over anhydrous Na₂SO₄ and concentrated under reduced pressure to give (1r,3S)—N—((S)-1-(4-bromophenyl)-2,2,2-trifluoroethyl)-3-cyano-N-methylcyclobutane-1-carboxamide [INT 5.9] (2.06 g, 5.49 mmol, 68.7% yield) as a brown gum. m/z: [M+H]+ Calcd for C15H15BrF3N2O 375.0; Found 375.0.

### Synthesis of 1-acetyl-N-[(1S)-1-(4-bromophenyl)-2,2,2-trifluoroethyl]-N-methylazetidine-3-carboxamide (Intermediate 5.10)

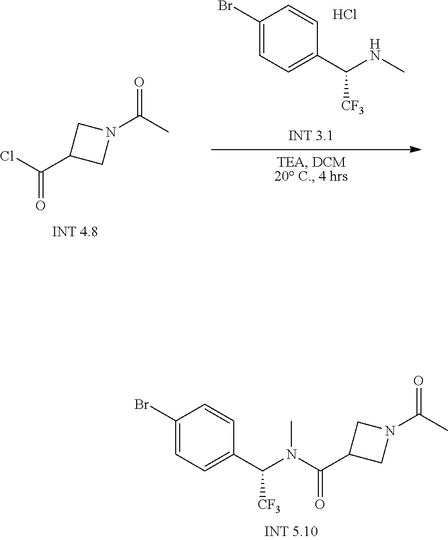

To a solution of 1-acetylazetidine-3-carbonyl chloride [INT 4.8] (300 mg, 1.85 mmol) and [(1S)-1-(4-bromophenyl)-2,2,2-trifluoroethyl](methyl)amine hydrochloride [INT 3.1] (563 mg, 1.85 mmol) in CH₂Cl₂ (3 mL) was added triethylamine (561 mg, 5.55 mmol). The mixture was stirred at 20° C. for 4 h. The reaction was concentrated under reduced pressure to give the crude product, which was purified by flash chromatography on silica gel (EtOAc/PE=0/1 to 1/5) to give 1-acetyl-N-[(1S)-1-(4-bromophenyl)-2,2,2-trifluoroethyl]-N-methylazetidine-3-carboxamide [INT 5.10] (250 mg, 635 μmol, 34.3% yield) as a yellow oil. m/z: [M+H]+ Calcd for C15H17BrF3N2O2 393.0, 395.0; Found 394.8.

### Synthesis of 1-acetyl-N-[(1S)-1-(4-bromophenyl)-2,2,2-trifluoroethyl]-N-methylpiperidine-4-carboxamide (Intermediate 5.11)

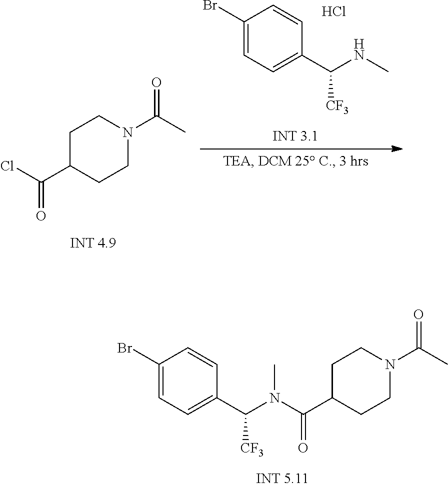 [SMILES: CC(=O)N1CCC(C(=O)Cl)CC1 ; CN[C@@H](c1ccc(Br)cc1)C(F)(F)F ; Cl ; CC(=O)N1CCC(C(=O)N(C)[C@@H](c2ccc(Br)cc2)C(F)(F)F)CC1]

To a mixture of [(1S)-1-(4-bromophenyl)-2,2,2-trifluoroethyl](methyl)amine hydrochloride [INT 3.1] (500 mg, 1.64 mmol) and Et₃N (829 mg, 8.20 mmol) in dichloromethane (10 mL) was added 1-acetylpiperidine-4-carbonyl chloride [INT 4.9] (550 mg, 2.90 mmol) and the mixture was stirred at 25° C. for 3 hr. The mixture was concentrated under reduced pressure to give the crude product, which was purified by flash chromatography on silica gel (methanol/dichloromethane=0/1 to 1/20) to give 1-acetyl-N-[(1S)-1-(4-bromophenyl)-2,2,2-trifluoroethyl]-N-methylpiperidine-4-carboxamide [INT 5.11] (680 mg, 1.61 mmol, 98.5% yield) as a yellow solid. m/z: [M+H]+ Calcd for C17H21BrF3N2O2 421.1, 423.1; Found 423.1. ¹H NMR (400 MHz, CDCl₃) δ=7.55 (d, J=8.4 Hz, 2H), 7.24 (d, J=8.4 Hz, 2H), 6.62 (q, J=8.8 Hz, 1H), 4.05-3.82 (m, 1H), 3.23-3.04 (m, 1H), 2.90 (s, 3H), 2.87-2.71 (m, 2H), 2.70-2.43 (m, 1H), 2.13 (s, 3H), 1.92-1.63 (m, 4H).

### Synthesis of 1-acetyl-N-[(1S)-1-(4-bromophenyl)-2,2,2-trifluoroethyl]-N-methylpiperidine-4-carboxamide (Intermediate 5.12)

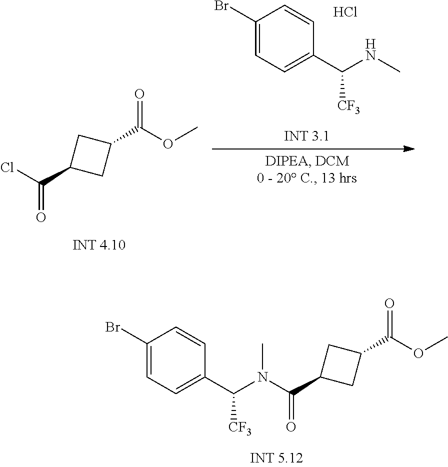 [SMILES: COC(=O)[C@H]1C[C@H](C(=O)Cl)C1 ; CN[C@@H](c1ccc(Br)cc1)C(F)(F)F ; Cl ; COC(=O)[C@H]1C[C@H](C(=O)N(C)[C@@H](c2ccc(Br)cc2)C(F)(F)F)C1]

To a mixture of methyl (1r,3r)-3-(carbonochloridoyl)cyclobutane-1-carboxylate [INT 4.10] (111 mg, 628 μmol) and [(1S)-1-(4-bromophenyl)-2,2,2-trifluoroethyl](methyl)amine hydrochloride [INT 3.1] (120 mg, 394 μmol) in CH₂Cl₂ (2 mL) was added N,N-diisopropylethylamine (254 mg, 1.97 mmol) at 0° C. The mixture was stirred at 0° C. for 1 hour and then was stirred at 20° C. for 12 h. The reaction was diluted with CH₂Cl₂ (50 mL) and 1 N HCl (20 mL). The organic phase was separated and washed with brine (20 mL), dried over anhydrous Na₂SO₄ and concentrated under reduced pressure to give the crude product, which was purified by flash chromatography on silica gel (EtOAc/PE=0/1 to 1/3) to give methyl (1S,3r)-3-(((S)-1-(4-bromophenyl)-2,2,2-trifluoroethyl)(methyl)carbamoyl)cyclobutane-1-carboxylate [INT 5.12] (84.8 mg, 207 μmol, 53.0% yield (53% purity)) as a yellow oil. m/z: [M+H]+ Calcd for C16H18BrF3NO3 408.0; Found 408.0.

### Synthesis of (S)-1-(4-bromophenyl)-2,2,2-trifluoroethan-1-amine (Intermediate 6.1)

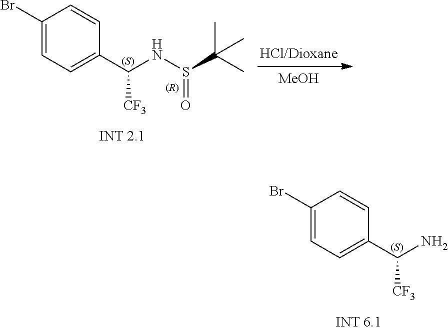

To a mixture of(S)—N-[(1S)-1-(4-bromophenyl)-2,2,2-trifluoroethyl]-2-methylpropane-2-sulfinamide [INT 2.1] (30 g, 83.7 mmol) in MeOH (100 mL) was added 4 M HCl/dioxane (30 mL). The mixture was stirred at 15° C. for 1 h. The mixture was concentrated under reduced pressure to afford the crude product. The mixture was diluted with water (50 mL) and extracted with EtOAc (100 mL*2). The combined organic layers were washed with 1 M HCl (100 ml×2). The aqueous phase was basified with 2N NaOH to pH=9-10 and extracted with CH₂Cl₂ (100 mL×2). The combined organic layers dried over anhydrous Na₂SO₄, filtered, concentrated under reduced pressure to give the (S)-1-(4-bromophenyl)-2,2,2-trifluoroethan-1-amine [INT 6.1] (11.0 g, 43.2 mmol, 51.6% yield) as an oil. m/z: [M+H]+ Calcd for C8H8BrF3N 254.0 256.0; Found 255.8.

### Synthesis of tert-butyl (S)-4-((1-(4-bromophenyl)-2,2,2-trifluoroethyl)(methyl)carbamoyl)piperidine-1-carboxylate (Intermediate 7.1)

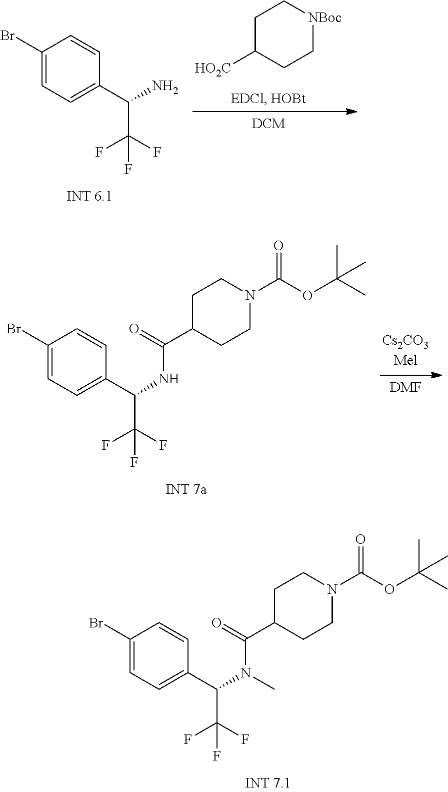

### Synthesis of tert-butyl (S)-4-((1-(4-bromophenyl)-2,2,2-trifluoroethyl)carbamoyl)piperidine-1-carboxylate (INT 7-a)

To a mixture of 1-(tert-butoxycarbonyl)piperidine-4-carboxylic acid (5.41 g, 23.6 mmol), EDCI (6.78 g, 35.4 mmol), and HOBT (4.78 g, 35.4 mmol) in CH₂Cl₂ (10 mL) was added (S)-1-(4-bromophenyl)-2,2,2-trifluoroethan-1-amine [INT 6.1] (6 g, 23.6 mmol) and the mixture was stirred at 25° C. for 16 hr. The reaction was concentrated under reduced pressure to give the crude product. The mixture was diluted with water (50 mL) and extracted with EtOAc (100 mL×2). The combined organic layers were washed with NaHCO₃ (100 mL×2). The combined organic layers were dried over anhydrous Na₂SO₄, filtered, and concentrated under reduced pressure. The resulting crude material was purified by flash chromatography on silica gel (EA/PE=0/1 to 1/5) to give tert-butyl (S)-4-((1-(4-bromophenyl)-2,2,2-trifluoroethyl)carbamoyl)piperidine-1-carboxylate [INT 7-a] (5.16 g, 11.0 mmol, 47.3% yield) as a yellow solid. ¹H NMR (400 MHz, CDCl₃) δ=7.59-7.55 (m, 2H), 7.29 (d, J=2.8 Hz, 2H), 6.18 (br d, J=9.2 Hz, 1H), 5.72 (quin, J=8.4 Hz, 1H), 4.16 (br d, J=7.2 Hz, 2H), 2.87-2.72 (m, 2H), 2.37 (tt, J=3.6, 12.0 Hz, 1H), 2.01-1.71 (m, 4H), 1.49 (s, 9H).

### Synthesis of tert-butyl (S)-4-((1-(4-bromophenyl)-2,2,2-trifluoroethyl)(methyl)carbamoyl)piperidine-1-carboxylate (INT 7.1)

To a solution of tert-butyl (S)-4-((1-(4-bromophenyl)-2,2,2-trifluoroethyl)carbamoyl)piperidine-1-carboxylate [INT 7-a] (2 g, 4.29 mmol) in DMF (30 mL) was added Cs₂CO₃ (2.79 g, 8.58 mmol) and the reaction mixture was stirred at 25° C. for 1 h. Methyl iodide (1.81 g, 12.8 mmol) was then added at 0° C. and was stirred at 25° C. for 4 h. Brine (50 mL) was added and the mixture was extracted with EtOAc (100 mL×2). The combined organic layers were washed with brine (50 mL×2), dried over anhydrous Na₂SO₄, filtered and concentrated under reduced pressure to give the crude product, which was purified by flash chromatography on silica gel (EtOAc/Petroleum ether=1/5 to 1/0) to give tert-butyl (S)-4-((1-(4-bromophenyl)-2,2,2-trifluoroethyl)(methyl)carbamoyl)piperidine-1-carboxylate [INT 7.1] (1.20 g, 2.50 mmol, 58.5% yield) as a white solid. ¹H NMR (400 MHz, CHLOROFORM-d) Shift=7.57-7.51 (m, 2H), 7.24 (d, J=8.4 Hz, 2H), 6.63 (q, J=9.2 Hz, 1H), 4.30-4.14 (m, 2H), 2.89 (s, 3H), 2.86-2.65 (m, 3H), 1.85-1.65 (m, 4H), 1.47 (s, 9H).

### Synthesis of tert-butyl 3-(((S)-1-(4-bromophenyl)-2,2,2-trifluoroethyl)(methyl)carbamoyl)piperidine-1-carboxylate (Intermediate 7.2)

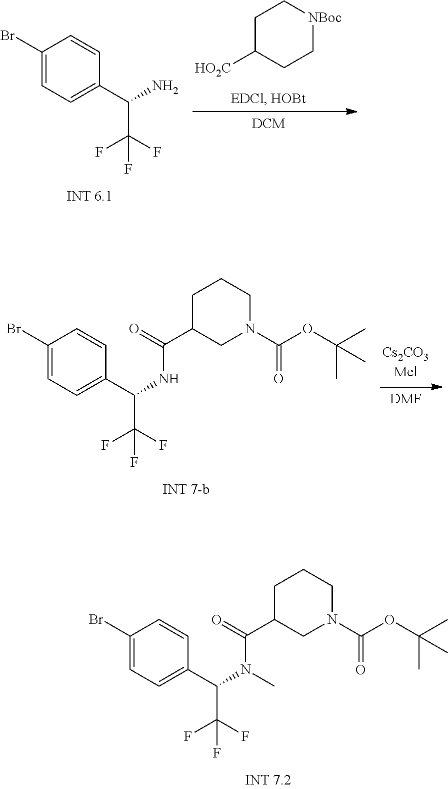

### Synthesis of tert-butyl 3-(((S)-1-(4-bromophenyl)-2,2,2-trifluoroethyl)carbamoyl)piperidine-1-carboxylate (INT 7-b)

To a mixture of 1-[(tert-butoxy)carbonyl]piperidine-3-carboxylic acid (1.94 g, 8.49 mmol), EDCI (2.03 g, 10.6 mmol), and HOBT (1.43 g, 10.6 mmol) in CH₂Cl₂ (20 mL) was added (S)-1-(4-bromophenyl)-2,2,2-trifluoroethan-1-amine [INT 6.1] (1.8 g, 7.08 mmol) and the mixture was stirred at 25° C. for 16 hr. The reaction was quenched by adding water (50 mL) and was extracted with EtOAc (50 mL×3). The combined organic layers were washed with brine (50 mL×2), dried over anhydrous Na₂SO₄ and concentrated under reduced pressure to give the crude product which was purified by flash chromatography on silica gel (EtOAc/PE=0/1 to 1/5) to give tert-butyl 3-(((S)-1-(4-bromophenyl)-2,2,2-trifluoroethyl)carbamoyl)piperidine-1-carboxylate [INT 7-b] (1.40 g, 3.00 mmol, 42.5% yield) as a yellow solid. m/z: [M+H−56]+ Calcd for C19H25BrF3N2O3 408.9; Found 408.8.

### Synthesis of tert-butyl 3-(((S)-1-(4-bromophenyl)-2,2,2-trifluoroethyl)(methyl)carbamoyl)piperidine-1-carboxylate (INT 7.2)

To a solution of tert-butyl 3-(((S)-1-(4-bromophenyl)-2,2,2-trifluoroethyl)carbamoyl)piperidine-1-carboxylate [INT 7-b] (1.4 g, 3.00 mmol) in DMF (10 mL) was added Cs₂CO₃ (1.95 g, 6.00 mmol) and the reaction mixture was stirred at 25° C. for 1 h. Methyl iodide (1.27 g, 9.00 mmol) was then added at 0° C. and the reaction was stirred at 25° C. for 4 h. The reaction was quenched by adding water (50 mL), then it was extracted with EtOAc (50 mL×3). The combined organic layers were washed with brine (50 mL×2), dried over anhydrous Na₂SO₄ and concentrated under reduced pressure to give the crude product which was purified by flash chromatography on silica gel (PE/EtOAc=1/O to 5/1) to give tert-butyl 3-(((S)-1-(4-bromophenyl)-2,2,2-trifluoroethyl)(methyl)carbamoyl)piperidine-1-carboxylate [INT 7.2] (800 mg, 1.66 mmol, 55.9% yield) as a colorless oil. m/z: [M−56+H]+ Calcd for C20H27BrF3N2O3 425.1; Found 424.7.

### Synthesis of Tert-butyl 3-(((S)-1-(4-bromophenyl)-2,2,2-trifluoroethyl)(methyl)carbamoyl)pyrrolidine-1-carboxylate (Intermediate 7.3)

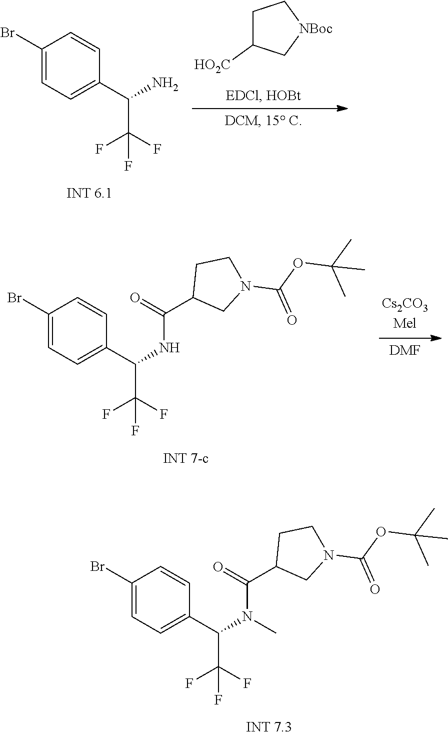

### Synthesis of tert-butyl 3-(((S)-1-(4-bromophenyl)-2,2,2-trifluoroethyl)carbamoyl)pyrrolidine-1-carboxylate (INT 7-c)

To a mixture of 1-[(tert-butoxy)carbonyl]pyrrolidine-3-carboxylic acid (1.69 g, 7.87 mmol), EDCI (2.26 g, 11.8 mmol), and HOBT (1.59 g, 11.8 mmol) in CH₂Cl₂ (30 mL) was added (S)-1-(4-bromophenyl)-2,2,2-trifluoroethan-1-amine [INT 6.1] (2 g, 7.87 mmol) and the mixture was stirred at 15° C. for 16 hr. The reaction was concentrated under reduced pressure to give the crude product. This crude product was combined with material from a separate identical reaction on the same scale. The mixture was diluted with water (50 mL) and extracted with EtOAc (100 mL×2). The combined organic layers were washed with NaHCO₃ (100 mL×2). The combined organic layers were dried over anhydrous Na₂SO₄, filtered, and concentrated under reduced pressure to give crude material which was purified by flash chromatography on silica gel (EA/PE=0/1 to 1/5) to give tert-butyl 3-(((S)-1-(4-bromophenyl)-2,2,2-trifluoroethyl)carbamoyl)pyrrolidine-1-carboxylate [INT 7-c] (4.26 g, 9.43 mmol, 59.9% yield) as a yellow oil. m/z: [M−56+H]+ Calcd for C18H23BrF3N2O3 395.0, 397.0; Found 396.6. ¹H NMR (400 MHz, CDCl₃) δ=7.55 (br d, J=8.0 Hz, 2H), 7.25 (br s, 2H), 6.22 (br s, 1H), 5.69 (quin, J=8.0 Hz, 1H), 3.72-3.42 (m, 3H), 3.40-3.29 (m, 1H), 3.03-2.88 (m, 1H), 2.06-2.03 (m, 2H), 1.46 (d, J=6.8 Hz, 9H).

### Synthesis of tert-butyl 3-(((S)-1-(4-bromophenyl)-2,2,2-trifluoroethyl)(methyl)carbamoyl)pyrrolidine-1-carboxylate (INT 7.3)

To a solution of tert-butyl 3-(((S)-1-(4-bromophenyl)-2,2,2-trifluoroethyl)carbamoyl)pyrrolidine-1-carboxylate [INT 7-c] (4.26 g, 9.43 mmol) in DMF (50 mL) was added Cs₂CO₃ (6.12 g, 18.8 mmol) and the reaction mixture was stirred at 25° C. for 1 h. Methyl iodide (4.00 g, 28.2 mmol) was then added at 0° C. and the reaction was stirred at 25° C. for 4 h. The reaction was quenched by adding water (50 mL), then it was extracted with EtOAc (100 mL×3). The combined organic layers were washed with brine (100 mL×2), dried over anhydrous Na₂SO₄ and concentrated under reduced pressure to give the crude product which was purified by flash chromatography on silica gel (EtOAc/Petroleum ether=1/5 to 1/0) to give tert-butyl 3-(((S)-1-(4-bromophenyl)-2,2,2-trifluoroethyl)(methyl)carbamoyl)pyrrolidine-1-carboxylate [INT 7.3] (2.20 g, 4.73 mmol, 50.2% yield) as a yellow oil. [M−56+H]+ Calcd for C19H25BrF3N2O3 409.1 Found 409.1. ¹H NMR (400 MHz, CDCl₃) δ=7.61-7.51 (m, 2H), 7.24 (br d, J=6.4 Hz, 2H), 6.61 (q, J=8.8 Hz, 1H), 3.77-3.49 (m, 3H), 3.46-3.36 (m, 1H), 3.33-3.21 (m, 1H), 3.14 (s, 1H), 2.89 (s, 2H), 2.25-2.07 (m, 2H), 1.48-1.41 (m, 9H).

### Tert-butyl (S)-((4-((1-(4-bromophenyl)-2,2,2-trifluoroethyl)(methyl)carbamoyl)cyclohexyl)methyl)carbamate (Intermediate 7.4)

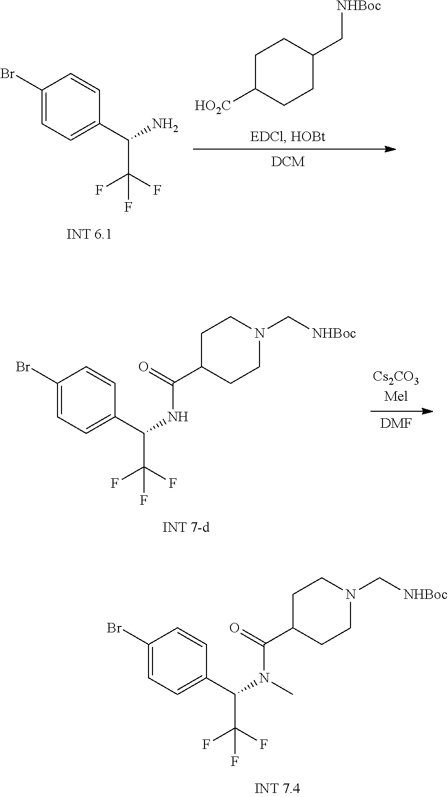

### Synthesis of tert-butyl (S)-((4-((1-(4-bromophenyl)-2,2,2-trifluoroethyl)carbamoyl)cyclohexyl)methyl)carbamate (INT 7-d)

To a mixture of 4-({[(tert-butoxy)carbonyl]amino}methyl)cyclohexane-1-carboxylic acid (1.92 g, 7.47 mmol), EDCI (2.16 g, 11.2 mmol), and HOBT (1.51 g, 11.2 mmol) in CH₂Cl₂ (20 mL) was added (S)-1-(4-bromophenyl)-2,2,2-trifluoroethan-1-amine [INT 6.1] (1.9 g, 7.47 mmol) and the mixture was stirred at 25° C. for 16 hr. The reaction was quenched by adding saturated Na₂CO₃ (100 mL) and extracted with EtOAc (100 mL×3). The combined organic layers were washed with brine (200 mL×2), dried over anhydrous Na₂SO₄ and concentrated under reduced pressure to give the crude product which was purified by flash chromatography on silica gel (EtOAc/dichloromethane=0/1 to 1/9) to give tert-butyl (S)-((4-((1-(4-bromophenyl)-2,2,2-trifluoroethyl)carbamoyl)cyclohexyl)methyl)carbamate [INT 7-d] (2.53 g, 5.12 mmol, 68.7% yield) as a white solid. m/z: [M+H−56]+ Calcd for C21H29BrF3N2O3 437.1, Found 436.9. ¹H NMR (400 MHz, CD₃OD) δ=7.58 (br d, J=8.4 Hz, 2H), 7.40 (br d, J=8.4 Hz, 2H), 5.68 (q, J=8.0 Hz, 1H), 4.61 (br s, 1H), 2.89 (br d, J=6.4 Hz, 2H), 2.31 (br t, J=12.0 Hz, 1H), 1.97-1.66 (m, 5H), 1.47-1.40 (m, 3H), 1.43 (s, 9H), 1.07-0.90 (m, 2H).

### Synthesis of tert-butyl (S)-((4-((1-(4-bromophenyl)-2,2,2-trifluoroethyl)(methyl)carbamoyl)cyclohexyl)methyl)carbamate (INT 7.4)

A suspension of tert-butyl (S)-((4-((1-(4-bromophenyl)-2,2,2-trifluoroethyl)carbamoyl)cyclohexyl)methyl)carbamate [INT 7-d] (1 g, 2.02 mmol) and Cs₂CO₃ (1.97 g, 6.06 mmol) in DMF (15 mL) was stirred for 1 h. Then MeI (860 mg, 6.06 mmol) was added and the resulting mixture was stirred for 3 h. The reaction mixture was poured into water (100 mL) and extracted with EtOAc (100 mL×2). The combined organic layers were washed with water (200 mL×2) and brine (200 mL), dried over Na₂SO₄, and filtered. The filtrate was concentrated to give a residue which was purified by prep-HPLC (column: Boston Prime C18 150*30 mm*5 μm, table: 54-84% B (A=water (0.05% ammonia hydroxide v/v), B=acetonitrile), flow rate: 30 mL/min, UV Detector 220 nm) to afford tert-butyl (S)-((4-((1-(4-bromophenyl)-2,2,2-trifluoroethyl)(methyl)carbamoyl)cyclohexyl)methyl)carbamate [INT 7.4] (400 mg, 788 μmol, 39.2% yield) as a white solid. m/z: [M−56+H]+ Calcd for C22H31 BrF3N2O3 451.1, 453.1; Found 452.9. ¹H NMR (400 MHz, CDCl₃) δ=7.58-7.50 (m, 2H), 7.25 (d, J=8.4 Hz, 2H), 6.65 (q, J=8.8 Hz, 1H), 4.60 (br s, 1H), 3.02 (br t, J=6.4 Hz, 2H), 2.87 (s, 3H), 2.53 (tt, J=3.2, 11.6 Hz, 1H), 1.96-1.77 (m, 4H), 1.71-1.54 (m, 3H), 1.46 (s, 9H), 1.08-0.95 (m, 2H).

### (1r,4S)—N—((S)-1-(4-bromophenyl)-2,2,2-trifluoroethyl)-4-methoxy-N-methylcyclohexane-1-carboxamide (Intermediate 7.5)

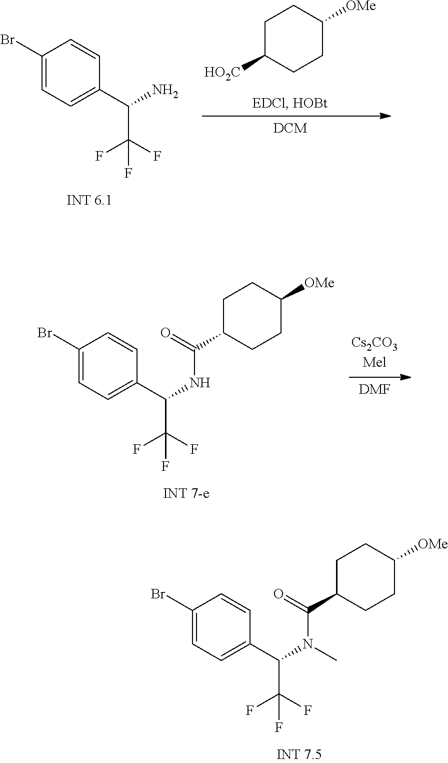

### Synthesis of (1r,4S)—N—((S)-1-(4-bromophenyl)-2,2,2-trifluoroethyl)-4-methoxycyclohexane-1-carboxamide (INT 7-e)

To a mixture of (1r,4r)-4-methoxycyclohexane-1-carboxylic acid (435 mg, 2.75 mmol), EDCI (790 mg, 4.12 mmol), and HOBt (556 mg, 4.12 mmol) in CH₂Cl₂ (20 mL) was added (S)-1-(4-bromophenyl)-2,2,2-trifluoroethan-1-amine [INT 6.1] (700 mg, 2.75 mmol) and the mixture was stirred at 25° C. for 16 hr. The reaction mixture was quenched by adding water (10 10 mL) and was extracted with EtOAc (10 mL×3). The combined organic layers were washed with saturated NaHCO₃ (20 mL) and brine (10 mL×2), dried over anhydrous Na₂SO₄ and concentrated under reduced pressure to give the crude product which was purified by flash chromatography on silica gel (EtOAc/PE=0/1 to 1/3) to give (1r,4S)—N—((S)-1-(4-bromophenyl)-2,2,2-trifluoroethyl)-4-methoxycyclohexane-1-carboxamide [INT 7-e] (456 mg, 1.15 mmol, 42.2% yield) as a white solid. m/z: [M+H]+ Calcd for C16H20BrF3NO2 394.1, 396.1; Found 395.9. ¹H NMR (400 MHz, CD₃OD) δ=7.58 (d, J=8.6 Hz, 2H), 7.40 (d, J=8.4 Hz, 2H), 5.68 (q, J=8.3 Hz, 1H), 3.34 (s, 3H), 3.24-3.09 (m, 1H), 2.41-2.24 (m, 1H), 2.20-2.05 (m, 2H), 1.95-1.84 (m, 1H), 1.83-1.71 (m, 1H), 1.64-1.39 (m, 2H), 1.30-1.09 (m, 2H).

### Synthesis of (1r,4S)—N—((S)-1-(4-bromophenyl)-2,2,2-trifluoroethyl)-4-methoxy-N-methylcyclohexane-1-carboxamide (INT 7.5)

A mixture of (1r,4S)—N—((S)-1-(4-bromophenyl)-2,2,2-trifluoroethyl)-4-methoxycyclohexane-1-carboxamide [INT 7-e] (455 mg, 1.15 mmol) and Cs₂CO₃ (749 mg, 2.30 mmol) in DMF (1 mL) was stirred at 25° C. for 1 h. CH₃I (816 mg, 5.75 mmol) was added and the mixture was stirred for 2 h. The reaction was quenched by adding water (10 mL) and was extracted with EtOAc (2×10 mL). The combined organic layers were washed with brine (2×10 mL), dried over anhydrous Na₂SO₄ and concentrated under reduced pressure to give the crude product which was purified by flash chromatography on silica gel (EtOAc/PE=0/1 to 1/3) to give (1r,4S)—N—((S)-1-(4-bromophenyl)-2,2,2-trifluoroethyl)-4-methoxy-N-methylcyclohexane-1-carboxamide [INT 7.5] (130 mg, 318 μmol, 27.7% yield) as a colorless oil. m/z: [M+H]+ Calcd for C17H22BrF3NO2 408.1; Found 407.8.

### (1r,4S)—N—((S)-1-(4-bromophenyl)-2,2,2-trifluoroethyl)-4-((tert-butyldimethylsilyl)oxy)-N-methylcyclohexane-1-carboxamide (Intermediate 8.1)

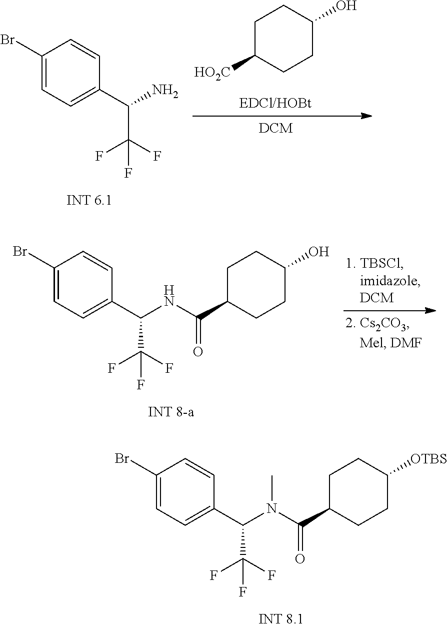 [SMILES: N[C@@H](c1ccc(Br)cc1)C(F)(F)F ; O=C(O)[C@H]1CC[C@H](O)CC1 ; O=C(N[C@@H](c1ccc(Br)cc1)C(F)(F)F)[C@H]1CC[C@H](O)CC1 ; CN(C(=O)[C@H]1CC[C@H](O[Si](C)(C)C(C)(C)C)CC1)[C@@H](c1ccc(Br)cc1)C(F)(F)F]

### Synthesis of (1r,4S)—N—((S)-1-(4-bromophenyl)-2,2,2-trifluoroethyl)-4-hydroxycyclohexane-1-carboxamide (INT 8-a)

To a mixture of (1r,4r)-4-hydroxycyclohexane-1-carboxylic acid (1.13 g, 7.87 mmol), EDCI (2.28 g, 11.8 mmol), and HOBt (1.59 g, 11.8 mmol) in CH₂Cl₂ (20 mL) was added (S)-1-(4-bromophenyl)-2,2,2-trifluoroethan-1-amine [INT 6.1] (2 g, 7.87 mmol) and the mixture was stirred at 25° C. for 16 hr. The reaction was quenched by adding water (100 mL) and was extracted with CH₂Cl₂ (100 mL×3). The combined organic layers were washed with saturated aqueous NaHCO₃ (200 mL×2) and brine (200 mL), dried over anhydrous Na₂SO₄ and concentrated under reduced pressure to give the crude product which was purified by flash chromatography on silica gel (EtOAc/PE=1/1 to 1/0) to give (1r,4S)—N—((S)-1-(4-bromophenyl)-2,2,2-trifluoroethyl)-4-hydroxycyclohexane-1-carboxamide [INT 8-a] (1.80 g, 4.73 mmol, 60.1% yield) as a white solid. m/z: [M+H]+ Calcd for C15H18BrF3NO2 380.0, 382.0; Found 381.7.

### Synthesis of (1r,4S)—N—((S)-1-(4-bromophenyl)-2,2,2-trifluoroethyl)-4-((tert-butyldimethylsilyl)oxy)-N-methylcyclohexane-1-carboxamide (INT 8.1)

A suspension of (1r,4S)—N—((S)-1-(4-bromophenyl)-2,2,2-trifluoroethyl)-4-hydroxycyclohexane-1-carboxamide [INT 8-a] (500 mg, 1.31 mmol), imidazole (178 mg, 2.62 mmol) and tert-butyl(chloro)dimethylsilane (295 mg, 1.96 mmol) in CH₂Cl₂ (5 mL) was stirred at 25° C. for 12 h. The reaction mixture was poured into water (30 mL) and extracted with EtOAc (30 mL×2). The combined organic layers was washed with saturated Na₂CO₃ (100 mL×2) and brine (100 mL), dried over Na₂SO₄, filtered and concentrated under reduced pressure to give the crude product, which was purified by flash chromatography on silica gel (EtOAc/Petroleum ether=0/1 to 5/95) to give (1r,4r)-N-[(1S)-1-(4-bromophenyl)-2,2,2-trifluoroethyl]-4-[(tertbutyldimethylsilyl)oxy]cyclohexane-1-carboxamide (440 mg, 889 μmol, 68.0% yield) as a white solid. m/z: [M+H]+ Calcd for C21H32BrF3NO2Si 496.1; Found 496.0. ¹H NMR (400 MHz, CDCl₃) δ=7.54 (d, J=8.4 Hz, 2H), 7.25 (d, J=8.4 Hz, 2H), 6.09 (br d, J=9.2 Hz, 1H), 5.75-5.62 (m, 1H), 3.62-3.51 (m, 1H), 2.18-2.05 (m, 1H), 1.98-1.79 (m, 4H), 1.30-1.22 (m, 4H), 0.89 (s, 9H), 0.06 (s, 6H).

A suspension of (1r,4r)-N-[(1S)-1-(4-bromophenyl)-2,2,2-trifluoroethyl]-4-[(tert-butyldimethylsilyl)oxy]cyclohexane-1-carboxamide (1.15 g, 2.32 mmol) and Cs₂CO₃ (2.26 g, 6.96 mmol) in DMF (3 mL) was stirred at 20° C. for 1 h. Then MeI (987 mg, 6.96 mmol) was added and the reaction mixture was stirred at 20° C. for 3 h. The reaction mixture was combined with a mixture from an additional identical reaction, poured into water (30 mL) and extracted with EtOAc (30 mL×2). The combined organic layers were washed with water (50 mL×2) and brine (50 mL), dried over Na₂SO₄, filtered and concentrated under reduced pressure to give the crude material. This material was purified by flash chromatography on silica gel (EtOAc/Petroleum ether=0/1 to 3/97) to give (1r,4S)—N—((S)-1-(4-bromophenyl)-2,2,2-trifluoroethyl)-4-((tert-butyldimethylsilyl)oxy)-N-methylcyclohexane-1-carboxamide [INT 8.1] (495 mg, 973 μmol, 42.3% yield) as a white solid. m/z: [M+H]+ Calcd for C22H34BrF3NO2Si 508.1, 510.1; Found 510.1. ¹H NMR (400 MHz, CDCl₃) δ=7.53 (d, J=8.4 Hz, 2H), 7.23 (d, J=8.4 Hz, 2H), 6.63 (q, J=8.8 Hz, 1H), 3.70-3.55 (m, 1H) 2.86 (s, 3H), 2.50 (tt, J=3.6, 11.2 Hz, 1H), 2.03-1.93 (m, 2H), 1.93-1.83 (m, 1H), 1.83-1.74 (m, 1H), 1.71-1.60 (m, 2H), 1.41-1.30 (m, 2H), 0.89 (s, 9H), 0.07 (s, 6H).

### Methyl (S)-(4-((1-(4-bromophenyl)-2,2,2-trifluoroethyl)(methyl)carbamoyl)cyclohexyl)carbamate (Intermediate 9.1)

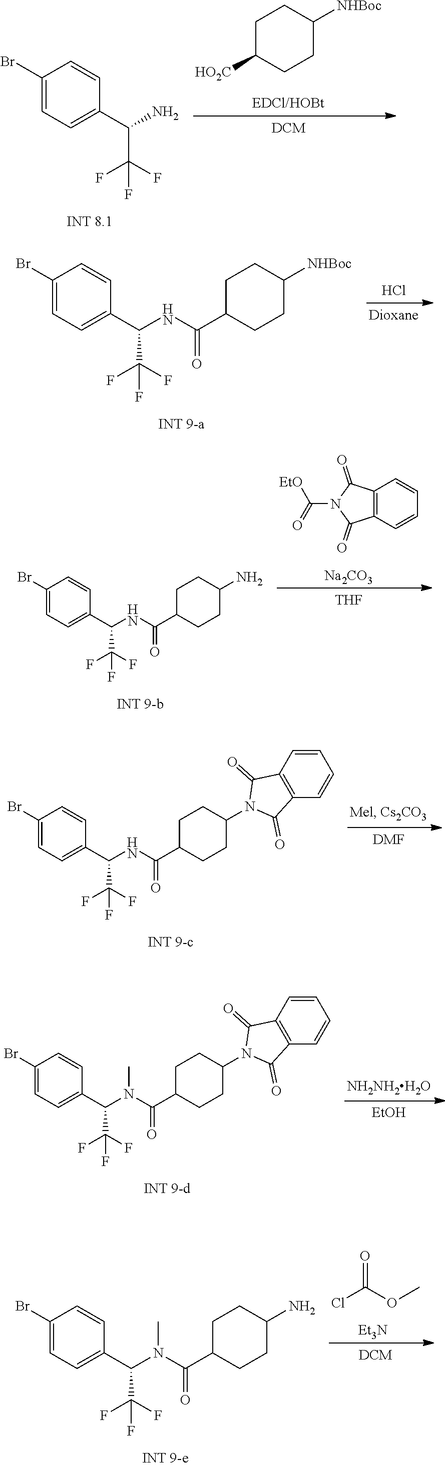

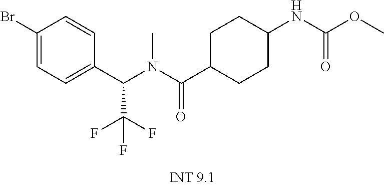 [SMILES: COC(=O)NC1CCC(C(=O)N(C)[C@@H](c2ccc(Br)cc2)C(F)(F)F)CC1]

### Synthesis of tert-butyl (S)-(4-((1-4-bromophenyl)-2,2,2-trifluoroethyl)carbamoyl)cyclohexyl)carbamate (INT 9-a)

To a mixture of 4-{[(tert-butoxy)carbonyl]amino}cyclohexane-1-carboxylic acid (1.43 g, 5.90 mmol), EDCI (1.69 g, 8.85 mmol), and HOBt (1.19 g, 8.85 mmol) in CH₂Cl₂ (2 mL) was added (S)-1-(4-bromophenyl)-2,2,2-trifluoroethan-1-amine [INT 6.1] (2.5 g, 5.90 mmol) and the mixture was stirred at 25° C. for 16 hr. The reaction mixture was quenched by adding water (30 mL) and was extracted with EtOAc (2×100 mL). The combined organic layers were washed with brine (2×50 mL), dried over anhydrous Na₂SO₄ and concentrated under reduced pressure to give the crude product which was purified by flash chromatography on silica gel (EtOAc/PE=0/1 to 1/3) to give tert-butyl (S)-(4-((1-(4-bromophenyl)-2,2,2-trifluoroethyl)carbamoyl)cyclohexyl)carbamate [INT 9-a] (2.70 g, 5.63 mmol, 95.7% yield) as a white solid. m/z: [M−Boc]+ Calcd for C20H26BrF3N2O3 378.1; Found 378.9.

### Synthesis of (S)-4-amino-N-(1-(4-bromophenyl)-2,2,2-trifluoroethyl)cyclohexane-1-carboxamide hydrochloride (INT 9-b)

A solution of tert-butyl (S)-(4-((1-(4-bromophenyl)-2,2,2-trifluoroethyl)carbamoyl)cyclohexyl)carbamate [INT 9-a] (3.1 g, 6.46 mmol) in 4 M HCl in dioxane (30 mL) was stirred at 25° C. for 1 h. The mixture was concentrated under reduced pressure to give (S)-4-amino-N-(1-(4-bromophenyl)-2,2,2-trifluoroethyl)cyclohexane-1-carboxamide hydrochloride [INT 9-b] (2.60 g, 6.25 mmol, 97.0% yield) as a white solid. m/z: [M+H]+ Calcd for C15H19BrF3N2O 379.1; Found 379.1.

### Synthesis of(S)—N-(1-(4-bromophenyl)-2,2,2-trifluoroethyl)-4-(1,3-dioxoisoindolin-2-yl)cyclohexane-1-carboxamide (INT 9-c)

A mixture of(S)-4-amino-N-(1-(4-bromophenyl)-2,2,2-trifluoroethyl)cyclohexane-1-carboxamide hydrochloride [INT 9-b] (1.35 g, 3.55 mmol), ethyl 1,3-dioxo-2,3-dihydro-1Hisoindole-2-carboxylate (1.16 g, 5.32 mmol), and Na₂CO₃ (1.12 g, 10.6 mmol) in THF (20 mL) was stirred at 25° C. for 1 h. The reaction was combined with a crude batch of the same reaction at the same scale and concentrated under reduced pressure to give a crude product. This crude product was triturated with EtOAc/PE(1/1, 50 mL) to give (S)—N-(1-(4-bromophenyl)-2,2,2-trifluoroethyl)-4-(1,3-dioxoisoindolin-2-yl)cyclohexane-1-carboxamide [INT 9-c] (3.60 g, 7.06 mmol, 99.4% yield) as a white solid. m/z: [M+Na]+ Calcd for C23H20BrF3N2O3 531.1, 533.1; Found 532.8.

### Synthesis of(S)—N-(1-(4-bromophenyl)-2,2,2-trifluoroethyl)-4-(1,3-dioxoisoindolin-2-yl)-N-methylcyclohexane-1-carboxamide (INT 9-d)

A mixture of (S)—N-(1-(4-bromophenyl)-2,2,2-trifluoroethyl)-4-(1,3-dioxoisoindolin-2-yl)cyclohexane-1-carboxamide [INT 9-c] (3.7 g, 7.26 mmol) and Cs₂CO₃ (4.72 g, 14.5 mmol) in DMF (40 mL) was stirred at 25° C. for 1 h. CH₃I (3.07 g, 21.7 mmol) was added and the reaction mixture was stirred at 25° C. for 1 h. The reaction was quenched by adding water (50 mL) and was extracted with EtOAc (2×100 mL). The combined organic layers were washed with brine (2×50 mL), dried over anhydrous Na₂SO₄ and concentrated under reduced pressure to give the crude product which was purified by flash chromatography on silica gel (EtOAc/PE=0/1 to 1/3) to give (S)—N-(1-(4-bromophenyl)-2,2,2-trifluoroethyl)-4-(1,3-dioxoisoindolin-2-yl)-N-methylcyclohexane-1-carboxamide [INT 9-d] (996 mg, 1.90 mmol, 26.2% yield) as a white solid. m/z: [M+H]+ Calcd for C24H23BrF3N2O3 523.1; Found 523.2.

### Synthesis of (S)-4-amino-N-(1-(4-bromophenyl)-2,2,2-trifluoroethyl)-N-methylcyclohexane-1-carboxamide (INT 9-e)

To a mixture of (S)—N-(1-(4-bromophenyl)-2,2,2-trifluoroethyl)-4-(1,3-dioxoisoindolin-2-yl)-N-methylcyclohexane-1-carboxamide [INT 9-d] (300 mg, 573 μmol) in EtOH (1 mL) was added NH₂NH₂·H₂O (143 mg, 2.86 mmol) and the mixture was stirred at 20° C. for 16 h. The reaction mixture was filtered and the filtrate was concentrated under reduced pressure to give (S)-4-amino-N-(1-(4-bromophenyl)-2,2,2-trifluoroethyl)-N-methylcyclohexane-1-carboxamide [INT 9-e] (220 mg, 559 μmol, 97.7% yield) as a yellow solid. m/z: [M−NH2]+ Calcd for C16H20BrF3N2O 376.1; Found 376.1.

### Synthesis of methyl (S)-(4-((1-(4-bromophenyl)-2,2,2-trifluoroethyl)(methyl)carbamoyl)cyclohexyl)carbamate (INT 9.1)

To a solution of (S)-4-amino-N-(1-(4-bromophenyl)-2,2,2-trifluoroethyl)-N-methylcyclohexane-1-carboxamide [INT 9-e] (90 mg, 228 μmol) and Et₃N (115 mg, 1.14 mmol) in CH₂Cl₂ (1 mL) was added methyl carbonochloridate (64.6 mg, 684 μmol) and the mixture was stirred for 1 h. The reaction was quenched by adding water (10 mL) and was extracted with EtOAc (2×10 mL). The combined organic layers were washed with brine (2×10 mL), dried over anhydrous Na₂SO₄ and concentrated under reduced pressure to give the crude product, which was purified by prep-TLC (EtOAc/PE=1/2) to give methyl (S)-(4-((1-(4-bromophenyl)-2,2,2-trifluoroethyl)(methyl)carbamoyl)cyclohexyl)carbamate [INT 9.1] (60.0 mg, 132 μmol, 58.8% yield) as a colorless oil. m/z: [M+H]+ Calcd for C18H23BrF3N2O3 451.1, 453.1; Found 452.9.

### Synthesis of (S)-4-acetamido-N-(1-(4-bromophenyl)-2,2,2-trifluoroethyl)-N-methylcyclohexane-1-carboxamide (Intermediate 9.2)

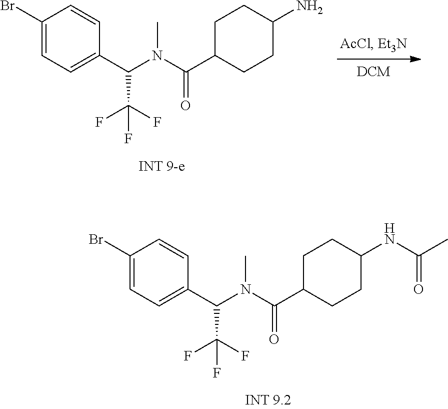

To a mixture of (S)-4-amino-N-(1-(4-bromophenyl)-2,2,2-trifluoroethyl)-N-methylcyclohexane-1-carboxamide [INT 9-e] (110 mg, 279 μmol) and triethylamine (140 mg, 1.39 mmol) in DCM (1 mL) was added acetyl chloride (76.6 mg, 976 μmol) at 25° C. and the mixture was stirred at 25° C. for 1 hr. The reaction mixture was concentrated under reduced pressure to give the crude product (S)-4-acetamido-N-(1-(4-bromophenyl)-2,2,2-trifluoroethyl)-N-methylcyclohexane-1-carboxamide [INT 9.2] (120 mg, 275 μmol, 99.1% yield) as a white solid. m/z: [M+H]+ Calcd for C18H23BrF3N2O2 435.1; Found 435.2.

### Synthesis of 1-(4-bromophenyl)-N-methylethan-1-amine (Intermediate 10.1)

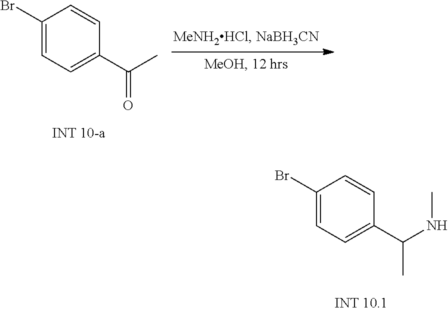

To a solution of 1-(4-bromophenyl)ethan-1-one [INT 10-a] (10 g, 50.3 mmol) in MeOH (100 mL) was added sodium cyanoborohydride (8.79 g, 140 mmol) and methanamine hydrochloride (31.5 g, 468 mmol). The mixture was stirred at 20° C. for 12 h. The reaction was quenched by adding water (100 mL) and was extracted with EtOAc (100 mL×2). The combined organic layers were washed with brine (2×50 mL), dried over anhydrous Na₂SO₄ and concentrated under reduced pressure to give the crude product, which was purified by flash chromatography on silica gel (EtOAc/PE=0/1 to 1/0) to give 1-(4-bromophenyl)-N-methylethan-1-amine [INT 10.1] (7.50 g, 32.8 mmol, 65.2% yield) as a colorless oil. m/z: [M+H]+ Calcd for C9H13BrN 214.0; Found 213.9.

### Synthesis of N-(1-(4-bromophenyl)ethyl)-N-methylcyclobutanecarboxamide (Intermediate 11.1)

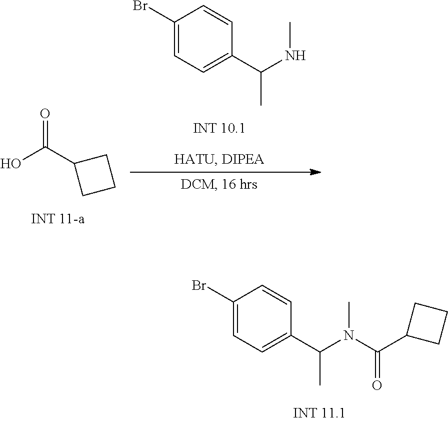 [SMILES: CNC(C)c1ccc(Br)cc1 ; O=C(O)C1CCC1 ; CC(c1ccc(Br)cc1)N(C)C(=O)C1CCC1]

To a mixture of cyclobutanecarboxylic acid [INT 11-a] (389 mg, 3.89 mmol) and HATU (2.21 g, 5.83 mmol) in CH₂Cl₂ (15 mL) was added DIPEA (1.49 g, 11.6 mmol) and 1-(4-bromophenyl)-N-methylethan-1-amine [INT 10.1] (1 g, 4.67 mmol). The reaction mixture was stirred at 20° C. under nitrogen for 16 hours. The reaction mixture was concentrated under reduced pressure to give crude product, which was purified by prep-HPLC (column: Phenomenex Gemini-NX 80*40 mm*3 um, table: 22-62% B (A=water(0.05% ammonia hydroxide)), B=acetonitrile), flow rate: 25 mL/min, UV Detector 220 nm) to afford N-(1-(4-bromophenyl)ethyl)-N-methylcyclobutanecarboxamide [INT 11.1] (150 mg, 506 μmol, 13.0% yield) as an off-white dry powder. m/z: [M+H]+ Calcd for C14H19BrNO 296.1, 298.1; Found 298.1.

### Synthesis of N-[1-(4-bromophenyl)ethyl]-N-methylcyclohexanecarboxamide (Intermediate 11.2)

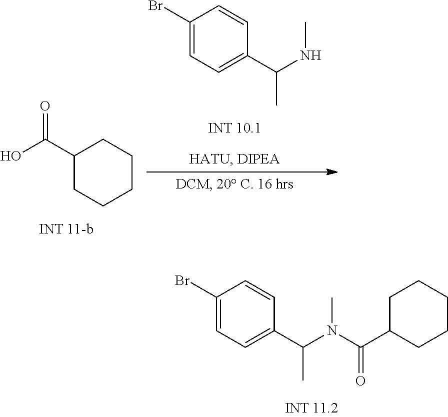 [SMILES: CNC(C)c1ccc(Br)cc1 ; O=C(O)C1CCCCC1 ; CC(c1ccc(Br)cc1)N(C)C(=O)C1CCCCC1]

To a mixture of [1-(4-bromophenyl)ethyl](methyl)amine [INT 10.1] (500 mg, 2.33 mmol) and HATU (1.32 g, 3.49 mmol) in CH₂Cl₂ (10 mL) was added DIPEA (902 mg, 6.98 mmol) and cyclohexanecarboxylic acid [INT 11-b] (447 mg, 3.49 mmol). The reaction mixture was stirred at 20° C. under nitrogen for 16 hours. The mixture was diluted with water (50 mL) and extracted with EtOAC (50 mL×3). The combined organic layers were washed with brine (50 mL×3), dried over Na₂SO₄, filtered and concentrated under reduced pressure to give a residue. The residue was purified by flash chromatography on silica gel (petroleum ether:ethyl acetate=5:1) to afford N-[1-(4-bromophenyl)ethyl]-N-methylcyclohexanecarboxamide [INT 11.2] (700 mg, 2.15 mmol, 92.7% yield) as a colorless oil. m/z: [M+H]+ Calcd for C16H23BrNO 324.1, 326.1; Found 326.1.

### Synthesis of N-[1-(4-bromophenyl)ethyl]-N-methylcyclopentanecarboxamide (Intermediate 11.3)

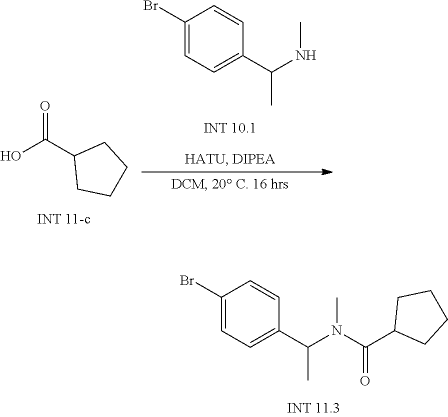 [SMILES: CNC(C)c1ccc(Br)cc1 ; O=C(O)C1CCCC1 ; CC(c1ccc(Br)cc1)N(C)C(=O)C1CCCC1]

To a mixture of cyclopentanecarboxylic acid [INT 11-c] (63.9 mg, 560 μmol) and HATU (266 mg, 700 μmol) in CH₂Cl₂ (3 mL) was added DIPEA (180 mg, 1.40 mmol) and [1-(4-bromophenyl)ethyl](methyl)amine [INT 10.1] (100 mg, 467 μmol). The reaction mixture was stirred at 20° C. under nitrogen for 16 hours. The reaction mixture was concentrated under reduced pressure to give a residue, which was diluted with water (20 mL) and extracted with EtOAc (20 mL×2). The combined organic layers were washed with brine (30 mL×2), dried over Na₂SO₄, and concentrated under reduced pressure to give N-[1-(4-bromophenyl)ethyl]-N-methylcyclopentanecarboxamide [INT 11.3] (110 mg, 354 μmol, 76.3% yield) as a colorless oil. m/z: [M+H]+ Calcd for C15H21BrNO 310.1, 312.1; Found 312.1.

### Synthesis of N-(1-(4-bromophenyl)ethyl)-N-methylacetamide (Intermediate 12.1)

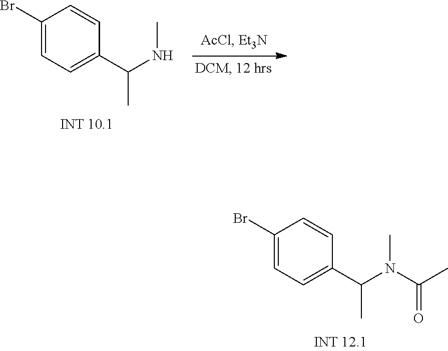

To a mixture of 1-(4-bromophenyl)-N-methylethan-1-amine [INT 10.1] (100 mg, 467 μmol) in DCM (2 mL) was added triethylamine (235 mg, 2.33 mmol) and acetyl chloride (127 mg, 1.63 mmol). The mixture was stirred at 25° C. for 12 hr. The reaction mixture was concentrated under reduced pressure to give the crude product, which was purified by flash chromatography on silica gel (EtOAc/PE=0/1 to 1/3) to give N-(1-(4-bromophenyl)ethyl)-N-methylacetamide [INT 12.1] (100 mg, 390 μmol, 84.0% yield) as a yellow oil. m/z: [M+H]+ Calcd for C11H15BrNO 256.0, 258.0; Found 257.9.

### Synthesis of N-[1-(4-bromophenyl)ethyl]-N-methylcyclopropanecarboxamide (Intermediate 12.2)

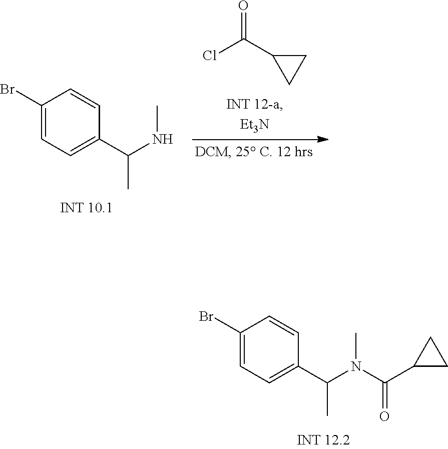 [SMILES: O=C(Cl)C1CC1 ; CNC(C)c1ccc(Br)cc1 ; CC(c1ccc(Br)cc1)N(C)C(=O)C1CC1]

To a mixture of [1-(4-bromophenyl)ethyl](methyl)amine [INT 10.1] (740 mg, 3.45 mmol) and triethylamine (349 mg, 3.45 mmol) in CH₂Cl₂ (10 mL) was added cyclopropanecarbonyl chloride [INT 12-a] (1.25 g, 12.0 mmol) at 25° C. and the mixture was stirred at 25° C. for 12 hr. The reaction mixture was concentrated under reduced pressure to give N-[1-(4-bromophenyl)ethyl]-N-methylcyclopropanecarboxamide [INT 12.2] (556 mg, 1.97 mmol) as a brown oil. m/z: [M+H]+ Calcd for C13H17BrNO 282.0; Found 282.1.

### Synthesis of N-(1-(4-bromophenyl)-2,2,2-trifluoroethyl)-N-methylacetamide (Intermediate 13.1)

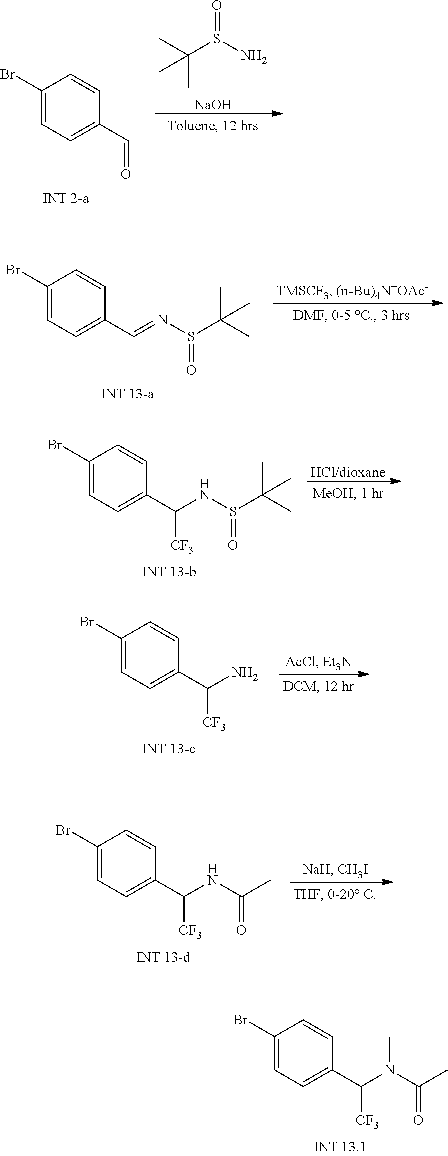

### Synthesis of (E)-N-(4-bromobenzylidene)-2-methylpropane-2-sulfinamide (INT 13-a)

To a solution of 4-bromobenzaldehyde [INT 2-a] (5.0 g, 27.0 mmol) in toluene (30 mL) was added 2-methylpropane-2-sulfinamide (3.5 g, 28.8 mmol). After 15 min, sodium hydroxide (1.1 g, 27.5 mmol) was added and the reaction mixture was stirred at 25° C. for 12 h. Sodium sulfate (1.3 g) and celite (1.3 g) were added and the suspension was stirred for 15 min. The mixture was filtered and concentrated under reduced pressure to give (E)-N-(4-bromobenzylidene)-2-methylpropane-2-sulfinamide [INT 13-a] (7.35 g, 25.5 mmol, 94.4% yield) as a colorless gum. ¹H NMR (400 MHz, DMSO-d6) δ=8.55 (s, 1H), 7.89 (d, J=8.4 Hz, 2H), 7.80-7.72 (m, 2H), 1.18 (s, 9H).

### Synthesis of N-(1-(4-bromophenyl)-2,2,2-trifluoroethyl)-2-methylpropane-2-sulfinamide (INT 13-b)

Neat trimethyl(trifluoromethyl)silane (4.29 g, 30.2 mmol) was added to a stirred solution of tetrabutylazanium acetate (3.64 g, 12.1 mmol) and (E)-N-(4-bromobenzylidene)-2-methylpropane-2-sulfinamide [INT 13-a] (3.5 g, 12.1 mmol) in DMF (30 mL) at 0° C. The mixture was stirred at 0-5° C. for 3 h. The mixture was poured into water (100 mL). A precipitate was collected by filtration and dried under reduced pressure to give N-(1-(4-bromophenyl)-2,2,2-trifluoroethyl)-2-methylpropane-2-sulfinamide [INT 13-b] (3.50 g, 9.77 mmol, 80.8% yield) as an off-white solid. ¹H NMR (400 MHz, DMSO-d6) δ=7.68-7.62 (m, 2H), 7.61-7.57 (m, 2H), 6.48 (d, J=9.5 Hz, 1H), 5.27 (quin, J=8.6 Hz, 1H), 1.14 (s, 9H).

### Synthesis of 1-(4-bromophenyl)-2,2,2-trifluoroethan-1-amine (INT 13-c)

4 M HCl in dioxane (9.75 mL, 39.0 mmol) was added to a suspension of N-(1-(4-bromophenyl)-2,2,2-trifluoroethyl)-2-methylpropane-2-sulfinamide [INT 13-b] (3.5 g, 9.77 mmol) in methanol (20 mL) and the reaction mixture was stirred at 20° C. for 1 h. The reaction was concentrated. The residue was diluted with water (20 mL) and adjusted to pH 10 with 1 N NaOH solution. The mixture was extracted with ethyl acetate (2×50 mL). The combined organic layers were dried over sodium sulfate, filtered and concentrated under reduced pressure to give crude 1-(4-bromophenyl)-2,2,2-trifluoroethan-1-amine [INT 13-c] (2.16 g, 8.50 mmol, 87.0% yield) as a brown oil. m/z: [M−NH2]+ Calcd for C8H7BrF3N 237.0; Found 237.0. ¹H NMR (400 MHz, DMSO-d6) δ=7.60 (d, J=8.3 Hz, 2H), 7.47 (d, J=8.3 Hz, 2H), 4.54 (q, J=8.2 Hz, 1H), 2.78 (br s, 2H).

### Synthesis of N-(1-(4-bromophenyl)-2,2,2-trifluoroethyl)acetamide [INT 13-d]

To a solution of 1-(4-bromophenyl)-2,2,2-trifluoroethan-1-amine [INT 13-c] (2.7 g, 8.50 mmol) and triethylamine (1.72 g, 17.0 mmol) in CH₂Cl₂ (30 mL) was added acetyl chloride (996 mg, 12.7 mmol) and the reaction mixture was stirred at 20° C. for 12 h. The reaction was diluted with CH₂Cl₂ (50 mL), washed with water (50 mL), dried over anhydrous Na₂SO₄ and concentrated under reduced pressure to give the crude product, which was purified by flash chromatography on silica gel (50% ethyl acetate in PE) to give N-(1-(4-bromophenyl)-2,2,2-trifluoroethyl)acetamide [INT 13-d] (2.50 g, 8.44 mmol, 99.6% yield) as an off-white solid. m/z: [M+H]+ Calcd for C10H10BrF3NO 296.0, 298.0; Found 295.8. ¹H NMR (400 MHz, DMSO-d6) δ=9.15 (d, J=9.7 Hz, 1H), 7.65 (d, J=8.6 Hz, 2H), 7.53 (d, J=8.4 Hz, 2H), 5.91-5.74 (m, 1H), 1.96 (s, 3H).

### Synthesis of N-(1-(4-bromophenyl)-2,2,2-trifluoroethyl)-N-methylacetamide (INT 13.1)

A mixture of sodium hydride (671 mg, 16.8 mmol) in THF (30 mL) was cooled to 0° C. N-(1-(4-bromophenyl)-2,2,2-trifluoroethyl)acetamide [INT 13-d] (2.5 g, 8.44 mmol) was added and the reaction mixture was stirred at 0° C. for 30 min. Iodomethane (3.59 g, 25.3 mmol) was then added and the reaction mixture was allowed to warm to 20° C. over 12 h under N₂. The reaction was quenched by adding sat. NH₄Cl (150 mL) and was extracted with EtOAc (2×100 mL). The combined organic layers were dried over anhydrous Na₂SO₄ and concentrated under reduced pressure to give the crude product, which was purified by flash chromatography on silica gel (20% ethyl acetate in PE) to give N-(1-(4-bromophenyl)-2,2,2-trifluoroethyl)-N-methylacetamide [INT 13.1] (1.83 g, 5.90 mmol, 70.1% yield) as a brown oil. m/z: [M+H]+ Calcd for C11H12BrF3NO 310.0, 312.0; Found 311.7. ¹H NMR (400 MHz, DMSO-d6) δ=7.71-7.66 (m, 2H), 7.37-7.31 (m, 2H), 6.62-6.06 (m, 1H), 2.97 (s, 1H), 2.83 (s, 2H), 2.16 (s, 2H), 1.91 (s, 1H).

### Synthesis of (R)—N-(1-(4-bromophenyl)-2,2,2-trifluoroethyl)-N-methyltetrahydro-2H-thiopyran-4-carboxamide 1,1-dioxide (Intermediate 14.1)

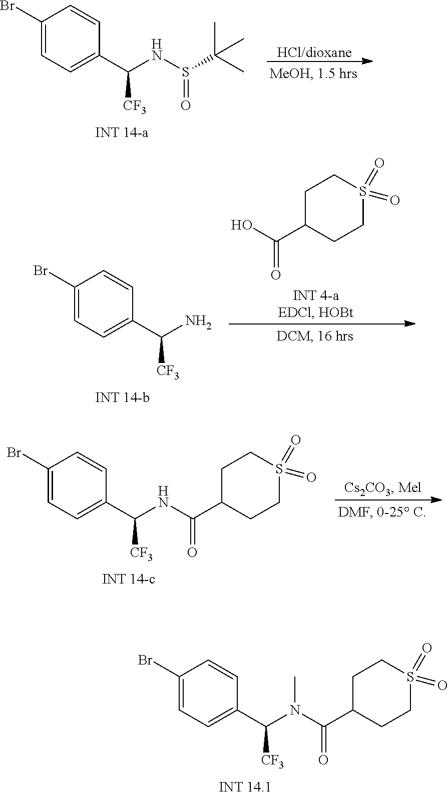

### Synthesis of (S)—N—((R)-1-(4-bromophenyl)-2,2,2-trifluoroethyl)-2-methylpropane-2-sulfinamide (INT 14-a)

(S)—N—((R)-1-(4-bromophenyl)-2,2,2-trifluoroethyl)-2-methylpropane-2-sulfinamide [INT 14-a] was prepared by the same synthetic route as outlined for (R)—N—((S)-1-(4-bromophenyl)-2,2,2-trifluoroethyl)-2-methylpropane-2-sulfinamide [INT 2.1] using (S)-(−)-2-Methyl-2-propanesulfinamide.

### Synthesis of (R)-1-(4-bromophenyl)-2,2,2-trifluoroethan-1-amine (INT 14-b)

To a mixture of (S)—N—((R)-1-(4-bromophenyl)-2,2,2-trifluoroethyl)-2-methylpropane-2-sulfinamide [INT 14-a] (8 g, 22.3 mmol) in MeOH (60 mL) was added 4 M HCl in dioxane (20 mL). The mixture was stirred at 20° C. for 1.5 h. The mixture was concentrated under reduced pressure to afford the crude product. The mixture was diluted with water (50 mL) and extracted with EtOAc (50 mL×2). The combined organic layers were washed with 1 M HCl (50 mL×2). The aqueous phase was basified with 2 N NaOH to pH=9-10 and extracted with CH₂Cl₂ (50 mL×2). The combined organic layers were dried over anhydrous Na₂SO₄, filtered, and concentrated under reduced pressure to give the crude product (R)-1-(4-bromophenyl)-2,2,2-trifluoroethan-1-amine [INT 14-b] (3.50 g, 13.7 mmol, 61.8% yield) as a yellow solid. m/z: [M+H]+ Calcd for C8H8BrF3N 254.0, 256.0; Found 254.1.

### Synthesis of (R)—N-(1-(4-bromophenyl)-2,2,2-trifluoroethyl)tetrahydro-2H-thiopyran-4-carboxamide 1,1-dioxide (INT 14-c)

To a mixture of tetrahydro-2H-thiopyran-4-carboxylic acid 1,1-dioxide [INT 4-a] (1.68 g, 9.44 mmol), EDCI (2.26 g, 11.8 mmol), and HOBt (1.59 g, 11.8 mmol) in CH₂Cl₂ (20 mL) was added (R)-1-(4-bromophenyl)-2,2,2-trifluoroethan-1-amine [INT 14-b] (2 g, 7.87 mmol). The mixture was stirred at 25° C. for 16 hr. The reaction was quenched by adding water (50 mL) and was extracted with EtOAc (50 mL×3). The combined organic layers were washed with brine (50 mL×2), dried over anhydrous Na₂SO₄ and concentrated under reduced pressure to give the crude product, which was purified by flash chromatography on silica gel (EtOAc/PE=0/1 to 1/5) to give (R)—N-(1-(4-bromophenyl)-2,2,2-trifluoroethyl)tetrahydro-2H-thiopyran-4-carboxamide 1,1-dioxide [INT 14-c] (2.20 g, 5.31 mmol, 67.4% yield) as a white solid. m/z: [M+H]+ Calcd for C14H16BrF3NO3S 414.0, 416.0; Found 416.2.

### Synthesis of (R)—N-(1-(4-bromophenyl)-2,2,2-trifluoroethyl)-N-methyltetrahydro-2H-thiopyran-4-carboxamide 1,1-dioxide (INT 14.1)

To a solution of (R)—N-(1-(4-bromophenyl)-2,2,2-trifluoroethyl)tetrahydro-2H-thiopyran-4-carboxamide 1,1-dioxide [INT 14-c] (1 g, 2.41 mmol) in DMF (10 mL) was added Cs₂CO₃ (1.57 g, 4.82 mmol) and the reaction mixture was stirred at 25° C. for 1 h. Methyl iodide (1.02 g, 7.23 mmol) was then added at 0° C. and the reaction was stirred at 25° C. for 2 h. The reaction was quenched by adding water (50 mL), then it was extracted with EtOAc (50 mL×3). The combined organic layers were washed with brine (50 mL×2), dried over anhydrous Na₂SO₄ and concentrated under reduced pressure to give the crude product, which was purified by flash chromatography on silica gel (PE/EtOAc=1/0 to 1/1) to give (R)—N-(1-(4-bromophenyl)-2,2,2-trifluoroethyl)-N-methyltetrahydro-2H-thiopyran-4-carboxamide 1,1-dioxide [INT 14.1] (700 mg, 1.63 mmol, 67.9% yield) as a colorless oil. m/z: [M+H]+ Calcd for C15H18BrF3NO3S 428.0, 430.0; Found 430.1.

### Synthesis of (S)-1-(4-bromo-2-methylphenyl)-2,2,2-trifluoro-N-methylethan-1-amine hydrochloride (Intermediate 15.1)

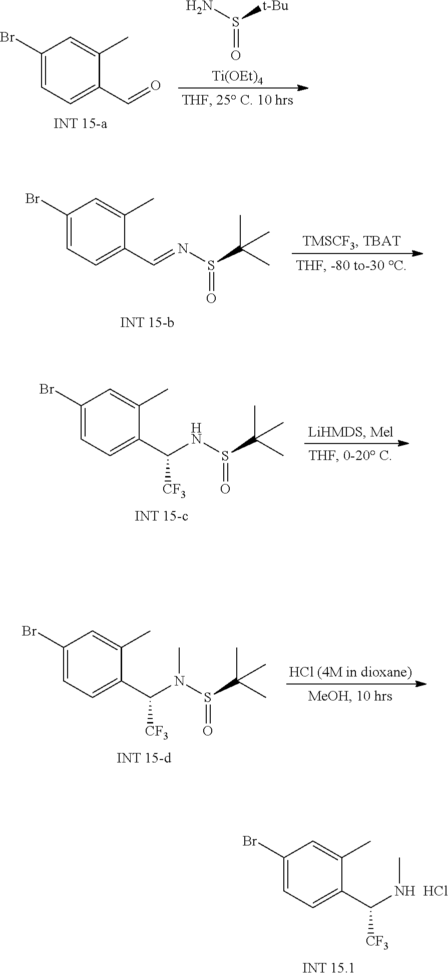

### Synthesis of (R,E)-N-(4-bromo-2-methylbenzylidene)-2-methylpropane-2-sulfinamide (INT 15-b)

(R)-2-methylpropane-2-sulfinamide (12.1 g, 100 mmol) and 4-bromo-2-methylbenzaldehyde [INT 15-a] (10 g, 50.2 mmol) were dissolved in tetrahydrofuran (50 mL) and titanium ethoxide (34.2 g, 150 mmol) was added. The mixture was stirred for 10 h at 25° C. Then the reaction mixture was poured into water (500 mL) and extracted with EtOAc (3×300 mL). The organic extracts were combined, dried under Na₂SO₄ and evaporated in vacuo. The residue was purified by flash chromatography (Hexane/MTBE=1/0 to 0/1) to give (R,E)-N-(4-bromo-2-methylbenzylidene)-2-methylpropane-2-sulfinamide [INT 15-b] (10.4 g, 34.5 mmol, 68.8% yield) as a yellow solid. ¹H NMR (400 MHz, CDCl₃) δ 8.76 (s, 1H), 7.75 (d, J=8.7 Hz, 1H), 7.46-7.37 (m, 2H), 2.56 (s, 3H), 1.24 (s, 9H).

### Synthesis of (R)—N—((S)-1-(4-bromo-2-methylphenyl)-2,2,2-trifluoroethyl)-2-methylpropane-2-sulfinamide (INT 15-c)

(R,E)-N-(4-bromo-2-methylbenzylidene)-2-methylpropane-2-sulfinamide [INT 15-b] (18.8 g, 62.2 mmol) and tetrabutylammonium triphenyldifluorosilicate (50.3 g, 93.3 mmol) were dissolved in THF (300 mL). Trifluoromethyltrimethylsilane (44.2 g, 311 mmol) was added dropwise at −80° C. The mixture was stirred for 30 min at −30° C. after which an aqueous solution of NH₄Cl (200 mL) was added. The mixture was extracted with EtOAc (2×200 mL). The organic phase was dried with sodium sulfate and evaporated in vacuo at 45° C. The residue was purified by flash chromatography to obtain (R)—N—((S)-1-(4-bromo-2-methylphenyl)-2,2,2-trifluoroethyl)-2-methylpropane-2-sulfinamide [INT 15-c] (19.0 g, 51.0 mmol, 82.2% yield) as a light yellow oil. m/z: [M+H]+ Calcd for C13H18BrF3NOS 372.0; Found 372.2. ¹H NMR (500 MHz, CDCl₃) δ 7.39 (s, 2H), 7.27 (d, J=7.6 Hz, 1H), 5.04 (p, J=7.1 Hz, 1H), 3.58 (d, J=5.8 Hz, 1H), 2.44 (s, 3H), 1.24 (s, 9H).

### Synthesis of (R)—N—((S)-1-(4-bromo-2-methylphenyl)-2,2,2-trifluoroethyl)-N,2-dimethylpropane-2-sulfinamide (INT 15-d)

(R)—N—((S)-1-(4-bromo-2-methylphenyl)-2,2,2-trifluoroethyl)-2-methylpropane-2-sulfinamide [INT 15-c] (10 g, 26.8 mmol) was dissolved in THF (300 mL). Lithium bis(trimethylsilyl)amide (74.3 mL, 80.3 mmol) was added at 0° C. The mixture was stirred for 20 min at 0° C. Methyl iodide (22.7 g, 160 mmol) was added. The mixture was stirred for 10 hr at 20° C. after which an aqueous solution of NH₄Cl (50 mL) was added. The mixture was extracted with EtOAc (2×30 mL). The organic phase was dried with sodium sulfate and evaporated in vacuo at 45° C. to obtain crude (R)—N—((S)-1-(4-bromo-2-methylphenyl)-2,2,2-trifluoroethyl)-N,2-dimethylpropane-2-sulfinamide [INT 15-d] (8.50 g, 22.0 mmol, 82.5% yield) as a brown oil. m/z: [M+H]+ Calcd for C14H20BrF3NOS 386.0, 388.0; Found 388.0. ¹H NMR (400 MHz, CDCl₃) δ 7.44-7.32 (m, 1H), 7.31-7.21 (m, 1H), 7.18 (d, J=8.2 Hz, 1H), 5.15-5.06 (m, 1H), 2.50-2.42 (m, 6H), 1.23 (s, 9H).

### Synthesis of(S)-1-(4-bromo-2-methylphenyl)-2,2,2-trifluoro-N-methylethan-1-amine hydrochloride (INT 15.1)

(R)—N—((S)-1-(4-bromo-2-methylphenyl)-2,2,2-trifluoroethyl)-N,2-dimethylpropane-2-sulfinamide [INT 15-d] (10 g, 25.8 mmol) was dissolved in methanol (20 mL). Hydrogen chloride (4 M in 1,4-dioxane, 100 mL, 2.36 mol) was added. The mixture was stirred for 10 hr 10 at 20° C. The mixture was evaporated in vacuo at 50° C. MTBE (100 mL) was added. The solid formed was filtered and washed with MTBE (100 mL) to obtain (S)-1-(4-bromo-2-methylphenyl)-2,2,2-trifluoro-N-methylethan-1-amine hydrochloride [INT 15.1] (6.27 g, 19.6 mmol, 76.3% yield) as a white solid. m/z: [M+H]+ Calcd for C10H12BrF3N 282.0, 284.0; Found 284.0. ¹H NMR (400 MHz, DMSO-d6) δ 7.71 (d, J=8.5 Hz, 1H), 7.65-7.55 (m, 2H), 5.51-5.46 (m, 1H), 2.49 (s, 3H), 2.40 (s, 3H).

### Synthesis of (S)-1-(4-bromo-3-methylphenyl)-2,2,2-trifluoro-N-methylethan-1-amine hydrochloride (Intermediate 15.2)

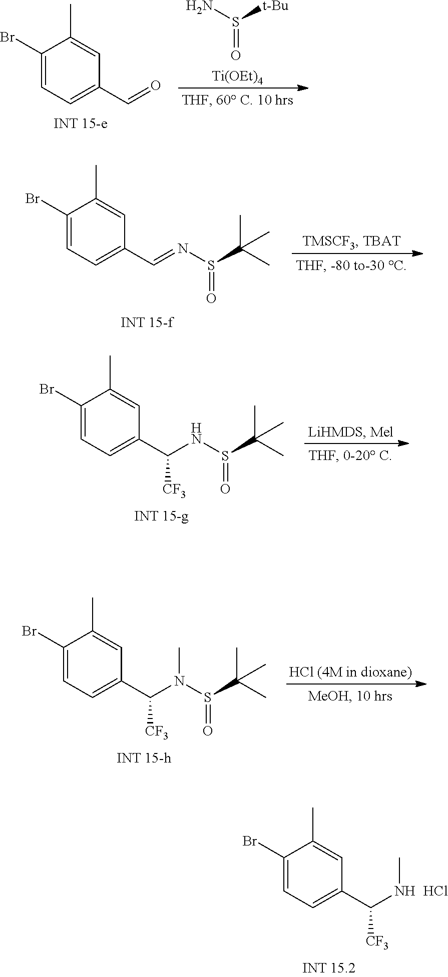

### Synthesis of (R,E)-N-(4-bromo-3-methylbenzylidene)-2-methylpropane-2-sulfinamide (INT 15-f)

(R)-2-methylpropane-2-sulfinamide (18.1 g, 150 mmol) and 4-bromo-3-methylbenzaldehyde [INT 15-e] (15 g, 75.3 mmol) were dissolved in tetrahydrofuran (100 mL) and titanium ethoxide (51.3 g, 225 mmol) was added. The mixture was stirred for 10 h at 60° C. Then the reaction mixture was poured into water (500 mL) and extracted with MTBE (3×300 mL). The organic extracts were re-extracted with water (3×200 mL), dried under Na₂SO₄ and evaporated in vacuo to give (R,E)-N-(4-bromo-3-methylbenzylidene)-2-methylpropane-2-sulfinamide [INT 15-f] (15.2 g, 50.2 mmol, 66.9% yield) as a yellow solid. ¹H NMR (400 MHz, CDCl₃) δ 8.49 (s, 1H), 7.67 (d, J=2.2 Hz, 1H), 7.65-7.52 (m, 1H), 7.51-7.43 (m, 1H), 2.43 (s, 3H), 1.23 (s, 9H).

### Synthesis of (R)—N—((S)-1-(4-bromo-3-methylphenyl)-2,2,2-trifluormethyl)-2-methylpropane-2-sulfinamide (INT 15-g)

(R,E)-N-(4-bromo-3-methylbenzylidene)-2-methylpropane-2-sulfinamide [INT 15-f] (21 g, 69.4 mmol) and tetrabutylammonium triphenyldifluorosilicate (56.1 g, 104 mmol) were dissolved in THF (500 mL). Trifluoromethyltrimethylsilane (49.3 g, 347 mmol) was added dropwise at −80° C. The mixture was stirred for 30 min at −30° C. after which an aqueous solution of NH₄Cl (300 mL) was added. The mixture was extracted with EtOAc (2×300 mL). The organic phase was dried with sodium sulfate and evaporated in vacuo at 45° C. The residue was purified by flash chromatography to obtain (R)—N—((S)-1-(4-bromo-3-methylphenyl)-2,2,2-trifluoroethyl)-2-methylpropane-2-sulfinamide [INT 15-g] (18.5 g, 49.7 mmol, 71.7% yield) as a white solid. m/z: [M+H]+ Calcd for C13H18BrF3NOS 372.0; Found 372.0. ¹H NMR (500 MHz, CDCl₃) δ 7.56 (d, J=8.2 Hz, 1H), 7.30-7.26 (m, 1H), 7.14-7.08 (m, 1H), 4.75 (p, J=7.1 Hz, 1H), 3.58 (d, J=6.4 Hz, 1H), 2.41 (s, 3H), 1.25 (s, 9H).

### Synthesis of (R)—N—((S)-1-(4-bromo-3-methylphenyl)-2,2,2-trifluoroethyl)-N,2-dimethylpropane-2-sulfinamide (INT 15-h)

(R)—N—((S)-1-(4-bromo-3-methylphenyl)-2,2,2-trifluoroethyl)-2-methylpropane-2-sulfinamide [INT 15-g] (10 g, 26.8 mmol) was dissolved in THF (200 mL). Lithium(1+) bis(trimethylsilyl)azanide (74.3 mL, 80.3 mmol) was added at 0° C. The mixture was stirred for 20 min at 0° C. Methyl iodide (22.7 g, 160 mmol) was added. The mixture was stirred for 10 hr at 20° C. after which an aqueous solution of NH₄Cl (200 mL) was added. The mixture was extracted with EtOAc (2×200 mL). The organic phase was dried with sodium sulfate and evaporated in vacuo at 45° C. to obtain crude (R)—N—((S)-1-(4-bromo-3-methylphenyl)-2,2,2-trifluoroethyl)-N,2-dimethylpropane-2-sulfinamide [INT 15-h] (9.09 g, 23.5 mmol, 88.2% yield) as a brown oil. m/z: [M+H]+ Calcd for C14H20BrF3NOS 386.0; Found 386.0. ¹H NMR (400 MHz, CDCl₃) δ 7.57 (d, J=8.3 Hz, 1H), 7.34 (s, 1H), 7.17 (d, J=8.6 Hz, 1H), 5.03 (q, J=8.5 Hz, 1H), 2.50-2.41 (m, 6H), 1.27 (s, 9H).

### Synthesis of (S)-1-(4-bromo-3-methylphenyl)-2,2,2-trifluoro-N-methylethan-1-amine hydrochloride (INT 15.2)

(R)—N—((S)-1-(4-bromo-3-methylphenyl)-2,2,2-trifluoroethyl)-N,2-dimethylpropane-2-sulfinamide [INT 15-h] (10.7 g, 27.7 mmol) was dissolved in methanol (20 mL), after which hydrogen chloride (4 M in 1,4-dioxane, 100 mL, 2.54 mol) was added. The mixture was stirred for 10 hr at 20° C., after which it was evaporated in vacuo at 50° C. MTBE (100 mL) was added. The solid formed was filtered and washed with MTBE (50 mL) to obtain (S)-1-(4-bromo-3-methylphenyl)-2,2,2-trifluoro-N-methylethan-1-amine hydrochloride [INT 15.2] (5.41 g, 16.9 mmol, 61.3% yield) as a beige solid. m/z: [M+H]+ Calcd for C10H12BrF3N 282.0, 284.0; Found 284.0. ¹H NMR (500 MHz, DMSO-d6) δ 10.51 (s, 2H), 7.76 (d, J=8.3 Hz, 1H), 7.63 (d, J=2.2 Hz, 1H), 7.41 (dd, J=8.3, 2.2 Hz, 1H), 5.42 (s, 1H), 2.43 (s, 3H), 2.37 (s, 3H).

### Synthesis of (S)—N-(1-(4-bromo-2-methylphenyl)-2,2,2-trifluoroethyl)-N-methylcyclobutanecarboxamide (Intermediate 16.1)

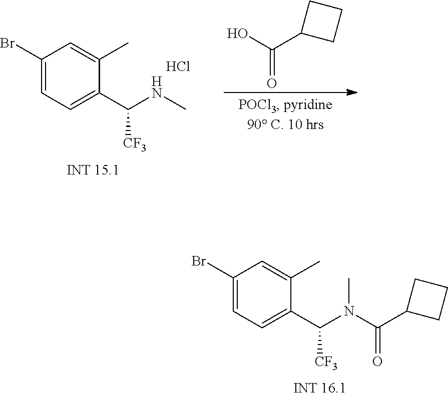 [SMILES: CN[C@@H](c1ccc(Br)cc1C)C(F)(F)F ; Cl ; O=C(O)C1CCC1 ; Cc1cc(Br)ccc1[C@H](N(C)C(=O)C1CCC1)C(F)(F)F]

(S)-1-(4-bromo-2-methylphenyl)-2,2,2-trifluoro-N-methylethan-1-amine hydrochloride [INT 15.1] (0.2 g, 0.6278 mmol) and cyclobutanecarboxylic acid (125 mg, 1.25 mmol) were mixed in pyridine (2 mL), after which phosphorus oxychloride (211 mg, 1.38 mmol) was added. The mixture was stirred for 10 hr at 90° C. EtOAc (20 mL) was added and the mixture was washed with an aqueous solution NaHSO₄ (3×5 mL). The organic phase was dried with sodium sulfate and evaporated in vacuo at 45° C. to obtain (S)—N-(1-(4-bromo-2-methylphenyl)-2,2,2-trifluoroethyl)-N-methylcyclobutanecarboxamide [INT 16.1] (190 mg, 0.5216 mmol, 83.3% yield) as a yellow oil. m/z: [M+H]+ Calcd for C15H18BrF3NO 364.1, 366.1; Found 366.0. ¹H NMR (400 MHz, CDCl₃) δ 7.37 (s, 3H), 6.53 (q, J=8.8 Hz, 1H), 3.70 (q, J=7.0 Hz, 1H), 3.28 (q, J=8.5 Hz, 1H), 2.65 (s, 3H), 2.39 (q, J=9.5 Hz, 1H), 2.28 (q, J=10.1 Hz, 1H), 2.21-2.16 (m, 4H), 2.05-1.86 (m, 2H).

### Synthesis of N-[(1S)-1-(4-bromo-2-methylphenyl)-2,2,2-trifluoroethyl]-N-methylcyclopropanecarboxamide (Intermediate 16.2)

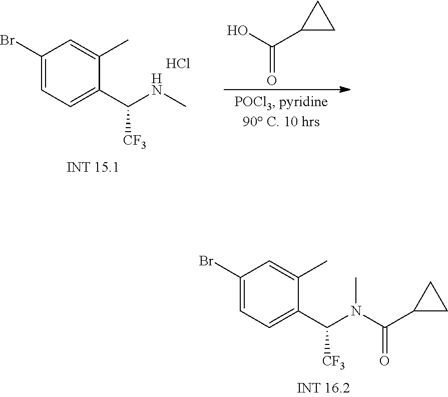 [SMILES: CN[C@@H](c1ccc(Br)cc1C)C(F)(F)F ; Cl ; O=C(O)C1CC1 ; Cc1cc(Br)ccc1[C@H](N(C)C(=O)C1CC1)C(F)(F)F]

[(1S)-1-(4-bromo-2-methylphenyl)-2,2,2-trifluoroethyl](methyl)amine hydrochloride [INT 15.1] (0.2 g, 0.6278 mmol) and cyclopropanecarboxylic acid (107 mg, 1.25 mmol) were mixed in pyridine (2 mL). Phosphorus oxychloride (211 mg, 1.38 mmol) was then added. The mixture was stirred for 10 hr at 90° C. EtOAc (20 mL) was added. The mixture was washed with an aqueous NaHSO₄ solution (3×5 mL). The organic phase was dried with sodium sulfate and evaporated in vacuo at 45° C. to obtain N-[(1S)-1-(4-bromo-2-methylphenyl)-2,2,2-trifluoroethyl]-N-methylcyclopropanecarboxamide [INT 16.2] (190 mg, 0.5425 mmol, 86.7% yield) as a light yellow solid. m/z: [M+H]+ Calcd for C14H16BrF3NO 350.0; Found 350.0. ¹H NMR (400 MHz, CDCl₃) δ=7.38 (s, 3H), 6.53 (q, J=8.9 Hz, 1H), 2.91 (s, 3H), 2.15 (s, 3H), 1.77-1.72 (m, 1H), 1.11 (s, 1H), 0.99 (s, 1H), 0.84 (d, J=7.1 Hz, 2H).

### Synthesis of N-[(1S)-1-(4-bromo-2-methylphenyl)-2,2,2-trifluoroethyl]-N-methyl-1,1-dioxo-1λ⁶-thiane-4-carboxamide (Intermediate 16.3)

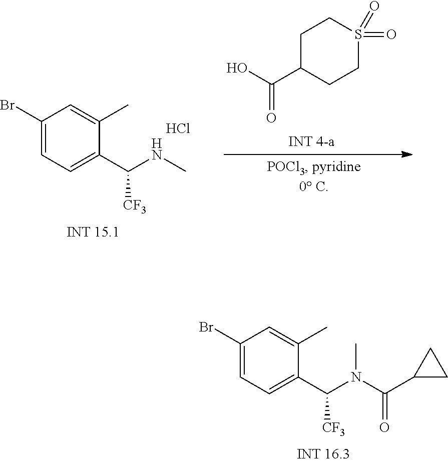 [SMILES: CN[C@@H](c1ccc(Br)cc1C)C(F)(F)F ; Cl ; O=C(O)C1CCS(=O)(=O)CC1 ; Cc1cc(Br)ccc1[C@H](N(C)C(=O)C1CC1)C(F)(F)F]

Phosphorus oxychloride (788 mg, 5.14 mmol) was added to a solution of [(1S)-1-(4-bromo-2-methylphenyl)-2,2,2-trifluoroethyl](methyl)amine hydrochloride [INT 15.1] (500 mg, 1.56 mmol) and 1,1-dioxo-1λ⁶-thiane-4-carboxylic acid [INT 4-a] (833 mg, 4.68 mmol) in pyridine (3 mL) at 0° C. The reaction mixture was stirred overnight. An aqueous solution of sodium bicarbonate (3 mL) was added, and the mixture was extracted with EtOAc (3×10 mL) and washed with NaHSO₄ (3×10 mL). The combined organic layers were dried over anhydrous Na₂SO₄ and evaporated under reduced pressure. The crude product was purified by HPLC (see conditions below) to give N-[(1S)-1-(4-bromo-2-methylphenyl)-2,2,2-trifluoroethyl]-N-methyl-1,1-dioxo-1λ⁶-thiane-4-carboxamide [INT 16.3] (132 mg, 0.2985 mmol, 19.1% yield) as a pink solid. m/z: [M+H]+ Calcd for C16H20BrF3NO3S 442.0, 444.0; Found 444.0.

HPLC conditions: System Agilent 1260 Infinity II LC coupled to an Agilent 6120B Single Quadrupole LC/MS System Column Description: Chromatorex SBM 100-5T 5 μm C18(2) 100 Å, LC Column 100×19 mm, Waters, Sun Fire Stationary Phase: C18 Solid Support: Fully Porous Silica Separation Mode: Reversed Phase Mobile Phase Mobile phase A: water Mobile phase B: acetonitrile Flow rate: 30 ml/min; loading pump 4 ml/min B Gradient conditions: 20-30-60-100% (B) 0-2-10-11.2 min.

### Synthesis of (S)—N-(1-(4-bromo-3-methylphenyl)-2,2,2-trifluoroethyl)-N-methylcyclobutanecarboxamide (Intermediate 17.1)

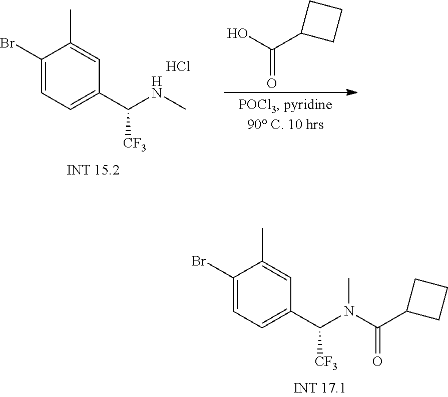 [SMILES: CN[C@@H](c1ccc(Br)c(C)c1)C(F)(F)F ; Cl ; O=C(O)C1CCC1 ; Cc1cc([C@H](N(C)C(=O)C2CCC2)C(F)(F)F)ccc1Br]

(S)-1-(4-bromo-3-methylphenyl)-2,2,2-trifluoro-N-methylethan-1-amine hydrochloride [INT 15.2] (0.2 g, 0.6278 mmol) and cyclobutanecarboxylic acid (125 mg, 1.25 mmol) were mixed in pyridine (2 mL), after which phosphorus oxychloride (211 mg, 1.38 mmol) was added at 20° C. The mixture was stirred for 10 hr at 90° C. EtOAc (20 mL) was added and the mixture was washed with an aqueous solution of NaHSO₄ (3×5 mL). The organic extract was dried with sodium sulfate and evaporated in vacuo at 35° C. to obtain (S)—N-(1-(4-bromo-3-methylphenyl)-2,2,2-trifluoroethyl)-N-methylcyclobutanecarboxamide [INT 17.1] (228 mg, 0.6260 mmol, 100% yield) as a yellow oil. m/z: [M+H]+ Calcd for C15H18BrF3NO 364.0, 366.0; Found 366.0. ¹H NMR (400 MHz, CDCl₃) δ 7.52 (d, J=8.3 Hz, 1H), 7.19 (s, 1H), 7.04 (d, J=8.6 Hz, 1H), 6.55 (q, J=8.9 Hz, 1H), 3.37-3.27 (m, 1H), 2.71 (s, 3H), 2.40-2.35 (m, 4H), 2.27-2.17 (m, 2H), 2.05-1.88 (m, 3H).

### Synthesis of N-[(1S)-1-(4-bromo-3-methylphenyl)-2,2,2-trifluoroethyl]-N-methylcyclopropanecarboxamide (Intermediate 17.2)

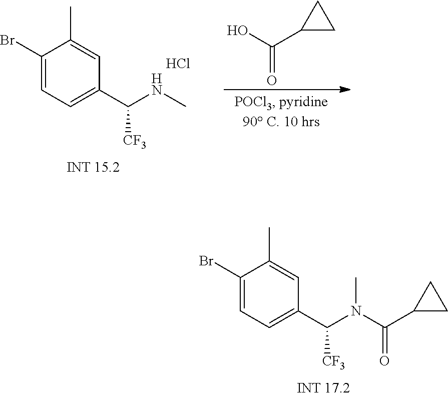 [SMILES: CN[C@@H](c1ccc(Br)c(C)c1)C(F)(F)F ; Cl ; O=C(O)C1CC1 ; Cc1cc([C@H](N(C)C(=O)C2CC2)C(F)(F)F)ccc1Br]

[(1S)-1-(4-bromo-3-methylphenyl)-2,2,2-trifluoroethyl](methyl)amine hydrochloride [INT 15.2] (1.25 g, 3.92 mmol) and cyclopropanecarboxylic acid (674 mg, 7.84 mmol) were mixed in pyridine (20 mL). Phosphoryl chloride (1.32 g, 8.62 mmol) was added. The mixture was stirred for 10 hr at 90° C. MTBE (300 mL) was added. The mixture was washed with an aqueous solution of NaHSO₄ (3×50 mL). The organic phase was dried under sodium sulfate and evaporated in vacuo to give N-[(1S)-1-(4-bromo-3-methylphenyl)-2,2,2-trifluoroethyl]-N-methylcyclopropanecarboxamide [INT 17.2] (480 mg, 1.37 mmol, 35.0% yield) as a yellow solid. m/z: [M+H]+ Calcd for C14H16BrF3NO 350.0; Found 350.0.

### Synthesis of N-[(1S)-1-(4-bromo-3-methylphenyl)-2,2,2-trifluoroethyl]-N-methylcyclopropanecarboxamide (Intermediate 17.3)

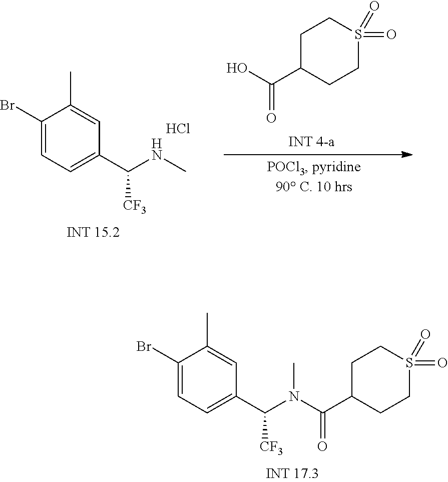 [SMILES: CN[C@@H](c1ccc(Br)c(C)c1)C(F)(F)F ; Cl ; O=C(O)C1CCS(=O)(=O)CC1 ; Cc1cc([C@H](N(C)C(=O)C2CCS(=O)(=O)CC2)C(F)(F)F)ccc1Br]

[(1S)-1-(4-bromo-3-methylphenyl)-2,2,2-trifluoroethyl](methyl)amine hydrochloride [INT 15.2] (0.5 g, 1.56 mmol) and 1,1-dioxo-1λ⁶-thiane-4-carboxylic acid [INT 4-a] (833 mg. 4.68 mmol) were mixed in pyridine (2 mL). Phosphorus oxychloride (788 mg, 5.14 mmol) was added. The mixture was stirred for 10 hr at 90° C. EtOAc (20 mL) was added, and the mixture was washed with an aqueous NaHSO₄ solution (3×5 mL). The organic phase was dried with sodium sulfate and evaporated in vacuo at 45° C. to obtain crude N-[(1S)-1-(4-bromo-3-methylphenyl)-2,2,2-trifluoroethyl]-N-methyl-1,1-dioxo-1λ⁶-thiane-4-carboxamide [INT 17.3] (656 mg, 1.48 mmol) as a yellow solid. m/z: [M+H]+ Calcd for C16H20BrF3NO3S 442.0; Found 442.0.

### Synthesis of (1r,4S)-4-((tert-butyldimethylsilyl)oxy)-N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylcyclohexane-1-carboxamide (Intermediate 18.1)

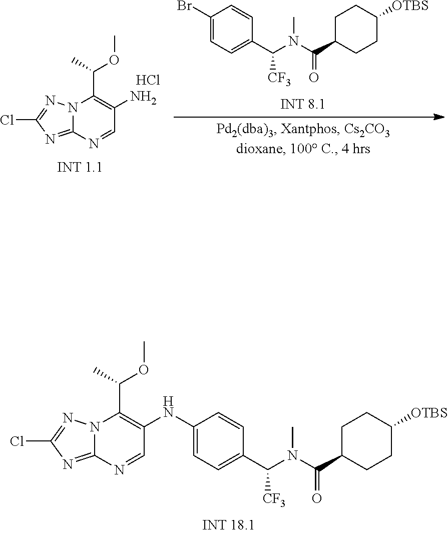 [SMILES: CO[C@@H](C)c1c(N)cnc2nc(Cl)nn12 ; Cl ; CN(C(=O)[C@H]1CC[C@H](O[Si](C)(C)C(C)(C)C)CC1)[C@@H](c1ccc(Br)cc1)C(F)(F)F ; CO[C@@H](C)c1c(Nc2ccc([C@H](N(C)C(=O)[C@H]3CC[C@H](O[Si](C)(C)C(C)(C)C)CC3)C(F)(F)F)cc2)cnc2nc(Cl)nn12]

To a suspension of (1r,4S)—N—((S)-1-(4-bromophenyl)-2,2,2-trifluoroethyl)-4-((tert-butyldimethylsilyl)oxy)-N-methylcyclohexane-1-carboxamide [INT 8.1] (100 mg, 196 μmol), 2-chloro-7-[(1S)-1-methoxyethyl]-[1,2,4]triazolo[1,5-a]pyrimidin-6-amine hydrochloride [INT 1.1] (67.0 mg, 254 μmol), Cs₂CO₃ (127 mg, 392 μmol) and xantphos (22.6 mg, 39.2 μmol) in dioxane (3 mL) was added Pd₂(dba)₃ (17.9 mg, 19.6 μmol). The resulting mixture was stirred at 100° C. for 4 h under N₂. The reaction mixture was poured into water (20 mL) and extracted with EtOAc (15 mL×3). The combined organic layers were washed with brine (50 mL×3), dried over Na₂SO₄, and filtered. The filtrate was concentrated under reduced pressure to give (1r,4S)-4-((tert-butyldimethylsilyl)oxy)-N—((S)-1-(4-((2-chloro-7-((S)-1-methoxyethyl)-[1,2,4]triazolo[1,5-a]pyrimidin-6-yl)amino)phenyl)-2,2,2-trifluoroethyl)-N-methylcyclohexane-1-carboxamide [INT 18.1] (100 mg, 152 μmol, 78.1% yield) as a yellow oil. m/z: [M+H]+ Calcd for C30H43ClF3N6O3Si 655.3; Found 655.2.

### Synthesis of methyl (1S,4r)-4-(((S)-1-(4-((2-chloro-7-isopropyl-[1,2,4]triazolo[1,5-a]pyrimidin-6-yl)amino)phenyl)-2,2,2-trifluoroethyl)(methyl)carbamoyl)cyclohexane-1-carboxylate (Intermediate 18.2)

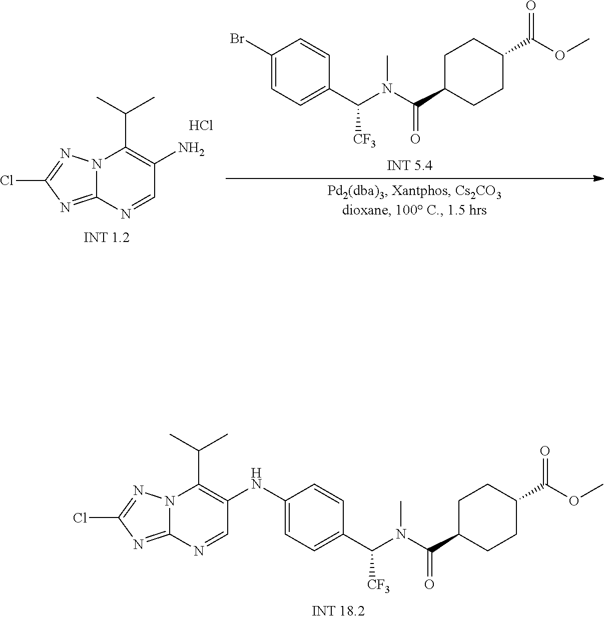 [SMILES: CC(C)c1c(N)cnc2nc(Cl)nn12 ; Cl ; COC(=O)[C@H]1CC[C@H](C(=O)N(C)[C@@H](c2ccc(Br)cc2)C(F)(F)F)CC1 ; COC(=O)[C@H]1CC[C@H](C(=O)N(C)[C@@H](c2ccc(Nc3cnc4nc(Cl)nn4c3C(C)C)cc2)C(F)(F)F)CC1]

A suspension of 2-chloro-7-(propan-2-yl)-[1,2,4]triazolo[1,5-a]pyrimidin-6-amine hydrochloride [INT 1.2] (35 mg, 141 μmol), methyl (1S,4r)-4-(((S)-1-(4-bromophenyl)-2,2,2-trifluoroethyl)(methyl)carbamoyl)cyclohexane-1-carboxylate [INT 5.4] (61.5 mg, 141 μmol), caesium carbonate (137 mg, 423 μmol), tris(dibenzylideneacetone) dipalladium (6.45 mg, 7.05 μmol), and xantphos (8.15 mg, 14.1 μmol) in dioxane (2 mL) was stirred at 100° C. for 1.5 h under N₂. The mixture was concentrated under reduced pressure to give the crude product, which was purified by flash chromatography on silica gel (MeOH/dichloromethane=0/1 to 1/99) to give methyl (1S,4r)-4-(((S)-1-(4-((2-chloro-7-isopropyl-[1,2,4]triazolo[1,5-a]pyrimidin-6-yl)amino)phenyl)-2,2,2-trifluoroethyl)(methyl)carbamoyl)cyclohexane-1-carboxylate [INT 18.2] (38.5 mg, 67.9 μmol, 48.1% yield) as a yellow solid. m/z: [M+Na]+ Calcd for C26H30ClF3N6O3Na 589.2; Found 589.3.

### Synthesis of methyl (1S,3r)-3-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclobutane-1-carboxylate (Intermediate 18.3)

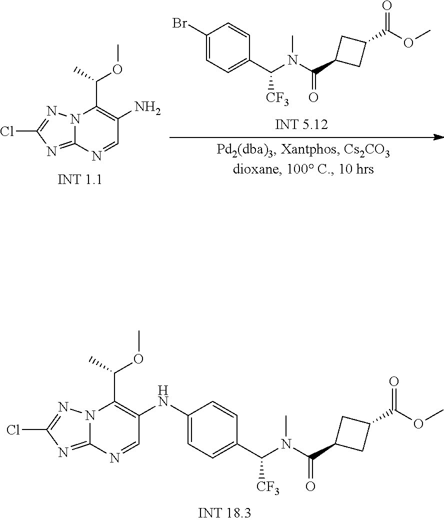 [SMILES: CO[C@@H](C)c1c(N)cnc2nc(Cl)nn12 ; COC(=O)[C@H]1C[C@H](C(=O)N(C)[C@@H](c2ccc(Br)cc2)C(F)(F)F)C1 ; COC(=O)[C@H]1C[C@H](C(=O)N(C)[C@@H](c2ccc(Nc3cnc4nc(Cl)nn4c3[C@H](C)OC)cc2)C(F)(F)F)C1]

A mixture of methyl (1S,3r)-3-(((S)-1-(4-bromophenyl)-2,2,2-trifluoroethyl)(methyl)carbamoyl)cyclobutane-1-carboxylate [INT 5.12] (0.1 g, 0.2449 mmol), 2-chloro-7-[(1S)-1-methoxyethyl]-[1,2,4]triazolo[1,5-a]pyrimidin-6-amine [free base of INT 1.1] (55.5 mg, 244 μmol), and caesium carbonate (239 mg, 734 μmol) in dioxane (3 mL) was purged with Ar. Then tris(dibenzylideneacetone) dipalladium (11.1 mg, 12.2 μmol) and xantphos (14.1 mg, 24.4 μmol) were added under Ar and the reaction mixture was stirred at 100° C. for 10 h. After cooling the reaction mixture was diluted with MTBE (50 mL), filtrated, and the filtrate was concentrated under reduced pressure to obtain methyl (1S,3r)-3-(((S)-1-(4-((2-chloro-7-((S)-1-methoxyethyl)-[1,2,4]triazolo[1,5-a]pyrimidin-6-yl)amino)phenyl)-2,2,2-trifluoroethyl)(methyl)carbamoyl)cyclobutane-1-carboxylate [INT 18.3] (68.0 mg, 0.1225 mmol, 50.3% yield) as a yellow oil. m/z: [M+H]+ Calcd for C24H27ClF3N6O4 555.2; Found 555.0.

### Exemplary Compounds of Formula (I)

The following compounds in Table 1 were synthesized according to Schemes 1-11, as described above with the identified intermediates.

TABLE 1

| Cmpd | | | | | |
|---|---|---|---|---|---|
| No. | Structure | Scheme | Intermed. | LCMS | ¹H NMR |
| 1.1 | 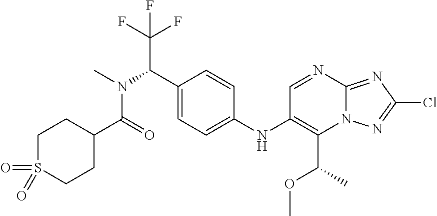 [SMILES: CO[C@@H](C)c1c(Nc2ccc([C@H](N(C)C(=O)C3CCS(=O)(=O)CC3)C(F)(F)F)cc2)cnc2nc(Cl)nn12] | 1 | INT 1.1 INT 2.1 INT 3.1 INT 4.1 INT 5.1 | [M + H]+ C23H27Cl F3N604S 575.2 575.3 | (400 MHz, DMSO-d6) δ = 8.80 (s, 1H), 8.02-7.94 (m, 1H), 7.26- 7.12 (m, 2H), 6.99-6.89 (m, 2H), 6.45- 6.02 (m, 1H), 5.12 (q, J = 6.8 Hz, 1H), 3.25- 3.14 (m, 3H), 3.12 (s, 3H), |
| | | | | | 3.10-3.05 (m, |
| | | | | | 2H), 2.86 (s, |
| | | | | | 3H), 2.08- |
| | | | | | 1.94 (m, 4H), |
| | | | | | 1.55 (d, J = 6.8 |
| | | | | | Hz, 3H) |
| 1.2 | 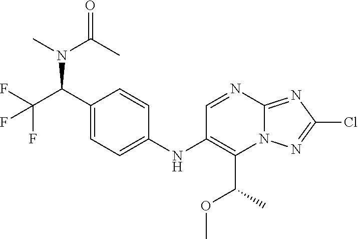 [SMILES: CO[C@@H](C)c1c(Nc2ccc([C@H](N(C)C(C)=O)C(F)(F)F)cc2)cnc2nc(Cl)nn12] | 1 | INT 1.1 INT 2.1 INT 3.1 INT 5.2 | [M + H]+ Cl9H21Cl F3N602 457.1 457.2 | (400 MHz, MeOD) δ = 8.90-8.85 (m, 1H), 7.40- 7.27 (m, 2H), 7.11-7.01 (m, 2H), 6.50 (q, J = 9.2 Hz, 1H), 5.36 (q, J = 6.4 Hz, 1H), 3.37- 3.35 (m, 3H), 2.95-2.72 (m, 3H), 2.36- 2.19 (m, |
| | | | | | 3H), 1.64 (d, |
| | | | | | J = 6.8 Hz, 3H) |
| 1.3 | 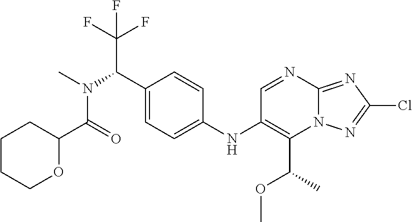 [SMILES: CO[C@@H](C)c1c(Nc2ccc([C@H](N(C)C(=O)C3CCCCO3)C(F)(F)F)cc2)cnc2nc(Cl)nn12] | 1 | INT 1.1 INT 2.1 INT 3.1 INT 4.2 INT 5.3 | [M + H]+ C23H27Cl F3N603 527.2 527.3 | (400 MHz, MeOD) δ = 8.91-8.83 (m, 1H), 7.47- 7.26 (m, 2H), 7.06 (d, J = 8.4 Hz, 2H), 6.54- 6.04 (m, 1H), 5.36 (q, J = 6.8 Hz, 1H), 4.41- 4.25 (m, 1H), 4.08-3.95 (m, 1H), 3.71-3.51 |
| | | | | | (m, 1H), 3.36 |
| | | | | | (s, 3H), 2.98- |
| | | | | | 2.69 (m, 3H), |
| | | | | | 1.98-1.90 |
| | | | | | (m, 1H), 1.85- |
| | | | | | 1.66 (m, |
| | | | | | 3H), 1.64 (d, |
| | | | | | J = 6.8 Hz, |
| | | | | | 3H), 1.63- |
| | | | | | 1.54 (m, 2H) |
| 1.4 | 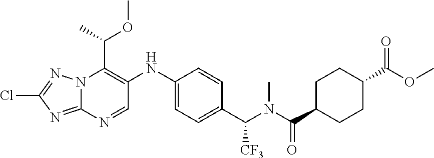 [SMILES: COC(=O)[C@H]1CC[C@H](C(=O)N(C)[C@@H](c2ccc(Nc3cnc4nc(Cl)nn4c3[C@H](C)OC)cc2)C(F)(F)F)CC1] | 1 | INT 1.1 INT 2.1 INT 3.1 INT 4.3 INT 5.4 | [M + H]+ C26H31Cl F3N604 583.2 583.3 | (400 MHz, CDCl₃) δ = 8.90 (s, 1H), 7.32 (d, J = 8.4 Hz, 2H), 7.13- 7.09 (m, 1H), 7.04 (d, J = 8.4 Hz, 2H), 6.63 (q, J = 8.2 Hz, 1H), 5.48 (q, |
| | | | | | J = 6.8 Hz, |
| | | | | | 1H), 3.69 (s. |
| | | | | | 3H), 3.49 (s, |
| | | | | | 3H), 2.92 (s, |
| | | | | | 3H), 2.63- |
| | | | | | 2.54 (m, 1H), |
| | | | | | 2.44-2.35 |
| | | | | | (m, 1H), 2.17- |
| | | | | | 2.06 (m, |
| | | | | | 2H), 2.00- |
| | | | | | 1.92 (m, 1H), |
| | | | | | 1.91-1.83 |
| | | | | | (m, 1H), 1.72- |
| | | | | | 1.63 (m, |
| | | | | | 2H), 1.61 (d, |
| | | | | | J = 6.8 Hz, |
| | | | | | 3H), 1.54- |
| | | | | | 1.42 (m, 2H) |
| 1.5 | 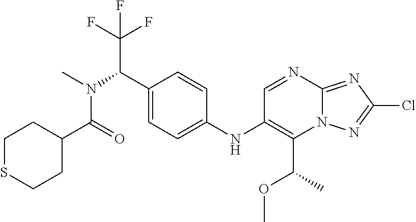 [SMILES: CO[C@@H](C)c1c(Nc2ccc([C@H](N(C)C(=O)C3CCSCC3)C(F)(F)F)cc2)cnc2nc(Cl)nn12] | 1 | INT 1.1 INT 2.1 INT 3.1 INT 4.4 INT 5.5 | [M + H]+ C23H27Cl F3N602S 543.1 543.4 | (400 MHz, CDCl₃) δ = 8.89 (s, 1H), 7.31 (d, J = 8.4 Hz, 2H), 7.11 (s, 1H), 7.04 (d, J = 8.4 Hz, 2H), 6.62 (q, J = 8.4 Hz, 1H), 5.48 (q, J = 6.8 Hz, 1H), 3.49 (s, 3H), 2.90 (s, 3H), 2.81- |
| | | | | | 2.70 (m, 4H), |
| | | | | | 2.70-2.62 |
| | | | | | (m, 1H), 2.17- |
| | | | | | 2.11 (m, |
| | | | | | 1H), 2.08- |
| | | | | | 1.99 (m, 3H), |
| | | | | | 1.61 (d, J = 6.8 |
| | | | | | Hz. 3H) |
| 1.6 | 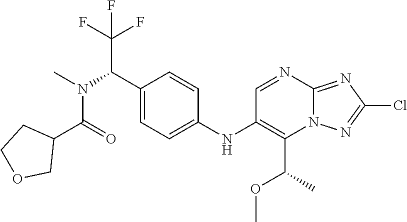 [SMILES: CO[C@@H](C)c1c(Nc2ccc([C@H](N(C)C(=O)C3CCOC3)C(F)(F)F)cc2)cnc2nc(Cl)nn12] | 1 | INT 1.1 INT 2.1 INT 3.1 INT 4.5 INT 5.6 | [M + H]+ C22H25Cl F3N603 513.2 513.3 | (400 MHz, CDCl₃) δ = 8.90 (s, 1H), 7.37-7.31 (m, 2H), 7.12 (s, 1H), 7.05 (d. J = 8.4 Hz, 2H), 6.62 (q, J = 8.8 Hz, 1H), 5.49 (q, J = 6.8 Hz. 1H), 4.18- 4.01 (m, 1H), 4.00-3.86 (m, 3H), 3.49 |
| | | | | | (s, 3H), 3.40- |
| | | | | | 3.28 (m, 1H), |
| | | | | | 2.97-2.82 |
| | | | | | (m, 3H), 2.32- |
| | | | | | 2.09 (m, |
| | | | | | 2H), 1.62 (d, |
| | | | | | J = 6.8 Hz, 3H) |
| 1.7 | 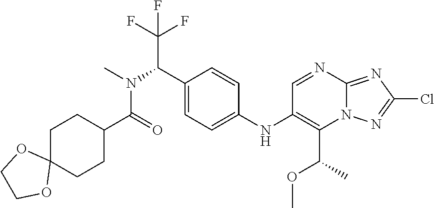 [SMILES: CO[C@@H](C)c1c(Nc2ccc([C@H](N(C)C(=O)C3CCC4(CC3)OCCO4)C(F)(F)F)cc2)cnc2nc(Cl)nn12] | | INT 1.1 INT 2.1 INT 3.1 INT 4.6 INT 5.7 | [M + H]+ C26H31Cl F3N604 583.2 583.4 | (400 MHz, CDCl₃) δ = 8.89 (s, 1H), 7.32 (d, J = 8.4 Hz, 2H), 7.10 (s, 1H), 7.03 (d, J = 8.4 Hz, 2H), 6.65 (q, J = 8.8 Hz, 1H), 5.48 (q, J = 6.8 Hz, 1H), 3.97 (s, 4H), 3.49 (s, |
| | | | | | 3H), 2.92 (s. |
| | | | | | 3H), 2.66- |
| | | | | | 2.55 (m, 1H), |
| | | | | | 1.97-1.93 |
| | | | | | (m, 1H), 1.93- |
| | | | | | 1.83 (m, |
| | | | | | 4H), 1.83- |
| | | | | | 1.75 (m, 1H), |
| | | | | | 1.61 (d, J = 6.8 |
| | | | | | Hz, 3H), 1.57- |
| | | | | | 1.52 (m, 2H) |
| 1.8 | 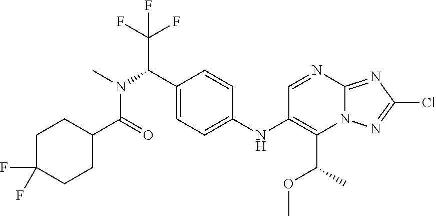 [SMILES: CO[C@@H](C)c1c(Nc2ccc([C@H](N(C)C(=O)C3CCC(F)(F)CC3)C(F)(F)F)cc2)cnc2nc(Cl)nn12] | 1 | INT 1.1 INT 2.1 INT 3.1 INT 4.7 INT 5.8 | [M + H]+ C24H27Cl F5N602 561.2 561.2 | (400 MHz, DMSO-d6) δ = 8.84 (s, 1H), 8.08-7.95 (m, 1H), 7.29- 7.13 (m, 2H), 7.04- 6.90 (m, 2H), 6.71-6.10 (m, 1H), 5.15 (q, J = 6.8 Hz, 1H), 3.16 (s, 3H), 2.90 (s, |
| | | | | | 4H), 2.13- |
| | | | | | 1.62 (m, 8H), |
| | | | | | 1.59 (d, J = 6.8 |
| | | | | | Hz, 3H) |
| 19 | 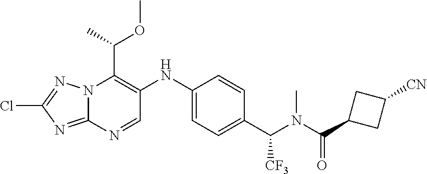 [SMILES: CO[C@@H](C)c1c(Nc2ccc([C@H](N(C)C(=O)[C@H]3C[C@H](C#N)C3)C(F)(F)F)cc2)cnc2nc(Cl)nn12] | 1 | INT 1.1 INT 2.1 INT 3.1 INT 5.9 | [M + H]+ C23H24Cl F3N702 522.2 522.2 | (400 MHz, Methanol-d₄) 6 = 8.87 (d, J = 1.7 Hz, 1H), 7.31 (d, J = 8.4 Hz, 2H). 7.07 (d. J = 8.4 Hz, 2H), 6.49 (q, J = 9.1 Hz, 1H), 5.37 |
| | | | | | (q, J = 6.7 Hz, |
| | | | | | 1H), 3.76 |
| | | | | | (h, J = 7.3, 6.7 |
| | | | | | Hz, 1H), 3.37- |
| | | | | | 3.24 (m, |
| | | | | | 2H), 2.82 (s, |
| | | | | | 3H), 2.77- |
| | | | | | 2.58 (m, 7H), |
| | | | | | 1.65 (d, J = 6.7 |
| | | | | | Hz, 3H) |
| 1.10 | 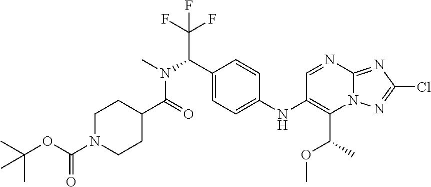 [SMILES: CO[C@@H](C)c1c(Nc2ccc([C@H](N(C)C(=O)C3CCN(C(=O)OC(C)(C)C)CC3)C(F)(F)F)cc2)cnc2nc(Cl)nn12] | 1 | INT 1.1 INT 2.1 INT 6.1 INT 7.1 | [M + H − 100]+ C28H36Cl F3N704 526.2 526.3 | (400 MHz, CDCl₃) δ = 8.90 (s, 1H), 7.32 (d. J = 8.8 Hz, 2H), 7.19- 7.08 (m, 1H), 7.04 (d, J = 8.8 Hz, 2H), 6.63 (q, J = 8.8 Hz, 1H), 5.48 (q, J = 6.8 Hz, |
| | | | | | 1H), 4.19 (br |
| | | | | | d, J = 13.2 Hz, |
| | | | | | 2H), 3.49 (s, |
| | | | | | 3H), 3.00- |
| | | | | | 2.91 (m, 3H), |
| | | | | | 2.85-2.69 |
| | | | | | (m, 3H), 1.82- |
| | | | | | 1.68 (m, |
| | | | | | 4H), 1.62 (s, |
| | | | | | 3H), 1.47 (s, |
| | | | | | 9H) |
| 1.11 | 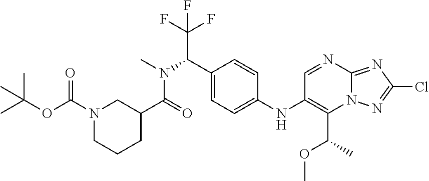 [SMILES: CO[C@@H](C)c1c(Nc2ccc([C@H](N(C)C(=O)C3CCCN(C(=O)OC(C)(C)C)C3)C(F)(F)F)cc2)cnc2nc(Cl)nn12] | 1 | INT 1.1 INT 2.1 INT 6.1 INT 7.2 | [M + H]+ C28H36Cl F3N704 626.2 626.3 | (400 MHz, DMSO-d6) δ = 8.84 (s, 1H), 8.07-7.92 (m, 1H), 7.31- 7.14 (m, 2H), 6.97 (br d, J = 8.4 Hz, 2H), 6.43 (q, J = 9.6 Hz, 1H), 5.16 (q, |
| | | | | | J = 6.8 Hz, |
| | | | | | 1H), 4.08- |
| | | | | | 3.84 (m, 2H), |
| | | | | | 3.17 (s, 3H), |
| | | | | | 2.91 (s, 3H), |
| | | | | | 2.77 (br s, |
| | | | | | 2H), 2.68 (br |
| | | | | | s, 1H), 1.82 |
| | | | | | (br d, J = 12.4 |
| | | | | | Hz, 1H), 1.69- |
| | | | | | 1.54 (m, |
| | | | | | 5H), 1.40 (s, |
| | | | | | 10H) |
| 1.12 | 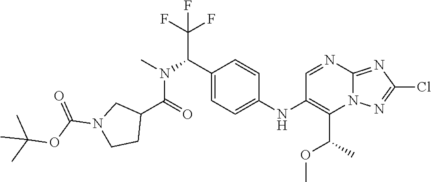 [SMILES: CO[C@@H](C)c1c(Nc2ccc([C@H](N(C)C(=O)C3CCN(C(=O)OC(C)(C)C)C3)C(F)(F)F)cc2)cnc2nc(Cl)nn12] | 1 | INT 1.1 INT 2.1 INT 6.1 INT 7.3 | [M + H]+ C27H34Cl F3N704 612.2 612.4 | (400 MHz, CDCl₃) δ = 8.90 (d, J = 3.2 Hz, 1H), 7.32 (br d, J = 8.0 Hz, 2H), 7.13 (s, 1H), 7.04 (br d, J = 8.4 Hz, 2H), 6.66- 6.55 (m, 1H), 5.48 (q, |
| | | | | | J = 6.8 Hz. |
| | | | | | 1H), 3.84- |
| | | | | | 3.51 (m, 3H), |
| | | | | | 3.49 (s, 3H), |
| | | | | | 3.45-3.25 |
| | | | | | (m, 2H), 2.97- |
| | | | | | 2.78 (m, |
| | | | | | 3H), 2.36- |
| | | | | | 2.05 (m, 2H), |
| | | | | | 1.62 (d, J = 6.8 |
| | | | | | Hz, 3H), 1.49- |
| | | | | | 1.45 (m, 9H) |
| 1.13 | 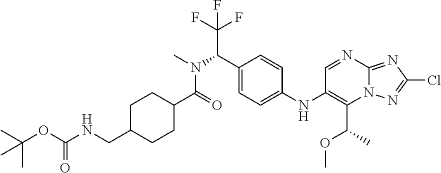 [SMILES: CO[C@@H](C)c1c(Nc2ccc([C@H](N(C)C(=O)C3CCC(CNC(=O)OC(C)(C)C)CC3)C(F)(F)F)cc2)cnc2nc(Cl)nn12] | 1 | INT 1.1 INT 2.1 INT 6.1 INT 7.4 | [M + H]+ C30H40Cl F3N704 654.3 654.6 | (400 MHz, DMSO-d6) δ = 8.85 (s, 1H), 8.05-7.97 (m, 1H), 7.26- 7.10 (m, 2H), 7.04- 6.92 (m, 2H), 6.82 (brt, J = 5.6 Hz, 1H), 6.51- |
| | | | | | 6.07 (m, 1H), |
| | | | | | 5.16 (q, J = 6.8 |
| | | | | | Hz, 1H), 3.16 |
| | | | | | (s, 3H), 2.87 |
| | | | | | (s, 3H), 2.78 |
| | | | | | (br t, J = 6.4 |
| | | | | | Hz, 2H), 2.70- |
| | | | | | 2.56 (m, |
| | | | | | 1H), 1.83- |
| | | | | | 1.64 (m, 4H), |
| | | | | | 1.59 (d, J = 6.8 |
| | | | | | Hz, 3H), 1.37 |
| | | | | | (s, 12H), 1.04- |
| | | | | | 0.83 (m, 2H) |
| 1.14 | 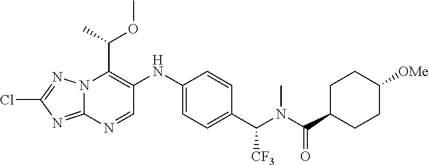 [SMILES: CO[C@@H](C)c1c(Nc2ccc([C@H](N(C)C(=O)[C@H]3CC[C@H](OC)CC3)C(F)(F)F)cc2)cnc2nc(Cl)nn12] | 1 | INT 1.1 INT 2.1 INT 6.1 INT 7.5 | [M + H]+ C25H31Cl F3N603 555.2 555.3 | (400 MHz, DMSO-d6) δ = 8.84 (s, 1H), 7.98 (s, 1H), 7.16 (br d, J = 8.4 Hz, 2H), 6.96 (d, J = 8.7 Hz, 2H), 6.50- 6.09 (m, 1H), |
| | | | | | 5.15 (q, J = 6.7 |
| | | | | | Hz, 1H), 3.23 |
| | | | | | (s, 3H), 3.16 |
| | | | | | (s, 3H), 3.10 |
| | | | | | (br s, 1H), |
| | | | | | 2.91-2.59 |
| | | | | | (m, 4H), 2.11- |
| | | | | | 1.93 (m, |
| | | | | | 2H), 1.84- |
| | | | | | 1.66 (m, 2H), |
| | | | | | 1.59 (d, J = 6.7 |
| | | | | | Hz, 3H), 1.49- |
| | | | | | 1.32 (m, |
| | | | | | 2H), 1.25- |
| | | | | | 1.08 (m, 2H) |
| 1.15 | 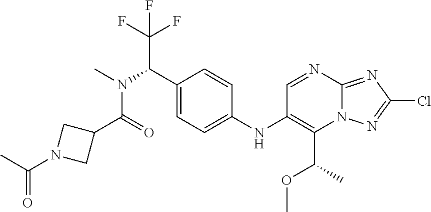 [SMILES: CO[C@@H](C)c1c(Nc2ccc([C@H](N(C)C(=O)C3CN(C(C)=O)C3)C(F)(F)F)cc2)cnc2nc(Cl)nn12] | 1 | INT 1.1 INT 2.1 INT 3.1 INT 4.8 INT 5.10 | [M + H]+ C23H26Cl F3N703 540.2 540.2 | (400 MHz, DMSO-d6) δ = 8.85 (s, 1H), 8.02 (s, 1H), 7.33-7.15 (m, 2H), 6.97 (br d, J = 8.4 Hz, 2H), 6.42 (q, J = 9.2 Hz, 1H), 5.16 (q, J = 6.8 Hz, 1H), 4.38- 4.17 (m, 2H), |
| | | | | | 4.10-3.99 |
| | | | | | (m, 1H), 3.92- |
| | | | | | 3.81 (m, |
| | | | | | 2H), 3.17 (s, |
| | | | | | 3H), 2.76- |
| | | | | | 2.69 (m, 3H), |
| | | | | | 1.76 (d, J = 2.8 |
| | | | | | Hz, 3H), 1.62- |
| | | | | | 1.58 (m, 3H) |
| 1.16 | 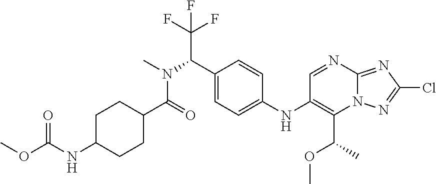 [SMILES: COC(=O)NC1CCC(C(=O)N(C)[C@@H](c2ccc(Nc3cnc4nc(Cl)nn4c3[C@H](C)OC)cc2)C(F)(F)F)CC1] | 1 | INT 1.1 INT 2.1 INT 6.1 INT 9.1 | [M + H]+ C26H32Cl F3N704 598.2 598.3 | (400 MHz, DMSO-d6) δ = 8.83 (s, 1H), 7.98 (s, 1H), 7.16 (br d, J = 8.4 Hz, 2H), 6.96 (d, J = 8.7 Hz, 3H), 6.55- 6.07 (m, 1H), 5.15 (q, J = 6.7 |
| | | | | | Hz, 1H), 3.50 |
| | | | | | (s, 3H), 3.28- |
| | | | | | 3.20 (m, 1H), |
| | | | | | 3.16 (s, 3H), |
| | | | | | 2.87 (s, 3H), |
| | | | | | 2.61 (br s, |
| | | | | | 1H), 1.90- |
| | | | | | 1.66 (m, 4H), |
| | | | | | 1.59 (d, J = 6.7 |
| | | | | | Hz, 3H), 1.51- |
| | | | | | 1.32 (m, |
| | | | | | 2H), 1.31- |
| | | | | | 1.13 (m, 2H) |
| 1.17 | 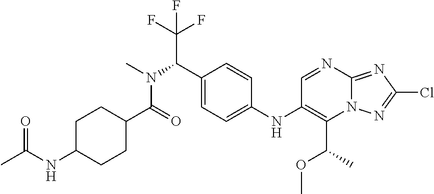 [SMILES: CO[C@@H](C)c1c(Nc2ccc([C@H](N(C)C(=O)C3CCC(NC(C)=O)CC3)C(F)(F)F)cc2)cnc2nc(Cl)nn12] | 1 | INT 1.1 INT 2.1 INT 6.1 INT 9.2 | [M + H]+ C26H32Cl F3N703 582.2 582.2 | (400 MHz, DMSO-d6) δ = 8.83 (s, 1H), 7.98 (s, 1H), 7.72 (d, J = 7.6 Hz, 1H), 7.17 (br d, J = 8.4 Hz, 2H), 6.96 (d, J = 8.7 Hz, 2H), 6.45 (br d. J = 9.5 Hz, 1H), 5.15 (q, |
| | | | | | J = 6.7 Hz |
| | | | | | 1H), 3.46 (br |
| | | | | | s, 1H), 3.16 |
| | | | | | (s, 3H), 2.88 |
| | | | | | (s, 3H), 2.63 |
| | | | | | (br d, J = 7.2 |
| | | | | | Hz, 1H), 1.91- |
| | | | | | 1.66 (m, |
| | | | | | 7H), 1.59 (d, |
| | | | | | J = 6.6 Hz, |
| | | | | | 3H), 1.51- |
| | | | | | 1.31 (m, 2H), |
| | | | | | 1.29-1.11 |
| | | | | | (m, 2H) |
| 1.18 | 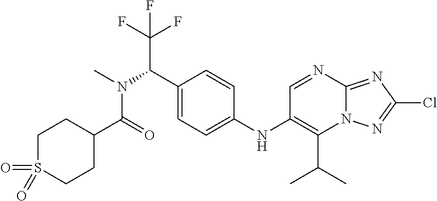 [SMILES: CC(C)c1c(Nc2ccc([C@H](N(C)C(=O)C3CCS(=O)(=O)CC3)C(F)(F)F)cc2)cnc2nc(Cl)nn12] | 1 | INT 1.2 INT 2.1 INT 3.1 INT 4.1 INT 5.1 | [M + H]+ C23H27Cl F3N603S 559.1 559.1 | (400 MHz, DMSO-d6) δ = 8.77-8.66 (m, 1H), 8.20- 8.10 (m, 1H), 7.26- 7.10 (m, 2H), 6.86-6.74 (m, 2H), 6.46- 6.04 (m, 1H), 3.71 (q, J = 7.2 Hz, |
| | | | | | 1H), 3.26- |
| | | | | | 3.05 (m, 5H), |
| | | | | | 2.94-2.61 |
| | | | | | (m, 3H), 2.08- |
| | | | | | 1.90 (m, |
| | | | | | 4H), 1.42 (d, |
| | | | | | J = 7.2 Hz, 6H) |
| 1.19 | 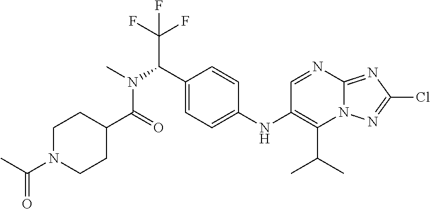 [SMILES: CC(=O)N1CCC(C(=O)N(C)[C@@H](c2ccc(Nc3cnc4nc(Cl)nn4c3C(C)C)cc2)C(F)(F)F)CC1] | 1 | INT 1.2 INT 2.1 INT 3.1 INT 4.9 INT 5.11 | [M + H]+ C25H30Cl F3N702 552.2 552.2 | (400 MHz, CDCL₃) δ = 8.68 (s, 1H), 7.24 (br t, J = 7.6 Hz. 2H), 6.66 (d, J = 8.4 Hz, 2H), 6.57 (q, J = 9.2 Hz, 1H), 5.47 (br s, 1H), 4.61 (br d, J = 13.6 Hz, 1H), 3.97- |
| | | | | | 3.86 (m, |
| | | | | | 2H), 3.22- |
| | | | | | 3.08 (m, 1H), |
| | | | | | 2.92 (s, 3H), |
| | | | | | 2.85-2.62 |
| | | | | | (m, 2H), 2.11 |
| | | | | | (d. J = 2.4 Hz, |
| | | | | | 3H), 1.91- |
| | | | | | 1.81 (m, 2H), |
| | | | | | 1.80-1.69 |
| | | | | | (m, 2H), 1.54 |
| | | | | | (s, 3H), 1.53 |
| | | | | | (s, 3H) |
| 1.20 | 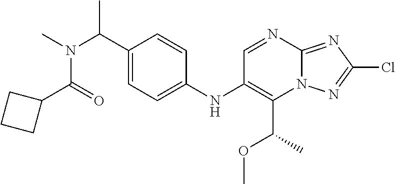 [SMILES: CO[C@@H](C)c1c(Nc2ccc(C(C)N(C)C(=O)C3CCC3)cc2)cnc2nc(Cl)nn12] | 1 | INT 1.1 INT 10.1 INT 11.1 | [M + H]+ C22H28Cl N602 443.2 443.3 | (400 MHz, DMSO-d6) δ = 8.80 (s, 1H), 7.81-7.69 (m, 1H), 7.11- 7.05 (m, 2H), 6.94- 6.89 (m, 2H), 5.73 (q, J = 6.8 Hz, 0.7H), 5.17 (q, J = 6.8 |
| | | | | | Hz, 1H), 4.92 |
| | | | | | (q, J = 6.4 Hz, |
| | | | | | 0.3H), 3.53- |
| | | | | | 3.35 (m, 1H), |
| | | | | | 3.17 (s, 3H), |
| | | | | | 2.52 (s, 2H), |
| | | | | | 2.48 (s, 1H), |
| | | | | | 2.34-2.07 |
| | | | | | (m, 4H), 1.97- |
| | | | | | 1.85 (m, |
| | | | | | 1H), 1.80- |
| | | | | | 1.71 (m, 1H), |
| | | | | | 1.58 (d, J = 6.8 |
| | | | | | Hz, 3H), 1.46- |
| | | | | | 1.32 (m, 3H) |
| 1.21 | 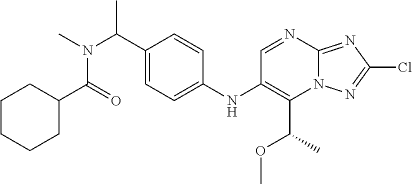 [SMILES: CO[C@@H](C)c1c(Nc2ccc(C(C)N(C)C(=O)C3CCCCC3)cc2)cnc2nc(Cl)nn12] | 1 | INT 1.1 INT 10.1 INT 11.2 | [M + H]+ C24H32Cl N602 471.2 471.3 | (400 MHz, DMSO-d6) δ = 8.79 (s, 1H), 7.79-7.70 (m, 1H), 7.11- 7.03 (m, 2H), 6.96- 6.88 (m, 2H), 5.83-5.12 (m, 2H), 3.17 (s, 3H), 2.75- |
| | | | | | 2.53 (m, 3H), |
| | | | | | 1.74-1.60 |
| | | | | | (m, 5H), 1.58 |
| | | | | | (d, J = 6.8 Hz, |
| | | | | | 3H), 1.49 (br |
| | | | | | d, J = 6.8 Hz, |
| | | | | | 1H), 1.42 (br |
| | | | | | s, 1H), 1,34 |
| | | | | | (br d, J = 7.2 |
| | | | | | Hz, 3H), 1.27 |
| | | | | | (br d, J = 12.8 |
| | | | | | Hz, 2H), 1.24- |
| | | | | | 1.09 (m, 2H) |
| 1.22 | 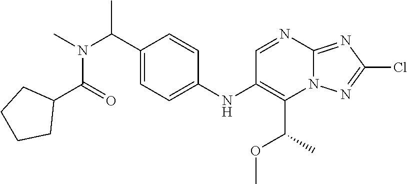 [SMILES: CO[C@@H](C)c1c(Nc2ccc(C(C)N(C)C(=O)C3CCCC3)cc2)cnc2nc(Cl)nn12] | 1 | INT 1.1 INT 10.1 INT 11.3 | [M + H]+ C23H30Cl N602 457.2 457.3 | (400 MHz, DMSO-d6) δ = 8.80 (s, 1H), 7.80-7,70 (m, 1H), 7.14- 7.04 (m, 2H), 6.99- 6.87 (m, 2H), 5.81-5.13 (m, 2H), 3.17 (s, 3H), 3.13- |
| | | | | | 2.91 (m, 1H), |
| | | | | | 2.70-2.51 |
| | | | | | (m, 3H), 1.86- |
| | | | | | 1.62 (m, |
| | | | | | 6H), 1.58 (d, |
| | | | | | J = 6.8 Hz. |
| | | | | | 3H), 1.55- |
| | | | | | 1.45 (m, 3H), |
| | | | | | 1.35 (d, J = 7.2 |
| | | | | | Hz, 2H) |
| 1.23 | 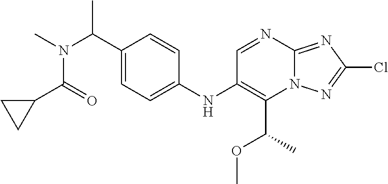 [SMILES: CO[C@@H](C)c1c(Nc2ccc(C(C)N(C)C(=O)C3CC3)cc2)cnc2nc(Cl)nn12] | 1 | INT 1.1 INT 10.1 INT 12.2 | [M + H]+ C21H26Cl N602 429.2 429.3 | (400 MHz, DMSO-d6) δ = 8.86 (s, 1H), 7.92-7.74 (m, 1H), 7.46- 6.96 (m, 4H), 5.86- 5.52 (m, 1H), 5.23 (g, J = 6.8 Hz. 1H), 3.24 (s, 3H), 2.87- |
| | | | | | 2.58 (m, 3H), |
| | | | | | 2.19-1.89 |
| | | | | | (m, 1H), 1.64 |
| | | | | | (d, J = 6.8 Hz, |
| | | | | | 3H), 1.59 (br |
| | | | | | d, J = 6.4 Hz, |
| | | | | | 1H), 1.43 (br |
| | | | | | d, J = 7.2 Hz, |
| | | | | | 2H), 0.85- |
| | | | | | 0.76 (m, 4H) |
| 1.24 | 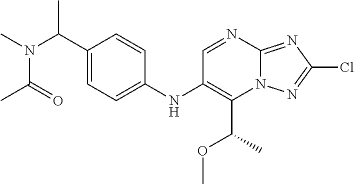 [SMILES: CO[C@@H](C)c1c(Nc2ccc(C(C)N(C)C(C)=O)cc2)cnc2nc(Cl)nn12] | 1 | INT 1.1 INT 10.1 INT 12.1 | [M + H]+ Cl9H24Cl N602 403.2 403.2 | (400 MHz, DMSO-d6) δ = 8.85-8.53 (m, 1H), 7.89- 7.72 (m, 1H), 7.49- 6.91 (m, 4H), 5.80-5.01 (m, 2H), 3.18 (s, 3H), 2.65- 2.54 (m, 3H), |
| | | | | | 2.16-2.01 |
| | | | | | (m, 3H), 1.59 |
| | | | | | (d, J = 6.8 Hz, |
| | | | | | 3H), 1.50- |
| | | | | | 1.34 (m, 3H) |
| 1.25 | 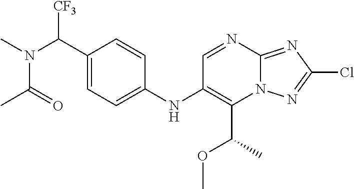 [SMILES: CO[C@@H](C)c1c(Nc2ccc(C(N(C)C(C)=O)C(F)(F)F)cc2)cnc2nc(Cl)nn12] | 1 | INT 1.1 INT 13.1 | [M + H]+ Cl9H21Cl F3N602 457.1 457.1 | (400 MHz, DMSO-d6) δ = 8.84 (s, 1H), 8.07-7.90 (m, 1H), 7.34- 7.13 (m, 2H), 6.97 (br d, J = 8.6 Hz, 2H), 6.50- 5.82 (m, 1H), 5.16 (q, J = 6.7 Hz, 1H), 3.17 |
| | | | | | (s, 3H), 2.90- |
| | | | | | 2.59 (m, 3H), |
| | | | | | 2.31 - 2.10 |
| | | | | | (m, 3H), 1.60 |
| | | | | | (d, J = 6.7 Hz, |
| | | | | | 3H) |
| 1.26 | 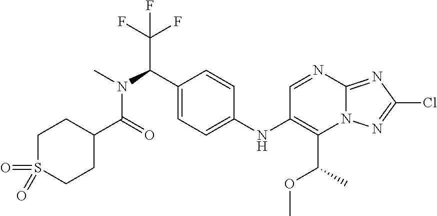 [SMILES: CO[C@@H](C)c1c(Nc2ccc([C@@H](N(C)C(=O)C3CCS(=O)(=O)CC3)C(F)(F)F)cc2)cnc2nc(Cl)nn12] | 1 | INT 1.1 INT 14.1 | [M + H]+ C23H27Cl F3N604S 575.1 575.3 | (400 MHz, DMSO-d6) δ = 8.83 (s, 1H), 8.07-7.97 (m, 1H), 7.29- 7.17 (m, 2H), 7.03- 6.92 (m, 2H), 6.49-6.05 (m, 1H), 5.16 (q, J = 6.8 Hz, 1H), 3.26- 3.19 (m, 2H) |
| | | | | | 3.16 (s, 3H), |
| | | | | | 3.13-3.08 |
| | | | | | (m, 2H), 2.90 |
| | | | | | (s, 3H), 2.65 |
| | | | | | (s, 1H), 2.10- |
| | | | | | 1.95 (m, 4H), |
| | | | | | 1.59 (d. J = 6.8 |
| | | | | | Hz, 3H) |
| 1.27 | 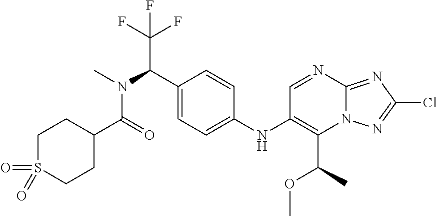 [SMILES: CO[C@H](C)c1c(Nc2ccc([C@@H](N(C)C(=O)C3CCS(=O)(=O)CC3)C(F)(F)F)cc2)cnc2nc(Cl)nn12] | 1 | INT 1.3 INT 14.1 | [M + H]+ C23H27Cl F3N604S 575.1 575.3 | (400 MHz, MeOD) δ = 8.86 (s, 1H), 7.30 (d, J = 8.4 Hz, 2H), 7.06 (d, J = 8.4 Hz, 2H), 6.52 (q, J = 9.2 Hz, 1H), 5.36 (q, J = 6.8 Hz, 1H), 3.36 (s, 3H), 3.25- 3.10 (m, 5H), |
| | | | | | 3.02-2.75 |
| | | | | | (m, 3H), 2.35- |
| | | | | | 2.12 (m, |
| | | | | | 4H), 1.64 (d, |
| | | | | | J = 6.8 Hz, 3H) |
| 1.28 | 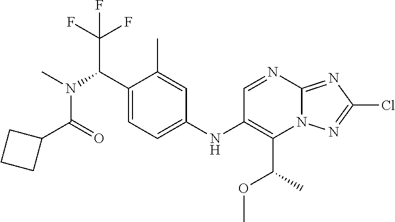 [SMILES: CO[C@@H](C)c1c(Nc2ccc([C@H](N(C)C(=O)C3CCC3)C(F)(F)F)c(C)c2)cnc2nc(Cl)nn12] | 1 | INT 1.1 INT 15.1 INT 16.1 | [M + H]+ C23H27Cl F3N602 511.2 511.2 | (400 MHz, DMSO-d6) δ = 8.83 (d, J = 1.4 Hz, 1H), 7.91 (s, 1H), 7.29 (d, J = 8.4 Hz, 1H), 6.85- 6.78 (m, 2H), 6.43 (q, J = 9.3 Hz, 1H), 5.14 (q, J = 6.6 Hz, 1H), 3.46 (p, J = 8.5 Hz, |
| | | | | | 1H), 3.16 (s, |
| | | | | | 3H), 2.63 (s, |
| | | | | | 3H), 2.14 (dp, |
| | | | | | J = 24.5, 8.1, |
| | | | | | 7.5 Hz, 4H), |
| | | | | | 2.05 (s, 3H), |
| | | | | | 1.93 (dt, |
| | | | | | J = 18.4, 9.2 |
| | | | | | Hz, 1H), 1,76 |
| | | | | | (s, 1H), 1.58 |
| | | | | | (d, J = 6.7 Hz, |
| | | | | | 3H) |
| 1.29 | 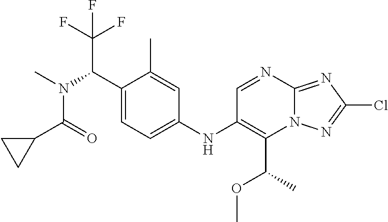 [SMILES: CO[C@@H](C)c1c(Nc2ccc([C@H](N(C)C(=O)C3CC3)C(F)(F)F)c(C)c2)cnc2nc(Cl)nn12] | 1 | INT 1.1 INT 15.1 INT 16.2 | [M + H]+ C22H25Cl F3N602 497.2 497.2 | (400 MHz, CD₃OD) δ = 8.89 (d, J = 2.4 Hz, 1H), 7.48 (d, J = 8.4 Hz, 1H), 6.94 (dt, J = 8.7, 4.3 Hz, 2H), 6.51 (q, J = 8.9 Hz, 1H), 5.38 (q, J = 6.7 Hz, 1H), 3.38 (s, 3H), 3.01 (s, 3H), 2.71- |
| | | | | | 2.58 (m, 1H), |
| | | | | | 2.33-2.11 |
| | | | | | (m, 3H), 2.07- |
| | | | | | 1.95 (m, |
| | | | | | 1H), 1.65 (d, |
| | | | | | J = 6.7 Hz, |
| | | | | | 3H), 1.03- |
| | | | | | 0.86 (m, 4H) |
| 1.30 | 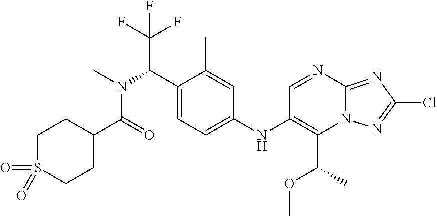 [SMILES: CO[C@@H](C)c1c(Nc2ccc([C@H](N(C)C(=O)C3CCS(=O)(=O)CC3)C(F)(F)F)c(C)c2)cnc2nc(Cl)nn12] | 1 | INT 1.1 INT 15.1 INT 16.3 | [M + H]+ C24H29Cl F3N604S 589.2: Found 589.0. | (500 MHz, DMSO-d6) δ = 8.80 (s, 1H), 7.90 (s, 1H), 7.29 (d. J = 8.3 Hz, 1H), 6.87- 6.75 (m, 2H), 6.43 (q, J = 9.1 Hz, 1H), 5.13 (q, J = 6.8 Hz, 1H), 3.27- 3.14 (m, 2H), |
| | | | | | 3.15 (s, 3H), |
| | | | | | 3.12-3.04 |
| | | | | | (m, 3H), 2.79 |
| | | | | | (s, 3H), 2.15- |
| | | | | | 2.03 (m, 2H), |
| | | | | | 2.01 (s, 3H), |
| | | | | | 1.99-1,94 |
| | | | | | (m, 2H), 1.57 |
| | | | | | (d, J = 6.7 Hz, |
| | | | | | 3H) |
| 1.31 | 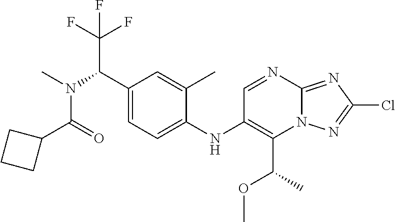 [SMILES: CO[C@@H](C)c1c(Nc2ccc([C@H](N(C)C(=O)C3CCC3)C(F)(F)F)cc2C)cnc2nc(Cl)nn12] | 1 | INT 1.1 INT 15.2 INT 17.1 | [M + H]+ C23H27Cl F3N602 511.2 511.2 | (500 MHz, DMSO-d6) δ = 8.74 (s, 1H), 7.23 (s, 1H), 7.14 (s, 1H), 7.02 (d. J = 8.5 Hz, 1H), 6.83 (d, J = 8.4 Hz, 1H), 6.40 (q, J = 9.5 Hz. 1H), 5.15 (q, J = 6.7 Hz, 1H), 3.50- 3.43 (m, 1H), |
| | | | | | 3.23 (s, 3H), |
| | | | | | 2.71 (s, 3H), |
| | | | | | 2.30 (s, 3H), |
| | | | | | 2.21-2.06 |
| | | | | | (m, 4H), 1.91 |
| | | | | | (q, J = 9.5 Hz, |
| | | | | | 1H), 1.79- |
| | | | | | 1.73 (m, 1H), |
| | | | | | 1.54 (d, J = 6.7 |
| | | | | | Hz, 3H) |
| 1.32 | 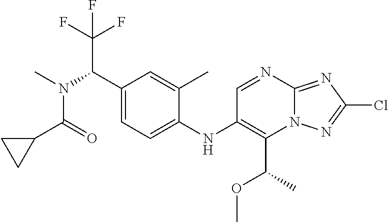 [SMILES: CO[C@@H](C)c1c(Nc2ccc([C@H](N(C)C(=O)C3CC3)C(F)(F)F)cc2C)cnc2nc(Cl)nn12] | 1 | INT 1.1 INT 15.2 INT 17.2 | [M + H]+ C22H25Cl F3N602 497.2 497.0 | (400 MHz, DMSO-d6) δ = 8.81-8.75 (m, 1H), 7.26 (s, 1H), 7.17 (s, 1H), 7.05 (d, J = 8.4 Hz, 1H), 6.90- 6.83 (m, 1H), 6.43 (q, J = 9.5 Hz, 1H), 5.17 (q, J = 6.6 Hz, 1H), 3.25 (s, 3H), 3.01 (s, |
| | | | | | 3H), 2.32 (s, |
| | | | | | 3H), 2.06- |
| | | | | | 1.97 (m, 1H), |
| | | | | | 1.56 (d, J = 6.7 |
| | | | | | Hz, 3H), 0.90- |
| | | | | | 0.81 (m, 4H) |
| 1.33 | 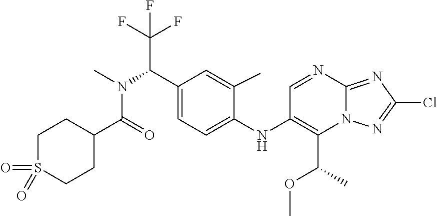 [SMILES: CO[C@@H](C)c1c(Nc2ccc([C@H](N(C)C(=O)C3CCS(=O)(=O)CC3)C(F)(F)F)cc2C)cnc2nc(Cl)nn12] | 1 | INT 1.1 INT 15.2 INT 17.3 | [M + H]+ C24H29Cl F3N604S 589.2 589.0 | (600 MHz, DMSO-d6) δ = 8.74 (s, 1H), 7.24 (s, 1H), 7.15 (s, 1H), 7.03 (d, J = 8.6 Hz, 1H), 6.84 (d, J = 8.4 Hz, 1H), 6.41 (q, J = 9.4 Hz. 1H), 5.16 (q, J = 6.7 Hz, 1H), 3.21 (d, |
| | | | | | J = 13,9 Hz, |
| | | | | | 3H), 3.18- |
| | | | | | 3.05 (m, 5H), |
| | | | | | 2.89 (s, 3H), |
| | | | | | 2.34-2.28 |
| | | | | | (m, 3H), 2.11- |
| | | | | | 1.95 (m, |
| | | | | | 4H), 1.54 (d, |
| | | | | | J = 6.7 Hz, 3H) |
| 2.1 | 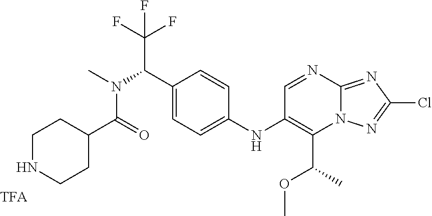 [SMILES: CO[C@@H](C)c1c(Nc2ccc([C@H](N(C)C(=O)C3CCNCC3)C(F)(F)F)cc2)cnc2nc(Cl)nn12] | 2 | INT 1.1 INT 2.1 INT 6.1 INT 7.1 1.10 | [M + H]+ C23H28Cl F3N702 526.2 526.3 | (400 MHz, CDCl₃) δ = 9.66 (br s, 1H), 9.16 (br s, 1H), 8.89 (s, 1H), 7.31 (d, J = 8.4 Hz, 2H), 7.14 (s, 1H), 7.05 (d, J = 8.8 Hz, 2H), 6.57 (q, J = 8.8 Hz, 1H), 5.48 (q, |
| | | | | | J = 6.8 Hz, |
| | | | | | 1H), 3.57 (br |
| | | | | | s, 5H), 3.14 |
| | | | | | (br d, J = 5,2 |
| | | | | | Hz, 2H), 2.99 |
| | | | | | (br s, 1H), |
| | | | | | 2.93 (s, 3H), |
| | | | | | 2.23-1.94 |
| | | | | | (m, 4H), 1.61 |
| | | | | | (d, J = 6.8 Hz, |
| | | | | | 3H) |
| 2.2 | 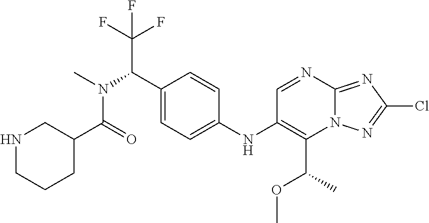 [SMILES: CO[C@@H](C)c1c(Nc2ccc([C@H](N(C)C(=O)C3CCCNC3)C(F)(F)F)cc2)cnc2nc(Cl)nn12] | 2 | INT 1.1 INT 2.1 INT 6.1 INT 7.2 1.11 | [M + H]+ C23H28Cl F3N702 526.2 526.3 | (400 MHz, CDCl₃) δ = 8.89 (s, 1H), 7.31 (d, J = 8.4 Hz, 2H), 7.11 (s, 1H), 7.04 (d, J = 8.8 Hz, 2H), 6.61 (q, J = 9.2 Hz, 1H), 5.48 (q, J = 6.8 Hz, 1H), 3.51- 3.45 (m, 3H), 3.15-2.98 |
| | | | | | (m, 2H), 2.93 |
| | | | | | (s, 3H), 2.85- |
| | | | | | 2.67 (m, 2H), |
| | | | | | 2.62 (s, 2H). |
| | | | | | 2.05-1.96 |
| | | | | | (m, 1H), 1.82- |
| | | | | | 1.70 (m, |
| | | | | | 2H), 1.61 (d, |
| | | | | | J = 6.8 Hz, |
| | | | | | 3H), 1.59- |
| | | | | | 1.50 (m, 1H) |
| 2.3 | 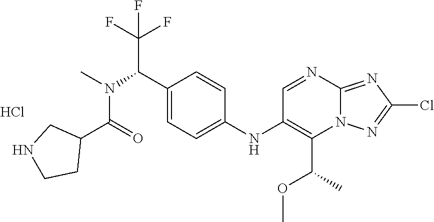 [SMILES: Cl ; CO[C@@H](C)c1c(Nc2ccc([C@H](N(C)C(=O)C3CCNC3)C(F)(F)F)cc2)cnc2nc(Cl)nn12] | 2 | INT 1.1 INT 2.1 INT 6.1 INT 7.3 1.12 | [M + H]+ C22H26Cl F3N702 512.2; 514.2 512.3; 514.2 | (400 MHz, CDCl3) δ = 10.16 (br s, 1H), 9.42 (br s, 1H), 8.90 (br s, 1H), 7.47-7.27 (m, 2H), 7.14- 7.00 (m, 2H), 6.62- 6.43 (m, 1H), 5.48 (br d, J = 6.0 Hz, 1H), 3.68 (br |
| | | | | | d, J = 14.8 Hz, |
| | | | | | 4H), 3.49 (s. |
| | | | | | 3H), 3.04- |
| | | | | | 2.79 (m, 3H), |
| | | | | | 2.47 (br s, |
| | | | | | 1H), 2.22- |
| | | | | | 2.09 (m, 2H), |
| | | | | | 1.61 (br d, |
| | | | | | J = 6.0 Hz, 3H) |
| 2.4 | 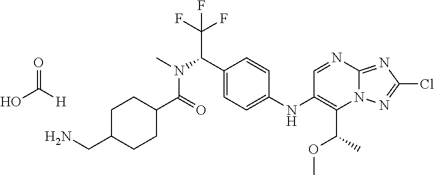 | 2 | INT 1.1 INT 2.1 INT 6.1 INT 7.4 1.13 | [M + H]+ C25H32Cl F3N702 554.2 554.4 | (400 MHz, DMSO-d6) δ = 8.84 (s, 1H), 8.43 (s, 1H). 8.13-8.00 (m, 1H), 7.29- 7.12 (m, 2H), 7.02- 6.93 (m, 2H), 6.52-6.08 (m, 1H), 5.16 |
| | | | | | (q, J = 6.8 Hz, |
| | | | | | 1H), 3.16 (s, |
| | | | | | 3H), 2.87 (s, |
| | | | | | 3H), 2.67- |
| | | | | | 2.56 (m, 2H), |
| | | | | | 1.88-1.67 |
| | | | | | (m, 4H), 1.59 |
| | | | | | (d, J = 6.4 Hz, |
| | | | | | 3H), 1.55- |
| | | | | | 1.21 (m, 4H), |
| | | | | | 1.10-0.91 |
| | | | | | (m, 2H) |
| 3.1 | 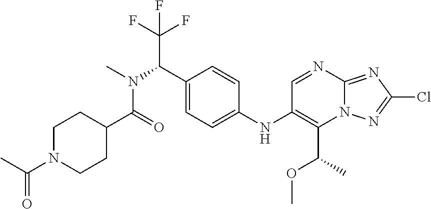 [SMILES: CO[C@@H](C)c1c(Nc2ccc([C@H](N(C)C(=O)C3CCN(C(C)=O)CC3)C(F)(F)F)cc2)cnc2nc(Cl)nn12] | 3 | INT 1.1 INT 2.1 INT 6.1 INT 7.1 1.10 2.1 | [M + H]+ C25H30Cl F3N703 568.2 568.4 | (400 MHz, CDCl₃) δ = 8.90 (s, 1H), 7.35-7.29 (m, 2H), 7.17- 7.01 (m, 3H), 6.68- 6.58 (m, 1H), 5.49 (q, J = 6.8 Hz, 1H), 4.71- 4.54 (m, 1H), 3.93 (br d, J = 13.6 Hz, |
| | | | | | 1H), 3.49 (s, |
| | | | | | 3H), 3.20- |
| | | | | | 3.09 (m, 1H), |
| | | | | | 2.95 (s, 3H), |
| | | | | | 2.88-2.66 |
| | | | | | (m, 2H), 2.12 |
| | | | | | (s, 3H), 1.95- |
| | | | | | 1.67 (m, 4H), |
| | | | | | 1.62 (d, J = 6.7 |
| | | | | | Hz, 3H) |
| 3.2 | 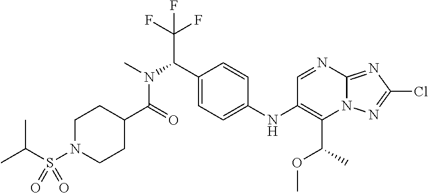 [SMILES: CO[C@@H](C)c1c(Nc2ccc([C@H](N(C)C(=O)C3CCN(S(=O)(=O)C(C)C)CC3)C(F)(F)F)cc2)cnc2nc(Cl)nn12] | 3 | INT 1.1 INT 2.1 INT 6.1 INT 7.1 1.10 2.1 | [M + H]+ C26H34Cl F3N704S 632.2 632.3 | (400 MHz, CDCl₃) δ = 8.89 (s, 1H), 7.32 (br d, J = 8.4 Hz, 2H), 7.18- 7.01 (m, 3H), 6.62 (q, J = 9.2 Hz, 1H), 5.48 (q, J = 6.8 Hz, 1H), 3.85 (dt, J = 4.4, 8.8 Hz, |
| | | | | | 2H), 3.49 (s, |
| | | | | | 3H), 3.19 (td, |
| | | | | | J = 6.8, 13.6 |
| | | | | | Hz, 1H), 3,08- |
| | | | | | 2.99 (m, |
| | | | | | 2H), 2.93 (s, |
| | | | | | 3H), 2.85- |
| | | | | | 2.67 (m, 1H), |
| | | | | | 1.98-1.87 |
| | | | | | (m, 3H), 1.87- |
| | | | | | 1.79 (m, |
| | | | | | 1H), 1.62 (s. |
| | | | | | 3H), 1.37 (d, |
| | | | | | J = 6.8 Hz, 6H) |
| 3.3 | 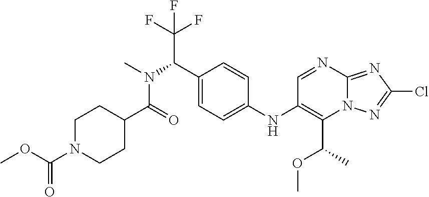 [SMILES: COC(=O)N1CCC(C(=O)N(C)[C@@H](c2ccc(Nc3cnc4nc(Cl)nn4c3[C@H](C)OC)cc2)C(F)(F)F)CC1] | 3 | INT 1.1 INT 2.1 INT 6.1 INT 7.1 1.10 2.1 | [M + H]+ C25H30Cl F3N704 584.2 584.5 | (400 MHz, CDCl₃) δ = 8.89 (s, 1H), 7.32 (d, J = 8.4 Hz, 2H), 7.12 (s, 1H), 7.04 (d, J = 8.8 Hz, 2H), 6.62 (q, J = 9.2 Hz, 1H), 5.48 (q, J = 6.8 Hz, 1H), 4.22 (br |
| | | | | | s, 2H), 3.72 |
| | | | | | (s, 3H), 3.49 |
| | | | | | (s, 3H), 2.94 |
| | | | | | (s, 3H), 2.91- |
| | | | | | 2.69 (m, 3H), |
| | | | | | 1.89-1.69 |
| | | | | | (m, 4H), 1.62 |
| | | | | | (d, J = 6.7 Hz, |
| | | | | | 3H) |
| 3.4 | 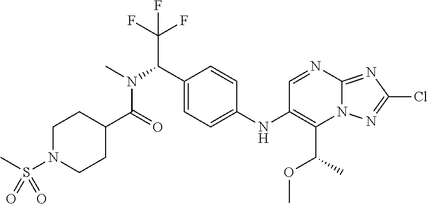 [SMILES: CO[C@@H](C)c1c(Nc2ccc([C@H](N(C)C(=O)C3CCN(S(C)(=O)=O)CC3)C(F)(F)F)cc2)cnc2nc(Cl)nn12] | 3 | INT 1.1 INT 2.1 INT 6.1 INT 7.1 1.10 2.1 | [M + H]+ C24H30Cl F3N704S 604.2 604.3 | (400 MHz, CDCl₃) δ = 8.89 (s, 1H), 7.35-7.29 (m, 2H), 7.12 (s, 1H), 7.05 (d, J = 8.8 Hz, 2H), 6.62 (q, J = 8.8 Hz, 1H), 5.49 (q, J = 6.8 Hz, 1H), 3.91- 3.75 (m, 2H), |
| | | | | | 3.54-3.47 |
| | | | | | (m, 3H), 3.16- |
| | | | | | 2.86 (m, |
| | | | | | 5H), 2.83 (s, |
| | | | | | 3H), 2.78- |
| | | | | | 2.71 (m, 1H), |
| | | | | | 2.05-1.91 |
| | | | | | (m, 3H), 1.91- |
| | | | | | 1.84 (m, |
| | | | | | 1H), 1.62 (d, |
| | | | | | J = 6.8 Hz, 3H) |
| 3.5 | 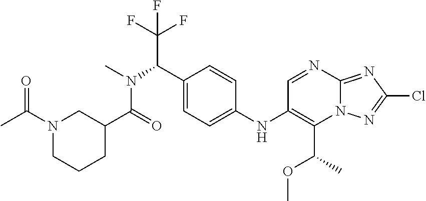 [SMILES: CO[C@@H](C)c1c(Nc2ccc([C@H](N(C)C(=O)C3CCCN(C(C)=O)C3)C(F)(F)F)cc2)cnc2nc(Cl)nn12] | 3 | INT 1.1 INT 2.1 INT 6.1 INT 7.2 1.11 2.2 | [M+ H]+ C25H30Cl F3N703 568.2 568.3 | (400 MHz, CDCl₃) δ = 8.97-8.88 (m, 1H), 7.38- 7.29 (m, 2H), 7.15- 7.10 (m, 1H), 7.08-7.02 (m, 2H), 6.66- 6.51 (m, 1H), 5.48 (q, J = 6.8 Hz, |
| | | | | | 1H), 4.73- |
| | | | | | 4.55 (m, 1H), |
| | | | | | 3.90-3.78 |
| | | | | | (m, 1H), 3.49 |
| | | | | | (s, 3H), 3.22- |
| | | | | | 3.03 (m, 1H), |
| | | | | | 2.94 (s, 3H), |
| | | | | | 2.82-2.64 |
| | | | | | (m, 2H), 2.11 |
| | | | | | (s, 3H), 2.07- |
| | | | | | 1.99 (m, 1H), |
| | | | | | 1.96-1.82 |
| | | | | | (m, 2H), 1.81- |
| | | | | | 1.66 (m, |
| | | | | | 1H), 1.63- |
| | | | | | 1.60 (m, 2H), |
| | | | | | 1.58-1.45 |
| | | | | | (m, 1H) |
| 3.6 | 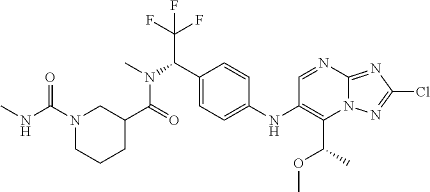 [SMILES: CNC(=O)N1CCCC(C(=O)N(C)[C@@H](c2ccc(Nc3cnc4nc(Cl)nn4c3[C@H](C)OC)cc2)C(F)(F)F)C1] | 3 | INT 1.1 INT 2.1 INT 6.1 INT 7.2 1.11 2.2 | [M + H]+ C25H31Cl F3N803 583.2 583.4 | (400 MHz, DMSO-d6) δ = 8.87-8.83 (m, 1H), 8.05- 7.94 (m, 1H), 7.37- 7.15 (m, 2H), 7.02-6.94 (m, 2H), 6.53- 6.37 (m, 2H), 5.15 (q, J = 6.8 Hz, |
| | | | | | 1H), 4.04 (br |
| | | | | | d, J = 9.6 Hz, |
| | | | | | 1H), 3.91 (br |
| | | | | | d, J = 13.2 Hz, |
| | | | | | 1H), 3.16 (s, |
| | | | | | 3H), 2.90 (s, |
| | | | | | 3H), 2.69 (br |
| | | | | | dd, J = 3.6, |
| | | | | | 11.6 Hz, 2H), |
| | | | | | 2.66-2.61 |
| | | | | | (m, 1H), 2.57- |
| | | | | | 2.54 (m, |
| | | | | | 3H), 1.87- |
| | | | | | 1.72 (m, 1H), |
| | | | | | 1.59 (d, J = 6.8 |
| | | | | | Hz, 3H), 1.57- |
| | | | | | 1.45 (m, |
| | | | | | 2H), 1.43- |
| | | | | | 1.30 (m, 1H) |
| 3.7 | 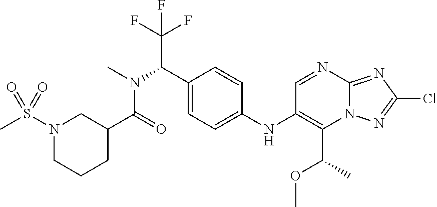 [SMILES: CO[C@@H](C)c1c(Nc2ccc([C@H](N(C)C(=O)C3CCCN(S(C)(=O)=O)C3)C(F)(F)F)cc2)cnc2nc(Cl)nn12] | 3 | INT 1.1 INT 2.1 INT 6.1 INT 7.2 1.11 2.2 | [M + H]+ C24H30Cl F3N704S 604.2 604.3 | (400 MHz, DMSO-d6) δ = 8.86-8.80 (m, 1H), 7.99 (s, 1H), 7.26- 7.14 (m, 2H), 7.00-6.90 (m, 2H), 6.50- 6.12 (m, 1H), 5.18- 4.94 (m, 1H), 3.64-3.52 (m, 2H), 3.33- |
| | | | | | 3.30 (m, |
| | | | | | 3H), 3.16- |
| | | | | | 3.07 (m, 3H), |
| | | | | | 2.90 (s, 3H), |
| | | | | | 2.88-2.84 |
| | | | | | (m, 1H), 2.82- |
| | | | | | 2.65 (m, |
| | | | | | 2H), 1.88- |
| | | | | | 1.71 (m, 2H), |
| | | | | | 1.58 (d, J = 6.8 |
| | | | | | Hz, 3H), 1.55- |
| | | | | | 1.49 (m, |
| | | | | | 1H), 1.48- |
| | | | | | 1.36 (m, 1H) |
| 3.8 | 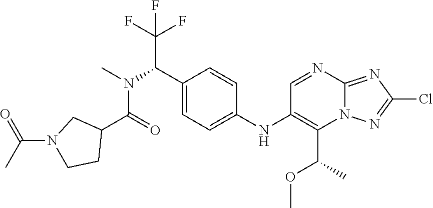 [SMILES: CO[C@@H](C)c1c(Nc2ccc([C@H](N(C)C(=O)C3CCN(C(C)=O)C3)C(F)(F)F)cc2)cnc2nc(Cl)nn12] | 3 | INT 1.1 INT 2.1 INT 6.1 INT 7.3 1.12 2.3 | [M + H]+ C24H28Cl F3N703 554.2 554.4 | (400 MHz, CDCl₃) δ = 8.94-8.88 (m, 1H), 7.35- 7.29 (m, 2H), 7.14 (s, 1H), 7.05 (dd, J = 4.0, 8.4 Hz, 2H), 6.65- 6.53 (m, 1H), 5.48 (q, J = 6.8 Hz, 1H), 4.01- 3.51 (m, |
| | | | | | 4H), 3.49 (s, |
| | | | | | 3H), 3.44- |
| | | | | | 3.25 (m, 1H), |
| | | | | | 3.00-2.91 |
| | | | | | (m, 3H), 2.53- |
| | | | | | 2.11 (m, |
| | | | | | 2H), 2.11- |
| | | | | | 2.06 (m, 3H), |
| | | | | | 1.62 (br s, |
| | | | | | 3H) |
| 3.9 | 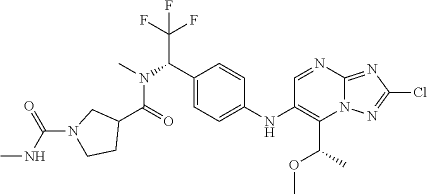 [SMILES: CNC(=O)N1CCC(C(=O)N(C)[C@@H](c2ccc(Nc3cnc4nc(Cl)nn4c3[C@H](C)OC)cc2)C(F)(F)F)C1] | 3 | INT 1.1 INT 2.1 INT 6.1 INT 7.3 1.12 2.3 | [M + H]+ C24H29Cl F3N803 569.2 569.4 | (400 MHz, CDCl₃) δ = 8.91 (d, J = 5.6 Hz, 1H), 7.35- 7.29 (m, 2H), 7.13 (s, 1H), 7.05 (d, J = 8.4 Hz, 2H), 6.64- 6.54 (m, 1H), 5.48 (g, J = 6.8 Hz, 1H), 4.21 |
| | | | | | (br d, J = 4.0 |
| | | | | | Hz, 1H), 3.81- |
| | | | | | 3.68 (m, |
| | | | | | 1H), 3.63- |
| | | | | | 3.54 (m, 2H), |
| | | | | | 3.49 (d, J = 1.2 |
| | | | | | Hz, 3H), 3.44- |
| | | | | | 3.28 (m, |
| | | | | | 2H), 2.94 (d, |
| | | | | | J = 4.4 Hz, |
| | | | | | 3H), 2.84 (dd, |
| | | | | | J = 2.4. 4.8 Hz, |
| | | | | | 3H), 2.42- |
| | | | | | 2.11 (m, 2H), |
| | | | | | 1.63 (br s, |
| | | | | | 3H) |
| 3.10 | 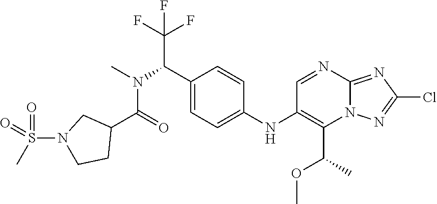 [SMILES: CO[C@@H](C)c1c(Nc2ccc([C@H](N(C)C(=O)C3CCN(S(C)(=O)=O)C3)C(F)(F)F)cc2)cnc2nc(Cl)nn12] | 3 | INT 1.1 INT 2.1 INT 6.1 INT 7.3 1.12 2.3 | [M + H]+ C23H28Cl F3N704S 590.1 590.4 | (400 MHz, CDCl₃) δ = 8.90 (s, 1H), 7.35-7.29 (m, 2H), 7.14 (s, 1H), 7.05 (d, J = 8.4 Hz, 2H), 6.57 (q, J = 9.2 Hz, 1H), 5.49 (q, J = 6.8 Hz, 1H), 3.78- 3.53 (m, 3H), |
| | | | | | 3.49 (s, 3H), |
| | | | | | 3.46-3.35 |
| | | | | | (m, 2H), 2.97- |
| | | | | | 2.91 (m, |
| | | | | | 6H), 2.37- |
| | | | | | 2.15 (m, 2H), |
| | | | | | 1.62 (s, 3H) |
| 3.11 | 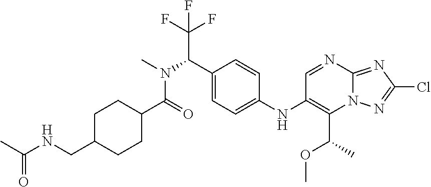 [SMILES: CO[C@@H](C)c1c(Nc2ccc([C@H](N(C)C(=O)C3CCC(CNC(C)=O)CC3)C(F)(F)F)cc2)cnc2nc(Cl)nn12] | 3 | INT 1.1 INT 2.1 INT 6.1 INT 7.4 1.13 2.4 | [M + H]+ C27H34Cl F3N703 596.2 596.2 | (400 MHz, CDCl₃) δ = 8.81 (s, 1H), 7.23 (br d, J = 8.4 Hz, 2H), 7.04 (s, 1H), 6.96 (d, J = 8.4 Hz, 2H), 6.55 (q, J = 9.2 Hz, 1H), 5.64- 5.52 (m, 1H), |
| | | | | | 5.40 (q, J = 6.8 |
| | | | | | Hz, 1H), 3.41 |
| | | | | | (s, 3H), 3.13- |
| | | | | | 3.00 (m, 2H), |
| | | | | | 2.83 (s, 3H), |
| | | | | | 2.52-2.41 |
| | | | | | (m, 1H), 1.96- |
| | | | | | 1.90 (m, |
| | | | | | 3H), 1.80- |
| | | | | | 1.66 (m, 3H), |
| | | | | | 1.60-1.41 |
| | | | | | (m, 6H), 1.06- |
| | | | | | 0.85 (m, 2H) |
| 3.12 | 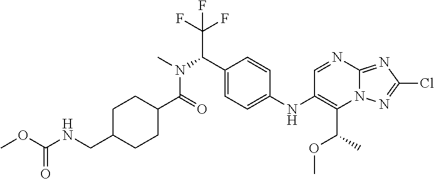 [SMILES: COC(=O)NCC1CCC(C(=O)N(C)[C@@H](c2ccc(Nc3cnc4nc(Cl)nn4c3[C@H](C)OC)cc2)C(F)(F)F)CC1] | 3 | INT 1.1 INT 2.1 INT 6.1 INT 7.4 1.13 2.4 | [M + H]+ C27H34Cl F3N704 612.2 612.2 | (400 MHz, CDCl₃) δ = 8.81 (s, 1H), 7.23 (br d. J = 8.0 Hz, 2H), 7.04 (s, 1H), 6.96 (br d, J = 8.4 Hz, 2H), 6.55 (q, J = 8.8 Hz, 1H), 5.40 (q, |
| | | | | | J = 6.8 Hz, |
| | | | | | 1H), 4.72 (br |
| | | | | | s, 1H), 3.59 |
| | | | | | (s, 3H), 3.41 |
| | | | | | (s, 3H), 3.08- |
| | | | | | 2.92 (m, 2H), |
| | | | | | 2.83 (s, 3H), |
| | | | | | 2.46 (brt, |
| | | | | | J = 11.6 Hz, |
| | | | | | 1H), 1.83- |
| | | | | | 1.67 (m, 4H), |
| | | | | | 1.65-1.38 |
| | | | | | (m, 6H), 0.95 |
| | | | | | (brt, J = 11.2 |
| | | | | | Hz, 2H) |
| 4.1 | 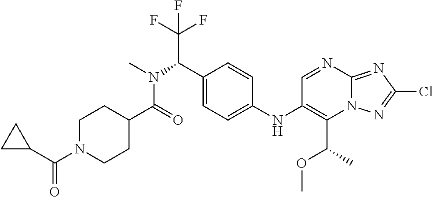 [SMILES: CO[C@@H](C)c1c(Nc2ccc([C@H](N(C)C(=O)C3CCN(C(=O)C4CC4)CC3)C(F)(F)F)cc2)cnc2nc(Cl)nn12] | 4 | INT 1.1 INT 2.1 INT 6.1 INT 7.1 1.10 2.1 | [M + H]+ C27H32Cl F3N703 594.2 594.5 | (400 MHz, CDCl₃) δ = 8.89 (s, 1H), 7.32 (br d, J = 8.0 Hz, 2H), 7.17- 7.02 (m, 3H), 6.63 (q, J = 9.2 Hz, 1H), 5.48 (q, J = 6.8 Hz, 1H), 4.62 (br s, 1H), 4.32 |
| | | | | | (br s, 1H), |
| | | | | | 3.49 (s, 3H), |
| | | | | | 3.29-3.10 |
| | | | | | (m, 1H), 2.95 |
| | | | | | (s, 3H), 2.90- |
| | | | | | 2.70 (m, 2H), |
| | | | | | 1.98-1.67 |
| | | | | | (m, 5H), 1.62 |
| | | | | | (d, J = 6.8 Hz, |
| | | | | | 3H), 0.99 (br |
| | | | | | s, 2H), 0.78 |
| | | | | | (br d, J = 7.8 |
| | | | | | Hz, 2H) |
| 4.2 | 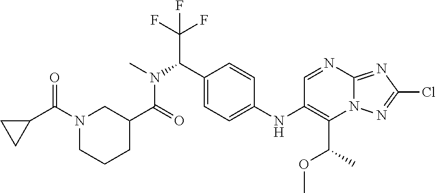 [SMILES: CO[C@@H](C)c1c(Nc2ccc([C@H](N(C)C(=O)C3CCCN(C(=O)C4CC4)C3)C(F)(F)F)cc2)cnc2nc(Cl)nn12] | 4 | INT 1.1 INT 2.1 INT 6.1 INT 7.2 1.11 2.2 | [M + H]+ C27H32Cl F3N703 594.2 594.4 | (400 MHz, DMSO-d6) δ = 8.85 (s, 1H), 8.02 (br s, 1H), 7.30- 7.16 (m, 2H), 6.97 (br d, J = 8.0 Hz. 2H), 6.44 (br d, J = 8.8 Hz, 1H), 5.16 (br d, J = 6.4 Hz, |
| | | | | | 1H), 4.47- |
| | | | | | 4.19 (m, 2H), |
| | | | | | 3.16 (s, 3H), |
| | | | | | 2.89 (br s, |
| | | | | | 3H), 2.70 (br |
| | | | | | d. J = 18.8 Hz, |
| | | | | | 1H), 2.62 (br |
| | | | | | s, 2H), 2.00 |
| | | | | | (br s, 1H), |
| | | | | | 1.91-1.72 |
| | | | | | (m, 2H), 1.71- |
| | | | | | 1.63 (m, |
| | | | | | 1H), 1.59 (br |
| | | | | | d, J = 6.4 Hz, |
| | | | | | 3H), 1.24 (br |
| | | | | | s, 1H), 0.72 |
| | | | | | (br s, 4H) |
| 4.3 | 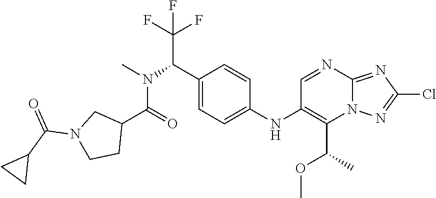 [SMILES: CO[C@@H](C)c1c(Nc2ccc([C@H](N(C)C(=O)C3CCN(C(=O)C4CC4)C3)C(F)(F)F)cc2)cnc2nc(Cl)nn12] | 4 | INT 1.1 INT 2.1 INT 6.1 INT 7.3 1.12 2.3 | [M + H]+ C26H30Cl F3N703 580.2 580.5 | (400 MHz, CDCl₃) δ = 8.92-8.90 (m, 1H), 7.37- 7.30 (m, 2H), 7.13 (s, 1H), 7.08- 7.02 (m, 2H), 6.67-6.55 (m, 1H), 5.48 (q, J = 6.8 Hz, 1H), 4.09- |
| | | | | | 3.53 (m, 4H), |
| | | | | | 3.49 (s, 3H), |
| | | | | | 3.45-3.26 |
| | | | | | (m, 1H), 3.00- |
| | | | | | 2.92 (m, |
| | | | | | 3H), 2.55- |
| | | | | | 2.02 (m, 2H), |
| | | | | | 1.64 (br d, |
| | | | | | J = 5.4 Hz, |
| | | | | | 1H), 1.63 (s, |
| | | | | | 3H), 1.09- |
| | | | | | 0.96 (m, 2H), |
| | | | | | 0.79 (br d, |
| | | | | | J = 7.6 Hz, 2H) |
| 5.1 | 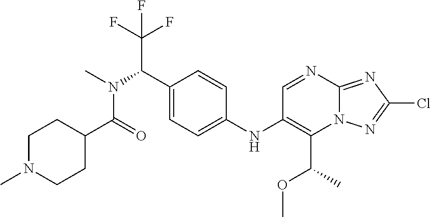 [SMILES: CO[C@@H](C)c1c(Nc2ccc([C@H](N(C)C(=O)C3CCN(C)CC3)C(F)(F)F)cc2)cnc2nc(Cl)nn12] | 5 | INT 1.1 INT 2.1 INT 6.1 INT 7.1 1.10 2.1 | [M + H]+ C24H30Cl F3N702 540.2 540.5 | ¹H NMR (400 MHz, CDCl₃) δ = 8.90 (s, 1H), 7.33 (br d, J = 8.4 Hz, 2H), 7.11 (s, 1H), 7.04 (d, J = 8.4 Hz, 2H), 6.64 (q, J = 9.2 Hz, 1H), 5.48 (q, J = 6.8 Hz, |
| | | | | | 1H), 3.49 (s, |
| | | | | | 3H), 3.03- |
| | | | | | 2.85 (m, 5H), |
| | | | | | 2.54 (br s, |
| | | | | | 1H), 2.30 (s, |
| | | | | | 3H), 2.07- |
| | | | | | 1.89 (m, 4H), |
| | | | | | 1.87-1.79 |
| | | | | | (m, 1H), 1.78- |
| | | | | | 1.70 (m, |
| | | | | | 1H), 1.62 (s. |
| | | | | | 3H) |
| 5.2 | 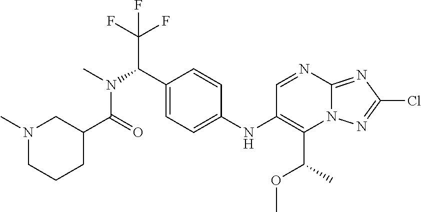 [SMILES: CO[C@@H](C)c1c(Nc2ccc([C@H](N(C)C(=O)C3CCCN(C)C3)C(F)(F)F)cc2)cnc2nc(Cl)nn12] | 5 | INT 1.1 INT 2.1 INT 6.1 INT 7.2 1.11 2.2 | [M + H]+ C24H30Cl F3N702 540.2 540.3 | (400 MHz, DMSO-d6) δ = 8.83 (s, 1H), 8.20 (s, 1H), 8.04-7.94 (m, 1H), 7.28- 7.12 (m, 2H), 6.97 (br d. J = 8.4 Hz, 2H), 6.43 (q, J = 9.2 Hz, 1H), 5.15 (q, J = 6.8 Hz, |
| | | | | | 1H), 3.16 (s, |
| | | | | | 3H), 3.00- |
| | | | | | 2.78 (m, 6H), |
| | | | | | 2.25 (s, 3H), |
| | | | | | 2.19-1.91 |
| | | | | | (m, 2H), 1.87- |
| | | | | | 1.74 (m, |
| | | | | | 1H), 1.73- |
| | | | | | 1.60 (m, 2H), |
| | | | | | 1.59 (d, J = 6.8 |
| | | | | | Hz, 3H), 1,40- |
| | | | | | 1.24 (m, 1H) |
| 5.3 | 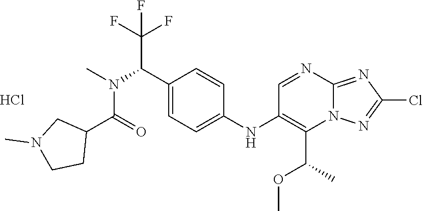 [SMILES: CO[C@@H](C)c1c(Nc2ccc([C@H](N(C)C(=O)C3CCN(C)C3)C(F)(F)F)cc2)cnc2nc(Cl)nn12 ; Cl] | 5 | INT 1.1 INT 2.1 INT 6.1 INT 7.3 1.12 2.3 | [M + H]+ C23H28Cl F3N702 526.2 526.3 | (400 MHz, CDCl₃) δ = 12.84 (br s, 1H), 8.91 (s, 1H), 7.33- 7.28 (m, 2H), 7.15 (br d, J = 3.6 Hz, 1H), 7.05 (br d, J = 8.4 Hz, 2H), 6.58- 6.43 (m, 1H), 5.48 (q, J = 6.8 |
| | | | | | Hz, 1H), 4.04- |
| | | | | | 3.87 (m, |
| | | | | | 1H). 3.80 (br |
| | | | | | s, 2H), 3.49 |
| | | | | | (s, 3H), 3.48 |
| | | | | | 3.36 (m, 1H), |
| | | | | | 3.04 (br s, |
| | | | | | 1H), 3.00- |
| | | | | | 2.96 (m, 3H), |
| | | | | | 2.94 (s, 3H), |
| | | | | | 2.71 (brd, |
| | | | | | J = 17.2 Hz, |
| | | | | | 1H), 2.11 (br |
| | | | | | s, 1H), 1.60 |
| | | | | | (s, 3H) |
| 6.1 | 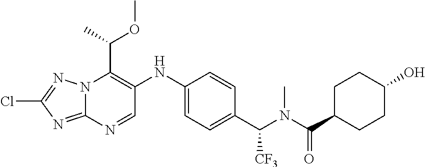 [SMILES: CO[C@@H](C)c1c(Nc2ccc([C@H](N(C)C(=O)[C@H]3CC[C@H](O)CC3)C(F)(F)F)cc2)cnc2nc(Cl)nn12] | 6 | INT 1.1 INT 2.1 INT 6.1 INT 8.1 INT 18.1 | [M + H]+ C24H29Cl F3N603 541.2 541.3 | (400 MHz, CDCl₃) δ = 9.05 (s, 1H), 7.47 (br d, J = 8.0 Hz, 2H), 7.43 (s, 1H), 7.20 (br d, J = 8.4 Hz, 2H), 6.78 (q, J = 8.8 Hz, 1H), |
| | | | | | 5.64 (q, J = |
| | | | | | 6.4 Hz, 1H), |
| | | | | | 3.93-3.81 |
| | | | | | (m, 1H), 3.65 |
| | | | | | (s, 3H), 3.08 |
| | | | | | (s, 3H), 2.78- |
| | | | | | 2.64 (m, 1H), |
| | | | | | 2.27 (br dd, |
| | | | | | J = 3.6, 8.4 Hz, |
| | | | | | 2H), 2.13- |
| | | | | | 2.05 (m, 1H), |
| | | | | | 2.03-1.96 |
| | | | | | (m, 1H), 1.91- |
| | | | | | 1.85 (m, |
| | | | | | 2H), 1.77 (d, |
| | | | | | J = 6.8 Hz, 3H), |
| | | | | | 1.57-1.42 |
| | | | | | (m, 2H) |
| 7.1 | 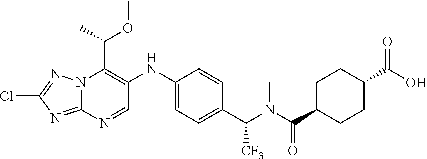 [SMILES: CO[C@@H](C)c1c(Nc2ccc([C@H](N(C)C(=O)[C@H]3CC[C@H](C(=O)O)CC3)C(F)(F)F)cc2)cnc2nc(Cl)nn12] | 7 | INT 1.1 INT 2.1 INT 3.1 INT 4.3 INT 5.4 1.4 | [M + H]+ C25H29Cl F3N604 569.2 569.2 | (400 MHz, DMSO-d6) δ = 12.06 (br s, 1H), 8.84 (s, 1H), 8.03- 7.97 (m, 1H), 7.25-7.13 (m, 2H), 7.01- 6.93 (m, 2H), 6.51- |
| | | | | | 6.10 (m, 1H), |
| | | | | | 5.15 (q, J = 6.4 |
| | | | | | Hz, 1H), 3.16 |
| | | | | | (s, 3H), 2.90- |
| | | | | | 2.68 (m, 3H), |
| | | | | | 2.49-2.38 |
| | | | | | (m, 1H), 2.27- |
| | | | | | 2.13 (m, |
| | | | | | 1H), 2.01- |
| | | | | | 1.87 (m, 2H), |
| | | | | | 1,84-1.68 |
| | | | | | (m, 2H), 1.59 |
| | | | | | (d, J = 6.8 Hz, |
| | | | | | 3H), 1.49- |
| | | | | | 1.29 (m, 4H) |
| 7.2 | 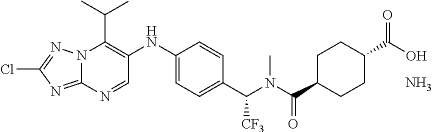 | 7 | INT 1.2 INT 2.1 INT 3.1 INT 4.3 INT 5.4 INT 18.2 | [M + H]+ C25H29Cl F3N603 553.2 553.1 | (400 MHz, DMSO-d6) δ = 8.73 (s, 1H), 8.13 (s, 1H), 7.13 (br d, J = 8.4 Hz. 2H), 6.79 (br d, J = 8.8 Hz, |
| | | | | | 2H), 6.51- |
| | | | | | 6.36 (m, 1H), |
| | | | | | 3.72 (td, |
| | | | | | J = 7.2, 14.0 |
| | | | | | Hz, 1H), 2.87 |
| | | | | | (s, 2H), 2.71- |
| | | | | | 2.66 (m, 2H), |
| | | | | | 2.20 (br s, |
| | | | | | 1H), 1.98- |
| | | | | | 1.86 (m, 2H), |
| | | | | | 1.83-1.68 |
| | | | | | (m, 2H), 1.52- |
| | | | | | 1.32 (m, |
| | | | | | 10H) |
| 7.3 | 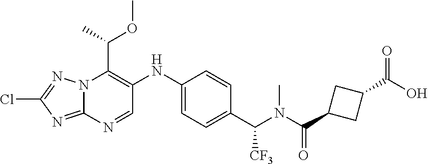 [SMILES: CO[C@@H](C)c1c(Nc2ccc([C@H](N(C)C(=O)[C@H]3C[C@H](C(=O)O)C3)C(F)(F)F)cc2)cnc2nc(Cl)nn12] | 7 | INT 1.1 INT 2.1 INT 3.1 INT 4.10 INT 5.12 INT 18.3 | [M + H]+ C23H25Cl F3N604 541.2 541.0 | (400 MHz, CD₃CN) δ = 8.81 (s, 1H), 7.32-7.20 (m, 2H), 7.09- 7.00 (m, 3H), 6.59- 6.47 (m, 1H), 5.39-5.30 (m, 1H), 3.46- |
| | | | | | 3,32 (m, |
| | | | | | 4H), 2.97- |
| | | | | | 2.84 (m, 1H), |
| | | | | | 2.71 (s, 3H), |
| | | | | | 2.54 (s, 1H), |
| | | | | | 2.37-2.32 |
| | | | | | (m, 2H), 2.13 |
| | | | | | (s, 2H), 1.58- |
| | | | | | 1.52 (m, 3H) |
| 8.1 | 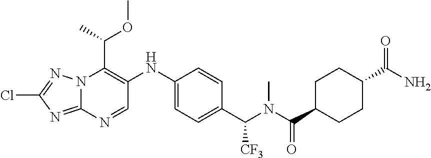 [SMILES: CO[C@@H](C)c1c(Nc2ccc([C@H](N(C)C(=O)[C@H]3CC[C@H](C(N)=O)CC3)C(F)(F)F)cc2)cnc2nc(Cl)nn12] | 8 | INT 1.1 INT 2.1 INT 3.1 INT 4.3 INT 5.4 1.4 7.1 | [M + H]+ C25H30Cl F3N703 568.2 568.1 | (400 MHz, MeOD) δ = 8.87 (s, 1H). 7.29 (d, J = 8.4 Hz, 2H), 7.06 (d, J = 8.8 Hz, 2H), 6.59- 6.48 (m, 1H), 5.36 (q, J = 6.8 Hz, 1H), 3.36 |
| | | | | | (s, 3H), 2.96 |
| | | | | | (s, 3H), 2.79- |
| | | | | | 2.74 (m, 1H). |
| | | | | | 2.35-2.23 |
| | | | | | (m, 1H), 1.98- |
| | | | | | 1.83 (m, |
| | | | | | 4H), 1.64 (d, |
| | | | | | J = 6.8 Hz, |
| | | | | | 3H), 1.61- |
| | | | | | 1.51 (m, 4H). |
| 8.2 | 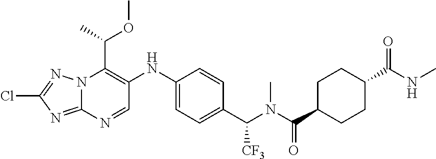 [SMILES: CNC(=O)[C@H]1CC[C@H](C(=O)N(C)[C@@H](c2ccc(Nc3cnc4nc(Cl)nn4c3[C@H](C)OC)cc2)C(F)(F)F)CC1] | 8 | INT 1.1 INT 2.1 INT 3.1 INT 4.3 INT 5.4 1.4 7.1 | [M + H]+ C26H32Cl F3N703 582.2 582.2 | (400 MHz, MeOD) δ = 8.87 (s, 1H), 7.38-7.25 (m, 2H), 7.06 (d, J=8.8 Hz, 2H), 6.53 (q, J = 8.8 Hz, 1H), 5.36 (q, J = 6.8 Hz, |
| | | | | | 1H), 3.36 (s, |
| | | | | | 3H), 2.96 (s, |
| | | | | | 3H), 2.82- |
| | | | | | 2.72 (m, 1H), |
| | | | | | 2.70 (s, 3H), |
| | | | | | 2.29-2.17 |
| | | | | | (m, 1H), 1.96- |
| | | | | | 1.78 (m, |
| | | | | | 4H), 1.64 (d, |
| | | | | | J = 6.8 Hz, |
| | | | | | 3H), 1.62- |
| | | | | | 1.51 (m, 4H) |
| 8.3 | 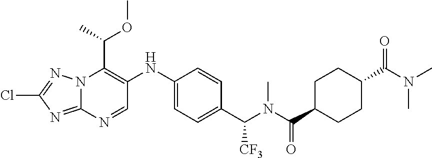 [SMILES: CO[C@@H](C)c1c(Nc2ccc([C@H](N(C)C(=O)[C@H]3CC[C@H](C(=O)N(C)C)CC3)C(F)(F)F)cc2)cnc2nc(Cl)nn12] | 8 | INT 1.1 INT 2.1 INT 3.1 INT 4.3 INT 5.4 1.4 7.1 | [M + H]+ C27H34Cl F3N703 596.2 596.2 | (400 MHz, MeOD) δ = 8.87 (s, 1H), 7.40-7.25 (m, 2H), 7.13- 7.01 (m, 2H), 6.53 (q, J = 8.8 Hz, 1H), 5.36 (q, J = 6.8 Hz, |
| | | | | | 1H), 3.36 (s, |
| | | | | | 3H), 3.12 (s, |
| | | | | | 3H), 2.98- |
| | | | | | 2.95 (m, 3H), |
| | | | | | 2.93 (s, 3H), |
| | | | | | 2.82-2.73 |
| | | | | | (m, 2H), 1.97- |
| | | | | | 1.80 (m, |
| | | | | | 4H), 1.64 (d, |
| | | | | | J = 6.8 Hz, |
| | | | | | 3H), 1.63- |
| | | | | | 1.51 (m, 4H) |
| 9.1 | 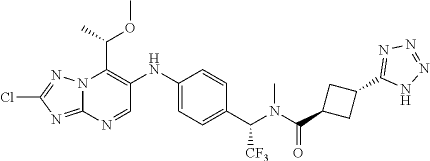 [SMILES: CO[C@@H](C)c1c(Nc2ccc([C@H](N(C)C(=O)[C@H]3C[C@H](c4nnn[nH]4)C3)C(F)(F)F)cc2)cnc2nc(Cl)nn12] | 9 | INT 1.1 INT 2.1 INT 3.1 INT 5.9 1.9 | [M + H]+ C23H25Cl F3N1002 565.2 565.0 | (400 MHz, Methanol-d₄) δ = 8.90- 8.88 (m, 1H), 7.36-7.32 (m, 2H), 7.10- 7.05 (m, 2H), 6.60- 6.51 (m, 1H), 5.37 (q, J = |
| | | | | | 6.6 Hz, 1H), |
| | | | | | 3.92-3.70 |
| | | | | | (m, 2H), 3.37 |
| | | | | | (s, 3H), 2.91- |
| | | | | | 2.79 (m, 4H), |
| | | | | | 2.73-2.59 |
| | | | | | (m, 3H), 1.67- |
| | | | | | 1.63 (m, 3H) |
| 10.1 | 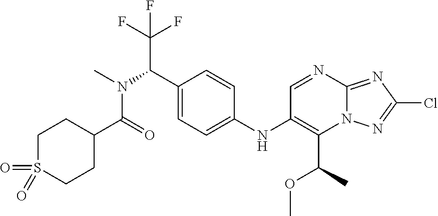 [SMILES: CO[C@H](C)c1c(Nc2ccc([C@H](N(C)C(=O)C3CCS(=O)(=O)CC3)C(F)(F)F)cc2)cnc2nc(Cl)nn12] | 10 | 1.1 | [M + H]+ C23H27Cl F3N604S 575.1 575.0 | (400 MHz, DMSO) δ = 8.83 (s, 1H), 7.99 (s, 1H), 7.19 (d, J = 8.0 Hz, 2H), 6.97 (d, J = 8.0 Hz, 2H), 6.43 (q, J = 8.0 Hz, 1H), 5.15 (q, J = 8.0 Hz, 1H), 3.26- 3.05 (m, 8H), |
| | | | | | 2.90 (s, 3H), |
| | | | | | 2.14-1.93 |
| | | | | | (m, 4H), 1.59 |
| | | | | | (d, J = 8.0 Hz, |
| | | | | | 3H) |
| 11.1 | 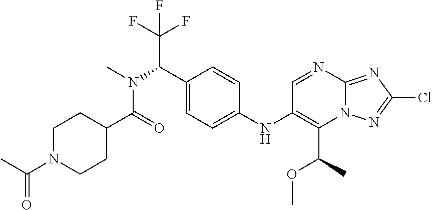 [SMILES: CO[C@H](C)c1c(Nc2ccc([C@H](N(C)C(=O)C3CCN(C(C)=O)CC3)C(F)(F)F)cc2)cnc2nc(Cl)nn12] | 11 | 3.1 | [M + H]+ C25H30Cl F3N703 568.2 568.1 | (400 MHz, CDCl₃) δ = 8.90 (s, 1H), 7.35-7.29 (m, 2H), 7.11 (s, 1H), 7.04 (d, J = 8.8 Hz, 2H), 6.62 (q, J = 8.8 Hz, 1H), 5.48 (q, J = 6.8 Hz, 1H), 4.69- 4.55 (m, 1H), |
| | | | | | 3.99-3.87 |
| | | | | | (m, 1H), 3.49 |
| | | | | | (s, 3H), 3.22- |
| | | | | | 3.08 (m, 1H), |
| | | | | | 2.95 (s, 3H), |
| | | | | | 2.88-2.66 |
| | | | | | (m, 2H), 2.12 |
| | | | | | (s, 3H), 1.96- |
| | | | | | 1.70 (m, 4H), |
| | | | | | 1.63-1.61 |
| | | | | | (m, 3H) |

---

### Example 2. MALT1 Biochemical Assay

Inhibitor potency was evaluated by measuring enzymatic activity of full length MALT1 at varying concentrations of compound. The enzymatic assay consists of a single substrate reaction that monitors the release of a fluorescent dye upon cleavage of the peptide substrate. The peptide substrate has the following sequence: Ac-Leu-Arg-Ser-Arg-Rh110-dPro (custom synthesis from WuXi AppTec, Shanghai, China). The assay buffer consists of 50 mM Hepes, pH 7.5, 0.8 M sodium citrate, 1 mM DTT, 0.004% tween-20, and 0.005% bovine serum albumin (BSA). Steady-state kinetic analysis of peptide substrate binding resulted in a Michaelis-Menten constant (K_{M}) of 150 μM. The assay was performed in a 384-well F-bottom polypropylene, black microplate (Greiner Bio_One, Catalog no. 781209) at 15 nM enzyme and 30 μM peptide substrate. The reaction was quenched after 60 minutes with the addition of iodoacetate at a final concentration of 10 mM. Total fluorescence was measured using an Envision (PerkinElmer) with fluorescence excitation at 485 nm and emission at 520 mm.

For potency determination, 1 μL of serially diluted compound (in 100% DMSO) was pre-incubated with 40 μL of enzyme for 30 minutes. The reaction was initiated with the addition of 10 μL of peptide substrate. The relative fluorescence units were transformed to percent inhibition by using 0% and 100% inhibition controls as reference. The 100% inhibition control consisted of 1 μM final concentration of (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea (IC₅₀=15 nM), while the 0% inhibition control consisted of 2% DMSO. IC₅₀ values were calculated by fitting the concentration-response curves to a four-parameter logistic equation in GraphPad Prism.

Results from this assay are summarized in Table 2 below. In this table, “A” indicates IC₅₀ of less than 0.1 μM; “B” indicates IC₅₀ from 0.1 μM up to 1 μM; and “C” indicates IC₅₀ of greater than 1 μM; “N/A” indicates not tested. For a compound that was tested in more than one experiment, the result shown represents the mean value.

TABLE 2

| Compound | IC₅₀ (μM) |
|---|---|
| 1.1 | A |
| 1.2 | B |
| 1.3 | B |
| 1.4 | B |
| 1.5 | A |
| 1.6 | A |
| 1.7 | A |
| 1.8 | B |
| 1.9 | B |
| 1.10 | B |
| 1.11 | B |
| 1.12 | B |
| 1.13 | B |
| 1.14 | A |
| 1.15 | B |
| 1.16 | A |
| 1.17 | A |
| 1.18 | A |
| 1.19 | A |
| 1.20 | C |
| 1.21 | C |
| 1.22 | C |
| 1.23 | C |
| 1.24 | C |
| 1.25 | B |
| 1.26 | C |
| 1.27 | C |
| 1.28 | C |
| 1.29 | C |
| 1.30 | B |
| 1.31 | C |
| 1.32 | B |
| 1.33 | B |
| 2.1 | B |
| 2.2 | B |
| 2.3 | B |
| 2.4 | A |
| 3.1 | A |
| 3.2 | A |
| 3.3 | A |
| 3.4 | A |
| 3.5 | A |
| 3.6 | B |
| 3.7 | B |
| 3.8 | B |
| 3.9 | A |
| 3.10 | B |
| 3.11 | A |
| 3.12 | A |
| 4.1 | A |
| 4.2 | B |
| 4.3 | A |
| 5.1 | B |
| 5.2 | B |
| 5.3 | B |
| 6.1 | A |
| 7.1 | B |
| 7.2 | A |
| 7.3 | B |
| 8.1 | A |
| 8.2 | B |
| 8.3 | B |
| 9.1 | A |
| 10.1 | B |
| 11.1 | A |

### Example 3. Jurkat IL-2 Assay

Inhibition was determined in a cell-based assay using Jurkat (ATCC, clone E6.1), an immortalized T-cell line, exposed to a dose response of compound and assessed for viability, and Il-2 inhibition by ELISA. Cells were cultured in RPMI/10% FBS (Invitrogen 11875093, Atlanta Biologicals S12450H), maintained below 3E6/mL and only used in assays while below passage 25. Compounds were stamped by ECHO onto 384w plates (PerkinElmer Culturplate, 6007680). The cells were plated in fresh media on top of compound and incubated for 30 minutes before stimulation with soluble anti-CD3/28/2 (Stemcell, 10970) for 24 hours. Supernatant was collected and assessed for Il-2 (MSD, 384w, L21SA-1). To assess viability of cells treated with compound, cells were lysed with CTG reagent (Promega, G7570), and measured by luminometer. IL-2 curves were calculated as percent of DMSO (100%) and signal-inhibiting (0%, (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea) controls. IC₅₀s calculated using 4-parameter fit in GraphPad Prism.

Results from this assay are summarized in Table 3 below. In this table, “A” indicates IC₅₀ of less than 0.1 μM; “B” indicates IC₅₀ from 0.1 μM up to 1 μM; and “C” indicates IC₅₀ of greater than 1 μM. “N/A” indicates not tested. For a compound that was tested in more than one experiment, the result shown represents the mean value.

TABLE 3

| Compound | IC₅₀ (μM) |
|---|---|
| 1.1 | B |
| 1.2 | B |
| 1.3 | B |
| 1.4 | B |
| 1.5 | B |
| 1.6 | B |
| 1.7 | B |
| 1.8 | B |
| 1.9 | B |
| 1.10 | B |
| 1.11 | C |
| 1.12 | C |
| 1.13 | B |
| 1.14 | B |
| 1.15 | B |
| 1.16 | B |
| 1.17 | B |
| 1.18 | A |
| 1.19 | A |
| 1.20 | C |
| 1.21 | C |
| 1.22 | C |
| 1.23 | C |
| 1.24 | C |
| 1.25 | C |
| 1.26 | C |
| 1.27 | C |
| 1.28 | C |
| 1.29 | C |
| 1.30 | C |
| 1.31 | C |
| 1.32 | C |
| 1.33 | C |
| 2.1 | B |
| 2.2 | C |
| 2.3 | B |
| 2.4 | B |
| 3.1 | B |
| 3.2 | B |
| 3.3 | B |
| 3.4 | B |
| 3.5 | B |
| 3.6 | C |
| 3.7 | C |
| 3.8 | C |
| 3.9 | B |
| 3.10 | B |
| 3.11 | B |
| 3.12 | B |
| 4.1 | B |
| 4.2 | B |
| 4.3 | B |
| 5.1 | B |
| 5.2 | C |
| 5.3 | C |
| 6.1 | B |
| 7.1 | C |
| 7.2 | B |
| 7.3 | C |
| 8.1 | B |
| 8.2 | B |
| 8.3 | B |
| 9.1 | C |
| 10.1 | B |
| 11.1 | B |

### Example 4. High Throughput Dialysis (HTD) Human Plasma Protein Binding Assay

Human plasma protein binding of various compounds of formula (I) were evaluated using the equilibrium dialysis method described herein.

### Dialysis Membrane and Matrix Preparation

The dialysis membrane strips (HTD % a/b, catalog number 1101, HTDialysis LLC, Gales Ferry, CT, USA) were soaked in ultra-pure water at room temperature for approximately 1 hour. Each membrane strip containing 2 membranes was separated and soaked in 20:80 ethanol/water (v/v) for approximately 20 minutes, after which they were ready for use or were stored in the solution at 2-8° C. for up to a month. Prior to the experiment, the membrane was rinsed three times and soaked for 20 minutes in ultra-pure water.

On the day of experiment, the human plasma (BiolVT, catalog number HUMANPLNHPNN, sodium heparin or EDTA-K₂ anticoagulant, multiple individuals pooled) was thawed by running under cold tap water and centrifuged at 3220 rpm for 5 minutes to remove any clots. The pH value of the resulting plasma was confirmed to be 7.0-8.0.

### Dialysis Procedure

The test compounds and warfarin control (Stru Chem, catalog number SC-16139) were dissolved in dimethyl sulfoxide (DMSO) to obtain 10 mM stock solutions. DMSO working solutions were prepared at 400 μM. To prepare the loading matrix, the compound working solutions (5 μL) were added in a 1:200 ratio to blank human plasma (995 μL) and mixed thoroughly.

The time zero (T0) samples to be used for recovery determination were prepared as follows: 50 μL aliquots of loading matrix were transferred in triplicate to the sample collection plate. The samples were immediately matched with opposite blank buffer (basic solution (14.2 g/L Na₂HPO₄ and 8.77 g/L NaCl in deionized water) titrated with acidic solution (15.6 g/L NaH₂PO₄·2H₂O and 8.77 g/L NaCl in deionized water) to pH 7.4±0.1) to obtain a final volume of 100 μL of 1:1 matrix/dialysis buffer (v/v) in each well. 500 μL stop solution (200 ng/mL tolbutamide and 200 ng/mL labetalol in acetonitrile) were added to these TO samples. They were then stored at 2-8° C. for further processing along with other post-dialysis samples.

To load the dialysis device (96-well equilibrium dialysis plate, model 96b, catalog number 1006. HTDialysis LLC, Gales Ferry, CT, USA), an aliquot of 150 μL of the loading matrix was transferred to the donor side of each dialysis well in triplicate, and 150 μL of the dialysis buffer was loaded to the receiver side of the well. The dialysis plate was placed in a humidified incubator at 37° C. with 5% CO₂ on a shaking platform that rotated slowly (about 100 rpm) for 4 hours.

At the end of the dialysis, aliquots of 50 μL of the samples were taken from both the buffer side and the matrix side of the dialysis device. These samples were transferred into new 96-well plates (polypropylene, 2.2 mL/well, catalog number DWP-22-96-SQ-U-C-L. Apricot). Each sample was mixed with an equal volume of opposite blank matrix (buffer or matrix) to reach a final volume of 100 μL of 1:1 matrix/dialysis buffer (v/v) in each well. All samples were further processed by adding 500 μL of stop solution containing internal standards. The mixture was vortexed and centrifuged at 4000 rpm for about 20 minutes. An aliquot of 100 μL of supernatant of all the samples was then removed for LC-MS/MS analysis. Test compound concentrations in matrix and buffer samples were expressed as peak area ratios (PAR) of analyte/internal standard (no standard curve).

The single blank samples were prepared by transferring 50 μL of blank matrix to a 96 well plate (polypropylene. 2.2 mL/well, catalog number DWP-22-96-SQ-U-C-L, Apricot) and adding 50 μL of blank PBS buffer to each well. The blank plasma must match the species of plasma used in the plasma side of the well. Then the matrix-matched samples were further processed by adding 500 μL of stop solution containing internal standards, following the same sample processing method as the dialysis samples.

### Data Calculation

The % Unbound values were calculated using the following equation: % Unbound=100×F/T; where [F] is the analyte concentration or peak area ratio of analyte/internal standard on the buffer (receiver) side of the membrane and [T] is the analyte concentration or peak area ratio of analyte/internal standard on the matrix (donor) side of the membrane.

Results from this assay are summarized in Table 4 below. In this table, “A” indicates % Unbound compound less than 8%; “B” indicates % Unbound compound from 8% to 20%; and “C” indicates % Unbound compound greater than 20%.

TABLE 4

| | Human PPB |
|---|---|
| Compound | (% Unbound) |
| 1.1 | C |
| 1.8 | A |
| 1.15 | C |
| 1.18 | A |
| 1.19 | A |
| 3.1 | C |
| 3.4 | A |
| 3.10 | B |
| 4.1 | B |
| 5.1 | C |
| 7.1 | B |
| 8.1 | B |
| 8.2 | B |
| 8.3 | B |
| 9.1 | A |
| 11.1 | B |

### Example 5. Thermodynamic Solubility (Ts) Assay in Fasted State Simulated Intestinal Fluid (FaSSIF)

The solubilities of various compounds of formula (I) were determined using a thermodynamic solubility assay. The thermodynamic solubility assay employed the shake flask method followed by HPLC-UV analysis. The following stepwise procedure was followed:

Samples of no less than 2.0 mg were weighed into the lower chambers of Whatman mini-uniprep vials (catalog number UN203NPUORG) and 450 μL of FaSSIF media (0.056% (w/v) lecithin, 0.161% (w/v) sodium taurocholate, 0.39% (w/v) monobasic potassium phosphate, 0.77% (w/v) potassium chloride, deionized H₂O, pH 6.5) was added into each vial.

After the buffer was added, the filter pistons of the mini-uniprep vials were placed to the position of the liquid level to ensure the full contact of buffer and compound with the filter during the incubation.

The samples were then vortexed for 2 minutes, after which they were incubated at 25° C. with shaking (880 rpm) for 24 hours.

The samples were then centrifuged at 4000 rpm (20° C.) for 10 min.

The miniunipreps were then compressed to prepare filtrates, and the test concentration of the compound in the filtrate was determined using HPLC-UV (Waters XBridge C18, 4.6*100 mm column; mobile phase A: 0.1% TFA in H₂O; mobile phase B: 0.1% TFA in acetonitrile). At least 5 UV standard solutions (e.g. amiodarone hydrochloride, carbamazepine, dexamethasone) were injected into the HPLC from low to high concentration subsequently followed by testing of the thermodynamic solubility supernatants in duplicate. The solubilities of the tested compounds are given in μg/mL.

Results from this assay are summarized in Table 5 below. In this table, “A” indicates a solubility of less than 250 μg/mL; “B” indicates a solubility from 250 μg/mL to 1750 μg/mL; and “C” indicates a solubility of greater than 1750 μg/mL.

TABLE 5

| | Solubility in |
|---|---|
| | FaSSIF |
| Compound | (μg/mL) |
| 1.1 | B |
| 1.8 | A |
| 1.15 | C |
| 1.18 | A |
| 1.19 | A |
| 3.1 | B |
| 3.4 | A |
| 4.1 | A |
| 5.1 | C |
| 7.1 | C |
| 8.2 | B |
| 8.3 | B |
| 9.1 | C |
| 11.1 | B |

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound represented by Formula (I): 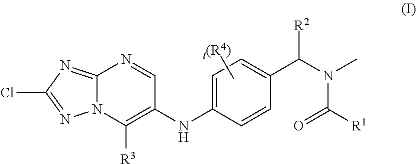
or a pharmaceutically acceptable salt thereof, wherein:
R¹ is selected from the group consisting of C₁₋₆alkyl, C₁₋₆alkoxy, C₃₋₆cycloalkyl, and 5-10 membered heterocyclyl, wherein the C₁₋₆alkyl, C₃₋₆cycloalkyl, and 5-10 membered heterocyclyl may be optionally substituted on one or more available carbons by one, two, three, or more substituents each independently selected from R^{1a}, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R² is CH₃ or CF₃;
R³ is hydrogen; or
R³ is selected from the group consisting of C₁₋₆alkyl, C₁₋₆alkoxy, C₃₋₇cycloalkyl, 5-6 membered heterocyclyl, 5-6 membered heterocyclyl-C₁₋₃alkyl-, 5-6 membered heterocyclyl-O—, phenyl, and 5-6 membered heteroaryl, any of which may be optionally substituted with one, two or three substituents each independently selected from R^{3a};
R⁴ is C₁₋₆alkyl;
R^{1a} is independently, for each occurrence, selected from the group consisting of cyano, halogen, hydroxyl, oxo, C₁₋₆alkyl, —C(O)OR^{A}, —C(O)N(R^{A})₂, —N(R^{A})₂, C₁₋₆alkoxy, 5-6 membered heterocyclyl, and 5-6 membered heteroaryl, wherein the C₁₋₆alkyl is optionally substituted with —N(R^{A})₂, and wherein if the 5-6 membered heterocyclyl contains a substitutable ring nitrogen atom, that ring nitrogen atom may optionally be substituted by R^{B};
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl;
R^{3a} is independently, for each occurrence, selected from the group consisting of halogen, C₁₋₄alkyl, C₁₋₄haloalkyl, C₁₋₄alkoxy, C₁₋₄haloalkoxy, hydroxy, C₁₋₄alkenyl, cyano, azido, —NR^{C}R^{D}, C₃₋₆cycloalkyl, C₁₋₄alkoxyC₁₋₄alkoxy, 5-6 membered heterocyclyl-O—, 5-6 membered heterocyclyl, and phenyl, wherein C₃₋₆cycloalkyl, 5-6 membered heterocyclyl-O—, 5-6 membered heterocyclyl, and phenyl are optionally substituted with one, two or three substituents each independently selected from R^{p}:
R^{p} is independently, for each occurrence, selected from the group consisting of halogen, C₁₋₄alkyl, C₁₋₄haloalkyl, hydroxy, C₁₋₄alkoxy, C₁₋₄alkoxyC₁₋₄alkyl, NR^{C}R^{D}, and aminoC₁₋₃alkyl;
R^{A} is independently, for each occurrence, selected from the group consisting of hydrogen, C₁₋₆alkyl, —C(O)C₁₋₆alkyl, and —C(O)OC₁₋₆alkyl;
R^{B} is selected from the group consisting of C₁₋₆alkyl, C₃₋₆cycloalkyl, and —C(O)OC₁₋₆alkyl;
R^{C} and R^{D} are independently, for each occurrence, selected from the group consisting of hydrogen, C₁₋₆alkyl, haloC₁₋₆alkyl, and C₃₋₄cycloalkyl, or
R^{C} and R^{D} together with the nitrogen atom to which they are attached form 4-6 membered heterocyclyl or 4-6 membered heteroaryl, wherein the 4-6 membered heterocyclyl or 4-6 membered heteroaryl may contain a further nitrogen atom or an oxygen atom and is optionally substituted with one or two fluoro; and
t is 0 or 1.

2. The compound of claim 1, wherein R² is CH₃.

3. The compound of claim 1, wherein R² is CF₃.

4. The compound of any one of claims 1-3, wherein R³ is C₁₋₆alkyl, wherein R³ is optionally substituted with C₁₋₄alkoxy.

5. The compound of any one of claims 1-4, wherein R³ is C₁₋₆alkyl.

6. The compound of any one of claims 1-5, wherein R³ is

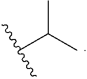 [SMILES: *C(C)C]

7. The compound of any one of claims 1-4, wherein R³ is C₁₋₆alkyl, wherein R³ is substituted with C₁₋₄alkoxy.

8. The compound of any one of claims 1-4 and 7, wherein R³ is

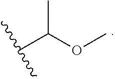 [SMILES: *C(C)OC]

9. The compound of any one of claims 1-8, wherein t=0.

10. The compound of any one of claims 1-8, wherein t=1.

11. The compound of claim 10, wherein R⁴ is C₁₋₆alkyl.

12. The compound of claim 10 or 11, wherein R⁴ is CH₃.

13. A compound represented by Formula (Ib) 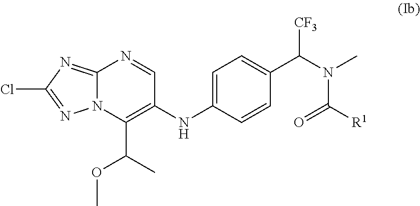
or a pharmaceutically acceptable salt thereof, wherein:
R¹ is C₁₋₆alkyl, C₃₋₆cycloalkyl, or 5-10 membered heterocyclyl, wherein the C₃₋₆cycloalkyl may be optionally substituted on one or more available carbons by one, two, three, or more substituents each independently selected from R^{1a}, wherein if the 5-10 membered heterocyclyl contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms;
R^{1a} is independently, for each occurrence, selected from the group consisting of cyano, halogen, hydroxyl, C₁₋₆alkyl, —C(O)OR^{A}, —C(O)N(R^{A})₂, —N(R^{A})₂, C₁₋₆alkoxy, and 5-6 membered heteroaryl, wherein the C₁₋₆alkyl is optionally substituted with —N(R^{A})₂;
R^{1b} is selected from the group consisting of C₁₋₆alkyl, —C(O)OR^{A}, —C(O)C₁₋₆alkyl, —C(O)C₃₋₆cycloalkyl, —C(O)N(R^{A})₂, and —S(O)₂C₁₋₆alkyl; and
R^{A} is independently, for each occurrence, selected from the group consisting of hydrogen, C₁₋₆alkyl, —C(O)C₁₋₆alkyl, and —C(O)OC₁₋₆alkyl.

14. The compound of any one of claims 1-13, wherein R¹ is C₁₋₆alkyl.

15. The compound of any one of claims 1-14, wherein R¹ is CH₃.

16. The compound of any one of claims 1-13, wherein R¹ is C₃₋₆cycloalkyl, wherein R¹ may be optionally substituted on one or more available carbons by one, two, three, or more substituents each independently selected from R^{1a}.

17. The compound of any one of claims 1-13 and 16, wherein R¹ is C₃₋₆cycloalkyl.

18. The compound of any one of claims 1-13, 16, and 17, wherein R¹ is selected from the group consisting of

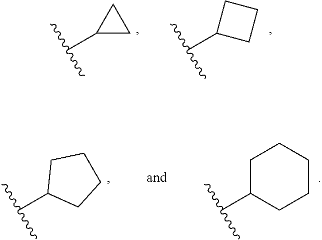

19. The compound of any one of claims 1-13 and 16, wherein R¹ is C₃₋₆cycloalkyl, wherein R¹ is substituted on one or more available carbons by one, two, three, or more substituents each independently selected from R^{1a}.

20. The compound of any one of claims 1-13, 16, and 19, wherein R¹ is

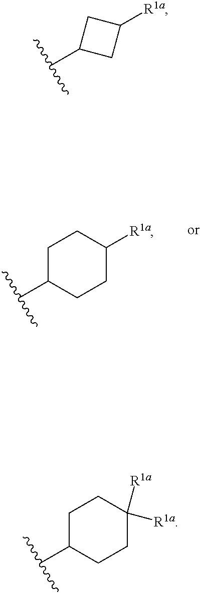

21. The compound of any one of claims 1-13, 16, 19, and 20, wherein R^{1a} is selected from the group consisting of cyan, fluoro, hydroxyl, —O—CH₃, —C(O)OH, —C(O)NH₂,

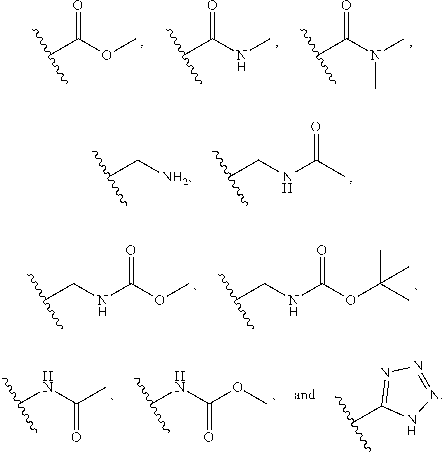

22. The compound of any one of claims 1-13, 16, and 19-21, wherein R¹ is selected from the group consisting of

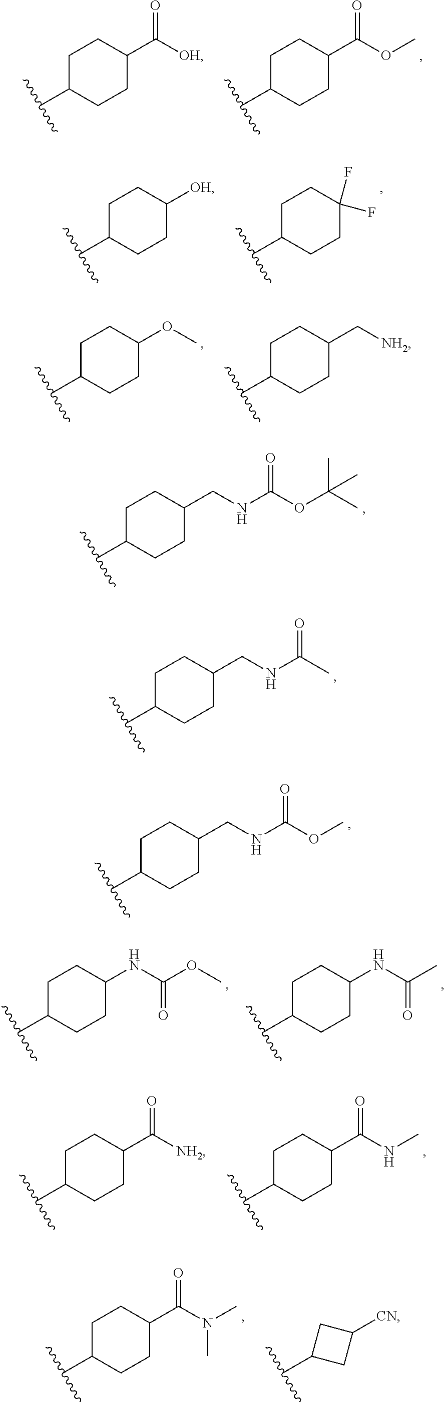

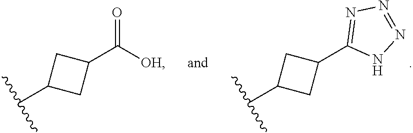 [SMILES: *C1CC(C(=O)O)C1 ; *C1CC(c2nnn[nH]2)C1]

23. The compound of any one of claims 1-12, wherein R¹ is selected from the group consisting of

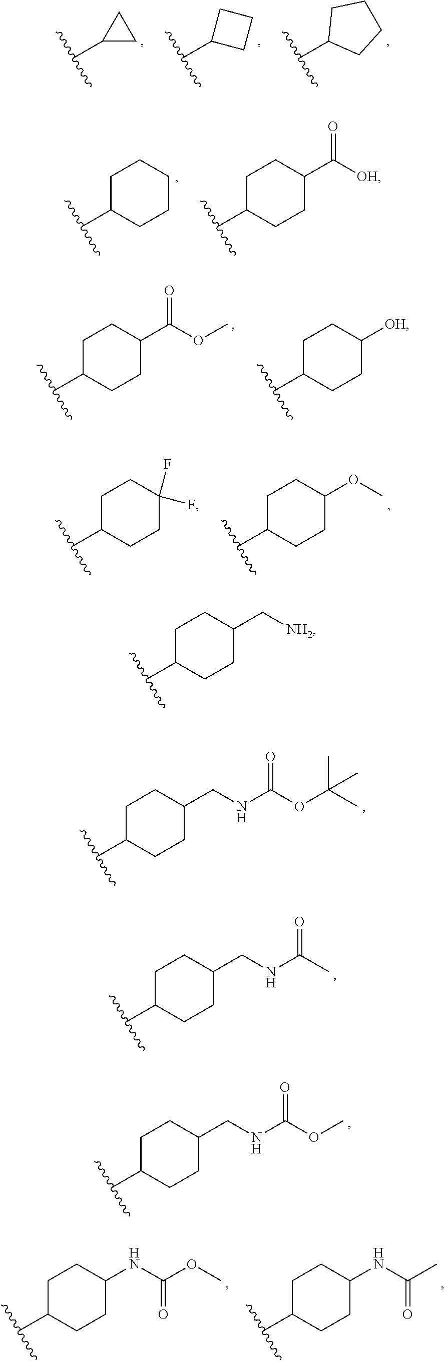

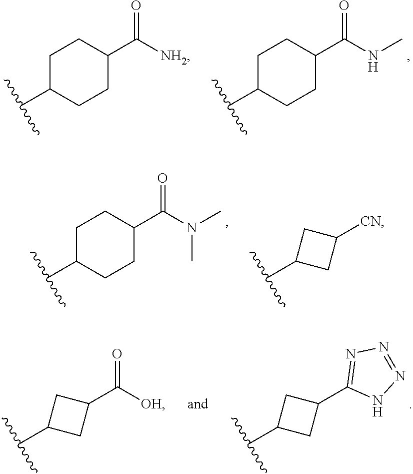 [SMILES: *C1CCC(C(N)=O)CC1 ; *C1CCC(C(=O)NC)CC1 ; *C1CCC(C(=O)N(C)C)CC1 ; *C1CC(C#N)C1 ; *C1CC(C(=O)O)C1 ; *C1CC(c2nnn[nH]2)C1]

24. The compound of any one of claims 1-13, wherein R¹ is 5-10 membered heterocyclyl, wherein if R¹ contains a substitutable ring nitrogen atom, that ring nitrogen atom may optionally be substituted by R^{1b}, and wherein if the 5-10 membered heterocyclyl contains a substitutable ring sulfur atom, that ring sulfur atom may be optionally substituted with two O atoms.

25. The compound of any one of claims 1-13 and 24, wherein R¹ is 5-10 membered heterocyclyl.

26. The compound of any one of claims 1-13, 24, and 25, wherein R¹ is selected from the group consisting of

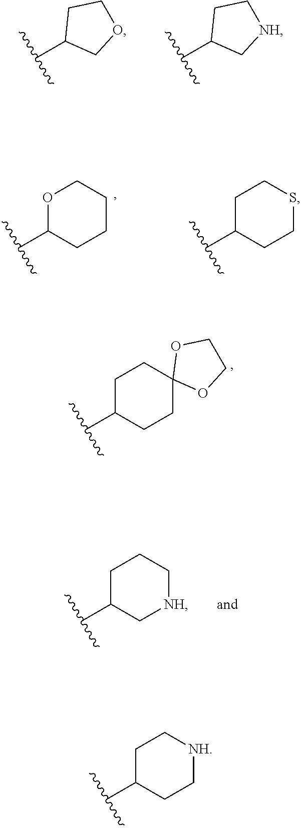

27. The compound of any one of claims 1-13 and 24, wherein R¹ is

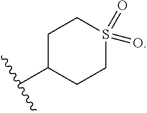 [SMILES: *C1CCS(=O)(=O)CC1]

28. The compound of any one of claims 1-13 and 24, wherein R¹ is selected from the group consisting of

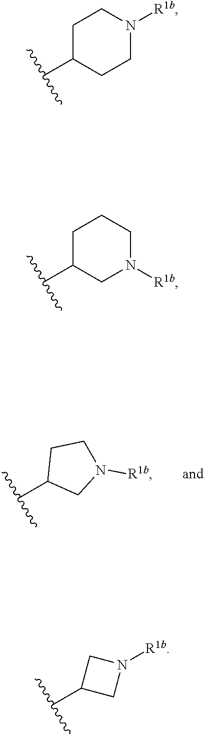

29. The compound of any one of claims 1-13, 24, and 28, wherein R^{1b} is selected from the group consisting of CH₃,

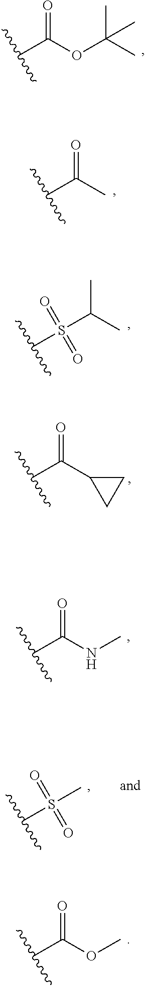

30. The compound of any one of claims 1-13, 24, 28, and 29, wherein R¹ is selected from the group consisting of

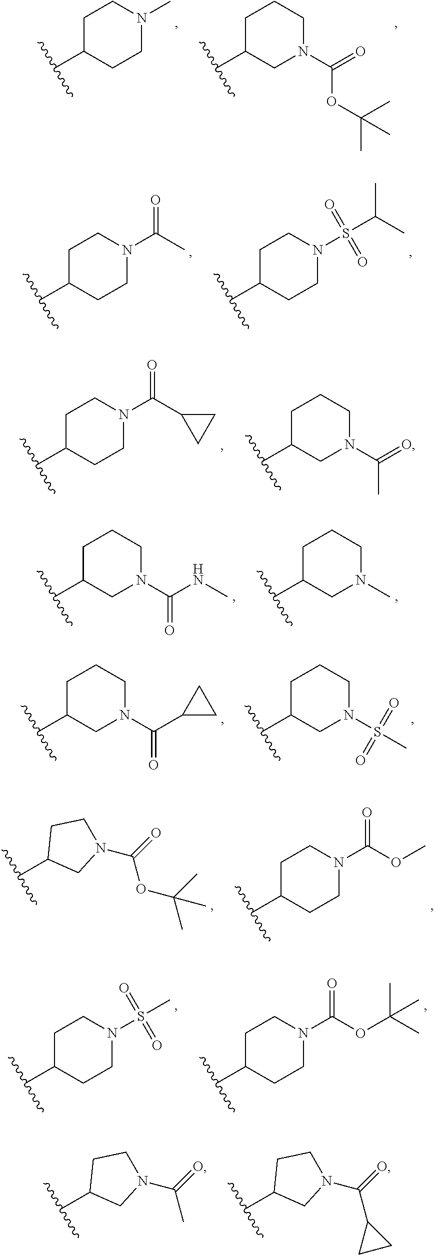

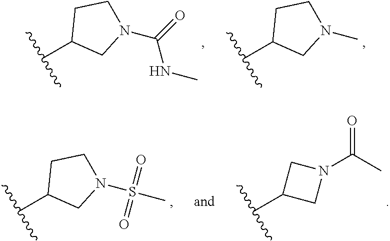

31. The compound of any one of claims 1-13, wherein R¹ is selected from group consisting of

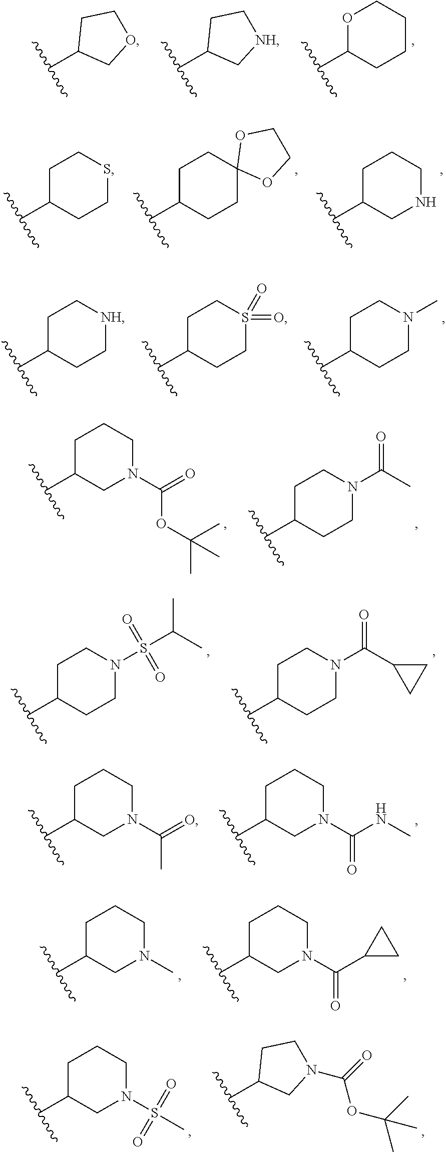

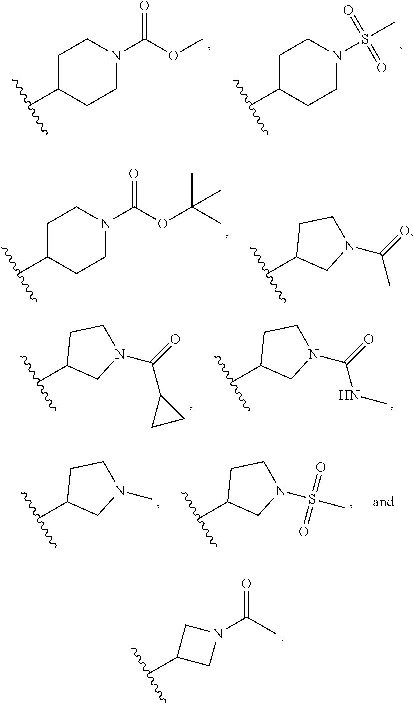

32. The compound of claim 1-12, wherein R¹ is selected from the group consisting of CH₃,

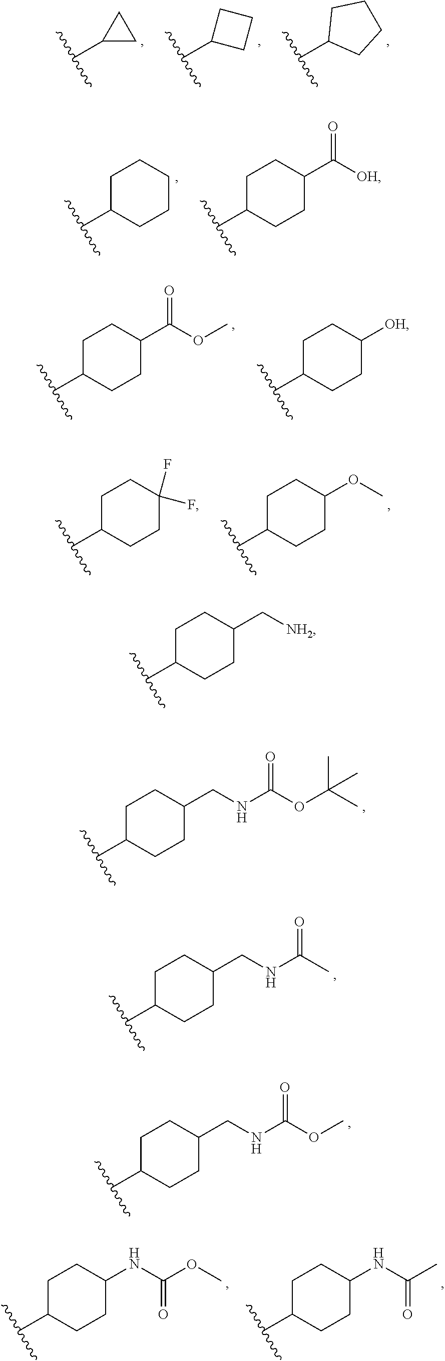

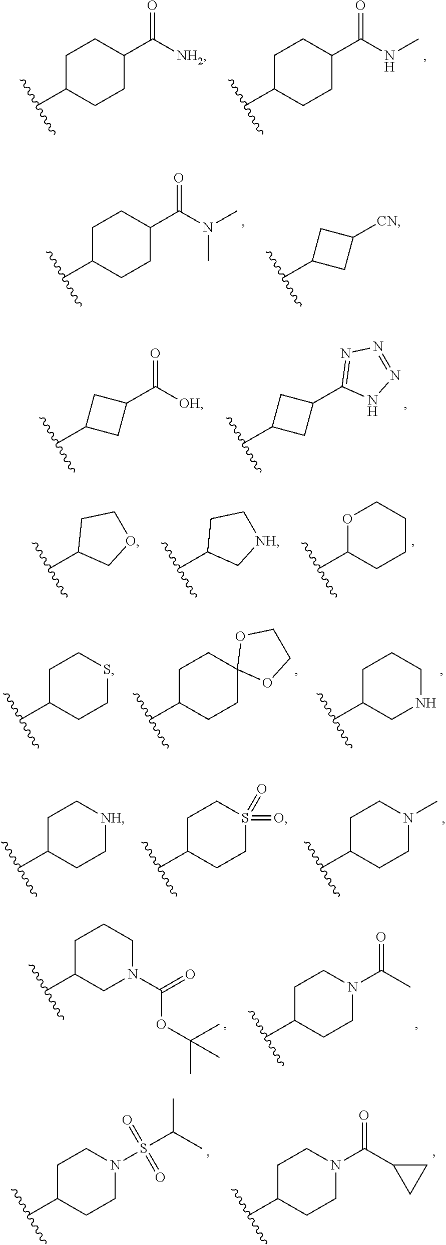

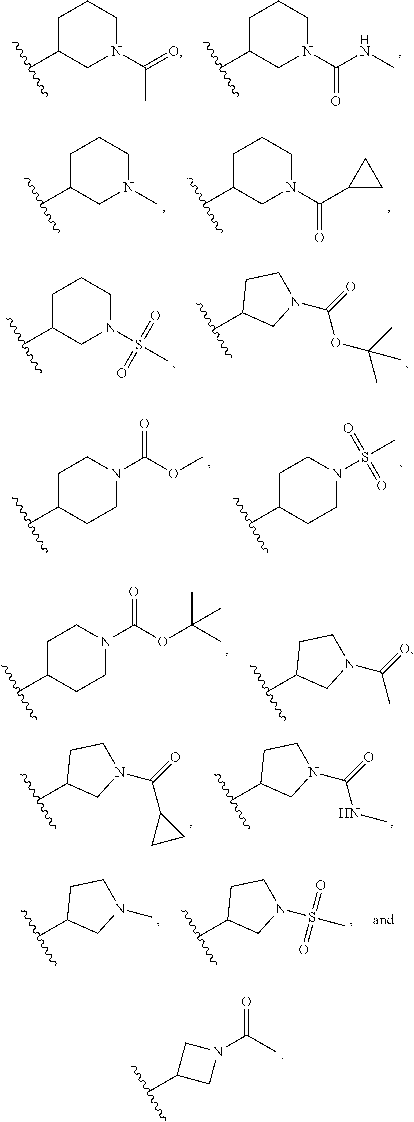

33. The compound of claim 13, wherein R¹ is selected from the group consisting of CH₃,

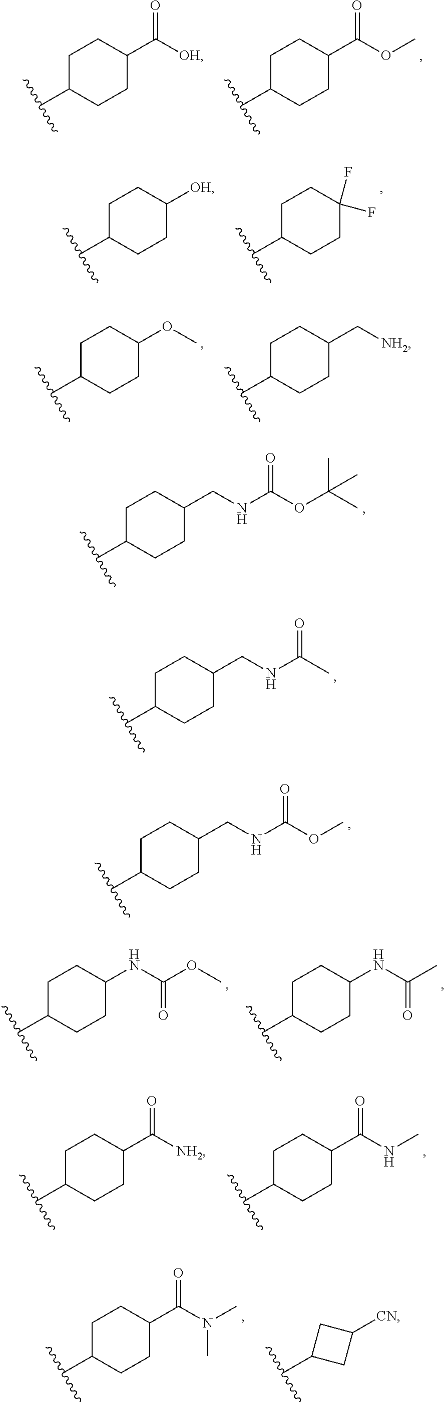

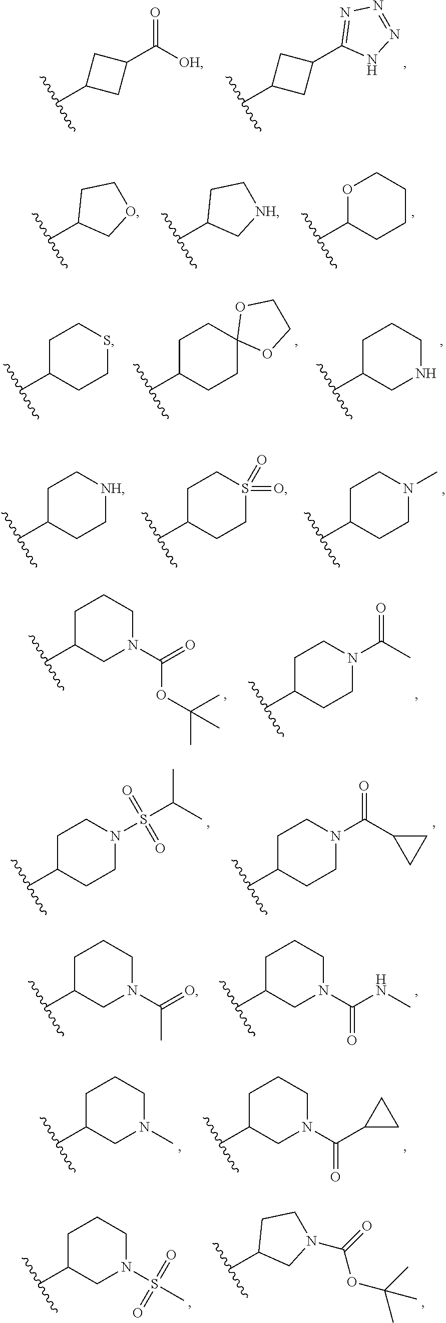

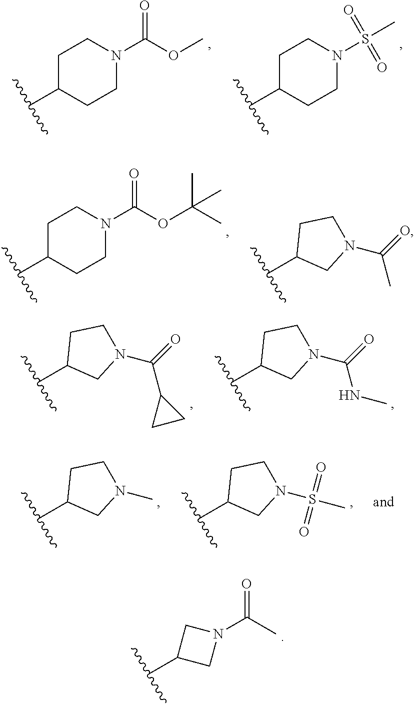

34. A compound selected from any compound set forth in Table 1, or a pharmaceutically acceptable salt thereof.

35. A pharmaceutical composition comprising a compound of any one of claims 1-34 and a pharmaceutically acceptable carrier.

36. A method of treating a cancer in a subject in need thereof, the method comprising administering to the subject a therapeutically effective amount of a compound of any one of claims 1-34 or a pharmaceutical composition of claim 35.

37. A method of treating an autoimmune or inflammatory disorder or disease in a subject in need thereof, the method comprising administering to the subject a therapeutically effective amount of a compound of any one of claims 1-34 or a pharmaceutical composition of claim 35.

38. The method of claim 37, wherein the autoimmune or inflammatory disorder or disease is selected from the group consisting of acute graft-versus-host disease, chronic graft-versus-host disease, lupus, scleroderma, psoriatic arthritis, primary sclerosing cholangitis, rheumatoid arthritis, and an inflammatory bowel disease.
